SEQUENCE LISTING (199 sequences)

SEQ ID NO: 1 - DNA, 348 bp, Artificial Sequence
  source: /note=“Description of Artificial Sequence Synthetic polynucleotide”
caggtccagctgcaagagtctggccctggactggttcagccctctcagaccctgtctctgacctgtaccgtgtccggcttctccctgaccgacttctctgtgcactgggtccgacagcctccaggcaaaggactggaatggatgggcagaatcagatccgagggcaacaccgactacaacagcgccctgaagtcccggctgtctatcagcagagacacctccaagagccaggtgttcctgaagatgaactccctgcagaccgaggacaccgccatctatttctgcaccagaggcgacatcctcggcttcggctattggggacagggcgtgatggtcaccgttagctct

SEQ ID NO: 2 - DNA, 336 bp, Artificial Sequence
  source: /note=“Description of Artificial Sequence Synthetic polynucleotide”
gacatcgtgatgacccagtctccactgtccgtgtctgtgacccctggcgagtctgcctccatctcctgcagatcctccaagagcctgctgcacttcaagggcatcaccttcgtgtactggtatctgcagaagcccggccagtctcctcagctgctgatcttcagaatgtccagcctggcctctggcgtgcccgatagattttctggctccggctccgagacagacttcaccctgaagatctccagagtggaagccgaggacgtgggcacctactattgtggccagctgctggaaaacccctacacctttggcgctggcaccaagctggaactgaag

SEQ ID NO: 3 - DNA, 294 bp, Artificial Sequence
  source: /note=“Description of Artificial Sequence Synthetic polynucleotide”
gctcagaccaccgctcctagcgtgtaccctttggctcctggctgtggcgacaccacctcttctacagtgaccctgggctgtctggtcaagggctactttcctgagcctgtgaccgtgacctggaactctggtgccctgtcctccgacgtgcacacctttccagctgtgctgcagtccggcctgtacaccctgacatcctccgtgacctcttccacctggcctagccagaccgtgacatgcaatgtggctcaccctgcctccagcaccaaggtggacaagaaggtggaacggcgg

SEQ ID NO: 4 - DNA, 321 bp, Artificial Sequence
  source: /note=“Description of Artificial Sequence Synthetic polynucleotide”
agagctgacgctgcccctaccgtgtctatcttccctccatccatggaacagctgacctctggcggagctaccgtcgtgtgcttcgtgaacaacttctaccctcgggacatctccgtgaagtggaagatcgacggctctgagcagcgagatggcgtgctggattctgtgaccgaccaggactccaaggacagcacctactccatgtctagcaccctgagcctgaccaaggtggaatacgagcggcacaacctgtatacctgcgaggtggtgcacaagacctccagctctcccgtggtcaagtccttcaaccggaacgagtgc

SEQ ID NO: 5 - DNA, 354 bp, Artificial Sequence
  source: /note=“Description of Artificial Sequence Synthetic polynucleotide”
caggtccagttgcagcagtctggcgctgagctggtcaagcctggatcctccgtgaagatctcctgcaaggcctccggctacaccttcacctccaacttcatgcactggatcaagcagcagcccggcaacggcctggaatggatcggatggatctatcctggcgacggcgacaccgagtacaaccagaagttcaacggcaaggctaccctgaccgccgacaagtcctcttccaccgcttacatgcagctgtccagcctgacctctgaggactccgccgtgtacttctgcgccgtgaattatggcggctacgtgctggatgcttggggccaaggcgcttctgtgacagtgtcctct

SEQ ID NO: 6 - DNA, 309 bp, Artificial Sequence
  source: /note=“Description of Artificial Sequence Synthetic polynucleotide”
gccgagacaaccgctcctagcgtttaccctctggctcctggcacagccctgaagtccaactctatggtcaccctgggctgcctggtcaagggctactttcctgagcctgtgaccgtgacctggaactctggtgctctgtctagcggcgtgcacacctttccagctgtgctgcagagcggcctgtacaccctgacatctagcgtgaccgtgccttccagcacctggtctagtcaggctgtgacctgcaacgtggcccatcctgcctcttctaccaaggtggacaagaaaatcgtgcccagagagtgcaac

SEQ ID NO: 7 - DNA, 318 bp, Artificial Sequence
  source: /note=“Description of Artificial Sequence Synthetic polynucleotide”
gagatcgtgctgacccagtctcctaccaccatggctgctagccctggcgagaaagtgacaattacctgccgggcctcctcctccaccaactacatgtcctggtatcagcagaagtccggcgcctctcctaagccttggatctacgagacatccaagctggcctctggcgtgcccgatagattttccggctctggctccggcacctcctacagcttcaccatctccagcatggaaacagaggacgccgccacctactactgccaccagtggtcatctacccctctgacctttggcagcggcaccaagctggaaatcaag

SEQ ID NO: 8 - DNA, 321 bp, Artificial Sequence
  source: /note=“Description of Artificial Sequence Synthetic polynucleotide”
agagctgacgccgctcctaccgtgtctatcttccctccatccatggaacagctgacctccggcggagctaccgtcgtgtgtttcgtgaacaacttctaccctcgggacatctccgtgaagtggaagatcgacggctctgagcagcgagatggcgtgctggattctgtgaccgaccaggactccaaggacagcacctactccatgtctagcaccctgagcctgaccaaggtggaatacgagcggcacaacctgtatacctgcgaggtggtgcacaagacctccagctctcccgtggtcaagtccttcaaccggaacgagtgc

SEQ ID NO: 9 - DNA, 684 bp, Artificial Sequence
  source: /note=“Description of Artificial Sequence Synthetic polynucleotide”
accattaagccttgtcctccatgcaagtgccccgctcctaatctgctcggaggcccttccgtgttcatctttccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaactctaccctgagagtggtgtctgccctgcctatccagcatcaggactggatgtccggcaaagaattcaagtgcaaagtgaacaacaaggacctgcctgctccaatcgagcggaccatctctaagcctaagggctctgtcagggcccctcaggtgtacgttctgcctccttgcgaggaagagatgaccaagaaacaagtgacactgtggtgcatggtcacagacttcatgcccgaggacatctacgtggaatggaccaacaacggcaagaccgagctgaactacaagaacaccgagcctgtgctggactccgacggctcctacttcatgtactccaagctgcgcgtcgagaagaagaactgggtcgagagaaactcctactcctgctccgtggtgcacgagggcctgcacaatcaccacaccaccaagtccttctctcggacccctggcaag

SEQ ID NO: 10 - DNA, 684 bp, Artificial Sequence
  source: /note=“Description of Artificial Sequence Synthetic polynucleotide”
accatcaagccctgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgccaatccagcaccaggattggatgtccggcaaagaattcaagtgcaaagtgaacaacaaggacctgcctgctccaatcgagcggaccatctctaagcctaagggctctgtgcgggctccccaagtttgtgttctgcctccacctgaggaagagatgaccaagaaacaagtgaccctgtcttgtgccgtgaccgacttcatgcccgaggacatctacgtggaatggaccaacaatggcaagaccgagctgaactacaagaacaccgagcctgtgctggactccgacggctcctacttcatggtgtctaagctgcgcgtcgagaagaagaactgggtcgagagaaactcctactcctgctccgtggtgcacgagggcctgcacaatcaccacaccaccaagtccttctctcggacccctggcaag

SEQ ID NO: 11 - DNA, 474 bp, Artificial Sequence
  source: /note=“Description of Artificial Sequence Synthetic polynucleotide”
gaggtgcagctggttgaatctggcggaggactggttaagcctggcggctctctgagactgtcttgtgccgcttctggcttcaccttctccgcctacgccatgaactgggtccgacaggctcctggcaaaggcctggaatgggtcggaagaatccggaccaagaacaacaactacgccacctactacgccgactccgtgaaggaccggttcaccatctctcgggacgactccaagaacaccctgtacctgcagatgaactccctgaaaaccgaggacaccgccgtgtactactgcaccaccttctacggcaatggcgtgtggggacagggcacactggttaccgtttcttccgcctccaccaagggaccctctgtgtttcctctggctccctccagcaagtctacctctggtggaacagctgccctgggctgcctggtcaaggattactttcctgagcctgtgaccgtgtcctgg

SEQ ID NO: 12 - DNA, 309 bp, Artificial Sequence
  source: /note=“Description of Artificial Sequence Synthetic polynucleotide”
gcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgc

SEQ ID NO: 13 - DNA, 681 bp, Artificial Sequence
  source: /note=“Description of Artificial Sequence Synthetic polynucleotide”
gataagacccacacatgtcctccatgccctgcccctgagctgctgggcggaccttccgtgttcctgttccctccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaacagcacctaccgggtggtgtccgtgctgaccgtgctgcatcaggactggctgaacggcaaagagtacaagtgcaaggtgtccaacaaggccctgcctgcccctatcgagaaaaccatcagcaaggccaagggccagccccgcgaacctcaggtgtacacactgcctccctgccgggaagagatgaccaagaaccaggtgtccctgtggtgcctggtcaagggcttctacccctccgatatcgccgtggaatgggagagcaacggccagcccgagaacaactacaagaccacccctcccgtgctggacagcgacggcagcttcttcctgtactccaaactgaccgtggacaagagccggtggcagcagggcaatgtgttcagctgtagcgtgatgcacgaggccctgcacaaccactacacccagaagtccctgagcctgtctcctggcaaa

SEQ ID NO: 14 - DNA, 336 bp, Artificial Sequence
  source: /note=“Description of Artificial Sequence Synthetic polynucleotide”
gacgtggtcatgacacagagccctctgtctctgcccgtgacattgggacagcctgcctccatctcctgcaagtcctctcagtccctgctggactctgacggcaagaccttcctgaactggttccagcagcggcctggccagtctcctagaaggctgatctacctggtgtccaagctggattctggcgtgcccgacagattctccggctctggctctggcaccgacttcaccctgaagatctccagagtggaagccgaggacgtgggcgtgtactactgttggcagggcacccactttccatacaccttcggccagggcaccagactggaaatcaag

SEQ ID NO: 15 - DNA, 321 bp, Artificial Sequence
  source: /note=“Description of Artificial Sequence Synthetic polynucleotide”
agaacagtggccgctccttccgtgttcatcttcccaccctccgacgagcagctgaagtccggcaccgcttctgtcgtgtgcctgctcaacaacttctaccctcgggaagccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgagcagccccgtgaccaagtccttcaaccggggcgagtgc

SEQ ID NO: 16 - DNA, 342 bp, Artificial Sequence
  source: /note=“Description of Artificial Sequence Synthetic polynucleotide”
gaagtgcagctggttcagtctggcgccgaagtgaagaaacctggcgcctctgtgaaggtgtcctgcaaggcttctggctacacctttaccggctaccacatgcactgggtccgacaggctccaggacaaggcttggaatggatgggctggatcaaccccaactccggcgtgaccaaatacgcccagaaattccagggcagagtgaccatgaccagagacacctccatcaacaccgcctacatggaactgtcccggctgagattcgacgacaccgacgtgtactactgtgccaccggcggctttggctattggggagagggaacactggtcaccgtgtcctcc

SEQ ID NO: 17 - DNA, 330 bp, Artificial Sequence
  source: /note=“Description of Artificial Sequence Synthetic polynucleotide”
ctgcccgtgttgacccagcctcctagcgtttccaagggcctgagacagaccgccacactgacctgtaccggcaactctaacaacgtgggcaatcagggcgctgcctggttgcagcagcatcagggacagcctccaaagctgctgtcctaccggaaccacaacagacctagcggcgtgtccgagcggttcagcccttctagatctggcgacacctccagcctgaccatcactggactgcagcctgaggacgaggccgactactattgtctggcctgggacagctccctgcgggcctttgtttttggcaccggcaccaagctgaccgtgctg

SEQ ID NO: 18 - DNA, 318 bp, Artificial Sequence
  source: /note=“Description of Artificial Sequence Synthetic polynucleotide”
ggacaacctaaggccaatcctaccgtgacactgttccctccatcctccgaggaactgcaggccaacaaggctaccctcgtgtgcctgatctccgacttttaccctggcgctgtgaccgtggcctggaaggctgatggatctcctgtgaaggctggcgtggaaaccaccaagccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcc

SEQ ID NO: 19 - DNA, 372 bp, Artificial Sequence
  source: /note=“Description of Artificial Sequence Synthetic polynucleotide”
caggtgcagctggttcagtctggcgccgaagtgaagaaacctggcgcctctgtgaaggtgtcctgcaaggcttccggctacaccttctccagctactacatgcactgggtccgacaggcccctggacaaggattggagtggatgggcatcatcaacccctctggcggcaacacctcttacgcccagaaattccagggcagagtgaccatgaccagagacacctccaccagcaccgtgtacatggaactgtccagcctgagatccgaggacaccgccgtgtactactgtgccagaggcggataccagctgcctcacggtagagccagagccttcgatatgtggggccagggcacaatggtcaccgtgtcctct

SEQ ID NO: 20 - DNA, 336 bp, Artificial Sequence
  source: /note=“Description of Artificial Sequence Synthetic polynucleotide”
gccatcagaatgacccagtctccactgagcctgcctgtgacattgggccagcctgcctctatctcctgcacctcctctcagtctctggtgtacagagatggcaccacctacctgaactggttccagcagaggcctggccagtctcctagacggctgatctacaaggtgtccaacagagactctggcgtgcccgacagattcaccggctctggctctggcaccacattcaccctgaccatctccagagtggaagccgaggacgtgggcatctactactgtatgcagggcacccactggcctctgacctttggccagggaacaaaggtggaaatcaag

SEQ ID NO: 21 - DNA, 336 bp, Artificial Sequence
  source: /note=“Description of Artificial Sequence Synthetic polynucleotide”
gaggtgcagctggttgaatctggcggaggattggttcagcctggcggctctctgagactgtcttgtgtggcctctggcttcaccttctccgactactggatgtcctgggtccgacaggctcctggcaaaggactggaatgggtcgccaacatcaagaaagacggctccgtgaactactacgtggactccgtgaagggcagattcaccatctctcgggacaacgccaagaactccctgtacctgcagatgaacagcctgagagccgaggacaccgccgtgtactactgcaccagattcgattactggggccagggcaccctggtcacagtgtcctct

SEQ ID NO: 22 - DNA, 330 bp, Artificial Sequence
  source: /note=“Description of Artificial Sequence Synthetic polynucleotide”
caggctggcttgacccagcctcctagcgtttccaagggcctgagacagaccgccacactgacctgtaccggcaactctaacaacgtgggcaatcagggcgctgcctggttgcagcagcatcagggacatcctccaaagctgctgttctaccggaacaacaacagagcctccggcatctccgagcggctgtctgcttctagatccggcaataccgccagcctgaccatcactggactgcagcctgaggacgaggccgactactattgcctgacctgggactcctctctgtccgtggtggtttttggcggaggcaccaagctgacagtgctg

SEQ ID NO: 23 - DNA, 348 bp, Artificial Sequence
  source: /note=“Description of Artificial Sequence Synthetic polynucleotide”
caggtgcagctggttcagtctggcgccgaagtgaagaaacctggcgcctctgtgaaggtgtcctgcaaggcttccggctacacctttaccagctacgacatctcctgggtccgacaggctcctggacaaggcttggaatggatgggcgtgatctggaccgatggcggcaccaattacgcccagaaactgcagggcagagtgaccatgaccaccgacacctctacctccaccgcctacatggaactgcggtccctgagatctgacgacaccgccgtgtactactgcgccagagatcagcggctgtacttcgatgtgtggggccagggcacaaccgtgacagtgtcctct

SEQ ID NO: 24 - DNA, 318 bp, Artificial Sequence
  source: /note=“Description of Artificial Sequence Synthetic polynucleotide”
gacatccagatgacccagtctccatcctctctgtccgcctctgtgggcgacagagtgaccatcacctgtagagcctccgaggacgtgaacacctacgtgtcctggtatcagcagaagcccggcaaggctcccaagctgctgatctacgccgcctctaacagatacaccggcgtgccctctagattctccggctctggctctggcaccgactttaccctgacaatctccagcctgcagcctgaggacttcgccacctactactgccagcagtccttcagctaccccacctttggccagggcaccaagctggaaatcaag

SEQ ID NO: 25 - DNA, 681 bp, Artificial Sequence
  source: /note=“Description of Artificial Sequence Synthetic polynucleotide”
gataagacccacacctgtcctccctgccctgcccctgaactgctgggcggacctagcgtgttcctgttccctccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccagcccctatcgagaaaaccatcagcaaggccaagggccagcctagagagcctcaggtctgcaccctgcctcccagccgggaagagatgaccaagaaccaggtgtccctgagctgcgccgtgaagggcttctacccctccgatatcgccgtggaatgggagagcaacggccagcccgagaacaactacaagaccacccctcccgtgctggacagcgacggcagcttcttcctggtgtccaaactgaccgtggacaagagccggtggcagcagggcaatgtgttcagctgtagcgtgatgcacgaggccctgcacaaccactacacccagaagtctctgagcctgagccctggcaaa

SEQ ID NO: 26 - DNA, 366 bp, Artificial Sequence
  source: /note=“Description of Artificial Sequence Synthetic polynucleotide”
caggtgcagctggttcagtctggcgccgaagtgaagaaacctggctcctccgtgaaggtgtcctgcaaggcttctggctacacctttaccgacaactacatgatctgggtccgacaggctcctggacagggacttgagtggatgggcgacatcaacccttacaacggcggcaccaccttcaaccagaaattcaagggcagagtgaccatcaccgccgacaagtctacctccaccgcctacatggaactgtccagcctgagatctgaggacaccgccgtgtactactgcgccagagagtccccttacttctccaacctgtacgtgatggactactggggccagggcacactggtcacagtgtcctct

SEQ ID NO: 27 - DNA, 333 bp, Artificial Sequence
  source: /note=“Description of Artificial Sequence Synthetic polynucleotide”
gagatcgtgctgacccagtctcctgccacactgtcactgtctccaggcgagagagctaccctgtcctgcaaggcttctcagtccgtggactacgacggcgacaactacatgaactggtatcagcagaagcccggccaggctcctagactgctgatctacgccgcctccaacctggaatctggcatccccgctagattctccggctctggctctggcacagactttaccctgaccatctccagcctggaacctgaggacttcgccgtgtactactgccacctgtccaacgaggacctgtccacatttggcggaggcaccaaggtggaaatcaag

SEQ ID NO: 28 - DNA, 1392 bp, Artificial Sequence
  source: /note=“Description of Artificial Sequence Synthetic polynucleotide”
atggaaaccgacacactgctgctgtgggtgctgctcttgtgggtgccaggatctacaggacaggtccagctgcaagagtctggccctggactggttcagccctctcagaccctgtctctgacctgtaccgtgtccggcttctccctgaccgacttctctgtgcactgggtccgacagcctccaggcaaaggactggaatggatgggcagaatcagatccgagggcaacaccgactacaacagcgccctgaagtcccggctgtctatcagcagagacacctccaagagccaggtgttcctgaagatgaactccctgcagaccgaggacaccgccatctatttctgcaccagaggcgacatcctcggcttcggctattggggacagggcgtgatggtcaccgttagctctgctcagaccaccgctcctagcgtgtaccctttggctcctggctgtggcgacaccacctcttctacagtgaccctgggctgtctggtcaagggctactttcctgagcctgtgaccgtgacctggaactctggtgccctgtcctccgacgtgcacacctttccagctgtgctgcagtccggcctgtacaccctgacatcctccgtgacctcttccacctggcctagccagaccgtgacatgcaatgtggctcaccctgcctccagcaccaaggtggacaagaaggtggaacggcggaccattaagccttgtcctccatgcaagtgccccgctcctaatctgctcggaggcccttccgtgttcatctttccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaactctaccctgagagtggtgtctgccctgcctatccagcatcaggactggatgtccggcaaagaattcaagtgcaaagtgaacaacaaggacctgcctgctccaatcgagcggaccatctctaagcctaagggctctgtcagggcccctcaggtgtacgttctgcctccttgcgaggaagagatgaccaagaaacaagtgacactgtggtgcatggtcacagacttcatgcccgaggacatctacgtggaatggaccaacaacggcaagaccgagctgaactacaagaacaccgagcctgtgctggactccgacggctcctacttcatgtactccaagctgcgcgtcgagaagaagaactgggtcgagagaaactcctactcctgctccgtggtgcacgagggcctgcacaatcaccacaccaccaagtccttctctcggacccctggcaagtgatga

SEQ ID NO: 29 - DNA, 723 bp, Artificial Sequence
  source: /note=“Description of Artificial Sequence Synthetic polynucleotide”
atggaaaccgacacactgctgctgtgggtgctgctcttgtgggtgccaggatctaccggcgacatcgtgatgacccagtctccactgtccgtgtctgtgacccctggcgagtctgcctccatctcctgcagatcctccaagagcctgctgcacttcaagggcatcaccttcgtgtactggtatctgcagaagcccggccagtctcctcagctgctgatcttcagaatgtccagcctggcctctggcgtgcccgatagattttctggctccggctccgagacagacttcaccctgaagatctccagagtggaagccgaggacgtgggcacctactattgtggccagctgctggaaaacccctacacctttggcgctggcaccaagctggaactgaagagagctgacgctgcccctaccgtgtctatcttccctccatccatggaacagctgacctctggcggagctaccgtcgtgtgcttcgtgaacaacttctaccctcgggacatctccgtgaagtggaagatcgacggctctgagcagcgagatggcgtgctggattctgtgaccgaccaggactccaaggacagcacctactccatgtctagcaccctgagcctgaccaaggtggaatacgagcggcacaacctgtatacctgcgaggtggtgcacaagacctccagctctcccgtggtcaagtccttcaaccggaacgagtgctgatga

SEQ ID NO: 30 - DNA, 1413 bp, Artificial Sequence
  source: /note=“Description of Artificial Sequence Synthetic polynucleotide”
atggaaaccgacacactgctgctgtgggtgctgctcttgtgggtgccaggatctacaggacaggtccagttgcagcagtctggcgctgagctggtcaagcctggatcctccgtgaagatctcctgcaaggcctccggctacaccttcacctccaacttcatgcactggatcaagcagcagcccggcaacggcctggaatggatcggatggatctatcctggcgacggcgacaccgagtacaaccagaagttcaacggcaaggctaccctgaccgccgacaagtcctcttccaccgcttacatgcagctgtccagcctgacctctgaggactccgccgtgtacttctgcgccgtgaattatggcggctacgtgctggatgcttggggccaaggcgcttctgtgacagtgtcctctgccgagacaaccgctcctagcgtttaccctctggctcctggcacagccctgaagtccaactctatggtcaccctgggctgcctggtcaagggctactttcctgagcctgtgaccgtgacctggaactctggtgctctgtctagcggcgtgcacacctttccagctgtgctgcagagcggcctgtacaccctgacatctagcgtgaccgtgccttccagcacctggtctagtcaggctgtgacctgcaacgtggcccatcctgcctcttctaccaaggtggacaagaaaatcgtgcccagagagtgcaacaccatcaagccctgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgccaatccagcaccaggattggatgtccggcaaagaattcaagtgcaaagtgaacaacaaggacctgcctgctccaatcgagcggaccatctctaagcctaagggctctgtgcgggctccccaagtttgtgttctgcctccacctgaggaagagatgaccaagaaacaagtgaccctgtcttgtgccgtgaccgacttcatgcccgaggacatctacgtggaatggaccaacaatggcaagaccgagctgaactacaagaacaccgagcctgtgctggactccgacggctcctacttcatggtgtctaagctgcgcgtcgagaagaagaactgggtcgagagaaactcctactcctgctccgtggtgcacgagggcctgcacaatcaccacaccaccaagtccttctctcggacccctggcaagtgatga

SEQ ID NO: 31 - DNA, 705 bp, Artificial Sequence
  source: /note=“Description of Artificial Sequence Synthetic polynucleotide”
atggaaaccgacacactgctgctgtgggtgctgctcttgtgggtgccaggatctacaggcgagatcgtgctgacccagtctcctaccaccatggctgctagccctggcgagaaagtgacaattacctgccgggcctcctcctccaccaactacatgtcctggtatcagcagaagtccggcgcctctcctaagccttggatctacgagacatccaagctggcctctggcgtgcccgatagattttccggctctggctccggcacctcctacagcttcaccatctccagcatggaaacagaggacgccgccacctactactgccaccagtggtcatctacccctctgacctttggcagcggcaccaagctggaaatcaagagagctgacgccgctcctaccgtgtctatcttccctccatccatggaacagctgacctccggcggagctaccgtcgtgtgtttcgtgaacaacttctaccctcgggacatctccgtgaagtggaagatcgacggctctgagcagcgagatggcgtgctggattctgtgaccgaccaggactccaaggacagcacctactccatgtctagcaccctgagcctgaccaaggtggaatacgagcggcacaacctgtatacctgcgaggtggtgcacaagacctccagctctcccgtggtcaagtccttcaaccggaacgagtgctgatga

SEQ ID NO: 32 - DNA, 1407 bp, Artificial Sequence
  source: /note=“Description of Artificial Sequence Synthetic polynucleotide”
atggaaaccgacacactgctgctgtgggtgctgctcttgtgggtgccaggatctacaggcgaggtgcagctggttgaatctggcggaggactggttaagcctggcggctctctgagactgtcttgtgccgcttctggcttcaccttctccgcctacgccatgaactgggtccgacaggctcctggcaaaggcctggaatgggtcggaagaatccggaccaagaacaacaactacgccacctactacgccgactccgtgaaggaccggttcaccatctctcgggacgactccaagaacaccctgtacctgcagatgaactccctgaaaaccgaggacaccgccgtgtactactgcaccaccttctacggcaatggcgtgtggggacagggcacactggttaccgtttcttccgcctccaccaagggaccctctgtgtttcctctggctccctccagcaagtctacctctggtggaacagctgccctgggctgcctggtcaaggattactttcctgagcctgtgaccgtgtcctggaactctggcgctctgacatctggcgtgcacacctttccagctgtgctgcagtcctctggcctgtactctctgtcctccgtcgtgaccgtgccttctagctctctgggcacccagacctacatctgcaatgtgaaccacaagccttccaacaccaaggtggacaagagagtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaatcaggtgtccctgtggtgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaagtgatga

SEQ ID NO: 33 - DNA, 723 bp, Artificial Sequence
  source: /note=“Description of Artificial Sequence Synthetic polynucleotide”
atggaaaccgacacactgctgctgtgggtgctgctcttgtgggtgccaggatctacaggcgacgtggtcatgacacagagccctctgtctctgcccgtgacattgggacagcctgcctccatctcctgcaagtcctctcagtccctgctggactctgacggcaagaccttcctgaactggttccagcagcggcctggccagtctcctagaaggctgatctacctggtgtccaagctggattctggcgtgcccgacagattctccggctctggctctggcaccgacttcaccctgaagatctccagagtggaagccgaggacgtgggcgtgtactactgttggcagggcacccactttccatacaccttcggccagggcaccagactggaaatcaagagaaccgtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtccagcacactgaccctgtccaaggccgactacgagaagcacaaggtgtacgcctgcgaagtgacccatcagggcctgtctagccctgtgaccaagtctttcaaccggggcgagtgctgatga

SEQ ID NO: 34 - DNA, 1398 bp, Artificial Sequence
  source: /note=“Description of Artificial Sequence Synthetic polynucleotide”
atggaaaccgacacactgctgctgtgggtgctgctcttgtgggtgccaggatctacaggcgaagtgcagctggttcagtctggcgccgaagtgaagaaacctggcgcctctgtgaaggtgtcctgcaaggcttctggctacacctttaccggctaccacatgcactgggtccgacaggctccaggacaaggcttggaatggatgggctggatcaaccccaactccggcgtgaccaaatacgcccagaaattccagggcagagtgaccatgaccagagacacctccatcaacaccgcctacatggaactgtcccggctgagattcgacgacaccgacgtgtactactgtgccaccggcggctttggctattggggagagggaacactggtcaccgtgtcctccgcttctaccaagggaccctccgtgtttcctctggctccttccagcaagtctacctccggtggaacagctgctctgggctgcctggtcaaggactactttcctgagcctgtgaccgtgtcttggaactctggcgctctgacatccggcgtgcacacctttccagctgtgctgcaatcctccggcctgtactctctgtcctccgtcgtgaccgtgccttctagctctctgggcacccagacctacatctgcaatgtgaaccacaagccttccaacaccaaggtggacaagagagtggaacccaagtcctgcgacaagacccacacctgtcctccatgtcctgctccagaactgctcggcggaccttctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtgatga

SEQ ID NO: 35 - DNA, 714 bp, Artificial Sequence
  source: /note=“Description of Artificial Sequence Synthetic polynucleotide”
atggaaaccgacacactgctgctgtgggtgctgctcttgtgggtgccaggatctacaggactgcccgtgttgacccagcctcctagcgtttccaagggcctgagacagaccgccacactgacctgtaccggcaactctaacaacgtgggcaatcagggcgctgcctggttgcagcagcatcagggacagcctccaaagctgctgtcctaccggaaccacaacagacctagcggcgtgtccgagcggttcagcccttctagatctggcgacacctccagcctgaccatcactggactgcagcctgaggacgaggccgactactattgtctggcctgggacagctccctgcgggcctttgtttttggcaccggcaccaagctgaccgtgctgggacaacctaaggccaatcctaccgtgacactgttccctccatcctccgaggaactgcaggccaacaaggctaccctcgtgtgcctgatctccgacttttaccctggcgctgtgaccgtggcctggaaggctgatggatctcctgtgaaggctggcgtggaaaccaccaagccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcctgatga

SEQ ID NO: 36 - DNA, 1428 bp, Artificial Sequence
  source: /note=“Description of Artificial Sequence Synthetic polynucleotide”
atggaaaccgacacactgctgctgtgggtgctgctcttgtgggtgccaggatctacaggacaggtgcagctggttcagtctggcgccgaagtgaagaaacctggcgcctctgtgaaggtgtcctgcaaggcttccggctacaccttctccagctactacatgcactgggtccgacaggcccctggacaaggattggagtggatgggcatcatcaacccctctggcggcaacacctcttacgcccagaaattccagggcagagtgaccatgaccagagacacctccaccagcaccgtgtacatggaactgtccagcctgagatccgaggacaccgccgtgtactactgtgccagaggcggataccagctgcctcacggtagagccagagccttcgatatgtggggccagggcacaatggtcaccgtgtcctctgcttccaccaagggaccctctgtgttccctctggctccttccagcaagtccacatccggtggaacagctgctctgggctgcctggtcaaggactactttcctgagcctgtgaccgtgtcttggaactctggcgctctgacatccggcgtgcacacatttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgacaagacccacacctgtccaccatgtcctgctccagaactgctcggcggaccttccgtgttcctgtttcctccaaagcctaaggacaccctgatgatctctcggacccctgaagtgacctgcgtggtggtggatgtgtcccacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctcgtgaagggcttctacccttccgatatcgccgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtgatga

SEQ ID NO: 37 - DNA, 723 bp, Artificial Sequence
  source: /note=“Description of Artificial Sequence Synthetic polynucleotide”
atggaaaccgacacactgctgctgtgggtgctgctcttgtgggtgccaggatctaccggcgccatcagaatgacccagtctccactgagcctgcctgtgacattgggccagcctgcctctatctcctgcacctcctctcagtctctggtgtacagagatggcaccacctacctgaactggttccagcagaggcctggccagtctcctagacggctgatctacaaggtgtccaacagagactctggcgtgcccgacagattcaccggctctggctctggcaccacattcaccctgaccatctccagagtggaagccgaggacgtgggcatctactactgtatgcagggcacccactggcctctgacctttggccagggaacaaaggtggaaatcaagcggaccgtggccgctccttccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatga

SEQ ID NO: 38 - DNA, 1392 bp, Artificial Sequence
  source: /note=“Description of Artificial Sequence Synthetic polynucleotide”
atggaaaccgacacactgctgctgtgggtgctgctcttgtgggtgccaggatctacaggcgaggtgcagctggttgaatctggcggaggattggttcagcctggcggctctctgagactgtcttgtgtggcctctggcttcaccttctccgactactggatgtcctgggtccgacaggctcctggcaaaggactggaatgggtcgccaacatcaagaaagacggctccgtgaactactacgtggactccgtgaagggcagattcaccatctctcgggacaacgccaagaactccctgtacctgcagatgaacagcctgagagccgaggacaccgccgtgtactactgcaccagattcgattactggggccagggcaccctggtcacagtgtcctctgcttctaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggttaagggcttctacccctccgatatcgccgtggaatgggagtctaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaagtgatga

SEQ ID NO: 39 - DNA, 714 bp, Artificial Sequence
  source: /note=“Description of Artificial Sequence Synthetic polynucleotide”
atggaaaccgacacactgctgctgtgggtgctgctcttgtgggtgccaggatctacaggacaggctggcttgacccagcctcctagcgtttccaagggcctgagacagaccgccacactgacctgtaccggcaactctaacaacgtgggcaatcagggcgctgcctggttgcagcagcatcagggacatcctccaaagctgctgttctaccggaacaacaacagagcctccggcatctccgagcggctgtctgcttctagatccggcaataccgccagcctgaccatcactggactgcagcctgaggacgaggccgactactattgcctgacctgggactcctctctgtccgtggtggtttttggcggaggcaccaagctgacagtgctgggacagcctaaggccaatcctaccgtgacactgttccctccatcctccgaggaactgcaggccaacaaggctaccctcgtgtgcctgatctccgacttttaccctggcgctgtgaccgtggcctggaaggctgatggatctcctgtgaaggctggcgtggaaaccaccaagccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcctgatga

SEQ ID NO: 40 - DNA, 1404 bp, Artificial Sequence
  source: /note=“Description of Artificial Sequence Synthetic polynucleotide”
atggaaaccgacacactgctgctgtgggtgctgctcttgtgggtgccaggatctacaggacaggtgcagctggttcagtctggcgccgaagtgaagaaacctggcgcctctgtgaaggtgtcctgcaaggcttccggctacacctttaccagctacgacatctcctgggtccgacaggctcctggacaaggcttggaatggatgggcgtgatctggaccgatggcggcaccaattacgcccagaaactgcagggcagagtgaccatgaccaccgacacctctacctccaccgcctacatggaactgcggtccctgagatctgacgacaccgccgtgtactactgcgccagagatcagcggctgtacttcgatgtgtggggccagggcacaaccgtgacagtgtcctctgcttccaccaagggacccagcgttttccctctggctccatcctccaagtctacctctggcggaacagctgctctgggctgcctggtcaaggactactttcctgagcctgtgaccgtgtcctggaactctggcgctctgacatctggcgtgcacacattccctgctgtgctgcagtcctccggcctgtactctctgtcctctgtggttaccgtgccttcctctagcctgggcacccagacctacatctgcaatgtgaaccacaagccttccaacaccaaggtggacaagagagtggaacccaagtcctgcgacaagacccacacctgtccaccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgagctgcgccgtgaagggcttctacccttctgatatcgccgtggaatgggagagcaacggccagcctgagaacaactacaagacaacccctcctgtgctggactccgacggctcattcttcctggtgtccaagctgacagtggacaagtccagatggcagcagggcaacgtgttctcctgctccgtgatgcacgaggccctgcacaatcactacacacagaagtccctgtctctgtcccctggcaagtgatga

SEQ ID NO: 41 - DNA, 705 bp, Artificial Sequence
  source: /note=“Description of Artificial Sequence Synthetic polynucleotide”
atggaaaccgacacactgctgctgtgggtgctgctcttgtgggtgccaggatctaccggcgacatccagatgacccagtctccatcctctctgtccgcctctgtgggcgacagagtgaccatcacctgtagagcctccgaggacgtgaacacctacgtgtcctggtatcagcagaagcccggcaaggctcccaagctgctgatctacgccgcctctaacagatacaccggcgtgccctctagattctccggctctggctctggcaccgactttaccctgacaatctccagcctgcagcctgaggacttcgccacctactactgccagcagtccttcagctaccccacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatga

SEQ ID NO: 42 - DNA, 1422 bp, Artificial Sequence
  source: /note=“Description of Artificial Sequence Synthetic polynucleotide”
atggaaaccgacacactgctgctgtgggtgctgctcttgtgggtgccaggatctacaggacaggtgcagctggttcagtctggcgccgaagtgaagaaacctggctcctccgtgaaggtgtcctgcaaggcttctggctacacctttaccgacaactacatgatctgggtccgacaggctcctggacagggacttgagtggatgggcgacatcaacccttacaacggcggcaccaccttcaaccagaaattcaagggcagagtgaccatcaccgccgacaagtctacctccaccgcctacatggaactgtccagcctgagatctgaggacaccgccgtgtactactgcgccagagagtccccttacttctccaacctgtacgtgatggactactggggccagggcacactggtcacagtgtcctctgcttccaccaagggacccagcgttttccctctggctccatcctccaagtccacctctggtggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtcgacaagagagtggaacccaagtcctgcgacaagacccacacctgtccaccttgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgagctgcgccgtgaagggcttctacccttctgatatcgccgtggaatgggagagcaacggccagccagagaacaactacaagacaacccctcctgtgctggactccgacggctcattcttcctggtgtccaagctgacagtggacaagtccagatggcagcagggcaacgtgttctcctgctccgtgatgcacgaggccctgcacaatcactacacacagaagtctctgtctctgagccccggcaagtgatga

SEQ ID NO: 43 - DNA, 720 bp, Artificial Sequence
  source: /note=“Description of Artificial Sequence Synthetic polynucleotide”
atggaaaccgacacactgctgctgtgggtgctgctcttgtgggtgccaggatctacaggcgagatcgtgctgacccagtctcctgccacactgtcactgtctccaggcgagagagctaccctgtcctgcaaggcttctcagtccgtggactacgacggcgacaactacatgaactggtatcagcagaagcccggccaggctcctagactgctgatctacgccgcctccaacctggaatctggcatccccgctagattctccggctctggctctggcacagactttaccctgaccatctccagcctggaacctgaggacttcgccgtgtactactgccacctgtccaacgaggacctgtccacatttggcggaggcaccaaggtggaaatcaagcggacagtggccgctccttccgtgttcatcttcccaccttccgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatga

SEQ ID NO: 44 - protein, 116 aa, Artificial Sequence
  source: /note=“Description of Artificial Sequence Synthetic polypeptide”
QVQLQESGPGLVQPSQTLSLTCTVSGFSLTDFSVHWVRQPPGKGLEWMGRIRSEGNTDYNSALKSRLSISRDTSKSQVFLKMNSLQTEDTAIYFCTRGDILGFGYWGQGVMVTVSS

SEQ ID NO: 45 - protein, 112 aa, Artificial Sequence
  source: /note=“Description of Artificial Sequence Synthetic polypeptide”
DIVMTQSPLSVSVTPGESASISCRSSKSLLHFKGITFVYWYLQKPGQSPQLLIFRMSSLASGVPDRFSGSGSETDFTLKISRVEAEDVGTYYCGQLLENPYTFGAGTKLELK

SEQ ID NO: 46 - protein, 98 aa, Artificial Sequence
  source: /note=“Description of Artificial Sequence Synthetic polypeptide”
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERR

SEQ ID NO: 47 - protein, 107 aa, Artificial Sequence
  source: /note=“Description of Artificial Sequence Synthetic polypeptide”
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 48 - protein, 118 aa, Artificial Sequence
  source: /note=“Description of Artificial Sequence Synthetic polypeptide”
QVQLQQSGAELVKPGSSVKISCKASGYTFTSNFMHWIKQQPGNGLEWIGWIYPGDGDTEYNQKFNGKATLTADKSSSTAYMQLSSLTSEDSAVYFCAVNYGGYVLDAWGQGASVTVSS

SEQ ID NO: 49 - protein, 103 aa, Artificial Sequence
  source: /note=“Description of Artificial Sequence Synthetic polypeptide”
AETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECN

SEQ ID NO: 50 - protein, 106 aa, Artificial Sequence
  source: /note=“Description of Artificial Sequence Synthetic polypeptide”
EIVLTQSPTTMAASPGEKVTITCRASSSTNYMSWYQQKSGASPKPWIYETSKLASGVPDRFSGSGSGTSYSFTISSMETEDAATYYCHQWSSTPLTFGSGTKLEIK

SEQ ID NO: 51 - protein, 107 aa, Artificial Sequence; identical to SEQ ID NO: 47
  source: /note=“Description of Artificial Sequence Synthetic polypeptide”

SEQ ID NO: 52 - protein, 228 aa, Artificial Sequence
  source: /note=“Description of Artificial Sequence Synthetic polypeptide”
TIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 53 - protein, 228 aa, Artificial Sequence
  source: /note=“Description of Artificial Sequence Synthetic polypeptide”
TIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 54 - protein, 117 aa, Artificial Sequence
  source: /note=“Description of Artificial Sequence Synthetic polypeptide”
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSS

SEQ ID NO: 55 - protein, 103 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 56 - protein, 227 aa, Artificial Sequence
  source: /note=“Description of Artificial Sequence Synthetic polypeptide”
  VARIANT (227)..(227): /replace=“ ”
  SITE (1)..(227): /note=“Variant residues given in the sequence have no preference with respect to those in the annotations for variant positions”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 112 aa, Artificial Sequence
  source: /note=“Description of Artificial Sequence Synthetic polypeptide”
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIK

SEQ ID NO: 58 - protein, 107 aa, Artificial Sequence [sequence omitted]

SEQ ID NO: 59 - protein, 114 aa, Artificial Sequence
  source: /note=“Description of Artificial Sequence Synthetic polypeptide”
EVQLVQSGAEVKKPGASVKVSCKASGYTFTGYHMHWVRQAPGQGLEWMGWINPNSGVTKYAQKFQGRVTMTRDTSINTAYMELSRLRFDDTDVYYCATGGFGYWGEGTLVTVSS

SEQ ID NO: 60 - protein, 110 aa, Artificial Sequence
  source: /note=“Description of Artificial Sequence Synthetic polypeptide”
LPVLTQPPSVSKGLRQTATLTCTGNSNNVGNQGAAWLQQHQGQPPKLLSYRNHNRPSGVSERFSPSRSGDTSSLTITGLQPEDEADYYCLAWDSSLRAFVFGTGTKLTVL

SEQ ID NO: 61 - protein, 106 aa, Artificial Sequence
  source: /note=“Description of Artificial Sequence Synthetic polypeptide”
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 62 - protein, 124 aa, Artificial Sequence
  source: /note=“Description of Artificial Sequence Synthetic polypeptide”
QVQLVQSGAEVKKPGASVKVSCKASGYTFSSYYMHWVRQAPGQGLEWMGIINPSGGNTSYAQKFQGRVTMTRDTSTSTVYMELSSLRSEDTAVYYCARGGYQLPHGRARAFDMWGQGTMVTVSS

SEQ ID NO: 63 - protein, 112 aa, Artificial Sequence
  source: /note=“Description of Artificial Sequence Synthetic polypeptide”
AIRMTQSPLSLPVTLGQPASISCTSSQSLVYRDGTTYLNWFQQRPGQSPRRLIYKVSNRDSGVPDRFTGSGSGTTFTLTISRVEAEDVGIYYCMQGTHWPLTFGQGTKVEIK

SEQ ID NO: 64 - protein, 112 aa, Artificial Sequence
  source: /note=“Description of Artificial Sequence Synthetic polypeptide”
EVQLVESGGGLVQPGGSLRLSCVASGFTFSDYWMSWVRQAPGKGLEWVANIKKDGSVNYYVDSVKGRFTISRDNAKNSLYLQMNSLRAEDTAVYYCTRFDYWGQGTLVTVSS

SEQ ID NO: 65 - protein, 110 aa, Artificial Sequence
  source: /note=“Description of Artificial Sequence Synthetic polypeptide”
QAGLTQPPSVSKGLRQTATLTCTGNSNNVGNQGAAWLQQHQGHPPKLLFYRNNNRASGISERLSASRSGNTASLTITGLQPEDEADYYCLTWDSSLSVVVFGGGTKLTVL

SEQ ID NO: 66 - protein, 116 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSS

SEQ ID NO: 67 - protein, 106 aa, Artificial Sequence
  source: /note=“Description of Artificial Sequence Synthetic polypeptide”
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 68 - protein, 227 aa, Artificial Sequence
  source: /note=“Description of Artificial Sequence Synthetic polypeptide”
  VARIANT (227)..(227): /replace=“ ”
  SITE (1)..(227): /note=“Variant residues given in the sequence have no preference with respect to those in the annotations for variant positions”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 122 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSS

SEQ ID NO: 70 - protein, 111 aa, Artificial Sequence
  source: /note=“Description of Artificial Sequence Synthetic polypeptide”
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 71 - protein, 442 aa, Artificial Sequence
  source: /note=“Description of Artificial Sequence Synthetic polypeptide”
QVQLQESGPGLVQPSQTLSLTCTVSGFSLTDFSVHWVRQPPGKGLEWMGRIRSEGNTDYNSALKSRLSISRDTSKSQVFLKMNSLQTEDTAIYFCTRGDILGFGYWGQGVMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 72 - protein, 219 aa, Artificial Sequence
  source: /note=“Description of Artificial Sequence Synthetic polypeptide”
DIVMTQSPLSVSVTPGESASISCRSSKSLLHFKGITFVYWYLQKPGQSPQLLIFRMSSLASGVPDRFSGSGSETDFTLKISRVEAEDVGTYYCGQLLENPYTFGAGTKLELKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 73 - protein, 449 aa, Artificial Sequence
  source: /note=“Description of Artificial Sequence Synthetic polypeptide”
QVQLQQSGAELVKPGSSVKISCKASGYTFTSNFMHWIKQQPGNGLEWIGWIYPGDGDTEYNQKFNGKATLTADKSSSTAYMQLSSLTSEDSAVYFCAVNYGGYVLDAWGQGASVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 74 - protein, 213 aa, Artificial Sequence
  source: /note=“Description of Artificial Sequence Synthetic polypeptide”
EIVLTQSPTTMAASPGEKVTITCRASSSTNYMSWYQQKSGASPKPWIYETSKLASGVPDRFSGSGSGTSYSFTISSMETEDAATYYCHQWSSTPLTFGSGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 75 - protein, 447 aa, Artificial Sequence
  source: /note=“Description of Artificial Sequence Synthetic polypeptide”
  VARIANT (447)..(447): /replace=“ ”
  SITE (1)..(447): /note=“Variant residues given in the sequence have no preference with respect to those in the annotations for variant positions”
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 219 aa, Artificial Sequence
  source: /note=“Description of Artificial Sequence Synthetic polypeptide”
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 444 aa, Artificial Sequence
  source: /note=“Description of Artificial Sequence Synthetic polypeptide”
  VARIANT (444)..(444): /replace=“ ”
  SITE (1)..(444): /note=“Variant residues given in the sequence have no preference with respect to those in the annotations for variant positions”
EVQLVQSGAEVKKPGASVKVSCKASGYTFTGYHMHWVRQAPGQGLEWMGWINPNSGVTKYAQKFQGRVTMTRDTSINTAYMELSRLRFDDTDVYYCATGGFGYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 216 aa, Artificial Sequence
  source: /note=“Description of Artificial Sequence Synthetic polypeptide”
LPVLTQPPSVSKGLRQTATLTCTGNSNNVGNQGAAWLQQHQGQPPKLLSYRNHNRPSGVSERFSPSRSGDTSSLTITGLQPEDEADYYCLAWDSSLRAFVFGTGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 79 - protein, 454 aa, Artificial Sequence
  source: /note=“Description of Artificial Sequence Synthetic polypeptide”
  VARIANT (454)..(454): /replace=“ ”
  SITE (1)..(454): /note=“Variant residues given in the sequence have no preference with respect to those in the annotations for variant positions”
QVQLVQSGAEVKKPGASVKVSCKASGYTFSSYYMHWVRQAPGQGLEWMGIINPSGGNTSYAQKFQGRVTMTRDTSTSTVYMELSSLRSEDTAVYYCARGGYQLPHGRARAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 219 aa, Artificial Sequence
  source: /note=“Description of Artificial Sequence Synthetic polypeptide”
AIRMTQSPLSLPVTLGQPASISCTSSQSLVYRDGTTYLNWFQQRPGQSPRRLIYKVSNRDSGVPDRFTGSGSGTTFTLTISRVEAEDVGIYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 442 aa, Artificial Sequence
  source: /note=“Description of Artificial Sequence Synthetic polypeptide”
  VARIANT (442)..(442): /replace=“ ”
  SITE (1)..(442): /note=“Variant residues given in the sequence have no preference with respect to those in the annotations for variant positions”
EVQLVESGGGLVQPGGSLRLSCVASGFTFSDYWMSWVRQAPGKGLEWVANIKKDGSVNYYVDSVKGRFTISRDNAKNSLYLQMNSLRAEDTAVYYCTRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 216 aa, Artificial Sequence
  source: /note=“Description of Artificial Sequence Synthetic polypeptide”
QAGLTQPPSVSKGLRQTATLTCTGNSNNVGNQGAAWLQQHQGHPPKLLFYRNNNRASGISERLSASRSGNTASLTITGLQPEDEADYYCLTWDSSLSVVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 83 - protein, 446 aa, Artificial Sequence
  source: /note=“Description of Artificial Sequence Synthetic polypeptide”
  VARIANT (446)..(446): /replace=“ ”
  SITE (1)..(446): /note=“Variant residues given in the sequence have no preference with respect to those in the annotations for variant positions”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 213 aa, Artificial Sequence
  source: /note=“Description of Artificial Sequence Synthetic polypeptide”
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 452 aa, Artificial Sequence
  source: /note=“Description of Artificial Sequence Synthetic polypeptide”
  VARIANT (452)..(452): /replace=“ ”
  SITE (1)..(452): /note=“Variant residues given in the sequence have no preference with respect to those in the annotations for variant positions”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 218 aa, Artificial Sequence
  source: /note=“Description of Artificial Sequence Synthetic polypeptide”
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 88 - protein, 306 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVGTPSPSLCPA

SEQ ID NO: 89 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 90 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 91 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 92 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 93 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 94 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 95 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 96 - protein, 507 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 97 - protein, 426 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 98 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 99 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 100 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 101 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 102 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 103 - protein, 101 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 104 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 105 - protein, 79 aa, Homo sapiens
ALQCFCHLCTKDNFTCVTDGLCFVSVTETTDKVIHNSMCIAEIDLIPRDRPFVCAPSSKTGSVTTTYCCNQDHCNKIEL

SEQ ID NO: 106 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 107 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 108 - protein, 767 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 109 - protein, 121 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 110 - protein, 108 aa, Artificial Sequence
  source: /note=“Description of Artificial Sequence Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 111 - protein, 118 aa, Artificial Sequence [sequence omitted]

SEQ ID NO: 112 - protein, 107 aa, Artificial Sequence [sequence omitted]

SEQ ID NO: 113 - protein, 120 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 114 - protein, 110 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 115 - DNA, 60 bp, Artificial Sequence
  source: /note=“Description of Artificial Sequence Synthetic oligonucleotide”
atggaaaccgacacactgctgctgtgggtgctgctcttgtgggtgccaggatctacagga

SEQ ID NO: 116 - protein, 20 aa, Artificial Sequence
  source: /note=“Description of Artificial Sequence Synthetic peptide”
METDTLLLWVLLLWVPGSTG

SEQ ID NO: 117 - protein, 361 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 118 - protein, 394 aa, Homo sapiens
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 119 - protein, 389 aa, Homo sapiens
LSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 120 - protein, 470 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 121 - protein, 474 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 122 - protein, 393 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 123 - protein, 545 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 124 - protein, 570 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 125 - protein, 831 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 126 - protein, 830 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 127 - protein, 1 aa, Artificial Sequence
  source: /note=“Description of Artificial Sequence Synthetic peptide”
G

SEQ ID NO: 128 - protein, 5 aa, Artificial Sequence
  source: /note=“Description of Artificial Sequence Synthetic peptide”
GGGGS

SEQ ID NO: 129 - protein, 5 aa, Artificial Sequence
  source: /note=“Description of Artificial Sequence Synthetic peptide”
GGSGG

SEQ ID NO: 130 - protein, 6 aa, Artificial Sequence
  source: /note=“Description of Artificial Sequence Synthetic peptide”
GGSGGS

SEQ ID NO: 131 - protein, 7 aa, Artificial Sequence
  source: /note=“Description of Artificial Sequence Synthetic peptide”
GGGSGGG

SEQ ID NO: 132 - protein, 9 aa, Artificial Sequence
  source: /note=“Description of Artificial Sequence Synthetic peptide”
GGGGSGGGG

SEQ ID NO: 133 - protein, 15 aa, Artificial Sequence
  source: /note=“Description of Artificial Sequence Synthetic peptide”
GGGGSGGGGSGGGGS

SEQ ID NO: 134 - protein, 20 aa, Artificial Sequence
  source: /note=“Description of Artificial Sequence Synthetic peptide”
GGGGSGGGGSGGGGSGGGGS

SEQ ID NO: 135 - protein, 99 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 136 - protein, 22 aa, Artificial Sequence
  source: /note=“Description of Artificial Sequence Synthetic peptide”
MGRGLLRGLWPLHIVLWTRIAS

SEQ ID NO: 137 - protein, 707 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 138 - protein, 216 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 139 - protein, 718 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSGGGGSGGGGSGGGGSGGGGSEIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 140 - protein, 434 aa, Artificial Sequence
  source: /note=“Description of Artificial Sequence Synthetic polypeptide”
  VARIANT (434)..(434): /replace=“ ”
  SITE (1)..(434): /note=“Variant residues given in the sequence have no preference with respect to those in the annotations for variant positions”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 141 - protein, 249 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 596 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 143 - protein, 626 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 144 - protein, 637 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSGGGGSGGGGSGGGGSGGGGSEIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 145 - protein, 711 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 146 - protein, 717 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 147 - protein, 213 aa, Artificial Sequence; identical to SEQ ID NO: 84
  source: /note=“Description of Artificial Sequence Synthetic polypeptide”

SEQ ID NO: 148 - protein, 218 aa, Artificial Sequence; identical to SEQ ID NO: 86
  source: /note=“Description of Artificial Sequence Synthetic polypeptide”

SEQ ID NO: 149 - protein, 688 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 150 - protein, 215 aa, Artificial Sequence
  source: /note=“Description of Artificial Sequence Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 685 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 152 - protein, 214 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 710 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 154 - protein, 706 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 155 - protein, 716 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 156 - protein, 712 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 157 - protein, 580 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 158 - protein, 591 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 159 - protein, 588 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 160 - protein, 599 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSGGGGSGGGGSGGGGSGGGGSEIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 161 - protein, 211 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 472 aa, Artificial Sequence
  source: /note=“Description of Artificial Sequence Synthetic polypeptide”
  VARIANT (472)..(472): /replace=“ ”
  SITE (1)..(472): /note=“Variant residues given in the sequence have no preference with respect to those in the annotations for variant positions”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 163 - protein, 634 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 164 - protein, 633 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 165 - protein, 629 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 166 - protein, 699 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 167 - protein, 698 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 168 - protein, 694 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 169 - protein, 737 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 170 - protein, 736 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSGGGGSGGGGSGGGGSGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 171 - protein, 732 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 172 - protein, 691 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 173 - protein, 702 aa, Artificial Sequence
  source: /note=“Description of Artificial Sequence Synthetic polypeptide”
LLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSGGGGSGGGGSGGGGSGGGGSEIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 174 - protein, 729 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 175 - protein, 720 aa, Artificial Sequence
  source: /note=“Description of Artificial Sequence Synthetic polypeptide”
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSEIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 176 - protein, 564 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 177 - protein, 565 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 178 - protein, 562 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 179 - protein, 602 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 180 - protein, 603 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 181 - protein, 600 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 182 - protein, 1232 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 183 - protein, 1233 aa, Artificial Sequence
  source: /note=“Description of Artificial Sequence Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 184 - protein, 1230 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 185 - protein, 560 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSLLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 186 - protein, 566 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSLLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 187 - protein, 598 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 188 - protein, 604 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 189 - protein, 1228 aa, Artificial Sequence
  source: /note=“Description of Artificial Sequence Synthetic polypeptide”
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 190 - protein, 1234 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTV

SEQ ID NO: 191 - protein, 26 aa, Artificial Sequence
  source: /note=“Description of Artificial Sequence Synthetic peptide”
MEAAVAAPRPRLLLLVLAAAAAAAAA

SEQ ID NO: 192 - protein, 481 aa, Artificial Sequence
  source: /note=“Description of Artificial Sequence Synthetic polypeptide”
  VARIANT (330)..(330): /replace=“ ”
  SITE (1)..(481): /note=“Variant residues given in the sequence have no preference with respect to those in the annotations for variant positions”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 193 - protein, 481 aa, Artificial Sequence
  source: /note=“Description of Artificial Sequence Synthetic polypeptide”
  VARIANT (330)..(330): /replace=“ ”
  SITE (1)..(481): /note=“Variant residues given in the sequence have no preference with respect to those in the annotations for variant positions”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 194 - protein, 378 aa, Artificial Sequence
  source: /note=“Description of Artificial Sequence Synthetic polypeptide”
  VARIANT (227)..(227): /replace=“ ”
  SITE (1)..(378): /note=“Variant residues given in the sequence have no preference with respect to those in the annotations for variant positions”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 195 - protein, 378 aa, Artificial Sequence
  source: /note=“Description of Artificial Sequence Synthetic polypeptide”
  VARIANT (227)..(227): /replace=“ ”
  SITE (1)..(378): /note=“Variant residues given in the sequence have no preference with respect to those in the annotations for variant positions”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 196 - protein, 481 aa, Artificial Sequence
  source: /note=“Description of Artificial Sequence Synthetic polypeptide”
  VARIANT (481)..(481): /replace=“ ”
  SITE (1)..(481): /note=“Variant residues given in the sequence have no preference with respect to those in the annotations for variant positions”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 197 - protein, 481 aa, Artificial Sequence
  source: /note=“Description of Artificial Sequence Synthetic polypeptide”
  VARIANT (481)..(481): /replace=“ ”
  SITE (1)..(481): /note=“Variant residues given in the sequence have no preference with respect to those in the annotations for variant positions”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 257 aa, Artificial Sequence
  source: /note=“Description of Artificial Sequence Synthetic polypeptide”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 199 - protein, 258 aa, Artificial Sequence
  source: /note=“Description of Artificial Sequence Synthetic polypeptide”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0377571 A1
(43) Pub. Date: Dec. 3, 2020

(54) MULTISPECIFIC MOLECULES AND USES THEREOF

(71) Applicant: ELSTAR THERAPEUTICS, INC., Cambridge, MA (US)

(72) Inventors: Andreas Loew, Boston, MA (US); Brian Edward Vash, Cambridge, MA (US); Stephanie J. Maiocco, Arlington, MA (US)

(21) Appl. No.: 16/770,467

(22) PCT Filed: Dec. 7, 2018
(86) PCT No.: PCT/US2018/064506

§ 371 Date: Jun. 5, 2020

Related U.S. Application Data
(60) Provisional application No. 62/596,173, filed on Dec. 8, 2017.

(51) Int. Cl. C07K 14/71; C07K 16/28

(52) U.S. Cl. CPC C07K 14/71; C07K 16/2866; C07K 16/2827; C07K 2317/522; C07K 2317/55; C07K 2317/569; C07K 2317/64; C07K 2317/515; C07K 2317/54; C07K 2317/51

(57) ABSTRACT

Multispecific molecules comprising (i) a TGF-beta inhibitor and (ii) a binding moiety that binds to CSF1R or CCR2, and methods of using the same, are disclosed.

### RELATED APPLICATION

This application claims priority to U.S. Ser. No. 62/596,173 filed Dec. 8, 2017, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 6, 2018, is named E2070-7020WO_SL.txt and is 603,541 bytes in size.

### BACKGROUND

Multispecific molecules targeting tumor associated macrophages (TAMs) or myeloid derived suppressor cells (MDSCs) and methods of using the same, are disclosed.

### SUMMARY OF THE INVENTION

The disclosure relates, inter alia, to novel multispecific molecules comprising: (i) a first immunosuppressive myeloid cell (IMC) binding moiety (e.g., a first tumor associated macrophage (TAM) binding moiety; or a first myeloid derived suppressor cell (MDSC) binding moiety) (e.g., an antibody molecule); and (ii) a second IMC binding moiety (e.g., a first TAM binding moiety; or a second MDSC binding moiety) (e.g., an antibody molecule), wherein the first and the second IMC (e.g., TAM or MDSC) binding moieties are different. Without being bound by theory, the multispecific molecules disclosed herein are expected to deplete TAMs and/or MDSCs. Accordingly, provided herein are, inter alia, multispecific molecules (e.g., multispecific antibody molecules) that include the aforesaid moieties, nucleic acids encoding the same, methods of producing the aforesaid molecules, and methods of treating a cancer using the aforesaid molecules.

In one aspect, provided herein are isolated multispecific, e.g., a bispecific, molecules, comprising: (i) a first immunosuppressive myeloid cell (IMC) binding moiety (e.g., a first tumor associated macrophage (TAM) binding moiety; or a first myeloid derived suppressor cell (MDSC) binding moiety) (e.g., an antibody molecule); and (ii) a second IMC binding moiety (e.g., a second TAM binding moiety; or a second MDSC binding moiety) (e.g., an antibody molecule), wherein the first and the second IMC (e.g., TAM or MDSC) binding moieties are different.

In some embodiments, the first IMC binding moiety is a first MDSC binding moiety; and the second IMC binding moiety is a second MDSC binding moiety. In some embodiments, the first IMC binding moiety is a first TAM binding moiety; and the second IMC binding moiety is a second TAM binding moiety. In some embodiments, the first TAM binding moiety binds to CSF1R, CCR2, CXCR2, CD86, CD163, CX3CR1, MARCO, CD204, CD52 or folate receptor beta; and the second TAM binding moiety binds to CSF1R, CCR2, CXCR2, CD86, CD163, CX3CR1, MARCO, CD204, CD52 or folate receptor beta. In some embodiments, the first TAM binding moiety binds to CSF1R, CCR2, or CXCR2 (e.g., human CSF1R, CCR2, or CXCR2) and the second TAM binding moiety binds to CSF1R, CCR2, or CXCR2 (e.g., human CSF1R, CCR2, or CXCR2). In some embodiments, the first TAM binding moiety binds to CSF1R and the second TAM binding moiety binds to CCR2. In some embodiments, the first TAM binding moiety binds to CSF1R and the second TAM binding moiety binds to CXCR2. In some embodiments, the first TAM binding moiety binds to CCR2 and the second TAM binding moiety binds to CXCR2.

In some embodiments, the first TAM binding moiety binds to CSF1R, CCR2, or CXCR2 with a dissociation constant of less than about 10 nM, and more typically, 10-100 pM; and the second TAM binding moiety binds to CSF1R, CCR2, or CXCR2 with a dissociation constant of less than about 10 nM, and more typically, 10-100 pM. In some embodiments, the first TAM binding moiety binds to a conformational or a linear epitope on CSF1R, CCR2, or CXCR2; and the second TAM binding moiety binds to a conformational or a linear epitope on CSF1R, CCR2, or CXCR2.

In some embodiments, the multispecific molecule comprises at least two non-contiguous polypeptide chains. In some embodiments, the first IMC binding moiety comprises a first anti-IMC antibody molecule and/or the second IMC binding moiety comprises a second anti-IMC antibody molecule. In some embodiments, the first anti-IMC antibody molecule and the second anti-IMC antibody molecule are, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)).

In some embodiments, the first anti-IMC antibody molecule and/or the second anti-IMC antibody molecule comprises a heavy chain constant region chosen from IgG1, IgG2, IgG3, or IgG4, or a fragment thereof.

In some embodiments, the first anti-IMC antibody molecule and/or the second anti-IMC antibody molecule comprises a light chain constant region chosen from the light chain constant regions of kappa or lambda, or a fragment thereof. In some embodiments, the first anti-IMC antibody molecule comprises a kappa light chain constant region, or a fragment thereof, and the second anti-IMC antibody molecule comprises a lambda light chain constant region, or a fragment thereof. In some embodiments, the first anti-IMC antibody molecule comprises a lambda light chain constant region, or a fragment thereof, and the second anti-IMC antibody molecule comprises a kappa light chain constant region, or a fragment thereof. In some embodiments, the first anti-IMC antibody molecule and the second anti-IMC antibody molecule have a common light chain variable region.

In some embodiments the multispecific molecule further comprises a heavy chain constant region (e.g., an Fc region) chosen from the heavy chain constant regions of IgG1, IgG2, and IgG4, more particularly, the heavy chain constant region of human IgG1, IgG2 or IgG4. In some embodiments, the heavy chain constant region (e.g., an Fc region) is linked to, e.g., covalently linked to, one or both of the first anti-IMC antibody molecule and the second anti-IMC antibody molecule. In some embodiments, the heavy chain constant region (e.g., an Fc region) is altered, e.g., mutated, to increase or decrease one or more of: Fc receptor binding, antibody glycosylation, the number of cysteine residues, effector cell function, or complement function. In some embodiments, an interface of a first and second heavy chain constant regions (e.g., Fc region) is altered, e.g., mutated, to increase or decrease dimerization, e.g., relative to a non-engineered interface. In some embodiments, the dimerization of the heavy chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface. In some embodiments, the heavy chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. In some embodiments, the heavy chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof, numbered based on the Eu numbering system.

In some embodiments, the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region. In some embodiments, the first anti-IMC antibody molecule comprises a first heavy chain constant region (e.g., a first Fc region) and the second anti-IMC antibody molecule comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In some embodiments, the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions. In some embodiments, the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system. In some embodiments, the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

In some embodiments, the multispecific molecule further comprises a linker, e.g., a linker between one or more of: the first anti-IMC antibody molecule and the second anti-IMC antibody molecule, the first anti-IMC antibody molecule and the heavy chain constant region (e.g., the Fc region), or the second anti-IMC antibody molecule and the heavy chain constant region. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser.

In some embodiments, the heavy chain constant region (e.g., Fc region) induces antibody dependent cellular cytotoxicity (ADCC).

In some embodiments, the first or the second TAM binding moiety is an antibody molecule that binds to CSF1R and comprises one, two, or three CDRs from the heavy chain variable region sequence of: SEQ ID NO: 48, SEQ ID NO: 66, or SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48, SEQ ID NO: 66, or SEQ ID NO: 69; and/or comprises one, two, or three CDRs from the light chain variable region sequence of: SEQ ID NO: 50, SEQ ID NO: 67, or SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50, SEQ ID NO: 67, or SEQ ID NO: 70. In some embodiments, the antibody molecule that binds to CSF1R comprises the heavy chain variable region sequence of: SEQ ID NO: 48, SEQ ID NO: 66, or SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48, SEQ ID NO: 66, or SEQ ID NO: 69; and/or comprises the light chain variable region sequence of: SEQ ID NO: 50, SEQ ID NO: 67, or SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50, SEQ ID NO: 67, or SEQ ID NO: 70.

In some embodiments, the first or the second TAM binding moiety is an antibody molecule that binds to CCR2 and comprises one, two, or three CDRs from the heavy chain variable region sequence of: SEQ ID NO: 44, SEQ ID NO: 54, SEQ ID NO: 59, SEQ ID NO: 62, SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 44, SEQ ID NO: 54, SEQ ID NO: 59, SEQ ID NO: 62, SEQ ID NO: 64; and/or comprises one, two, or three CDRs from the light chain variable region sequence of: SEQ ID NO: 45, SEQ ID NO: 57, SEQ ID NO: 60, SEQ ID NO: 63, SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 45, SEQ ID NO: 57, SEQ ID NO: 60, SEQ ID NO: 63, SEQ ID NO: 65. In some embodiments, the antibody molecule that binds to CCR2 comprises the heavy chain variable region sequence of: SEQ ID NO: 44, SEQ ID NO: 54, SEQ ID NO: 59, SEQ ID NO: 62, SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 44, SEQ ID NO: 54, SEQ ID NO: 59, SEQ ID NO: 62, SEQ ID NO: 64; and/or comprises the light chain variable region sequence of: SEQ ID NO: 45, SEQ ID NO: 57, SEQ ID NO: 60, SEQ ID NO: 63, SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 45, SEQ ID NO: 57, SEQ ID NO: 60, SEQ ID NO: 63, SEQ ID NO: 65.

In one embodiment, the first TAM binding moiety is an antibody molecule that binds to CCR2 and comprises one, two, or three CDRs from the heavy chain variable region sequence of: SEQ ID NO: 44, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 44; and/or comprises one, two, or three CDRs from the light chain variable region sequence of: SEQ ID NO: 45, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 45; and the second TAM binding moiety is an antibody molecule that binds to CSF1R and comprises one, two, or three CDRs from the heavy chain variable region sequence of: SEQ ID NO: 48, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48; and/or comprises one, two, or three CDRs from the light chain variable region sequence of: SEQ ID NO: 50, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50.

In one embodiment, the first TAM binding moiety is an antibody molecule that binds to CCR2 and comprises one, two, or three CDRs from the heavy chain variable region sequence of: SEQ ID NO: 54, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 54; and/or comprises one, two, or three CDRs from the light chain variable region sequence of: SEQ ID NO: 57, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 57; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 54, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 54; and/or comprises one, two, or three CDRs from the light chain variable region sequence of: SEQ ID NO: 57, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 57; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 59, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 59; and/or comprises one, two, or three CDRs from the light chain variable region sequence of: SEQ ID NO: 60, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 60; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 59, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 59; and/or comprises one, two, or three CDRs from the light chain variable region sequence of: SEQ ID NO: 60, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 60; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 62, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 62; and/or comprises one, two, or three CDRs from the light chain variable region sequence of: SEQ ID NO: 63, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 63; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 62, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 62; and/or comprises one, two, or three CDRs from the light chain variable region sequence of: SEQ ID NO: 63, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 63; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises one, two, or three CDRs from the heavy chain variable region sequence of: SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 64; and/or comprises one, two, or three CDRs from the light chain variable region sequence of: SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 65; and the second TAM binding moiety is an antibody molecule that binds to CSF1R and comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67.

In one embodiment, the first TAM binding moiety is an antibody molecule that binds to CCR2 and comprises one, two, or three CDRs from the heavy chain variable region sequence of: SEQ ID NO: 64, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 64; and/or comprises one, two, or three CDRs from the light chain variable region sequence of: SEQ ID NO: 65, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 65; and the second TAM binding moiety is an antibody molecule that binds to CSF1R and comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70.

In one embodiment, the first TAM binding moiety is an antibody molecule that binds to CCR2 and comprises the heavy chain variable region sequence of: SEQ ID NO: 44, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 44; and/or comprises the light chain variable region sequence of: SEQ ID NO: 45, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 45; and the second TAM binding moiety is an antibody molecule that binds to CSF1R and comprises the heavy chain variable region sequence of: SEQ ID NO: 48, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48; and/or comprises the light chain variable region sequence of: SEQ ID NO: 50, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50.

In one embodiment, the first TAM binding moiety is an antibody molecule that binds to CCR2 and comprises the heavy chain variable region sequence of: SEQ ID NO: 54, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 54; and/or comprises the light chain variable region sequence of: SEQ ID NO: 57, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 57; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 54, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 54; and/or comprises the light chain variable region sequence of: SEQ ID NO: 57, or a an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 57; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In one embodiment, the first TAM binding moiety is an antibody molecule that binds to CCR2 and comprises the heavy chain variable region sequence of: SEQ ID NO: 59, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 59; and/or comprises the light chain variable region sequence of: SEQ ID NO: 60, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 60; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 59, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 59; and/or comprises the light chain variable region sequence of: SEQ ID NO: 60, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 60; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In one embodiment, the first TAM binding moiety is an antibody molecule that binds to CCR2 and comprises the heavy chain variable region sequence of: SEQ ID NO: 62, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 62; and/or comprises the light chain variable region sequence of: SEQ ID NO: 63, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 63; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 62, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 62; and/or comprises the light chain variable region sequence of: SEQ ID NO: 63, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 63; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In one embodiment, the first TAM binding moiety is an antibody molecule that binds to CCR2 and comprises the heavy chain variable region sequence of: SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 64; and/or comprises the light chain variable region sequence of: SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 65; and the second TAM binding moiety is an antibody molecule that binds to CSF1R and comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66; and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67.

In one embodiment, the first TAM binding moiety is an antibody molecule that binds to CCR2 and comprises the heavy chain variable region sequence of: SEQ ID NO: 64, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 64; and/or comprises the light chain variable region sequence of: SEQ ID NO: 65, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 65; and the second TAM binding moiety is an antibody molecule that binds to CSF1R and comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69; and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70.

In some embodiments, the multispecific molecule further comprises one or more additional binding moieties (e.g., a third binding moiety, a fourth binding moiety, (e.g., a trispecific or a tetraspecific molecule). In some embodiments, the multispecific molecule further comprises one or more additional binding moieties (e.g., a third binding moiety, a fourth binding moiety, (e.g., a trispecific or a tetraspecific molecule). In some embodiments, the multispecific molecule comprises a third TAM binding moiety (e.g., an antibody molecule), wherein the third TAM binding moiety is different from the first and the second TAM binding moieties. In some embodiments, the first TAM binding moiety binds to human CSF1R, the second TAM binding moiety binds to human CCR2, and the third TAM binding moiety binds to CXCR2.

In some embodiments, the multispecific molecule comprises a third binding moiety (e.g., antibody molecule) that is a tumor targeting moiety. In some embodiments, the tumor targeting moiety binds to PD-L1, mesothelin, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pme117, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUMS, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In some embodiments, the multispecific molecule is a bispecific molecule, wherein:

(i) the first TAM binding moiety (e.g., a binding moiety that binds to a first TAM antigen, e.g., CSF1R, CCR2, or CXCR2) comprises a first and a second non-contiguous polypeptides, and

(ii) the second TAM binding moiety (e.g., a binding moiety that binds to a second TAM antigen, e.g., CSF1R, CCR2, or CXCR2) comprises a third and a fourth non-contiguous polypeptides, wherein:

(a) the first polypeptide comprises, e.g., in the N- to C-orientation, a first VH, a first CH1, connected, optionally via a linker, to a first domain (e.g., a first Fc region) that promotes association between the first and the third polypeptides,

(b) the second polypeptide comprises, e.g., in the N- to C-orientation, a first VL and a first CL,

(c) the third polypeptide comprises, e.g., in the N- to C-orientation, a second VH, a second CH1, connected, optionally via a linker, to a second domain (e.g., a second Fc region) that promotes association between the first and the third polypeptides, and

(d) the fourth polypeptide comprises, e.g., in the N- to C-orientation, a second VL and a second CL. In some embodiments, the first and the second domains (e.g., the first and the second Fc regions) form a homo- or heterodimer.

In certain embodiments of the foregoing aspects, the multispecific molecule further comprises a TGF-beta inhibitor. In some embodiments, the TGF-beta inhibitor sequesters TGF-beta such that it can no longer interact and signal through its endogenous membrane-bound receptor. In some embodiments, the TGF-beta inhibitor reduces the activity of one, two, or all of: (i) TGF-beta 1, (ii) TGF-beta 2, or (iii) TGF-beta 3. In some embodiments, the TGF-beta inhibitor reduces the activity of: TGF-beta 1 and TGF-beta 3. In some embodiments, the TGF-beta inhibitor reduces the activity of: TGF-beta 1, TGF-beta 2, and TGF-beta 3.

In some embodiments, the TGF-beta inhibitor is linked, e.g., via a linker, to the first IMC binding moiety (e.g., a first TAM binding moiety or a first MDSC binding moiety) or the second IMC binding moiety (e.g., a second TAM binding moiety or a second MDSC binding moiety). In some embodiments, the multispecific molecule comprises a first TGF-beta inhibitor and a second TGF-beta inhibitor, wherein the first TGF-beta inhibitor is linked, e.g., via a linker, to the first IMC binding moiety (e.g., a first TAM binding moiety or a first MDSC binding moiety) and wherein the second TGF-beta inhibitor is linked, e.g., via a linker, to the second IMC binding moiety (e.g., a second TAM binding moiety or a second MDSC binding moiety).

In some embodiments, the first IMC binding moiety (e.g., a first TAM binding moiety or a first MDSC binding moiety) comprises a first anti-IMC antibody molecule (e.g., a first anti-TAM antibody molecule or a first anti-MDSC antibody molecule) comprising a first heavy chain polypeptide (e.g., a first heavy chain polypeptide comprising a first heavy chain variable region and a first heavy chain constant region (e.g., a first Fc region)) and a first light chain polypeptide (e.g., a first light chain polypeptide comprising a first light chain variable region and a first light chain constant region), and the second IMC binding moiety (e.g., a second TAM binding moiety or a second MDSC binding moiety) comprises a second anti-IMC antibody molecule (e.g., a second anti-TAM antibody molecule or a second anti-MDSC antibody molecule) comprising a second heavy chain polypeptide (e.g., a second heavy chain polypeptide comprising a second heavy chain variable region and a second heavy chain constant region (e.g., a second Fc region)) and a second light chain polypeptide (e.g., a second light chain polypeptide comprising a second light chain variable region and a second light chain constant region), wherein:

(a) the TGF-beta inhibitor is linked, e.g., via a linker, to the first anti-IMC antibody molecule (e.g., a first anti-TAM antibody molecule or a first anti-MDSC antibody molecule) or the second anti-IMC antibody molecule (e.g., a second anti-TAM antibody molecule or a second anti-MDSC antibody molecule),

(b) the multispecific molecule comprises a first TGF-beta inhibitor and a second TGF-beta inhibitor, wherein the first TGF-beta inhibitor is linked, e.g., via a linker, to the first anti-IMC antibody molecule (e.g., a first anti-TAM antibody molecule or a first anti-MDSC antibody molecule) and wherein the second TGF-beta inhibitor is linked, e.g., via a linker, to the second anti-IMC antibody molecule (e.g., a second anti-TAM antibody molecule or a second anti-MDSC antibody molecule),

(c) the TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) or the second heavy chain polypeptide (e.g., the Fc region of the second heavy chain polypeptide, e.g., the C-terminus of the Fc region of the second heavy chain polypeptide),

(d) the multispecific molecule comprises a first TGF-beta inhibitor and a second TGF-beta inhibitor, wherein the first TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) and wherein the second TGF-beta inhibitor is linked, e.g., via a linker, to the second heavy chain polypeptide (e.g., the Fc region of the second heavy chain polypeptide, e.g., the C-terminus of the Fc region of the second heavy chain polypeptide),

(e) the TGF-beta inhibitor is linked, e.g., via a linker, to the first light chain polypeptide (e.g., the constant region of the first light chain polypeptide, e.g., the C-terminus of the constant region of the first light chain polypeptide) or the second light chain polypeptide (e.g., the constant region of the second light chain polypeptide, e.g., the C-terminus of the constant region of the second light chain polypeptide), or

(f) the multispecific molecule comprises a first TGF-beta inhibitor and a second TGF-beta inhibitor, wherein the first TGF-beta inhibitor is linked, e.g., via a linker, to the first light chain polypeptide (e.g., the constant region of the first light chain polypeptide, e.g., the C-terminus of the constant region of the first light chain polypeptide) and wherein the second TGF-beta inhibitor is linked, e.g., via a linker, to the second light chain polypeptide (e.g., the constant region of the second light chain polypeptide, e.g., the C-terminus of the constant region of the second light chain polypeptide).

In some embodiments, the multispecific molecule comprises:

(i) a first polypeptide comprising a first portion of the first IMC binding moiety (e.g., a first TAM binding moiety or a first MDSC binding moiety) comprising a first VL and a first CL;

(ii) a second polypeptide comprising (1) a second portion of the first IMC binding moiety (e.g., a first TAM binding moiety or a first MDSC binding moiety) comprising a first VH, a first CH1, a first CH2, and a first CH3, and optionally (2) a first TGF-beta inhibitor;

(iii) a third polypeptide comprising (1) a first portion of the second IMC binding moiety (e.g., a second TAM binding moiety or a second MDSC binding moiety) comprising a second VH, a second CH1, a second CH2, and a second CH3, and optionally (2) a second TGF-beta inhibitor; and

(iv) a fourth polypeptide comprising a second portion of the second IMC binding moiety (e.g., a second TAM binding moiety or a second MDSC binding moiety) comprising a second VL and a second CL, wherein:

the multispecific molecule comprises at least one of: the first TGF-beta inhibitor or the second TGF-beta inhibitor, optionally wherein the first and the second TGF-beta inhibitors form a homo-dimer or hetero-dimer. In some embodiments, the multispecific molecule has the configuration of any one of FIGS. 1A-1J.

In some embodiments, the multispecific molecule comprises:

(i) a first polypeptide comprising a first portion of the first IMC binding moiety (e.g., a first TAM binding moiety or a first MDSC binding moiety) comprising a first VL and a first CL; (ii) a second polypeptide comprising (1) a second portion of the first IMC binding moiety (e.g., a first TAM binding moiety or a first MDSC binding moiety) comprising a first VH, a first CH1, a first CH2, and a first CH3, and (2) the second IMC binding moiety (e.g., a second TAM binding moiety or a second MDSC binding moiety) comprising a second VH and a second VL (e.g., an scFv);

(iii) a third polypeptide comprising a first TGF-beta inhibitor, a second CH1, a second CH2, and a second CH3; and

(iv) a fourth polypeptide comprising a second TGF-beta inhibitor, and a second CL, optionally wherein the first and the second TGF-beta inhibitors form a homo-dimer or hetero-dimer. In some embodiments, the multispecific molecule has the configuration of any one of FIGS. 2A-2D and 3A-3D.

In some embodiments, the multispecific molecule comprises:

(i) a first polypeptide comprising a first TGF-beta inhibitor and a first CL;

(ii) a second polypeptide comprising (1) a second TGF-beta inhibitor, a first CH1, a first CH2, and a first CH3, and (2) the first IMC binding moiety (e.g., a first TAM binding moiety or a first MDSC binding moiety) comprising a first VH and a first VL (e.g., a first scFv);

(iii) a third polypeptide comprising (1) a third TGF-beta inhibitor, a second CH1, a second CH2, and a second CH3, and (2) the second IMC binding moiety (e.g., a second TAM binding moiety or a second MDSC binding moiety) comprising a second VH and a second VL (e.g., a second scFv);

(iv) a fourth polypeptide comprising a fourth TGF-beta inhibitor and a second CL,

optionally wherein the first and the second TGF-beta inhibitors form a homo-dimer or hetero-dimer, and/or the third and the fourth TGF-beta inhibitors form a homo-dimer or hetero-dimer. In some embodiments, the multispecific molecule has the configuration of any one of FIGS. 4A-4D.

In some embodiments, the multispecific molecule comprises:

(i) a first polypeptide comprising (1) a first TGF-beta inhibitor, a first CH2, and a first CH3, and (2) the first IMC binding moiety (e.g., a first TAM binding moiety or a first MDSC binding moiety) comprising a first VH and a first VL (e.g., a first scFv); and

(ii) a second polypeptide comprising (1) a second TGF-beta inhibitor, a second CH2, and a second CH3, and (2) the second IMC binding moiety (e.g., a second TAM binding moiety or a second MDSC binding moiety) comprising a second VH and a second VL (e.g., a second scFv). In some embodiments, the multispecific molecule has the configuration of any one of FIGS. 5A-5B.

In some embodiments, the multispecific molecule comprises a first TGF-beta inhibitor and a second TGF-beta inhibitor, wherein the first TGF-beta inhibitor and the second TGF-beta inhibitor form a dimer.

In some embodiments, the TGF-beta inhibitor, or the first, second, third, or fourth TGF-beta inhibitor comprises a TGF-beta receptor polypeptide (e.g., an extracellular domain of a TGF-beta receptor, or a functional variant thereof). In some embodiments, the TGF-beta inhibitor, or the first, second, third, or fourth TGF-beta inhibitor comprises one, two, or all of:

(i) a TGFBR1 polypeptide (e.g., 1, 2, 3, or more of a TGFBR1 polypeptide),

(ii) a TGFBR2 polypeptide (e.g., 1, 2, 3, or more of a TGFBR2 polypeptide), or

(iii) a TGFBR3 polypeptide (e.g., 1, 2, 3, or more of a TGFBR3 polypeptide).

In some embodiments, the TGF-beta inhibitor, or the first, second, third, or fourth TGF-beta inhibitor comprises a TGFBR1 polypeptide, e.g., the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR1 polypeptide, e.g., the TGF-beta inhibitor comprises an extracellular domain of SEQ ID NO: 95, 96, 97, 120, 121, or 122 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR1 polypeptide, e.g., the TGF-beta inhibitor comprises the amino acid sequence of SEQ ID NO: 104 or 105, or a sequence substantially identical thereto (e.g., a sequence that is at least 80%, 85%, 90%, or 95% identical thereto).

In some embodiments, the TGF-beta inhibitor, or the first, second, third, or fourth TGF-beta inhibitor comprises a TGFBR2 polypeptide, e.g., the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR2 polypeptide, e.g., the TGF-beta inhibitor comprises an extracellular domain of SEQ ID NO: 98, 99, 123, or 124, or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR2 polypeptide, e.g., the TGF-beta inhibitor comprises an amino acid sequence selected from the group consisting of SEQ ID NOs: 100, 101, 102, and 103, or a sequence substantially identical thereto (e.g., a sequence that is at least 80%, 85%, 90%, or 95% identical thereto).

In some embodiments, the TGF-beta inhibitor, or the first, second, third, or fourth TGF-beta inhibitor comprises a TGFBR3 polypeptide, e.g., the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR3 polypeptide, e.g., the TGF-beta inhibitor comprises an extracellular domain of SEQ ID NO: 106, 107, 125, or 126, or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR3 polypeptide, e.g., the TGF-beta inhibitor comprises the amino acid sequence of SEQ ID NO: 108, or a sequence substantially identical thereto (e.g., a sequence that is at least 80%, 85%, 90%, or 95% identical thereto).

In one aspect, disclosed herein is an isolated multispecific molecule comprising:

(i) a CSF1R binding moiety (e.g., an anti-CSF1R antibody molecule),

(ii) a PD-L1 binding moiety (e.g., an anti-PD-L1 antibody molecule), and

(iii) a TGF-beta inhibitor.

In some embodiments, the TGF-beta inhibitor sequesters TGF-beta such that it can no longer interact and signal through its endogenous membrane-bound receptor.

In some embodiments, the CSF1R binding moiety (e.g., an anti-CSF1R antibody molecule) is a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)). In some embodiments, the PD-L1 binding moiety (e.g., an anti-PD-L1 antibody molecule) is a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)). In some embodiments, the CSF1R binding moiety (e.g., an anti-CSF1R antibody molecule) comprises a light chain constant region chosen from the light chain constant regions of kappa or lambda, or a fragment thereof. In some embodiments, the PD-L1 binding moiety (e.g., an anti-PD-L1 antibody molecule) comprises a light chain constant region chosen from the light chain constant regions of kappa or lambda, or a fragment thereof. In some embodiments, the CSF1R binding moiety (e.g., an anti-CSF1R antibody molecule) comprises a kappa light chain constant region, or a fragment thereof, and the PD-L1 binding moiety (e.g., an anti-PD-L1 antibody molecule) comprises a lambda light chain constant region, or a fragment thereof. In some embodiments, the CSF1R binding moiety (e.g., an anti-CSF1R antibody molecule) comprises a lambda light chain constant region, or a fragment thereof, and the PD-L1 binding moiety (e.g., an anti-PD-L1 antibody molecule) comprises a kappa light chain constant region, or a fragment thereof. In some embodiments, the CSF1R binding moiety (e.g., an anti-CSF1R antibody molecule) and the PD-L1 binding moiety (e.g., an anti-PD-L1 antibody molecule) have a common light chain variable region.

In some embodiments, the CSF1R binding moiety (e.g., an anti-CSF1R antibody molecule) comprises a heavy chain constant region (e.g., a CH1 region and an Fc region) chosen from IgG1, IgG2, IgG3, or IgG4, or a fragment thereof. In some embodiments, the PD-L1 binding moiety (e.g., an anti-PD-L1 antibody molecule) comprises a heavy chain constant region (e.g., a CH1 region and an Fc region) chosen from IgG1, IgG2, IgG3, or IgG4, or a fragment thereof. In some embodiments, the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region.

In some embodiments, the CSF1R binding moiety (e.g., an anti-CSF1R antibody molecule) comprises a first heavy chain constant region (e.g., a first Fc region) and the PD-L1 binding moiety (e.g., an anti-PD-L1 antibody molecule) comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In some embodiments, the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions. In some embodiments, the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system. In some embodiments, the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

In some embodiments, the CSF1R binding moiety (e.g., an anti-CSF1R antibody molecule) comprises one, two, or three CDRs from the heavy chain variable region sequence of: SEQ ID NO: 48, SEQ ID NO: 66, or SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48, SEQ ID NO: 66, or SEQ ID NO: 69; and/or comprises one, two, or three CDRs from the light chain variable region sequence of: SEQ ID NO: 50, SEQ ID NO: 67, or SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50, SEQ ID NO: 67, or SEQ ID NO: 70. In some embodiments, the CSF1R binding moiety (e.g., an anti-CSF1R antibody molecule) comprises the heavy chain variable region sequence of: SEQ ID NO: 48, SEQ ID NO: 66, or SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48, SEQ ID NO: 66, or SEQ ID NO: 69); and/or comprises the light chain variable region sequence of: SEQ ID NO: 50, SEQ ID NO: 67, or SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50, SEQ ID NO: 67, or SEQ ID NO: 70).

In some embodiments, the CSF1R binding moiety (e.g., an anti-CSF1R antibody molecule) comprises one, two, or three CDRs from the heavy chain variable region sequence of: SEQ ID NO: 48, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 48; and/or comprises one, two, or three CDRs from the light chain variable region sequence of: SEQ ID NO: 50, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 50. In some embodiments, the CSF1R binding moiety (e.g., an anti-CSF1R antibody molecule) comprises the heavy chain variable region sequence of: SEQ ID NO: 48, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 48); and/or comprises the light chain variable region sequence of: SEQ ID NO: 50, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 50).

In some embodiments, the CSF1R binding moiety (e.g., an anti-CSF1R antibody molecule) comprises one, two, or three CDRs from the heavy chain variable region sequence of: SEQ ID NO: 66, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 66; and/or comprises one, two, or three CDRs from the light chain variable region sequence of: SEQ ID NO: 67, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 67. In some embodiments, the CSF1R binding moiety (e.g., an anti-CSF1R antibody molecule) comprises the heavy chain variable region sequence of: SEQ ID NO: 66, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 66); and/or comprises the light chain variable region sequence of: SEQ ID NO: 67, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 67).

In some embodiments, the CSF1R binding moiety (e.g., an anti-CSF1R antibody molecule) comprises one, two, or three CDRs from the heavy chain variable region sequence of: SEQ ID NO: 69, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 69; and/or comprises one, two, or three CDRs from the light chain variable region sequence of: SEQ ID NO: 70, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 70. In some embodiments, the CSF1R binding moiety (e.g., an anti-CSF1R antibody molecule) comprises the heavy chain variable region sequence of: SEQ ID NO: 69, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 69); and/or comprises the light chain variable region sequence of: SEQ ID NO: 70, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 70).

In some embodiments, the PD-L1 binding moiety (e.g., an anti-PD-L1 antibody molecule) comprises one, two, or three CDRs from the heavy chain variable region sequence of: SEQ ID NO: 109, SEQ ID NO: 111, or SEQ ID NO: 113, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 109, SEQ ID NO: 111, or SEQ ID NO: 113; and/or comprises one, two, or three CDRs from the light chain variable region sequence of: SEQ ID NO: 110, SEQ ID NO: 112, or SEQ ID NO: 114, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 110, SEQ ID NO: 112, or SEQ ID NO: 114 In some embodiments, the PD-L1 binding moiety (e.g., an anti-PD-L1 antibody molecule) comprises the heavy chain variable region sequence of: SEQ ID NO: 109, SEQ ID NO: 111, or SEQ ID NO: 113, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 109, SEQ ID NO: 111, or SEQ ID NO: 113); and/or comprises the light chain variable region sequence of: SEQ ID NO: 110, SEQ ID NO: 112, or SEQ ID NO: 114, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 110, SEQ ID NO: 112, or SEQ ID NO: 114).

In some embodiments, the PD-L1 binding moiety (e.g., an anti-PD-L1 antibody molecule) comprises one, two, or three CDRs from the heavy chain variable region sequence of: SEQ ID NO: 109, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 109; and/or comprises one, two, or three CDRs from the light chain variable region sequence of: SEQ ID NO: 110, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 110. In some embodiments, the PD-L1 binding moiety (e.g., an anti-PD-L1 antibody molecule) comprises the heavy chain variable region sequence of: SEQ ID NO: 109, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 109); and/or comprises the light chain variable region sequence of: SEQ ID NO: 110, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 110).

In some embodiments, the PD-L1 binding moiety (e.g., an anti-PD-L1 antibody molecule) comprises one, two, or three CDRs from the heavy chain variable region sequence of: SEQ ID NO: 111, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 111; and/or comprises one, two, or three CDRs from the light chain variable region sequence of: SEQ ID NO: 112, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 112. In some embodiments, the PD-L1 binding moiety (e.g., an anti-PD-L1 antibody molecule) comprises the heavy chain variable region sequence of: SEQ ID NO: 111, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 111); and/or comprises the light chain variable region sequence of: SEQ ID NO: 112, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 112).

In some embodiments, the PD-L1 binding moiety (e.g., an anti-PD-L1 antibody molecule) comprises one, two, or three CDRs from the heavy chain variable region sequence of: SEQ ID NO: 113, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 113; and/or comprises one, two, or three CDRs from the light chain variable region sequence of: SEQ ID NO: 114, or a closely related CDR, e.g., CDRs which have at least one amino acid alteration, but not more than two, three or four alterations (e.g., substitutions, deletions, or insertions, e.g., conservative substitutions) from a CDR of SEQ ID NO: 114. In some embodiments, the PD-L1 binding moiety (e.g., an anti-PD-L1 antibody molecule) comprises the heavy chain variable region sequence of: SEQ ID NO: 113, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 113); and/or comprises the light chain variable region sequence of: SEQ ID NO: 114, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 114).

In some embodiments, the TGF-beta inhibitor reduces the activity of one, two, or all of:

(i) TGF-beta 1,

(ii) TGF-beta 2, or

(iii) TGF-beta 3, optionally wherein the TGF-beta inhibitor reduces the activity of:

(a) TGF-beta 1 and TGF-beta 3, or

(b) TGF-beta 1, TGF-beta 2, and TGF-beta 3.

In some embodiments, the TGF-beta inhibitor is linked, e.g., via a linker, to the CSF1R binding moiety (e.g., an anti-CSF1R antibody molecule) or the PD-L1 binding moiety (e.g., an anti-PD-L1 antibody molecule). In some embodiments, the multispecific molecule comprises a first TGF-beta inhibitor and a second TGF-beta inhibitor, wherein the first TGF-beta inhibitor is linked, e.g., via a linker, to the CSF1R binding moiety (e.g., an anti-CSF1R antibody molecule) and wherein the second TGF-beta inhibitor is linked, e.g., via a linker, to the PD-L1 binding moiety (e.g., an anti-PD-L1 antibody molecule).

In some embodiments, the CSF1R binding moiety (e.g., an anti-CSF1R antibody molecule) comprises a first heavy chain polypeptide (e.g., a first heavy chain polypeptide comprising a first heavy chain variable region and a first heavy chain constant region (e.g., a first Fc region)) and a first light chain polypeptide (e.g., a first light chain polypeptide comprising a first light chain variable region and a first light chain constant region), and the PD-L1 binding moiety (e.g., an anti-PD-L1 antibody molecule) comprises a second heavy chain polypeptide (e.g., a second heavy chain polypeptide comprising a second heavy chain variable region and a second heavy chain constant region (e.g., a second Fc region)) and a second light chain polypeptide (e.g., a second light chain polypeptide comprising a second light chain variable region and a second light chain constant region), wherein:

(a) the TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) or the second heavy chain polypeptide (e.g., the Fc region of the second heavy chain polypeptide, e.g., the C-terminus of the Fc region of the second heavy chain polypeptide),

(b) the multispecific molecule comprises a first TGF-beta inhibitor and a second TGF-beta inhibitor, wherein the first TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) and wherein the second TGF-beta inhibitor is linked, e.g., via a linker, to the second heavy chain polypeptide (e.g., the Fc region of the second heavy chain polypeptide, e.g., the C-terminus of the Fc region of the second heavy chain polypeptide),

(c) the TGF-beta inhibitor is linked, e.g., via a linker, to the first light chain polypeptide (e.g., the constant region of the first light chain polypeptide, e.g., the C-terminus of the constant region of the first light chain polypeptide) or the second light chain polypeptide (e.g., the constant region of the second light chain polypeptide, e.g., the C-terminus of the constant region of the second light chain polypeptide), or

(d) the multispecific molecule comprises a first TGF-beta inhibitor and a second TGF-beta inhibitor, wherein the first TGF-beta inhibitor is linked, e.g., via a linker, to the first light chain polypeptide (e.g., the constant region of the first light chain polypeptide, e.g., the C-terminus of the constant region of the first light chain polypeptide) and wherein the second TGF-beta inhibitor is linked, e.g., via a linker, to the second light chain polypeptide (e.g., the constant region of the second light chain polypeptide, e.g., the C-terminus of the constant region of the second light chain polypeptide).

In some embodiments, the multispecific molecule comprises:

(i) a first polypeptide comprising a first portion of the CSF1R binding moiety comprising a first VL and a first CL;

(ii) a second polypeptide comprising (1) a second portion of the CSF1R binding moiety comprising a first VH, a first CH1, a first CH2, and a first CH3, and optionally (2) a first TGF-beta inhibitor;

(iii) a third polypeptide comprising (1) a first portion of the PD-L1 binding moiety comprising a second VH, a second CH1, a second CH2, and a second CH3, and optionally (2) a second TGF-beta inhibitor; and

(iv) a fourth polypeptide comprising a second portion of the PD-L1 binding moiety comprising a second VL and a second CL, wherein:

the multispecific molecule comprises at least one of: the first TGF-beta inhibitor or the second TGF-beta inhibitor, optionally wherein the first and the second TGF-beta inhibitors form a homo-dimer or hetero-dimer. In some embodiments, the multispecific molecule has the configuration of any one of FIGS. 1A-1J.

In some embodiments, the multispecific molecule comprises:

(i) a first polypeptide comprising a first portion of the CSF1R binding moiety comprising a first VL and a first CL;

(ii) a second polypeptide comprising (1) a second portion of the CSF1R binding moiety comprising a first VH, a first CH1, a first CH2, and a first CH3, and (2) the PD-L1 binding moiety comprising a second VH and a second VL (e.g., an scFv);

(iii) a third polypeptide comprising a first TGF-beta inhibitor, a second CH1, a second CH2, and a second CH3; and

(iv) a fourth polypeptide comprising a second TGF-beta inhibitor, and a second CL, optionally wherein the first and the second TGF-beta inhibitors form a homo-dimer or hetero-dimer. In some embodiments, the multispecific molecule has the configuration of any one of FIGS. 2A-2D.

In some embodiments, the multispecific molecule comprises:

(i) a first polypeptide comprising a first portion of the PD-L1 binding moiety comprising a first VL and a first CL;

(ii) a second polypeptide comprising (1) a second portion of the PD-L1 binding moiety comprising a first VH, a first CH1, a first CH2, and a first CH3, and (2) the CSF1R binding moiety comprising a second VH and a second VL (e.g., an scFv);

(iii) a third polypeptide comprising a first TGF-beta inhibitor, a second CH1, a second CH2, and a second CH3; and

(iv) a fourth polypeptide comprising a second TGF-beta inhibitor, and a second CL, optionally wherein the first and the second TGF-beta inhibitors form a homo-dimer or hetero-dimer. In some embodiments, the multispecific molecule has the configuration of any one of FIGS. 3A-3D.

In some embodiments, the multispecific molecule comprises:

(i) a first polypeptide comprising a first TGF-beta inhibitor and a first CL;

(ii) a second polypeptide comprising (1) a second TGF-beta inhibitor, a first CH1, a first CH2, and a first CH3, and (2) the PD-L1 binding moiety comprising a first VH and a first VL (e.g., a first scFv);

(iii) a third polypeptide comprising (1) a third TGF-beta inhibitor, a second CH1, a second CH2, and a second CH3, and (2) the CSF1R binding moiety comprising a second VH and a second VL (e.g., a second scFv);

(iv) a fourth polypeptide comprising a fourth TGF-beta inhibitor and a second CL,

optionally wherein the first and the second TGF-beta inhibitors form a homo-dimer or hetero-dimer, and/or the third and the fourth TGF-beta inhibitors form a homo-dimer or hetero-dimer. In some embodiments, the multispecific molecule has the configuration of any one of FIGS. 4A-4D.

In some embodiments, the multispecific molecule comprises:

(i) a first polypeptide comprising (1) a first TGF-beta inhibitor, a first CH2, and a first CH3, and (2) the PD-L1 binding moiety comprising a first VH and a first VL (e.g., a first scFv); and

(ii) a second polypeptide comprising (1) a second TGF-beta inhibitor, a second CH2, and a second CH3, and (2) the CSF1R binding moiety comprising a second VH and a second VL (e.g., a second scFv). In some embodiments, the multispecific molecule has the configuration of any one of FIGS. 5A-5B.

In some embodiments, the multispecific molecule comprises a first TGF-beta inhibitor and a second TGF-beta inhibitor, wherein the first TGF-beta inhibitor and the second TGF-beta inhibitor form a dimer.

In some embodiments, the TGF-beta inhibitor, or the first, second, third, or fourth TGF-beta inhibitor comprises a TGF-beta receptor polypeptide (e.g., an extracellular domain of a TGF-beta receptor, or a functional variant thereof). In some embodiments, the TGF-beta inhibitor, or the first, second, third, or fourth TGF-beta inhibitor comprises one, two, or all of:

(i) a TGFBR1 polypeptide (e.g., 1, 2, 3, or more of a TGFBR1 polypeptide),

(ii) a TGFBR2 polypeptide (e.g., 1, 2, 3, or more of a TGFBR2 polypeptide), or

(iii) a TGFBR3 polypeptide (e.g., 1, 2, 3, or more of a TGFBR3 polypeptide).

In some embodiments, the TGF-beta inhibitor, or the first, second, third, or fourth TGF-beta inhibitor comprises a TGFBR1 polypeptide, e.g., the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR1 polypeptide, e.g., the TGF-beta inhibitor comprises an extracellular domain of SEQ ID NO: 95, 96, 97, 120, 121, or 122, or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR1 polypeptide, e.g., the TGF-beta inhibitor comprises the amino acid sequence of SEQ ID NO: 104 or 105, or a sequence substantially identical thereto (e.g., a sequence that is at least 80%, 85%, 90%, or 95% identical thereto).

In some embodiments, the TGF-beta inhibitor, or the first, second, third, or fourth TGF-beta inhibitor comprises a TGFBR2 polypeptide, e.g., the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR2 polypeptide, e.g., the TGF-beta inhibitor comprises an extracellular domain of SEQ ID NO: 98, 99, 123, or 124, or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR2 polypeptide, e.g., the TGF-beta inhibitor comprises an amino acid sequence selected from the group consisting of SEQ ID NOs: 100, 101, 102, and 103, or a sequence substantially identical thereto (e.g., a sequence that is at least 80%, 85%, 90%, or 95% identical thereto).

In some embodiments, the TGF-beta inhibitor, or the first, second, third, or fourth TGF-beta inhibitor comprises a TGFBR3 polypeptide, e.g., the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR3 polypeptide, e.g., the TGF-beta inhibitor comprises an extracellular domain of SEQ ID NO: 106, 107, 125, or 126, or a sequence substantially identical thereto (e.g., a sequence that is at least 80%, 85%, 90%, or 95% identical thereto). In some embodiments, the TGF-beta inhibitor, or the first, second, third, or fourth TGF-beta inhibitor comprises a TGFBR3 polypeptide, e.g., the TGF-beta inhibitor comprises the amino acid sequence of SEQ ID NO: 108, or a sequence substantially identical thereto (e.g., a sequence that is at least 80%, 85%, 90%, or 95% identical thereto).

In some embodiments, the multispecific molecule comprises a first TGF-beta inhibitor (e.g., an extracellular domain of TGFBR2 or variant thereof) and a second TGF-beta inhibitor (e.g., an extracellular domain of TGFBR2 or variant thereof), wherein:

(i) the CSF1R binding moiety (e.g., an anti-CSF1R antibody molecule) comprises a first and a second non-contiguous polypeptides, and

(ii) the PD-L1 binding moiety (e.g., an anti-PD-L1 antibody molecule) comprises a third and a fourth non-contiguous polypeptides, wherein:

(a) the first polypeptide comprises, e.g., in the N- to C-orientation, a first VH, a first CH1, connected, optionally via a linker, to a first domain (e.g., a first Fc region) that promotes association between the first and the third polypeptides, wherein the C-terminus of the first domain (e.g., the C-terminus of the first Fc region) is connected, optionally via a linker, to the first TGF-beta inhibitor (e.g., an extracellular domain of TGFBR2 or variant thereof),

(b) the second polypeptide comprises, e.g., in the N- to C-orientation, a first VL and a first CL,

(c) the third polypeptide comprises, e.g., in the N- to C-orientation, a second VH, a second CH1, connected, optionally via a linker, to a second domain (e.g., a second Fc region) that promotes association between the first and the third polypeptides, wherein the C-terminus of the second domain (e.g., the C-terminus of the second Fc region) is connected, optionally via a linker, to the second TGF-beta inhibitor (e.g., an extracellular domain of TGFBR2 or variant thereof), and

(d) the fourth polypeptide comprises, e.g., in the N- to C-orientation, a second VL and a second CL. In some embodiments, the first and the second domains (e.g., the first and the second Fc regions) form a homo- or heterodimer. In some embodiments, the first and the second TGF-beta inhibitors (e.g., the first and the second extracellular domains of TGFBR2 or variants thereof) form a homo- or heterodimer.

In some embodiments, the multispecific molecule comprises a first, second, third, and fourth non-contiguous polypeptides, wherein the first, second, third, and fourth non-contiguous polypeptides comprise the amino acid sequences of: SEQ ID NOs: 176, 138, 185, and 147, respectively, or a sequence substantially identical thereto (e.g., 85% to 99% identical thereto); SEQ ID NOs: 176, 138, 186, and 148, respectively, or a sequence substantially identical thereto (e.g., 85% to 99% identical thereto); SEQ ID NOs: 176, 138, 187, and 147, respectively, or a sequence substantially identical thereto (e.g., 85% to 99% identical thereto); SEQ ID NOs: 176, 138, 188, and 148, respectively, or a sequence substantially identical thereto (e.g., 85% to 99% identical thereto); SEQ ID NOs: 176, 138, 189, and 147, respectively, or a sequence substantially identical thereto (e.g., 85% to 99% identical thereto); SEQ ID NOs: 176, 138, 190, and 148, respectively, or a sequence substantially identical thereto (e.g., 85% to 99% identical thereto); SEQ ID NOs: 177, 150, 185, and 147, respectively, or a sequence substantially identical thereto (e.g., 85% to 99% identical thereto); SEQ ID NOs: 177, 150, 186, and 148, respectively, or a sequence substantially identical thereto (e.g., 85% to 99% identical thereto); SEQ ID NOs: 177, 150, 187, and 147, respectively, or a sequence substantially identical thereto (e.g., 85% to 99% identical thereto); SEQ ID NOs: 177, 150, 188, and 148, respectively, or a sequence substantially identical thereto (e.g., 85% to 99% identical thereto); SEQ ID NOs: 177, 150, 189, and 147, respectively, or a sequence substantially identical thereto (e.g., 85% to 99% identical thereto); SEQ ID NOs: 177, 150, 190, and 148, respectively, or a sequence substantially identical thereto (e.g., 85% to 99% identical thereto); SEQ ID NOs: 178, 152, 185, and 147, respectively, or a sequence substantially identical thereto (e.g., 85% to 99% identical thereto); SEQ ID NOs: 178, 152, 186, and 148, respectively, or a sequence substantially identical thereto (e.g., 85% to 99% identical thereto); SEQ ID NOs: 178, 152, 187, and 147, respectively, or a sequence substantially identical thereto (e.g., 85% to 99% identical thereto); SEQ ID NOs: 178, 152, 188, and 148, respectively, or a sequence substantially identical thereto (e.g., 85% to 99% identical thereto); SEQ ID NOs: 178, 152, 189, and 147, respectively, or a sequence substantially identical thereto (e.g., 85% to 99% identical thereto); SEQ ID NOs: 178, 152, 190, and 147, respectively, or a sequence substantially identical thereto (e.g., 85% to 99% identical thereto); SEQ ID NOs: 179, 138, 185, and 147, respectively, or a sequence substantially identical thereto (e.g., 85% to 99% identical thereto); SEQ ID NOs: 179, 138, 186, and 148, respectively, or a sequence substantially identical thereto (e.g., 85% to 99% identical thereto); SEQ ID NOs: 179, 138, 187, and 148, respectively, or a sequence substantially identical thereto (e.g., 85% to 99% identical thereto); SEQ ID NOs: 179, 138, 188, and 148, respectively, or a sequence substantially identical thereto (e.g., 85% to 99% identical thereto); SEQ ID NOs: 179, 138, 189, and 147, respectively, or a sequence substantially identical thereto (e.g., 85% to 99% identical thereto); SEQ ID NOs: 179, 138, 190, and 148, respectively, or a sequence substantially identical thereto (e.g., 85% to 99% identical thereto); SEQ ID NOs: 180, 150, 185, and 147, respectively, or a sequence substantially identical thereto (e.g., 85% to 99% identical thereto); SEQ ID NOs: 180, 150, 186, and 148, respectively, or a sequence substantially identical thereto (e.g., 85% to 99% identical thereto); SEQ ID NOs: 180, 150, 187, and 147, respectively, or a sequence substantially identical thereto (e.g., 85% to 99% identical thereto); SEQ ID NOs: 180, 150, 188, and 148, respectively, or a sequence substantially identical thereto (e.g., 85% to 99% identical thereto); SEQ ID NOs: 180, 150, 189, and 147, respectively, or a sequence substantially identical thereto (e.g., 85% to 99% identical thereto); SEQ ID NOs: 180, 150, 190, and 148, respectively, or a sequence substantially identical thereto (e.g., 85% to 99% identical thereto); SEQ ID NOs: 181, 152, 185, and 147, respectively, or a sequence substantially identical thereto (e.g., 85% to 99% identical thereto); SEQ ID NOs: 181, 152, 186, and 148, respectively, or a sequence substantially identical thereto (e.g., 85% to 99% identical thereto); SEQ ID NOs: 181, 152, 187, and 147, respectively, or a sequence substantially identical thereto (e.g., 85% to 99% identical thereto); SEQ ID NOs: 181, 152, 188, and 148, respectively, or a sequence substantially identical thereto (e.g., 85% to 99% identical thereto); SEQ ID NOs: 181, 152, 189, and 147, respectively, or a sequence substantially identical thereto (e.g., 85% to 99% identical thereto); SEQ ID NOs: 181, 152, 190, and 148, respectively, or a sequence substantially identical thereto (e.g., 85% to 99% identical thereto); SEQ ID NOs: 145, 147, 140, and 161, respectively, or a sequence substantially identical thereto (e.g., 85% to 99% identical thereto); SEQ ID NOs: 153, 147, 140, and 161, respectively, or a sequence substantially identical thereto (e.g., 85% to 99% identical thereto); SEQ ID NOs: 154, 147, 140, and 161, respectively, or a sequence substantially identical thereto (e.g., 85% to 99% identical thereto); SEQ ID NOs: 146, 148, 140, and 161, respectively, or a sequence substantially identical thereto (e.g., 85% to 99% identical thereto); SEQ ID NOs: 155, 148, 140, and 161, respectively, or a sequence substantially identical thereto (e.g., 85% to 99% identical thereto); SEQ ID NOs: 156, 148, 140, and 161, respectively, or a sequence substantially identical thereto (e.g., 85% to 99% identical thereto); SEQ ID NOs: 145, 147, 140, and 141, respectively, or a sequence substantially identical thereto (e.g., 85% to 99% identical thereto); SEQ ID NOs: 153, 147, 140, and 141, respectively, or a sequence substantially identical thereto (e.g., 85% to 99% identical thereto); SEQ ID NOs: 154, 147, 140, and 141, respectively, or a sequence substantially identical thereto (e.g., 85% to 99% identical thereto); SEQ ID NOs: 146, 148, 140, and 141, respectively, or a sequence substantially identical thereto (e.g., 85% to 99% identical thereto); SEQ ID NOs: 155, 148, 140, and 141, respectively, or a sequence substantially identical thereto (e.g., 85% to 99% identical thereto); SEQ ID NOs: 156, 148, 140, and 141, respectively, or a sequence substantially identical thereto (e.g., 85% to 99% identical thereto); SEQ ID NOs: 145, 147, 162, and 161, respectively, or a sequence substantially identical thereto (e.g., 85% to 99% identical thereto); SEQ ID NOs: 153, 147, 162, and 161, respectively, or a sequence substantially identical thereto (e.g., 85% to 99% identical thereto); SEQ ID NOs: 154, 147, 162, and 161, respectively, or a sequence substantially identical thereto (e.g., 85% to 99% identical thereto); SEQ ID NOs: 146, 148, 162, and 161, respectively, or a sequence substantially identical thereto (e.g., 85% to 99% identical thereto); SEQ ID NOs: 155, 148, 162, and 161, respectively, or a sequence substantially identical thereto (e.g., 85% to 99% identical thereto); SEQ ID NOs: 156, 148, 162, and 161, respectively, or a sequence substantially identical thereto (e.g., 85% to 99% identical thereto); SEQ ID NOs: 145, 147, 162, and 141, respectively, or a sequence substantially identical thereto (e.g., 85% to 99% identical thereto); SEQ ID NOs: 153, 147, 162, and 141, respectively, or a sequence substantially identical thereto (e.g., 85% to 99% identical thereto); SEQ ID NOs: 154, 147, 162, and 141, respectively, or a sequence substantially identical thereto (e.g., 85% to 99% identical thereto); SEQ ID NOs: 146, 148, 162, and 141, respectively, or a sequence substantially identical thereto (e.g., 85% to 99% identical thereto); SEQ ID NOs: 155, 148, 162, and 141, respectively, or a sequence substantially identical thereto (e.g., 85% to 99% identical thereto); SEQ ID NOs: 156, 148, 162, and 141, respectively, or a sequence substantially identical thereto (e.g., 85% to 99% identical thereto); SEQ ID NOs: 137, 138, 140, and 161, respectively, or a sequence substantially identical thereto (e.g., 85% to 99% identical thereto); SEQ ID NOs: 139, 138, 140, and 161, respectively, or a sequence substantially identical thereto (e.g., 85% to 99% identical thereto); SEQ ID NOs: 149, 150, 140, and 161, respectively, or a sequence substantially identical thereto (e.g., 85% to 99% identical thereto); SEQ ID NOs: 151, 152, 140, and 161, respectively, or a sequence substantially identical thereto (e.g., 85% to 99% identical thereto); SEQ ID NOs: 137, 138, 140, and 141, respectively, or a sequence substantially identical thereto (e.g., 85% to 99% identical thereto); SEQ ID NOs: 139, 138, 140, and 141, respectively, or a sequence substantially identical thereto (e.g., 85% to 99% identical thereto); SEQ ID NOs: 149, 150, 140, and 141, respectively, or a sequence substantially identical thereto (e.g., 85% to 99% identical thereto); SEQ ID NOs: 151, 152, 140, and 141, respectively, or a sequence substantially identical thereto (e.g., 85% to 99% identical thereto); SEQ ID NOs: 137, 138, 162, and 161, respectively, or a sequence substantially identical thereto (e.g., 85% to 99% identical thereto); SEQ ID NOs: 139, 138, 162, and 161, respectively, or a sequence substantially identical thereto (e.g., 85% to 99% identical thereto); SEQ ID NOs: 149, 150, 162, and 161, respectively, or a sequence substantially identical thereto (e.g., 85% to 99% identical thereto); SEQ ID NOs: 151, 152, 162, and 161, respectively, or a sequence substantially identical thereto (e.g., 85% to 99% identical thereto); SEQ ID NOs: 137, 138, 162, and 141, respectively, or a sequence substantially identical thereto (e.g., 85% to 99% identical thereto); SEQ ID NOs: 139, 138, 162, and 141, respectively, or a sequence substantially identical thereto (e.g., 85% to 99% identical thereto); SEQ ID NOs: 149, 150, 162, and 141, respectively, or a sequence substantially identical thereto (e.g., 85% to 99% identical thereto); SEQ ID NOs: 151, 152, 162, and 141, respectively, or a sequence substantially identical thereto (e.g., 85% to 99% identical thereto); SEQ ID NOs: 166, 161, 172, and 161, respectively, or a sequence substantially identical thereto (e.g., 85% to 99% identical thereto); SEQ ID NOs: 167, 161, 172, and 161, respectively, or a sequence substantially identical thereto (e.g., 85% to 99% identical thereto); SEQ ID NOs: 168, 161, 172, and 161, respectively, or a sequence substantially identical thereto (e.g., 85% to 99% identical thereto); SEQ ID NOs: 166, 161, 173, and 161, respectively, or a sequence substantially identical thereto (e.g., 85% to 99% identical thereto); SEQ ID NOs: 167, 161, 173, and 161, respectively, or a sequence substantially identical thereto (e.g., 85% to 99% identical thereto); SEQ ID NOs: 168, 161, 173, and 161, respectively, or a sequence substantially identical thereto (e.g., 85% to 99% identical thereto); SEQ ID NOs: 169, 141, 174, and 141, respectively, or a sequence substantially identical thereto (e.g., 85% to 99% identical thereto); SEQ ID NOs: 170, 141, 174, and 141, respectively, or a sequence substantially identical thereto (e.g., 85% to 99% identical thereto); SEQ ID NOs: 171, 141, 174, and 141, respectively, or a sequence substantially identical thereto (e.g., 85% to 99% identical thereto); SEQ ID NOs: 169, 141, 175, and 141, respectively, or a sequence substantially identical thereto (e.g., 85% to 99% identical thereto); SEQ ID NOs: 170, 141, 175, and 141, respectively, or a sequence substantially identical thereto (e.g., 85% to 99% identical thereto); SEQ ID NOs: 171, 141, 175, and 141, respectively, or a sequence substantially identical thereto (e.g., 85% to 99% identical thereto); SEQ ID NOs: 166, 161, 174, and 141, respectively, or a sequence substantially identical thereto (e.g., 85% to 99% identical thereto); SEQ ID NOs: 167, 161, 174, and 141, respectively, or a sequence substantially identical thereto (e.g., 85% to 99% identical thereto); SEQ ID NOs: 168, 161, 174, and 141, respectively, or a sequence substantially identical thereto (e.g., 85% to 99% identical thereto); SEQ ID NOs: 166, 161, 175, and 141, respectively, or a sequence substantially identical thereto (e.g., 85% to 99% identical thereto); SEQ ID NOs: 167, 161, 175, and 141, respectively, or a sequence substantially identical thereto (e.g., 85% to 99% identical thereto); SEQ ID NOs: 168, 161, 175, and 141, respectively, or a sequence substantially identical thereto (e.g., 85% to 99% identical thereto); SEQ ID NOs: 166, 141, 174, and 161, respectively, or a sequence substantially identical thereto (e.g., 85% to 99% identical thereto); SEQ ID NOs: 167, 141, 174, and 161, respectively, or a sequence substantially identical thereto (e.g., 85% to 99% identical thereto); SEQ ID NOs: 168, 141, 174, and 161, respectively, or a sequence substantially identical thereto (e.g., 85% to 99% identical thereto); SEQ ID NOs: 166, 141, 175, and 161, respectively, or a sequence substantially identical thereto (e.g., 85% to 99% identical thereto); SEQ ID NOs: 167, 141, 175, and 161, respectively, or a sequence substantially identical thereto (e.g., 85% to 99% identical thereto); or SEQ ID NOs: 168, 141, 175, and 161, respectively, or a sequence substantially identical thereto (e.g., 85% to 99% identical thereto).

In some embodiments, the multispecific molecule comprises a first and a second non-contiguous polypeptides, wherein the first and the second non-contiguous polypeptides comprise the amino acid sequences of: SEQ ID NOs: 142 and 143, respectively, or a sequence substantially identical thereto (e.g., 85% to 99% identical thereto); SEQ ID NOs: 142 and 144, respectively, or a sequence substantially identical thereto (e.g., 85% to 99% identical thereto); SEQ ID NOs: 157 and 143, respectively, or a sequence substantially identical thereto (e.g., 85% to 99% identical thereto); SEQ ID NOs: 157 and 144, respectively, or a sequence substantially identical thereto (e.g., 85% to 99% identical thereto); SEQ ID NOs: 158 and 143, respectively, or a sequence substantially identical thereto (e.g., 85% to 99% identical thereto); SEQ ID NOs: 158 and 144, respectively, or a sequence substantially identical thereto (e.g., 85% to 99% identical thereto); SEQ ID NOs: 163 and 143, respectively, or a sequence substantially identical thereto (e.g., 85% to 99% identical thereto); SEQ ID NOs: 163 and 144, respectively, or a sequence substantially identical thereto (e.g., 85% to 99% identical thereto); SEQ ID NOs: 164 and 143, respectively, or a sequence substantially identical thereto (e.g., 85% to 99% identical thereto); SEQ ID NOs: 164 and 144, respectively, or a sequence substantially identical thereto (e.g., 85% to 99% identical thereto); SEQ ID NOs: 165 and 143, respectively, or a sequence substantially identical thereto (e.g., 85% to 99% identical thereto); or SEQ ID NOs: 165 and 144, respectively, or a sequence substantially identical thereto (e.g., 85% to 99% identical thereto).

Exemplary multispecific molecules that comprise (i) a CSF1R binding moiety (e.g., an anti-CSF1R antibody molecule), (ii) a PD-L1 binding moiety (e.g., an anti-PD-L1 antibody molecule), and (iii) one or more TGF-beta inhibitors are shown in FIGS. 1A-1J, 2A-2D, 3A-3D, 4A-4D, and 5A-5B.

FIGS. 1A-1J are schematics showing multispecific molecules comprising a Fab against CSF1R and a Fab against PD-L1. The CH1 domain of the anti-CSF1R Fab is linked, e.g., via a linker, to a first Fc region, which is optionally further linked, e.g., via a linker, to a first TGF-beta inhibitor. Similarly, the CH1 domain of the anti-PD-L1 Fab is linked, e.g., via a linker, to a second Fc region, which is optionally further linked, e.g., via a linker, to a second TGF-beta inhibitor.

In some embodiments, the multispecific molecule has the configuration of FIG. 1A. In FIG. 1A, the first TGF-beta inhibitor comprises (TGFBR1 ECD)ₐ, (TGFBR2 ECD)_{b}, and (TGFBR3 ECD)_{c}, or variant thereof, wherein a≥0, b≥0, and c≥0. The various extracellular domains can be linked, e.g., via one or more linkers, in any order. The second TGF-beta inhibitor comprises (TGFBR1 ECD)_{d}, (TGFBR2 ECD)ₑ, and (TGFBR3 ECD)_{f}, or variant thereof, wherein d≥0, e≥0, and f≥0. The various extracellular domains can be linked, e.g., via one or more linkers, in any order. At least one of a, b, c, d, e, or f is not zero. Exemplary arrangements of the extracellular domains include, but are not limited to, in the N- to C-orientation: TGFBR1 ECD and TGFBR2 ECD; TGFBR1 ECD, TGFBR2 ECD, and TGFBR2 ECD; TGFBR1 ECD, TGFBR2 ECD, TGFBR1 ECD, and TGFBR2 ECD; TGFBR1 ECD, TGFBR2 ECD, TGFBR2 ECD, and TGFBR1 ECD; TGFBR1 ECD, TGFBR1 ECD, TGFBR2 ECD, and TGFBR2 ECD; and TGFBR1 ECD, TGFBR2 ECD, TGFBR3 ECD.

In some embodiments, the multispecific molecule has the configuration of FIG. 1B. In FIG. 1B, the first TGF-beta inhibitor comprises TGFBR2 ECD, or variant thereof, and the second TGF-beta inhibitor comprises TGFBR2 ECD, or variant thereof. The first TGF-beta inhibitor and the second TGF-beta inhibitor can be the same or different. In some embodiments, the first and the second TGF-beta inhibitors form a dimer (e.g., a homo- or heterodimer).

In some embodiments, the multispecific molecule has the configuration of FIG. 1C. In FIG. 1C, the first TGF-beta inhibitor comprises TGFBR2 ECD, or variant thereof, and the second TGF-beta inhibitor comprises TGFBR1 ECD, or variant thereof. In some embodiments, the first and the second TGF-beta inhibitors form a dimer (e.g., a heterodimer).

In some embodiments, the multispecific molecule has the configuration of FIG. 1D. In FIG. 1D, the first TGF-beta inhibitor comprises TGFBR1 ECD, or variant thereof, and the second TGF-beta inhibitor comprises TGFBR2 ECD, or variant thereof. In some embodiments, the first and the second TGF-beta inhibitors form a dimer (e.g., a heterodimer).

In some embodiments, the multispecific molecule has the configuration of FIG. 1E. In FIG. 1E, the first TGF-beta inhibitor comprises TGFBR2 ECD, or variant thereof. The second TGF-beta inhibitor can be present or absent.

In some embodiments, the multispecific molecule has the configuration of FIG. 1F. In FIG. 1F, the second TGF-beta inhibitor comprises TGFBR2 ECD, or variant thereof. The first TGF-beta inhibitor can be present or absent.

In some embodiments, the multispecific molecule has the configuration of FIG. 1G. In FIG. 1G, the first TGF-beta inhibitor comprises (TGFBR2 ECD)₂, or variant thereof. In some embodiments, the two TGFBR2 ECDs are linked, e.g., via a linker. The two TGFBR2 ECDs can be the same or different. The second TGF-beta inhibitor can be present or absent.

In some embodiments, the multispecific molecule has the configuration of FIG. 1H. In FIG. 1H, the second TGF-beta inhibitor comprises (TGFBR2 ECD)₂, or variant thereof. In some embodiments, the two TGFBR2 ECDs are linked, e.g., via a linker. The two TGFBR2 ECDs can be the same or different. The first TGF-beta inhibitor can be present or absent.

In some embodiments, the multispecific molecule has the configuration of FIG. 1I. In FIG. 1I, the first TGF-beta inhibitor comprises TGFBR1 ECD and TGFBR2 ECD, or variant thereof. In some embodiments, the TGFBR1 ECD and TGFBR2 ECD are linked, e.g., via a linker, in either order (e.g., in the N- to C-orientation: TGFBR1 ECD followed by TGFBR2 ECD, or TGFBR2 ECD followed by TGFBR1 ECD). The second TGF-beta inhibitor can be present or absent.

In some embodiments, the multispecific molecule has the configuration of FIG. 1J. In FIG. 1J, the second TGF-beta inhibitor comprises TGFBR1 ECD and TGFBR2 ECD, or variant thereof. In some embodiments, the TGFBR1 ECD and TGFBR2 ECD are linked, e.g., via a linker, in either order (e.g., in the N- to C-orientation: TGFBR1 ECD followed by TGFBR2 ECD, or TGFBR2 ECD followed by TGFBR1 ECD). The first TGF-beta inhibitor can be present or absent.

FIGS. 2A-2D are schematics showing multispecific molecules comprising a Fab against CSF1R, an scFv against PD-L1, a first TGF-beta inhibitor, and a second TGF-beta inhibitor. The CH1 domain of the anti-CSF1R Fab is linked, e.g., via a linker, to a first Fc region, which is optionally further linked, e.g., via a linker, to the anti-PD-L1 scFv. The first TGF-beta inhibitor is linked, e.g., via a linker, to a CH1 domain, which is further linked, e.g., via a linker, to a second Fc region. The second TGF-beta inhibitor is linked, e.g., via a linker, to a CL domain.

In some embodiments, the multispecific molecule has the configuration of FIG. 2A. In FIG. 2A, the first TGF-beta inhibitor comprises TGFBR1 ECD, or variant thereof; and the second TGF-beta inhibitor comprises TGFBR1 ECD, or variant thereof. The first and the second TGF-beta inhibitors can be the same or different. In some embodiments, the first and the second TGF-beta inhibitors form a dimer (e.g., a homo- or heterodimer).

In some embodiments, the multispecific molecule has the configuration of FIG. 2B. In FIG. 2B, the first TGF-beta inhibitor comprises TGFBR1 ECD, or variant thereof; and the second TGF-beta inhibitor comprises TGFBR2 ECD, or variant thereof. In some embodiments, the first and the second TGF-beta inhibitors form a dimer (e.g., a heterodimer).

In some embodiments, the multispecific molecule has the configuration of FIG. 2C. In FIG. 2C, the first TGF-beta inhibitor comprises TGFBR2 ECD, or variant thereof; and the second TGF-beta inhibitor comprises TGFBR1 ECD, or variant thereof. In some embodiments, the first and the second TGF-beta inhibitors form a dimer (e.g., a heterodimer).

In some embodiments, the multispecific molecule has the configuration of FIG. 2D. In FIG. 2D, the first TGF-beta inhibitor comprises TGFBR2 ECD, or variant thereof; and the second TGF-beta inhibitor comprises TGFBR2 ECD, or variant thereof. The first and the second TGF-beta inhibitors can be the same or different. In some embodiments, the first and the second TGF-beta inhibitors form a dimer (e.g., a homo- or heterodimer).

FIGS. 3A-3D are schematics showing multispecific molecules comprising a Fab against PD-L1, an scFv against CSF1R, a first TGF-beta inhibitor, and a second TGF-beta inhibitor. The CH1 domain of the anti-PD-L1 Fab is linked, e.g., via a linker, to a first Fc region, which is optionally further linked, e.g., via a linker, to the anti-CSF1R scFv. The first TGF-beta inhibitor is linked, e.g., via a linker, to a CH1 domain, which is further linked, e.g., via a linker, to a second Fc region. The second TGF-beta inhibitor is linked, e.g., via a linker, to a CL domain.

In some embodiments, the multispecific molecule has the configuration of FIG. 3A. In FIG. 3A, the first TGF-beta inhibitor comprises TGFBR1 ECD, or variant thereof; and the second TGF-beta inhibitor comprises TGFBR1 ECD, or variant thereof. The first and the second TGF-beta inhibitors can be the same or different. In some embodiments, the first and the second TGF-beta inhibitors form a dimer (e.g., a homo- or heterodimer).

In some embodiments, the multispecific molecule has the configuration of FIG. 3B. In FIG. 3B, the first TGF-beta inhibitor comprises TGFBR1 ECD, or variant thereof; and the second TGF-beta inhibitor comprises TGFBR2 ECD, or variant thereof. In some embodiments, the first and the second TGF-beta inhibitors form a dimer (e.g., a heterodimer).

In some embodiments, the multispecific molecule has the configuration of FIG. 3C. In FIG. 3C, the first TGF-beta inhibitor comprises TGFBR2 ECD, or variant thereof; and the second TGF-beta inhibitor comprises TGFBR1 ECD, or variant thereof. In some embodiments, the first and the second TGF-beta inhibitors form a dimer (e.g., a heterodimer).

In some embodiments, the multispecific molecule has the configuration of FIG. 3D. In FIG. 3D, the first TGF-beta inhibitor comprises TGFBR2 ECD, or variant thereof; and the second TGF-beta inhibitor comprises TGFBR2 ECD, or variant thereof. The first and the second TGF-beta inhibitors can be the same or different. In some embodiments, the first and the second TGF-beta inhibitors form a dimer (e.g., a homo- or heterodimer).

FIGS. 4A-4D are schematics showing multispecific molecules comprising an scFv against PD-L1, an scFv against CSF1R, a first TGF-beta inhibitor, a second TGF-beta inhibitor, a third TGF-beta inhibitor, and a fourth TGF-beta inhibitor. The first TGF-beta inhibitor is linked, e.g., via a linker, to a first CL. The second TGF-beta inhibitor is linked, e.g., via a linker, to a first CH1, which is further linked, e.g., via a linker, to a first Fc region, which is further linked, e.g., via a linker, to the anti-PD-L1 scFv. The third TGF-beta inhibitor is linked, e.g., via a linker, to a second CH1, which is further linked, e.g., via a linker, to a second Fc region, which is further linked, e.g., via a linker, to the anti-CSF1R scFv. The fourth TGF-beta inhibitor is linked, e.g., via a linker, to a second CL.

In some embodiments, the multispecific molecule has the configuration of FIG. 4A. In FIG. 4A, the first, second, third, and fourth TGF-beta inhibitors comprise TGFBR1 ECD, or variant thereof. The first, second, third, and fourth TGF-beta inhibitors can be the same or different. In some embodiments, the first and the second TGF-beta inhibitors form a dimer (e.g., a homo- or heterodimer). In some embodiments, the third and the fourth TGF-beta inhibitors form a dimer (e.g., a homo- or heterodimer).

In some embodiments, the multispecific molecule has the configuration of FIG. 4B. In FIG. 4B, the first, second, third, and fourth TGF-beta inhibitors comprise TGFBR2 ECD, or variant thereof. The first, second, third, and fourth TGF-beta inhibitors can be the same or different. In some embodiments, the first and the second TGF-beta inhibitors form a dimer (e.g., a homo- or heterodimer). In some embodiments, the third and the fourth TGF-beta inhibitors form a dimer (e.g., a homo- or heterodimer).

In some embodiments, the multispecific molecule has the configuration of FIG. 4C. In FIG. 4C, the first and the second TGF-beta inhibitors comprise TGFBR1 ECD, or variant thereof; and the third and the fourth TGF-beta inhibitors comprise TGFBR2 ECD, or variant thereof. The first and second TGF-beta inhibitors can be the same or different. The third and fourth TGF-beta inhibitors can be the same or different. In some embodiments, the first and the second TGF-beta inhibitors form a dimer (e.g., a homo- or heterodimer). In some embodiments, the third and the fourth TGF-beta inhibitors form a dimer (e.g., a homo- or heterodimer).

In some embodiments, the multispecific molecule has the configuration of FIG. 4D. In FIG. 4D, the second and the third TGF-beta inhibitors comprise TGFBR1 ECD, or variant thereof; and the first and the fourth TGF-beta inhibitors comprise TGFBR2 ECD, or variant thereof. The second and third TGF-beta inhibitors can be the same or different. The first and fourth TGF-beta inhibitors can be the same or different. In some embodiments, the first and the second TGF-beta inhibitors form a dimer (e.g., a heterodimer). In some embodiments, the third and the fourth TGF-beta inhibitors form a dimer (e.g., a heterodimer).

FIGS. 5A-5B are schematics showing multispecific molecules comprising an scFv against PD-L1, an scFv against CSF1R, a first TGF-beta inhibitor, and a second TGF-beta inhibitor. The first TGF-beta inhibitor is linked, e.g., via a linker, to a first Fc region, which is further linked, e.g., via a linker, to the anti-PD-L1 scFv. The second TGF-beta inhibitor is linked, e.g., via a linker, to a second Fc region, which is further linked, e.g., via a linker, to the anti-CSF1R scFv.

In some embodiments, the multispecific molecule has the configuration of FIG. 5A. In FIG. 5A, the first TGF-beta inhibitor comprises TGFBR1 ECD, or variant thereof; and the second TGF-beta inhibitor comprises TGFBR2 ECD, or variant thereof.

In some embodiments, the multispecific molecule has the configuration of FIG. 5B. In FIG. 5B, the first TGF-beta inhibitor comprises TGFBR2 ECD, or variant thereof; and the second TGF-beta inhibitor comprises TGFBR2 ECD, or variant thereof. The first and second TGF-beta inhibitors can be the same or different.

In one aspect, provided herein is an isolated multispecific, e.g., a bispecific or trispecific, molecule, comprising: (i) a TGF-beta inhibitor; and (ii) an anti-CSF1R binding moiety (e.g., an anti-CSF1R antibody molecule) or an anti-CCR2 binding moiety (e.g., an anti-CCR2 antibody molecule). In one embodiment, the multispecific molecule further comprises a tumor targeting moiety (e.g., a tumor targeting antibody molecule).

In one embodiment, the TGF-beta inhibitor reduces the activity of one, two, or all of: (i) TGF-beta 1, (ii) TGF-beta 2, or (iii) TGF-beta 3, optionally wherein the TGF-beta inhibitor reduces the activity of: (a) TGF-beta 1 and TGF-beta 3, or (b) TGF-beta 1, TGF-beta 2, and TGF-beta 3, e.g., as measured using the methods described in Example 3 with respect to FIG. 7. In one embodiment, the TGF-beta inhibitor comprises a TGF-beta receptor polypeptide (e.g., an extracellular domain of a TGF-beta receptor, or a functional variant thereof). In one embodiment, the TGF-beta inhibitor comprises one, two, or all of: (i) a TGFBR1 polypeptide (e.g., 1, 2, 3, or more of a TGFBR1 polypeptide), (ii) a TGFBR2 polypeptide (e.g., 1, 2, 3, or more of a TGFBR2 polypeptide), or (iii) a TGFBR3 polypeptide (e.g., 1, 2, 3, or more of a TGFBR3 polypeptide).

In one embodiment, the TGF-beta inhibitor comprises a TGFBR1 polypeptide. In one embodiment, the TGF-beta inhibitor comprises an extracellular domain of TGFBR1 or a sequence substantially identical thereto (e.g., a sequence that is at least 80%, 85%, 90%, or 95% identical thereto). In one embodiment, the TGF-beta inhibitor comprises an extracellular domain of SEQ ID NO: 95, 96, 97, 120, 121, or 122, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 104 or 105, or a sequence substantially identical thereto (e.g., a sequence that is at least 80%, 85%, 90%, or 95% identical thereto).

In one embodiment, the TGF-beta inhibitor comprises a TGFBR2 polypeptide. In one embodiment, the TGF-beta inhibitor comprises an extracellular domain of TGFBR2 or a sequence substantially identical thereto (e.g., a sequence that is at least 80%, 85%, 90%, or 95% identical thereto). In one embodiment, the TGF-beta inhibitor comprises an extracellular domain of SEQ ID NO: 98, 99, 123, or 124, or a sequence substantially identical thereto (e.g., a sequence that is at least 80%, 85%, 90%, or 95% identical thereto). In one embodiment, the TGF-beta inhibitor comprises an amino acid sequence selected from the group consisting of SEQ ID NOs: 100, 101, 102, and 103, or a sequence substantially identical thereto (e.g., a sequence that is at least 80%, 85%, 90%, or 95% identical thereto).

In one embodiment, the TGF-beta inhibitor comprises a TGFBR3 polypeptide. In one embodiment, the TGF-beta inhibitor comprises an extracellular domain of TGFBR3 or a sequence substantially identical thereto (e.g., a sequence that is at least 80%, 85%, 90%, or 95% identical thereto). In one embodiment, the TGF-beta inhibitor comprises an extracellular domain of SEQ ID NO: 106, 107, 125, or 126, or a sequence substantially identical thereto (e.g., a sequence that is at least 80%, 85%, 90%, or 95% identical thereto). In one embodiment, the TGF-beta inhibitor comprises the amino acid sequence of SEQ ID NO: 108, or a sequence substantially identical thereto (e.g., a sequence that is at least 80%, 85%, 90%, or 95% identical thereto).

In one embodiment, the TGF-beta inhibitor comprises two TGF-beta receptor polypeptides that form a homodimer. In one embodiment, the TGF-beta inhibitor comprises two TGFBR1 polypeptides that form a homodimer. In one embodiment, the TGF-beta inhibitor comprises two TGFBR2 polypeptides that form a homodimer. In one embodiment, the TGF-beta inhibitor comprises two TGFBR3 polypeptides that form a homodimer. In one embodiment, the TGF-beta inhibitor comprises two TGF-beta receptor polypeptides that form a heterodimer. In one embodiment, the TGF-beta inhibitor comprises a TGFBR1 polypeptide and a TGFBR2 polypeptide that form a heterodimer. In one embodiment, the TGF-beta inhibitor comprises a TGFBR1 polypeptide and a TGFBR3 polypeptide that form a heterodimer. In one embodiment, the TGF-beta inhibitor comprises a TGFBR2 polypeptide and a TGFBR3 polypeptide that form a heterodimer.

In one embodiment, the TGF-beta inhibitor comprises a first TGF-beta receptor polypeptide and a second TGF-beta receptor polypeptide. In one embodiment, the multispecific molecule comprises a first Fc region (e.g., a first CH1-Fc region) and a second Fc region (e.g., a second CH1-Fc region). In one embodiment, the first TGF-beta receptor polypeptide is linked, e.g., via a linker, to the first Fc region (e.g., a first CH1-Fc region), e.g., the C-terminus of the first Fc region (e.g., a first CH1-Fc region). In one embodiment, the second TGF-beta receptor polypeptide is linked, e.g., via a linker, to the second Fc region (e.g., a second CH1-Fc region), e.g., the C-terminus of the second Fc region (e.g., a second CH1-Fc region). In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 6A or 6B. In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 192 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 193 (or a sequence substantially identical thereto, e.g., a sequence that is at least 80%, 85%, 90%, or 95% identical thereto). In one embodiment, the multispecific molecule comprises the amino acid sequence of SEQ ID NO: 194 (or a sequence substantially identical thereto, e.g., a sequence that is at least 80%, 85%, 90%, or 95% identical thereto) and the amino acid sequence of SEQ ID NO: 195 (or a sequence substantially identical thereto, e.g., a sequence that is at least 80%, 85%, 90%, or 95% identical thereto).

In one embodiment, the multispecific molecule comprises a heavy chain constant region 1 (CH1) and a light chain constant region (CL). In one embodiment, (i) the first TGF-beta receptor polypeptide is linked, e.g., via a linker, to the CH1, e.g., the N-terminus of the CH1, and (ii) the second TGF-beta receptor polypeptide is linked, e.g., via a linker, to the CL, e.g., the N-terminus of the CL. In one embodiment, the first TGF-beta receptor polypeptide and the second TGF-beta receptor polypeptide form a homodimer or heterodimer, e.g., a homodimer. In one embodiment, the first or second TGF-beta receptor polypeptide comprises an extracellular domain of TGFBR1, TGFBR2, or TGFBR3, e.g., an extracellular domain of TGFBR2. In one embodiment, the multispecific molecule has the configuration of FIG. 6C or 6D. In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 196 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 198 (or a sequence substantially identical thereto, e.g., a sequence that is at least 80%, 85%, 90%, or 95% identical thereto). In one embodiment, the multispecific molecule comprises the amino acid sequence of SEQ ID NO: 197 (or a sequence substantially identical thereto, e.g., a sequence that is at least 80%, 85%, 90%, or 95% identical thereto) and the amino acid sequence of SEQ ID NO: 199 (or a sequence substantially identical thereto, e.g., a sequence that is at least 80%, 85%, 90%, or 95% identical thereto).

In one embodiment, the multispecific molecule comprises an anti-CSF1R binding moiety (e.g., an anti-CSF1R antibody molecule). In one embodiment, the multispecific molecule comprises an anti-CCR2 binding moiety (e.g., an anti-CCR2 antibody molecule).

In one embodiment, the tumor targeting moiety (e.g., a tumor targeting antibody molecule) binds to PD-L1, mesothelin, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pme117, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDCl₂7, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUMS, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In one embodiment, the tumor targeting moiety (e.g., a tumor targeting antibody molecule) binds to CD19, CD33, CD47, CD123, CD20, CD99, CD30, BCMA, CD38, CD22, SLAMF7, or NY-ESO1.

In one embodiment, the anti-CSF1R antibody molecule, anti-CCR2 antibody molecule, or tumor targeting antibody molecule is, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)). In one embodiment, the anti-CSF1R antibody molecule, anti-CCR2 antibody molecule, or tumor targeting antibody molecule comprises a heavy chain constant region chosen from IgG1, IgG2, IgG3, or IgG4, or a fragment thereof. In one embodiment, the anti-CSF1R antibody molecule, anti-CCR2 antibody molecule, or tumor targeting antibody molecule comprises a light chain constant region chosen from the light chain constant regions of kappa or lambda, or a fragment thereof. In one embodiment, the anti-CSF1R antibody molecule or anti-CCR2 antibody molecule comprises a kappa light chain constant region, or a fragment thereof, and the tumor targeting antibody molecule comprises a lambda light chain constant region, or a fragment thereof. In one embodiment, the anti-CSF1R antibody molecule or anti-CCR2 antibody molecule comprises a lambda light chain constant region, or a fragment thereof, and the tumor targeting antibody molecule comprises a kappa light chain constant region, or a fragment thereof. In one embodiment, the anti-CSF1R antibody molecule or anti-CCR2 antibody molecule and the tumor targeting antibody molecule have a common light chain variable region.

In one embodiment, the multispecific molecule comprises a heavy chain constant region (e.g., an Fc region) chosen from the heavy chain constant regions of IgG1, IgG2, and IgG4, more particularly, the heavy chain constant region of human IgG1, IgG2 or IgG4. In one embodiment, the heavy chain constant region (e.g., an Fc region) is linked to, e.g., covalently linked to, anti-CSF1R antibody molecule, anti-CCR2 antibody molecule, or tumor targeting antibody molecule. In one embodiment, the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region. In one embodiment, the anti-CSF1R antibody molecule or anti-CCR2 antibody molecule comprises a first heavy chain constant region (e.g., a first Fc region) and the tumor targeting antibody molecule comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region. In one embodiment, the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions. In one embodiment, the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system. In one embodiment, the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system. In one embodiment, the multispecific molecule comprises a linker, optionally wherein the linker is chosen from: a cleavable linker, a non-cleavable linker, a peptide linker, a flexible linker, a rigid linker, a helical linker, or a non-helical linker, optionally wherein the linker is a peptide linker, optionally wherein the peptide linker comprises Gly and Ser.

In another aspect, provided herein are isolated nucleic acids encoding the multispecific molecule (e.g., antibody) of any one of the preceding claims.

In another aspect, provided herein are isolated nucleic acid molecules, which comprises the nucleotide sequence encoding any of the multispecific molecules described herein, or a nucleotide sequence substantially homologous thereto (e.g., at least 95% to 99.9% identical thereto).

In another aspect, provided herein are vectors, e.g., expression vectors, comprising one or more of the nucleic acid molecules described herein.

In another aspect, provided herein are host cells comprising the nucleic acid molecule described herein or the vector described herein.

In another aspect, provided herein are methods of making, e.g., producing, the multispecific molecules described herein, comprising culturing the host cell described herein, under suitable conditions, e.g., conditions suitable for gene expression and/or heterodimerization.

In another aspect, provided herein are pharmaceutical compositions comprising the multispecific molecule described herein and a pharmaceutically acceptable carrier, excipient, or stabilizer.

In another aspect, provided herein are methods of treating a cancer in a subject, comprising administering to the subject in need thereof the multispecific molecule described herein, wherein the multispecific molecule is administered in an amount effective to treat the cancer.

In another aspect, provided herein are method of treating a cancer in a subject, comprising administering to the subject in need thereof the multispecific molecule described herein, wherein the multispecific molecule is administered in an amount effective to reduce the number of TAMs (e.g., the number of TAMs in or near the a tumor in the subject), inhibit the proliferation of TAMs (e.g., the number of TAMs in or near the a tumor in the subject), or reduce or inhibit macrophage infiltration into a tumor in the subject.

In another aspect, provided herein are methods of treating a cancer in a subject by reducing a portion of a population of TAMs, comprising administering to the subject in need thereof the multispecific molecule described herein, wherein the multispecific molecule is administered in an amount effective to inhibit or deplete a portion of a population of TAMs.

In another aspect, provided herein are methods of reducing the proliferation of a portion of a population of TAMs in a subject (e.g., in a subject having cancer, e.g., a solid tumor), comprising, administering to the subject in need thereof the multispecific molecule described herein, wherein the multispecific molecule is administered in an amount effective to reduce the proliferation of a portion of the population of TAMs.

In another aspect, provided herein are methods of inhibiting or depleting a portion of a population of TAMs in a subject having a cancer (e.g., a tumor), comprising administering to the subject the multispecific molecule described herein, wherein the multispecific molecule is administered in an amount effective to reduce the number of tumor infiltrating macrophages, inhibit the proliferation of tumor infiltrating macrophages, or reduce macrophage infiltration into a tumor.

In some embodiments, the cancer is a solid tumor cancer or a metastatic lesion. In some embodiments, the solid tumor cancer is one or more of pancreatic cancer (e.g., pancreatic adenocarcinoma), breast cancer, colorectal cancer, lung cancer (e.g., small or non-small cell lung cancer), skin cancer (e.g., melanoma), ovarian cancer, liver cancer, or brain cancer (e.g., glioma). In some embodiments, the cancer is characterized as containing TAMs, is associated with the presence of TAMs, TAMs are in and/or form part of the cancer (e.g., tumor), or TAMs have been detected in or near the solid tumor. In some embodiments, the cancer is a hematological cancer or a metastatic lesion. In some embodiments, the hematological cancer is one or more of a Hodgkin's lymphoma, Non-Hodgkin's lymphoma, B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia, acute myeloid leukemia (AML), chronic myeloid leukemia, myelodysplastic syndrome (MDS), multiple myeloma, or acute lymphocytic leukemia.

In some embodiments, the methods further comprise identifying the presence of TAMs in or near the cancer (e.g., tumor) in the subject. In some embodiments, the TAMs express CXCR2 and CCR2, CCR2 and CSF1R, CSF1R and CXCR2, or CCR2, CXCR2, and CSF1R.

In some embodiments, the methods further comprise administering a second therapeutic treatment. In some embodiments, the second therapeutic treatment comprises a therapeutic agent (e.g., a chemotherapeutic agent, a biologic agent, hormonal therapy), radiation, or surgery. In some embodiments, the therapeutic agent is selected from: a chemotherapeutic agent, or a biologic agent. In some embodiments, the therapeutic agent is a checkpoint inhibitor. In some embodiments, the check point inhibitor is selected from the group consisting of an anti-CTLA4 antibody, an anti-PD1 antibody (e.g., Nivolumab, Pembrolizumab or Pidilizumab), an anti-PD-L1 antibody, an anti-PD-L2 antibody, an anti-TIM3 antibody, an anti-LAG3 antibody, an anti-CD160 antibody, an anti-2B4 antibody, an anti-CD80 antibody, an anti-CD86 antibody, an anti-B7-H3 (CD276) antibody, an anti-B7-H4 (VTCN1) antibody, an anti-HVEM (TNFRSF14 or CD270) antibody, an anti-BTLA antibody, an anti-KIR antibody, an anti-MHC class I antibody, an anti-MHC class II antibody, an anti-GALS antibody, an anti-VISTA antibody, an anti-BTLA antibody, an anti-TIGIT antibody, an anti-LAIR1 antibody, and an anti-A2aR antibody.

In another aspect, provided herein are methods of treating a cancer in a subject, comprising administering to the subject in need thereof the multispecific molecule described herein, wherein the multispecific molecule is administered in an amount effective to reduce the number of MDSCs (e.g., the number of MDSCs in or near the a tumor in the subject), inhibit the proliferation of MDSCs (e.g., the number of MDSCs in or near the a tumor in the subject), or reduce or inhibit MDSC infiltration into a tumor in the subject.

In another aspect, provided herein are methods of treating a cancer in a subject by reducing a portion of a population of TAMs, comprising administering to the subject in need thereof the multispecific molecule described herein, wherein the multispecific molecule is administered in an amount effective to inhibit or deplete a portion of the population of TAMs.

In another aspect, provided herein are methods of reducing the proliferation of a portion of a population of MDSCs in a subject (e.g., in a subject having cancer, e.g., a solid tumor), comprising, administering to the subject in need thereof the multispecific molecule described herein, wherein the multispecific molecule is administered in an amount effective to reduce the proliferation of a portion of the population of MDSCs.

In another aspect, provided herein are methods of inhibiting or depleting a portion of a population of MDSCs in a subject having a cancer (e.g., a tumor), comprising administering to the subject the multispecific molecule described herein, wherein the multispecific molecule is administered in an amount effective to reduce the number of MDSCs, inhibit the proliferation of MDSCs, or reduce MDSC infiltration into a tumor.

In some embodiments, the cancer is a solid tumor cancer or a metastatic lesion. In some embodiments, the solid tumor cancer is one or more of pancreatic cancer (e.g., pancreatic adenocarcinoma), breast cancer, colorectal cancer, lung cancer (e.g., small or non-small cell lung cancer), skin cancer (e.g., melanoma), ovarian cancer, liver cancer, or brain cancer (e.g., glioma). In some embodiments, the cancer is characterized as containing MDSCs, is associated with the presence of MDSCs, MDSCs are in and/or form part of the cancer (e.g., tumor), or MDSCs have been detected in or near the solid tumor. In some embodiments, the methods further comprise identifying the presence of MDSCs in or near the cancer (e.g., tumor) in the subject.

In some embodiments, the methods further comprise administering a second therapeutic treatment. In some embodiments, the second therapeutic treatment comprises a therapeutic agent (e.g., a chemotherapeutic agent, a biologic agent, hormonal therapy), radiation, or surgery. In some embodiments, the therapeutic agent is selected from: a chemotherapeutic agent, or a biologic agent. In some embodiments, the therapeutic agent is a checkpoint inhibitor. In some embodiments, the check point inhibitor is selected from the group consisting of an anti-CTLA4 antibody, an anti-PD1 antibody (e.g., Nivolumab, Pembrolizumab or Pidilizumab), an anti-PD-L1 antibody, an anti-PD-L2 antibody, an anti-TIM3 antibody, an anti-LAG3 antibody, an anti-CD160 antibody, an anti-2B4 antibody, an anti-CD80 antibody, an anti-CD86 antibody, an anti-B7-H3 (CD276) antibody, an anti-B7-H4 (VTCN1) antibody, an anti-HVEM (TNFRSF14 or CD270) antibody, an anti-BTLA antibody, an anti-KIR antibody, an anti-MHC class I antibody, an anti-MHC class II antibody, an anti-GALS antibody, an anti-VISTA antibody, an anti-BTLA antibody, an anti-TIGIT antibody, an anti-LAIR1 antibody, and an anti-A2aR antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1J are schematics showing exemplary multispecific molecules comprising a CSF1R binding moiety, a PD-L1 binding moiety, and one or more TGF-beta inhibitors. Shown in FIGS. 1A-1J are multispecific antibody molecules comprising: a first polypeptide comprising anti-CSF1R VL and CL; a second polypeptide comprising anti-CSF1R VH, CH1, CH2, CH3, and optionally a first TGF-beta inhibitor; a third polypeptide comprising anti-PD-L1 VH, CH1, CH2, CH3, and optionally a second TGF-beta inhibitor; and a fourth polypeptide comprising anti-PD-L1 VL and CL. In FIG. 1A, the first TGF-beta inhibitor comprises (TGFBR1 ECD)ₐ, (TGFBR2 ECD)_{b}, and (TGFBR3 ECD)_{c}, or variant thereof, linked in any order, wherein a≥0, b≥0, and c≥0. The second TGF-beta inhibitor comprises (TGFBR1 ECD)_{d}, (TGFBR2 ECD)ₑ, and (TGFBR3 ECD)_{f}, or variant thereof, linked in any order, wherein d≥0, e≥0, and f≥0. At least one of a, b, c, d, e, or f is not zero. In FIG. 1B, the first and the second TGF-beta inhibitors comprise TGFBR2 ECD or variant thereof. In FIG. 1C, the first TGF-beta inhibitor comprises TGFBR2 ECD, or variant thereof, and the second TGF-beta inhibitor comprises TGFBR1 ECD, or variant thereof. In FIG. 1D, the first TGF-beta inhibitor comprises TGFBR1 ECD, or variant thereof, and the second TGF-beta inhibitor comprises TGFBR2 ECD, or variant thereof. In FIG. 1E, the first TGF-beta inhibitor comprises TGFBR2 ECD or variant thereof, and the second TGF-beta inhibitor can be present or absent. In FIG. 1F, the second TGF-beta inhibitor comprises TGFBR2 ECD or variant thereof, and the first TGF-beta inhibitor can be present or absent. In FIG. 1G, the first TGF-beta inhibitor comprises two TGFBR2 ECDs or variant thereof, and the second TGF-beta inhibitor can be present or absent. In FIG. 1H, the second TGF-beta inhibitor comprises two TGFBR2 ECDs or variant thereof, and the first TGF-beta inhibitor can be present or absent. In FIG. 1I, the first TGF-beta inhibitor comprises TGFBR1 ECD and TGFBR2 ECD, or variant thereof, and the second TGF-beta inhibitor can be present or absent. In FIG. 1J, the second TGF-beta inhibitor comprises TGFBR1 ECD and TGFBR2 ECD, or variant thereof, and the first TGF-beta inhibitor can be present or absent.

FIGS. 2A-2D are schematics showing additional exemplary multispecific molecules comprising a CSF1R binding moiety, a PD-L1 binding moiety, and one or more TGF-beta receptors. Shown in FIGS. 2A-2D are multispecific antibody molecules comprising: a first polypeptide comprising anti-CSF1R VL and CL; a second polypeptide comprising anti-CSF1R VH, CH1, CH2, CH3, an anti-PDL1 VH, and an anti-PDL1 VL; a third polypeptide comprising a first TGF-beta receptor, CH1, CH2, and CH3; and a fourth polypeptide comprising a second TGF-beta receptor and a CL. In FIG. 2A, the first and the second TGF-beta receptors comprise TGFBR1 ECD or variant thereof. In FIG. 2B, the first TGF-beta receptor comprises TGFBR1 ECD or variant thereof and the second TGF-beta receptor comprises TGFBR2 ECD or variant thereof. In FIG. 2C, the first TGF-beta receptor comprises TGFBR2 ECD or variant thereof and the second TGF-beta receptor comprises TGFBR1 ECD or variant thereof. In FIG. 2D, the first and the second TGF-beta receptors comprise TGFBR2 ECD or variant thereof.

FIGS. 3A-3D are schematics showing additional exemplary multispecific molecules comprising a CSF1R binding moiety, a PD-L1 binding moiety, and one or more TGF-beta receptors. Shown in FIGS. 3A-3D are multispecific antibody molecules comprising: a first polypeptide comprising anti-PDL1 VL and CL; a second polypeptide comprising anti-PDL1 VH, CH1, CH2, CH3, an anti-CSF1R VH, and an anti-CSF1R VL; a third polypeptide comprising a first TGF-beta receptor, CH1, CH2, and CH3; and a fourth polypeptide comprising a second TGF-beta receptor and CL. In FIG. 3A, the first and the second TGF-beta receptors comprise TGFBR1 ECD or variant thereof. In FIG. 3B, the first TGF-beta receptor comprises TGFBR1 ECD or variant thereof and the second TGF-beta receptor comprises TGFBR2 ECD or variant thereof. In FIG. 3C, the first TGF-beta receptor comprises TGFBR2 ECD or variant thereof and the second TGF-beta receptor comprises TGFBR1 ECD or variant thereof. In FIG. 3D, the first and the second TGF-beta receptors comprise TGFBR2 ECD or variant thereof.

FIGS. 4A-4D are schematics showing additional exemplary multispecific molecules comprising a CSF1R binding moiety, a PD-L1 binding moiety, and one or more TGF-beta receptors. Shown in FIGS. 4A-4D are multispecific antibody molecules comprising: a first polypeptide comprising a first TGF-beta receptor and CL; a second polypeptide comprising a second TGF-beta receptor, CH1, CH2, CH3, an anti-PDL1 VH, and an anti-PDL1 VL; a third polypeptide comprising a third TGF-beta receptor, CH1, CH2, CH3, an anti-CSF1R VH, and an anti-CSF1R VL; and a fourth polypeptide comprising a fourth TGF-beta receptor and CL. In FIG. 4A, the first, second, third, and fourth TGF-beta receptors comprise TGFBR1 ECD or variant thereof. In FIG. 4B, the first, second, third, and fourth TGF-beta receptors comprise TGFBR2 ECD or variant thereof. In FIG. 4C, the first and the second TGF-beta receptors comprise TGFBR1 ECD or variant thereof and the third and the fourth TGF-beta receptors comprise TGFBR2 ECD or variant thereof. In FIG. 4D, the second and the third TGF-beta receptors comprise TGFBR1 ECD or variant thereof and the first and the fourth TGF-beta receptors comprise TGFBR2 ECD or variant thereof.

FIGS. 5A-5B are schematics showing additional exemplary multispecific molecules comprising a CSF1R binding moiety, a PD-L1 binding moiety, and one or more TGF-beta receptors. Shown in FIGS. 5A-5B are multispecific antibody molecules comprising: a first polypeptide comprising a first TGF-beta receptor, CH2, CH3, an anti-PDL1 VH, and an anti-PDL1 VL; and a second polypeptide comprising a second TGF-beta receptor, CH2, CH3, an anti-CSF1R VH, and an anti-CSF1R VL. In FIG. 5A, the first TGF-beta receptor comprises a TGFBR1 ECD or variant thereof and the second TGF-beta receptor comprises a TGFBR2 ECD or variant thereof. In FIG. 5B, the first and the second TGF-beta receptors comprise TGFBR2 ECD or variant thereof.

FIGS. 6A-6D are schematics showing exemplary multispecific molecules comprising a TGFβ inhibitor. In some embodiments, the TGFβ inhibitor comprises a TGF-beta receptor ECD homodimer. In some embodiments, the TGFβ inhibitor comprises a TGFBR2 ECD heterodimer. In FIGS. 6A and 6B, the two TGFBR ECD domains are linked to the C-terminus of two Fc regions. In some embodiments, the CH1-Fc-TGFBR ECD region shown in FIG. 6A or 6B comprises the amino acid sequence of SEQ ID NO: 192 or 193. In some embodiments, the Fc-TGFBR ECD region shown in FIG. 6A or 6B comprises the amino acid sequence of SEQ ID NO: 194 or 195. In FIGS. 6C and 6D, the two TGFBR ECD domains are linked to CH1 and CL, respectively. In some embodiments, the TGFBR ECD-CH1-Fc region shown in FIG. 6C or 6D comprises the amino acid sequence of SEQ ID NO: 196 or 197. In some embodiments, the TGFBR ECD-CL region shown in FIG. 6C or 6D comprises the amino acid sequence of SEQ ID NO: 198 or 199. In some embodiments, the multispecific molecule comprises a binding moiety A and a binding moiety B. In some embodiments, the binding moiety A or binding moiety B is an anti-CSF1R binding moiety (e.g., an anti-CSF1R antibody molecule). In some embodiments, the binding moiety A or binding moiety B is an anti-CCR2 binding moiety (e.g., an anti-CCR2 antibody molecule). In some embodiments, the binding moiety A or binding moiety B is a tumor targeting moiety (e.g., a tumor targeting antibody molecule).

FIG. 7 is a graph in which TGFβ/Smad activation is plotted against TGFβ-trap concentrations. Constructs tested in this study included: Single TGFβ Fab-trap, Anti-PDL1×TGFβ-trap, Anti-CCR2×anti-CSF1R, and Anti-CCR2×anti-CSF1R×TGFβ-trap.

### DETAILED DESCRIPTION OF THE INVENTION

TAMs originate from circulating monocytes and their recruitment into tumors is driven by tumor-derived chemotactic factors. TAMs can promote tumor cell proliferation and metastasis by causing such responses as inhibition of B and T cell activation, inhibition of tumor-associated antigen presentation, inhibition of cytotoxic granule release, increased angiogenesis, and secretion a wide range of growth and proangiogenic factors (see e.g., Liu et al Cellular & Molecular Immunology (2015) 12, 1-4; and Noy, Roy et al Immunity, Volume 41, Issue 1, 49-61; and Quatromoni et al. Am J Transl Res. 2012; 4(4): 376-389). Consequently, many tumors with a high number of TAMs have an increased tumor growth rate, local proliferation and distant metastasis. Thus, therapies that deplete TAMs or inhibit their activity would be useful.

### Definitions

As used herein, the term “transforming growth factor beta-1 (TGF-beta 1)” refers to a protein that in humans is encoded by the gene TGFB1, or its orthologs. Swiss-Prot accession number P01137 provides exemplary human TGF-beta 1 amino acid sequences. An exemplary immature human TGF-beta 1 amino acid sequence is provided in SEQ ID NO: 92. An exemplary mature human TGF-beta 1 amino acid sequence is provided in SEQ ID NO: 117.

As used herein, the term “transforming growth factor beta-2 (TGF-beta 2)” refers to a protein that in humans is encoded by the gene TGFB2, or its orthologs. Swiss-Prot accession number P61812 provides exemplary human TGF-beta 2 amino acid sequences. An exemplary immature human TGF-beta 2 amino acid sequence is provided in SEQ ID NO: 93. An exemplary mature human TGF-beta 2 amino acid sequence is provided in SEQ ID NO: 118.

As used herein, the term “transforming growth factor beta-3 (TGF-beta 3)” refers to a protein that in humans is encoded by the gene TGFB3, or its orthologs. Swiss-Prot accession number P10600 provides exemplary human TGF-beta 3 amino acid sequences. An exemplary immature human TGF-beta 3 amino acid sequence is provided in SEQ ID NO: 94. An exemplary mature human TGF-beta 3 amino acid sequence is provided in SEQ ID NO: 119.

As used herein, a “TGF-beta receptor polypeptide” refers to a TGF-beta receptor (e.g., TGFBR1, TGFBR2, or TGFBR3) or its fragment, or variant thereof.

As used herein, the term “transforming growth factor beta receptor type 1 (TGFBR1)” (also known as ALK-5 or SKR4) refers to a protein that in humans is encoded by the gene TGFBR1, or its orthologs. Swiss-Prot accession number P36897 provides exemplary human TGFBR1 amino acid sequences. Exemplary immature human TGFBR1 amino acid sequences are provided in SEQ ID NOs: 95, 96, and 97. Exemplary mature human TGFBR1 amino acid sequences are provided in SEQ ID NOs: 120, 121, and 122. As used herein, a “TGFBR1 polypeptide” refers to a TGFBR1 or its fragment, or variant thereof.

As used herein, the term “transforming growth factor beta receptor type 2 (TGFBR2)” refers to a protein that in humans is encoded by the gene TGFBR2, or its orthologs. Swiss-Prot accession number P37173 provides exemplary human TGFBR2 amino acid sequences. Exemplary immature human TGFBR2 amino acid sequences are provided in SEQ ID NOs: 98 and 99. Exemplary mature human TGFBR2 amino acid sequences are provided in SEQ ID NOs: 123 and 124. As used herein, a “TGFBR2 polypeptide” refers to a TGFBR2 or its fragment, or variant thereof.

As used herein, the term “transforming growth factor beta receptor type 3 (TGFBR3)” refers to a protein that in humans is encoded by the gene TGFBR3, or its orthologs. Swiss-Prot accession number Q03167 provides exemplary human TGFBR3 amino acid sequences. Exemplary immature human TGFBR3 amino acid sequences are provided in SEQ ID NOs: 106 and 107. Exemplary mature human TGFBR3 amino acid sequences are provided in SEQ ID NOs: 125 and 126. As used herein, a “TGFBR3 polypeptide” refers to a TGFBR3 or its fragment, or variant thereof.

As used herein, the term “variant” of a parent sequence refers to a sequence that has a substantially identical amino acid sequence to the parent sequence, or a fragment thereof. In some embodiments, the variant is a functional variant.

As used herein, an “extracellular domain” or “ECD” of a polypeptide refers to a portion of the polypeptide that lacks the intracellular and transmembrane domains. In some embodiments, an “extracellular domain” or “ECD” of a polypeptide includes the whole portion of the polypeptide that is in the extracellular space when the polypeptide is on the cell surface, a fragment thereof, or a variant thereof.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable region sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

As used herein, an “immunoglobulin variable region sequence” refers to an amino acid sequence which can form the structure of an immunoglobulin variable region. For example, the sequence may include all or part of the amino acid sequence of a naturally-occurring variable region. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable region sequences, where a first immunoglobulin variable region sequence has binding specificity for a first epitope and a second immunoglobulin variable region sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (e.g., SEQ ID NO: 1) molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “immunosuppressive myeloid cell” or “IMC” generally refers to a cell of myeloid lineage that promotes immunosuppression (e.g., in a tumor microenvironment) (e.g., by inhibiting T cell activation, inhibiting T cell viability, promoting T regulatory cell induction and recruitment). Immunosuppressive myeloid cells include, e.g., tumor associated macrophages (TAMs) and myeloid derived suppressor cells (MDSCs).

As used herein, the term “tumor associated macrophage” or “TAM” generally refers to a macrophage that exists in the microenvironment of a cancer, for example, a tumor.

As used herein, the term “reducing TAMs” generally refers to decreasing the number of TAMs. Reducing includes decreasing the number of TAMs in a tumor or near a tumor (e.g., as compared to the number of TAMs prior to administration of a multispecific molecule described herein (e.g., prior to 1, 2, 3, 4, 5, 6, 7, 8, 9, 10 or more administrations of a multispecific molecule described herein). Reducing includes decreasing any number of TAMs (e.g., 1%, 5%, 10%, 20%, 30%, 40%, 50%, 60%, 70%, 80%, 90%, 95%, 99%, 100%, all, or substantially) (e.g., as compared to the number of TAMs prior to administration of a multispecific molecule described herein (e.g., prior to 1, 2, 3, 4, 5, 6, 7, 8, 9, 10 or more administrations of a multispecific molecule described herein).

As used herein, the term “myeloid derived suppressor cell” or “MDSC” generally refers to a cell of myeloid origin that is capable of promoting immunosuppression and commonly express CD33, CD11b and CD45. Various subpopulations of MDSCs have been defined, for example monocytic-MDSCs (M-MDSCs) are commonly associated with expression of CD14 and CD124 and low expression of HLA-DR. In some embodiments, the MDSC population is an MO-MDSC population. Polymorphonuclear MDSCs (PMN-MDSCs) are associated with expression of CD15, CD66b, and CD124, and no expression of HLA-DR. Immature MDSCs (I-MDSCs) are associated with expression of CD117 and CD34 and no expression of LIN and HLA-DR. See e.g., Ugel et al. (2015) JCI Vol 125 (9), page 3365.

Various aspects of the invention are described in further detail below. Additional definitions are set out throughout the specification.

### Antigens

TAM targeting antigens of the present disclosure include, e.g., CSF1R, CCR2, CXCR2, CD68, CD163, CX3CR1, MARCO, CD204, CD52, and folate receptor beta. Exemplary amino acid sequences of TAM targeting antigens are provided herein.

### CSF1R

CSF1R (also known as Macrophage colony-stimulating factor 1 receptor) is a tyrosine-protein kinase that acts as cell-surface receptor for CSF1 and IL34 and plays an essential role in the regulation of survival, proliferation and differentiation of hematopoietic precursor cells, especially mononuclear phagocytes, such as macrophages and monocytes. CSF1R promotes the release of pro-inflammatory chemokines in response to IL34 and CSF1, and thereby plays an important role in innate immunity and in inflammatory processes. Exemplary CSF1R immature amino acid sequences are provided in SEQ ID NOs: 87 and 88.

CSF1R immature amino acid sequence isoform 1
(identifier: P07333-1):
SEQ ID NO: 87
MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVESAY
LNLSSEQNLIQEVTVGEGLNLKVMVEAYPGLQGFNWTYLGPFSDHQPEPK
LANATTKDTYRHTFTLSLPRLKPSEAGRYSFLARNPGGWRALTFELTLRY
PPEVSVIWTFINGSGTLLCAASGYPQPNVTWLQCSGHTDRCDEAQVLQVW
DDPYPEVLSQEPFHKVTVQSLLTVETLEHNQTYECRAHNSVGSGSWAFIP
ISAGAHTHPPDEFLFTPVVVACMSIMALLLLLLLLLLYKYKQKPKYQVRW
KIIESYEGNSYTFIDPTQLPYNEKWEFPRNNLQFGKTLGAGAFGKVVEAT
AFGLGKEDAVLKVAVKMLKSTAHADEKEALMSELKIMSHLGQHENIVNLL
GACTHGGPVLVITEYCCYGDLLNFLRRKAEAMLGPSLSPGQDPEGGVDYK
NIHLEKKYVRRDSGFSSQGVDTYVEMRPVSTSSNDSFSEQDLDKEDGRPL
ELRDLLHFSSQVAQGMAFLASKNCIHRDVAARNVLLTNGHVAKIGDFGLA
RDIMNDSNYIVKGNARLPVKWMAPESIFDCVYTVQSDVWSYGILLWEIFS
LGLNPYPGILVNSKFYKLVKDGYQMAQPAFAPKNIYSIMQACWALEPTHR
PTFQQICSFLQEQAQEDRRERDYTNLPSSSRSGGSGSSSSELEEESSSEH
LTCCEQGDIAQPLLQPNNYQFC
CSF1R immature amino acid sequence isoform 2
(identifier: P07333-2):
SEQ ID NO: 88
MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVGTPS
PSLCPA

### CCR2

CCR2 (also known as C-C chemokine receptor type 2) is a G protein coupled receptor for the CCL2, CCL7 and CCL13 chemokines. CCR2 is known to function in the recruitment of monocytes/macrophages and T cells. CCR2 is expressed is expressed on monocytes and a small subpopulation of T cells and exhibits an almost identical expression pattern in mice and humans (Mack et al. J Immunol 2001; 166:4697-4704). Exemplary CCR2 amino acid sequences are provided in SEQ ID NOs: 89 and 90.

CCR2 amino acid sequence isoform A (Identifier:
P41597-1):
SEQ ID NO: 89
MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRSLFHIALGCRIAPLQKPVCGGPGVRPGKNVKVTTQGL
LDGRGKGKSIGRAPEASLQDKEGA
CCR2 amino acid sequence isoform B (Identifier:
P41597-2):
SEQ ID NO: 90
MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRRYLSVFFRKHITKRFCKQCPVFYRETVDGVTSTNTPS
TGEQEVSAGL

### CXCR2

CXCR2 (also known as interleukin-8 receptor) is the G protein coupled receptor for IL8 which is a neutrophil chemotactic factor. Binding of IL8 to the receptor causes activation of neutrophils. This response is mediated via a G-protein that activates a phosphatidylinositol-calcium second messenger system. CXCR2 binds to IL-8 with high affinity, and also binds with high affinity to CXCL3, GRO/MGSA and NAP-2. CXCR2 is expressed at high levels on circulating neutrophils and is critical for directing their migration to sites of inflammation (J Clin Invest. 2012; 122(9):3127-3144). An exemplary CXCR2 amino acid sequence is provided in SEQ ID NO: 91.

CXCR2 amino acid sequence (Identifier: P25025-1):
SEQ ID NO: 91
MEDFNMESDSFEDFWKGEDLSNYSYSSTLPPFLLDAAPCEPESLEINKYF
VVIIYALVFLLSLLGNSLVMLVILYSRVGRSVTDVYLLNLALADLLFALT
LPIWAASKVNGWIFGTFLCKVVSLLKEVNFYSGILLLACISVDRYLAIVH
ATRTLTQKRYLVKFICLSIWGLSLLLALPVLLFRRTVYSSNVSPACYEDM
GNNTANWRMLLRILPQSFGFIVPLLIMLFCYGFTLRTLFKAHMGQKHRAM
RVIFAVVLIFLLCWLPYNLVLLADTLMRTQVIQETCERRNHIDRALDATE
ILGILHSCLNPLIYAFIGQKFRHGLLKILAIHGLISKDSLPKDSRPSFVG
SSSGHTSTTL

### Exemplary Antibodies

Exemplary antibodies binding TAM antigens are provided throughout the specification and below. Exemplary anti-CSF1R antibodies are described herein as well as in WO2009026303A1; WO2011123381A1; WO2016207312A1; WO2016106180A1; US20160220669A1; US20160326254A1; WO2013169264A1; WO2013087699A1; WO2011140249A2; WO2011131407A1; WO2011123381A1; WO2011107553A1; and WO2011070024A1, all of which are herein incorporated by reference in their entirety. Exemplary CCR2 antibodies are described herein as well as in WO2013192596A2; WO2010021697A2; WO2001057226A1; and WO1997031949A1, all of which are herein incorporated by reference in their entirety. Exemplary CXCR2 antibodies are described in WO2014170317A1 and US20160060347 (see e.g., a) SEQ ID NO: 14 (light chain) and SEQ ID NO: 15 (heavy chain); b) SEQ ID NO: 24 (light chain) and SEQ ID NO: 25 (heavy chain); c) SEQ ID NO: 34 (light chain) and SEQ ID NO: 35 (heavy chain); d) SEQ ID NO: 44 (light chain) and SEQ ID NO: 45 (heavy chain); e) SEQ ID NO: 54 (light chain) and SEQ ID NO: 55 (heavy chain); f) SEQ ID NO: 64 (light chain) and SEQ ID NO: 65 (heavy chain); g) SEQ ID NO: 74 (light chain) and SEQ ID NO: 75 (heavy chain); h) SEQ ID NO: 84 (light chain) and SEQ ID NO: 85 (heavy chain)), all of which are herein incorporated by reference in their entirety. Exemplary anti-CD163 antibodies are provided in US20120258107 (see e.g., MAC2158, MAC2-48), herein incorporated by reference in its entirety. Exemplary anti-CD52 antibodies are described in US20050152898, herein incorporated by reference in its entirety. Exemplary anti-folate antibodies are described in U.S. Pat. No. 9,522,196, herein incorporated by reference in its entirety. Exemplary anti-CD52 antibodies are described in US20050152898, herein incorporated by reference in its entirety. Exemplary anti-MARCO antibodies are described in WO2016196612, herein incorporated by reference in its entirety.

### Antibody Molecules

In one embodiment, the multispecific molecule comprises an antibody molecule that binds to a first tumor associated macrophage (TAM) antigen; and an antibody molecule that binds to a second TAM antigen. In some embodiments, the first and/or second TAM antigen is, e.g., a mammalian, e.g., a human. For example, the antibody molecule binds specifically to an epitope, e.g., linear or conformational epitope, on the TAM antigen.

In one embodiment, the multispecific molecule comprises an antibody molecule that binds to a first myeloid derived suppressor cell (MDSC) antigen; and an antibody molecule that binds to a second MDSC antigen. In some embodiments, the first and/or second MDSC antigen is, e.g., a mammalian, e.g., a human. For example, the antibody molecule binds specifically to an epitope, e.g., linear or conformational epitope, on the MDSC antigen.

In an embodiment, an antibody molecule is a monospecific antibody molecule and binds a single epitope. E.g., a monospecific antibody molecule having a plurality of immunoglobulin variable region sequences, each of which binds the same epitope.

In an embodiment an antibody molecule is a multispecific antibody molecule, e.g., it comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable region. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable region sequence which has binding specificity for a first epitope and a second immunoglobulin variable region sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable region sequence and a light chain variable region sequence which have binding specificity for a first epitope and a heavy chain variable region sequence and a light chain variable region sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable region sequence (abbreviated herein as VH), and a light (L) chain variable region sequence (abbreviated herein as VL). In an embodiment an antibody molecule comprises or consists of a heavy chain and a light chain (referred to herein as a half antibody. In another example, an antibody molecule includes two heavy (H) chain variable region sequences and two light (L) chain variable region sequence, thereby forming two antigen binding sites, such as Fab, Fab′, F(ab′)₂, Fc, Fd, Fd′, Fv, single chain antibodies (scFv for example), single variable region antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable region; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable region derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable region (VH) are numbered 31-35 (HCDR1), 50-65 (HCDR2), and 95-102 (HCDR3); and the CDR amino acid residues in the light chain variable region (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific Antibody Molecules

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BslgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable regions (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HERS. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant regions. For example, some BsAb lack an Fc region.

In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Multispecific Antibody Molecules Targeting CSF1R

In one aspect, disclosed herein is a multispecific antibody molecule comprising a CSF1R binding moiety. In some embodiments, the CSF1R binding moiety comprises an anti-CSF1R antibody molecule. Exemplary anti-CSF1R antibody molecule sequences are described in WO2009026303A1; WO2011123381A1; WO2016207312A1; WO2016106180A1; US20160220669A1; US20160326254A1; WO2013169264A1; WO2013087699A1; WO2011140249A2; WO2011131407A1; WO2011123381A1; WO2011107553A1; and WO2011070024A1, all of which are herein incorporated by reference in their entirety. In some embodiments, the CSF1R binding moiety comprises the CDR (e.g., one, two, three, four, five, or all six CDRs), VH, VL, heavy chain, or light chain sequences of emactuzumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of cabiralizumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of AMG820, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises the CDR (e.g., one, two, three, four, five, or all six CDRs), VH, VL, heavy chain, or light chain sequences of IMC-CS4, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the CSF1R binding moiety comprises a VH or VL amino acid sequence disclosed in Table 1, a CDR of a VH or VL amino acid sequence disclosed in Table 1, or a sequence substantially identical thereto.

TABLE 1
Exemplary anti-CSF1R antibody molecule sequences

| SEQ ID | | |
|---|---|---|
| NO | Description | Sequence |
| SEQ ID | αmCSF1R VH | QVQLQQSGAELVKPGSSVKISCKASGYTFTSNFMHWIKQQPGNG |
| NO: 48 | | LEWIGWIYPGDGDTEYNQKFNGKATLTADKSSSTAYMQLSSLTS |
| | | EDSAVYFCAVNYGGYVLDAWGQGASVTVSS |
| SEQ ID | αmCSF1R VL | EIVLTQSPTTMAASPGEKVTITCRASSSTNYMSWYQQKSGASPKP |
| NO: 50 | | WIYETSKLASGVPDRFSGSGSGTSYSFTISSMETEDAATYYCHQW |
| | | SSTPLTFGSGTKLEIK |
| SEQ ID | αhCSF1R | QVQLVQSGAEVKKPGASVKVSCKASGYTFTSYDISWVRQAPGQG |
| NO: 66 | emactuzumab | LEWMGVIWTDGGTNYAQKLQGRVTMTTDTSTSTAYMELRSLRS |
| | VH | DDTAVYYCARDQRLYFDVWGQGTTVTVSS |
| SEQ ID | αhCSF1R | DIQMTQSPSSLSASVGDRVTITCRASEDVNTYVSWYQQKPGKAP |
| NO: 67 | emactuzumab | KLLIYAASNRYTGVPSRFSGSGSGTDFTLTISSLQPEDFATYYCQQ |
| | VL | SFSYPTFGQGTKLEIK |
| SEQ ID | αhCSF1R | QVQLVQSGAEVKKPGSSVKVSCKASGYTFTDNYMIWVRQAPGQ |
| NO: 69 | cabiralizumab | GLEWMGDINPYNGGTTFNQKFKGRVTITADKSTSTAYMELSSLR |
| | VH | SEDTAVYYCARESPYFSNLYVMDYWGQGTLVTVSS |
| SEQ ID | αhCSF1R | EIVLTQSPATLSLSPGERATLSCKASQSVDYDGDNYMNWYQQKP |
| NO: 70 | cabiralizumab | GQAPRLLIYAASNLESGIPARFSGSGSGTDFTLTISSLEPEDFAVYY |
| | VL | CHLSNEDLSTFGGGTKVEIK |

### Multispecific Antibody Molecules Targeting CCR2

In one aspect, disclosed herein is a multispecific antibody molecule comprising a CCR2 binding moiety. Exemplary CCR2 antibodies are described herein as well as in WO2013192596A2; WO2010021697A2; WO2001057226A1; and WO1997031949A1, all of which are herein incorporated by reference in their entirety. In some embodiments, the CCR2 binding moiety comprises the CDR (e.g., one, two, three, four, five, or all six CDRs), VH, VL, heavy chain, or light chain sequences of plozalizumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the CCR2 binding moiety comprises a VH or VL amino acid sequence disclosed in Table 2, a CDR of a VH or VL amino acid sequence disclosed in Table 2, or a sequence substantially identical thereto.

TABLE 2
Exemplary anti-CCR2 antibody molecule sequences

| SEQ ID | | |
|---|---|---|
| NO | Description | Sequence |
| SEQ ID | αCCR2 MC12 | QVQLQESGPGLVQPSQTLSLTCTVSGFSLTDFSVHWVRQPPGKG |
| NO: 44 | VH | LEWMGRIRSEGNTDYNSALKSRLSISRDTSKSQVFLKMNSLQTE |
| | | DTAIYFCTRGDILGFGYWGQGVMVTVSS |
| SEQ ID | αCCR2 MC12 | DIVMTQSPLSVSVTPGESASISCRSSKSLLHFKGITFVYWYLQKPG |
| NO: 45 | VL | QSPQLLIFRMSSLASGVPDRFSGSGSETDFTLKISRVEAEDVGTYY |
| | | CGQLLENPYTFGAGTKLELK |
| SEQ ID | αhCCR2 | EVQLVESGGGLVKPGGSLRLSCAASGFTFSAYAMNWVRQAPGK |
| NO: 54 | plozalizumab | GLEWVGRIRTKNNNYATYYADSVKDRFTISRDDSKNTLYLQMN |
| | VH | SLKTEDTAVYYCTTFYGNGVWGQGTLVTVSS |
| SEQ ID | αhCCR2 | DVVMTQSPLSLPVTLGQPASISCKSSQSLLDSDGKTFLNWFQQRP |
| NO: 57 | plozalizumab | GQSPRRLIYLVSKLDSGVPDRFSGSGSGTDFTLKISRVEAEDVGV |
| | VL | YYCWQGTHFPYTFGQGTRLEIK |
| SEQ ID | αhCCR2 D1 | EVQLVQSGAEVKKPGASVKVSCKASGYTFTGYHMHWVRQAPG |
| NO: 59 | VH | QGLEWMGWINPNSGVTKYAQKFQGRVTMTRDTSINTAYMELS |
| | | RLRFDDTDVYYCATGGFGYWGEGTLVTVSS |
| SEQ ID | αhCCR2 D1 | LPVLTQPPSVSKGLRQTATLTCTGNSNNVGNQGAAWLQQHQGQ |
| NO: 60 | VL | PPKLLSYRNHNRPSGVSERFSPSRSGDTSSLTITGLQPEDEADYYC |
| | | LAWDSSLRAFVFGTGTKLTVL |
| SEQ ID | αhCCR2 | QVQLVQSGAEVKKPGASVKVSCKASGYTFSSYYMHWVRQAPG |
| NO: 62 | 42G7 VH | QGLEWMGIINPSGGNTSYAQKFQGRVTMTRDTSTSTVYMELSSL |
| | | RSEDTAVYYCARGGYQLPHGRARAFDMWGQGTMVTVSS |
| SEQ ID | αhCCR2 | AIRMTQSPLSLPVTLGQPASISCTSSQSLVYRDGTTYLNWFQQRP |
| NO: 63 | 42G7 VL | GQSPRRLIYKVSNRDSGVPDRFTGSGSGTTFTLTISRVEAEDVGIY |
| | | YCMQGTHWPLTFGQGTKVEIK |
| SEQ ID | αhCCR2 | EVQLVESGGGLVQPGGSLRLSCVASGFTFSDYWMSWVRQAPGK |
| NO: 64 | 43G12 VH | GLEWVANIKKDGSVNYYVDSVKGRFTISRDNAKNSLYLQMNSL |
| | | RAEDTAVYYCTRFDYWGQGTLVTVSS |
| SEQ ID | αhCCR2 | QAGLTQPPSVSKGLRQTATLTCTGNSNNVGNQGAAWLQQHQG |
| NO: 65 | 43G12 VL | HPPKLLFYRNNNRASGISERLSASRSGNTASLTITGLQPEDEADY |
| | | YCLTWDSSLSVVVFGGGTKLTVL |

### Multispecific Antibody Molecules Targeting PD-L1

In one aspect, disclosed herein is a multispecific antibody molecule comprising a PD-L1 binding moiety. In some embodiments, the PD-L1 binding moiety comprises an anti-PD-L1 antibody molecule. Exemplary anti-PD-L1 antibody molecule sequences are described in WO2013079174, WO 2010077634, WO2007/005874, and US20120039906, all of which are herein incorporated by reference in their entirety. In some embodiments, the PD-L1 binding moiety comprises the CDR (e.g., one, two, three, four, five, or all six CDRs), VH, VL, heavy chain, or light chain sequences of durvalumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the PD-L1 binding moiety comprises the CDR (e.g., one, two, three, four, five, or all six CDRs), VH, VL, heavy chain, or light chain sequences of atezolizumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the PD-L1 binding moiety comprises the CDR (e.g., one, two, three, four, five, or all six CDRs), VH, VL, heavy chain, or light chain sequences of avelumab, or a sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the PD-L1 binding moiety comprises a VH or VL amino acid sequence disclosed in Table 3, a CDR of a VH or VL amino acid sequence disclosed in Table 3, or a sequence substantially identical thereto.

TABLE 3
Exemplary anti-PD-L1 antibody molecule sequences

| SEQ ID | | |
|---|---|---|
| NO | Description | Sequence |
| SEQ ID | αPD-L1 | EVQLVESGGGLVQPGGSLRLSCAASGFTFSRYWMSWVRQAPGK |
| NO: 109 | durvalumab | GLEWVANIKQDGSEKYYVDSVKGRFTISRDNAKNSLYLQMNSL |
| | VH | RAEDTAVYYCAREGGWFGELAFDYWGQGTLVTVSS |
| SEQ ID | αPD-L1 | EIVLTQSPGTLSLSPGERATLSCRASQRVSSSYLAWYQQKPGQAP |
| NO: 110 | durvalumab | RLLIYDASSRATGIPDRFSGSGSGTDFTLTISRLEPEDFAVYYCQQ |
| | VL | YGSLPWTFGQGTKVEIK |
| SEQ ID | αPD-L1 | EVQLVESGGGLVQPGGSLRLSCAASGFTFSDSWIHWVRQAPGK |
| NO: 111 | atezolizumab | GLEWVAWISPYGGSTYYADSVKGRFTISADTSKNTAYLQMNSL |
| | VH | RAEDTAVYYCARRHWPGGFDYWGQGTLVTVSS |
| SEQ ID | αPD-L1 | DIQMTQSPSSLSASVGDRVTITCRASQDVSTAVAWYQQKPGKAP |
| NO: 112 | atezolizumab | KLLIYSASFLYSGVPSRFSGSGSGTDFTLTISSLQPEDFATYYCQQ |
| | VL | YLYHPATFGQGTKVEIK |
| SEQ ID | αPD-L1 | EVQLLESGGGLVQPGGSLRLSCAASGFTFSSYIMMWVRQAPGK |
| NO: 113 | avelumab VH | GLEWVSSIYPSGGITFYADTVKGRFTISRDNSKNTLYLQMNSLRA |
| | | EDTAVYYCARIKLGTVTTVDYWGQGTLVTVSS |
| SEQ ID | αPD-L1 | QSALTQPASVSGSPGQSITISCTGTSSDVGGYNYVSWYQQHPGK |
| NO: 114 | avelumab VL | APKLMIYDVSNRPSGVSNRFSGSKSGNTASLTISGLQAEDEADYY |
| | | CSSYTSSSTRVFGTGTKVTVL |

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable region of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable region of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

Exemplary structures of the multifunctional molecules defined herein are described below. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

### Heterodimerized Antibody Molecules

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, I368A and/or Y407V), numbered based on the Eu numbering system.

### Strand Exchange Engineered Domains (SEED)

SEED is based on sequence exchanges between IgG1 and IgA to create non-identical chains which heterodimerize preferentially. Alternating sequences from human IgA and IgG in the SEED CH3 domains generate two asymmetric but complementary domains, designated AG and GA. The SEED design allows efficient generation of AG/GA heterodimers, while disfavoring homodimerization of AG and GA SEED CH3 domains.

### Common Light Chain & CrossMab

Light chain mispairing must be avoided to generate homogenous preparations of bispecific IgGs. One way to achieve this is through the use of the common light chain principle, i.e. combining two binders that share one light chain but still have separate specificities. Another option is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable regions (without peptide linker) fused to the C-terminus of CH3 domains of IgGs.

### Fc-Less Bispecifics

Fc-less bispecifics are characterized by generally having smaller size than Fc-containing entities. Common bispecific of this class include Fab-scFv2 and Fab-scFv molecules. This class also includes, e.g., BiTEs (bispecific T-cell engagers), diabodies, TandAbs (tetravalent tandem antibodies), and DARTs (dual affinity retargeting molecules). BiTEs are created by fusing two scFvs via a flexible linker peptide. Diabodies consist of two VH and two VL domains from two different antibodies. Interaction only with complementary domains on another chain is achieved by attaching domains with short linker peptides which permits pairing only with VH and VL domains. VH of the first binder linked to the VL of the second binder is co-expressed with the VH of the second antibody linked to VL of the first antibody. TandAbs molecules are generated by functional dimerization of a protein consisting of four antibody variable H- and L-chains in an orientation that prevents intramolecular pairing. DARTs are entities that are stabilized by disulfide bonds which apply a similar design concept to that of diabodies.

### Kappa/Lambda Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US2017/53053 filed on Sep. 22, 2017, incorporated herein by reference in its entirety.

In embodiments, the multispecific molecules include a multispecific antibody molecule, e.g., an antibody molecule comprising two binding specificities, e.g., a bispecific antibody molecule. The multispecific antibody molecule includes:

a lambda light chain polypeptide 1 (LLCP1) specific for a first epitope;

a heavy chain polypeptide 1 (HCP1) specific for the first epitope;

a kappa light chain polypeptide 2 (KLCP2) specific for a second epitope; and

a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:

LLCP1 has a higher affinity for HCP1 than for HCP2; and/or

KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific antibody molecule molecules have a LLCP1complexed, or interfaced with, a HCP1.

In some embodiments of the multispecific antibody molecule disclosed herein:

the HCP1 has a greater affinity for HCP2, than for a second molecule of HCP1; and/or

the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1complexed, or interfaced with, a HCP2.

In another aspect, disclosed herein is a method for making, or producing, a multispecific antibody molecule. The method includes:

(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));

(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));

(iii) providing a lambda chain polypeptide (e.g., a lambda light variable region (VLλ), a lambda light constant chain (VLλ), or both) that preferentially associates with the first heavy chain polypeptide (e.g., the first VH); and

(iv) providing a kappa chain polypeptide (e.g., a lambda light variable region (VLκ), a lambda light constant chain (VLκ), or both) that preferentially associates with the second heavy chain polypeptide (e.g., the second VH),

under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:

(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;

(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;

(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and

(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Multispecific Molecules Comprising Non-Contiguous Polypeptides

In one embodiment, the multispecific molecule is not a single polypeptide chain.

In one embodiment, the antibody molecule includes two, complete heavy chains and two, complete light chains. In one embodiment, the multispecific molecules having at least two or at least three non-contiguous polypeptide chains include a first and second heavy chain constant regions (e.g., a first and second Fc region) in at least two non-contiguous polypeptide chains, e.g., as described herein.

In embodiments, the multispecific molecule is a bispecific or bifunctional molecule, wherein the first and second polypeptides (i) and (ii) are non-contiguous, e.g., are two separate polypeptide chains. In some embodiments, the first and second polypeptides (i) and (ii) include a paired amino acid substitution at a position chosen from one or more of 347, 349, 350, 351, 366, 368, 370, 392, 394, 395, 397, 398, 399, 405, 407, or 409, e.g., of the Fc region of human IgG1, numbered based on the Eu numbering system. For example, the first heavy chain constant region (e.g., the first Fc region) can include an amino acid substitution chosen from: T366S, L368A, or Y407V (e.g., corresponding to a cavity or hole), and the second heavy chain constant region (e.g., the second Fc region) includes a T366W (e.g., corresponding to a protuberance or knob), numbered based on the Eu numbering system. In some embodiments, the first and second polypeptides are a first and second member of a heterodimeric first and second Fc region.

In some embodiments, the first polypeptide has the following configuration from N-to-C:

(a) a first portion of a first antigen domain, e.g., a first VH-CH1 of a Fab molecule, that binds to a first antigen, e.g., CSF1R, connected, optionally via a linker to, the first heavy chain constant region (e.g., the CH2 connected to the CH3 region) (e.g., a first Fc region); (b) a first portion of a second antigen domain, e.g., a second VH-CH1 of a Fab molecule, that binds to a second antigen, e.g., CCR2 or CXCR2, connected, optionally via a linker to, the second heavy chain constant region (e.g., the CH2 connected to the CH3 region) (e.g., a first Fc region); (c) the third polypeptide has the following configuration from N-to-C: a second portion of the first antigen domain, e.g., a first VL-CL of the Fab, where the VL is of kappa subtype and binds to the first antigen, e.g., CSF1R (e.g., the same antigen bound by the first VH-CH1); (d) the fourth polypeptide has the following configuration from N-to-C: a second portion of the second antigen domain, e.g. a second VL-CL of the Fab, where the VL is of lambda subtype and binds to a second antigen, e.g., a cancer antigen, e.g., CCR2 or CXCR2 (e.g., the same antigen bound by the second VH-CH1).

In embodiments, the first heavy chain constant region (e.g., the first CH2-CH3 region) includes a protuberance or knob, e.g., as described herein. In embodiments, the second heavy chain constant region (e.g., the second CH2-CH3 region) includes a cavity or hole. In embodiments, the first and second heavy chain constant regions promote heterodimerization of the bispecific molecule.

### TGF-Beta Inhibitor

In one aspect, provided herein is a multispecific antibody molecule comprising a TGF-beta inhibitor. In some embodiments, the TGF-beta inhibitor binds to and inhibits TGF-beta, e.g., reduces the activity of TGF-beta. In some embodiments, the TGF-beta inhibitor inhibits (e.g., reduces the activity of) TGF-beta 1. In some embodiments, the TGF-beta inhibitor inhibits (e.g., reduces the activity of) TGF-beta 2. In some embodiments, the TGF-beta inhibitor inhibits (e.g., reduces the activity of) TGF-beta 3. In some embodiments, the TGF-beta inhibitor inhibits (e.g., reduces the activity of) TGF-beta 1 and TGF-beta 3. In some embodiments, the TGF-beta inhibitor inhibits (e.g., reduces the activity of) TGF-beta 1, TGF-beta 2, and TGF-beta 3.

In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises a TGFBR1 polypeptide (e.g., an extracellular domain of TGFBR1 or functional variant thereof). In some embodiments, the TGF-beta inhibitor comprises a TGFBR2 polypeptide (e.g., an extracellular domain of TGFBR2 or functional variant thereof). In some embodiments, the TGF-beta inhibitor comprises a TGFBR3 polypeptide (e.g., an extracellular domain of TGFBR3 or functional variant thereof). In some embodiments, the TGF-beta inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-beta inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-beta inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-beta receptor polypeptides that can be used as TGF-beta inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of SEQ ID NO: 95, or a sequence substantially identical thereto (e.g., a sequence that is at least 80%, 85%, 90%, or 95% identical thereto). In some embodiments, the TGF-beta inhibitor comprises an extracellular domain of SEQ ID NO: 96, or a sequence substantially identical thereto (e.g., a sequence that is at least 80%, 85%, 90%, or 95% identical thereto). In some embodiments, the TGF-beta inhibitor comprises an extracellular domain of SEQ ID NO: 97, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 104, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 105, or a sequence substantially identical thereto (e.g., a sequence that is at least 80%, 85%, 90%, or 95% identical thereto).

In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 98, or a sequence substantially identical thereto (e.g., a sequence that is at least 80%, 85%, 90%, or 95% identical thereto). In some embodiments, the TGF-beta inhibitor comprises an extracellular domain of SEQ ID NO: 99, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 100, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 101, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 102, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 103, or a sequence substantially identical thereto (e.g., a sequence that is at least 80%, 85%, 90%, or 95% identical thereto).

In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an extracellular domain of SEQ ID NO: 106, or a sequence substantially identical thereto (e.g., a sequence that is at least 80%, 85%, 90%, or 95% identical thereto). In some embodiments, the TGF-beta inhibitor comprises an extracellular domain of SEQ ID NO: 107, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 108, or a sequence substantially identical thereto (e.g., a sequence that is at least 80%, 85%, 90%, or 95% identical thereto).

In some embodiments, the TGF-beta inhibitor comprises no more than one TGF-beta receptor extracellular domain. In some embodiments, the TGF-beta inhibitor comprises two or more (e.g., two, three, four, five, or more) TGF-beta receptor extracellular domains, linked together, e.g., via a linker.

TABLE 4
Exemplary amino acid sequences of TGF-beta polypeptides
or TGF-beta receptor polypeptides

| SEQ ID | | |
|---|---|---|
| NO | Description | Amino acid sequence |
| SEQ ID | Immature | MPPSGLRLLLLLLPLLWLLVLTPGRPAAGLSTCKTIDMELVKRKRIE |
| NO: 92 | human | AIRGQILSKLRLASPPSQGEVPPGPLPEAVLALYNSTRDRVAGESAEP |
| | TGF-beta 1 | EPEPEADYYAKEVTRVLMVETHNEIYDKFKQSTHSIYMFFNTSELRE |
| | (P01137-1) | AVPEPVLLSRAELRLLRLKLKVEQHVELYQKYSNNSWRYLSNRLLA |
| | | PSDSPEWLSFDVTGVVRQWLSRGGEIEGFRLSAHCSCDSRDNTLQV |
| | | DINGFTTGRRGDLATIHGMNRPFLLLMATPLERAQHLQSSRHRRAL |
| | | DTNYCFSSTEKNCCVRQLYIDFRKDLGWKWIHEPKGYHANFCLGP |
| | | CPYIWSLDTQYSKVLALYNQHNPGASAAPCCVPQALEPLPIVYYVG |
| | | RKPKVEQLSNMIVRSCKCS |
| SEQ ID | Human | LSTCKTIDMELVKRKRIEAIRGQILSKLRLASPPSQGEVPPGPLPEAV |
| NO: 117 | TGF-beta 1 | LALYNSTRDRVAGESAEPEPEPEADYYAKEVTRVLMVETHNEIYDK |
| | (P01137-1) | FKQSTHSIYMFFNTSELREAVPEPVLLSRAELRLLRLKLKVEQHVEL |
| | | YQKYSNNSWRYLSNRLLAPSDSPEWLSFDVTGVVRQWLSRGGEIE |
| | | GFRLSAHCSCDSRDNTLQVDINGFTTGRRGDLATIHGMNRPFLLLM |
| | | ATPLERAQHLQSSRHRRALDTNYCFSSTEKNCCVRQLYIDFRKDLG |
| | | WKWIHEPKGYHANFCLGPCPYIWSLDTQYSKVLALYNQHNPGASA |
| | | APCCVPQALEPLPIVYYVGRKPKVEQLSNMIVRSCKCS |
| SEQ ID | Immature | MHYCVLSAFLILHLVTVALSLSTCSTLDMDQFMRKRIEAIRGQILSK |
| NO: 93 | human | LKLTSPPEDYPEPEEVPPEVISIYNSTRDLLQEKASRRAAACERERSD |
| | TGF-beta 2 | EEYYAKEVYKIDMPPFFPSENAIPPTFYRPYFRIVRFDVSAMEKNAS |
| | (P61812-1) | NLVKAEFRVFRLQNPKARVPEQRIELYQILKSKDLTSPTQRYIDSKV |
| | | VKTRAEGEWLSFDVTDAVHEWLHHKDRNLGFKISLHCPCCTFVPS |
| | | NNYIIPNKSEELEARFAGIDGTSTYTSGDQKTIKSTRKKNSGKTPHLL |
| | | LMLLPSYRLESQQTNRRKKRALDAAYCFRNVQDNCCLRPLYIDFKR |
| | | DLGWKWIHEPKGYNANFCAGACPYLWSSDTQHSRVLSLYNTINPE |
| | | ASASPCCVSQDLEPLTILYYIGKTPKIEQLSNMIVKSCKCS |
| SEQ ID | Human | LSTCSTLDMDQFMRKRIEAIRGQILSKLKLTSPPEDYPEPEEVPPEVIS |
| NO: 118 | TGF-beta 2 | IYNSTRDLLQEKASRRAAACERERSDEEYYAKEVYKIDMPPFFPSEN |
| | (P61812-1) | AIPPTFYRPYFRIVRFDVSAMEKNASNLVKAEFRVFRLQNPKARVPE |
| | | QRIELYQILKSKDLTSPTQRYIDSKVVKTRAEGEWLSFDVTDAVHE |
| | | WLHHKDRNLGFKISLHCPCCTFVPSNNYIIPNKSEELEARFAGIDGTS |
| | | TYTSGDQKTIKSTRKKNSGKTPHLLLMLLPSYRLESQQTNRRKKRA |
| | | LDAAYCFRNVQDNCCLRPLYIDFKRDLGWKWIHEPKGYNANFCAG |
| | | ACPYLWSSDTQHSRVLSLYNTINPEASASPCCVSQDLEPLTILYYIGK |
| | | TPKIEQLSNMIVKSCKCS |
| SEQ ID | Immature | MKMHLQRALVVLALLNFATVSLSLSTCTTLDFGHIKKKRVEAIRGQ |
| NO: 94 | human | ILSKLRLTSPPEPTVMTHVPYQVLALYNSTRELLEEMHGEREEGCTQ |
| | TGF-beta 3 | ENTESEYYAKEIHKFDMIQGLAEHNELAVCPKGITSKVFRFNVSSVE |
| | (P10600-1) | KNRTNLFRAEFRVLRVPNPSSKRNEQRIELFQILRPDEHIAKQRYIGG |
| | | KNLPTRGTAEWLSFDVTDTVREWLLRRESNLGLEISIHCPCHTFQPN |
| | | GDILENIHEVMEIKFKGVDNEDDHGRGDLGRLKKQKDHHNPHLIL |
| | | MMIPPHRLDNPGQGGQRKKRALDTNYCFRNLEENCCVRPLYIDFRQ |
| | | DLGWKWVHEPKGYYANFCSGPCPYLRSADTTHSTVLGLYNTLNPE |
| | | ASASPCCVPQDLEPLTILYYVGRTPKVEQLSNMVVKSCKCS |
| SEQ ID | Human | LSTCTTLDFGHIKKKRVEAIRGQILSKLRLTSPPEPTVMTHVPYQVL |
| NO: 119 | TGF-beta 3 | ALYNSTRELLEEMHGEREEGCTQENTESEYYAKEIHKFDMIQGLAE |
| | (P10600-1) | HNELAVCPKGITSKVFRFNVSSVEKNRTNLFRAEFRVLRVPNPSSKR |
| | | NEQRIELFQILRPDEHIAKQRYIGGKNLPTRGTAEWLSFDVTDTVRE |
| | | WLLRRESNLGLEISIHCPCHTFQPNGDILENIHEVMEIKFKGVDNED |
| | | DHGRGDLGRLKKQKDHHNPHLILMMIPPHRLDNPGQGGQRKKRAL |
| | | DTNYCFRNLEENCCVRPLYIDFRQDLGWKWVHEPKGYYANFCSGP |
| | | CPYLRSADTTHSTVLGLYNTLNPEASASPCCVPQDLEPLTILYYVGR |
| | | TPKVEQLSNMVVKSCKCS |
| SEQ ID | Immature | MEAAVAAPRPRLLLLVLAAAAAAAAALLPGATALQCFCHLCTKDN |
| NO: 95 | human | FTCVTDGLCFVSVTETTDKVIHNSMCIAEIDLIPRDRPFVCAPSSKTG |
| | TGFBR1 | SVTTTYCCNQDHCNKIELPTTVKSSPGLGPVELAAVIAGPVCFVCISL |
| | isoform 1 | MLMVYICHNRTVIHHRVPNEEDPSLDRPFISEGTTLKDLIYDMTTSG |
| | (P36897-1) | SGSGLPLLVQRTIARTIVLQESIGKGRFGEVWRGKWRGEEVAVKIFS |
| | | SREERSWFREAEIYQTVMLRHENILGFIAADNKDNGTWTQLWLVSD |
| | | YHEHGSLFDYLNRYTVTVEGMIKLALSTASGLAHLHMEIVGTQGKP |
| | | AIAHRDLKSKNILVKKNGTCCIADLGLAVRHDSATDTIDIAPNHRVG |
| | | TKRYMAPEVLDDSINMKHFESFKRADIYAMGLVFWEIARRCSIGGI |
| | | HEDYQLPYYDLVPSDPSVEEMRKVVCEQKLRPNIPNRWQSCEALR |
| | | VMAKIMRECWYANGAARLTALRIKKTLSQLSQQEGIKM |
| SEQ ID | Human | LQCFCHLCTKDNFTCVTDGLCFVSVTETTDKVIHNSMCIAEIDLIPR |
| NO: 120 | TGFBR1 | DRPFVCAPSSKTGSVTTTYCCNQDHCNKIELPTTVKSSPGLGPVELA |
| | isoform 1 | AVIAGPVCFVCISLMLMVYICHNRTVIHHRVPNEEDPSLDRPFISEGT |
| | (P36897-1) | TLKDLIYDMTTSGSGSGLPLLVQRTIARTIVLQESIGKGRFGEVWRG |
| | | KWRGEEVAVKIFSSREERSWFREAEIYQTVMLRHENILGFIAADNK |
| | | DNGTWTQLWLVSDYHEHGSLFDYLNRYTVTVEGMIKLALSTASGL |
| | | AHLHMEIVGTQGKPAIAHRDLKSKNILVKKNGTCCIADLGLAVRHD |
| | | SATDTIDIAPNHRVGTKRYMAPEVLDDSINMKHFESFKRADIYAMG |
| | | LVFWEIARRCSIGGIHEDYQLPYYDLVPSDPSVEEMRKVVCEQKLRP |
| | | NIPNRWQSCEALRVMAKIMRECWYANGAARLTALRIKKTLSQLSQ |
| | | QEGIKM |
| SEQ ID | Immature | MEAAVAAPRPRLLLLVLAAAAAAAAALLPGATALQCFCHLCTKDN |
| NO: 96 | human | FTCVTDGLCFVSVTETTDKVIHNSMCIAEIDLIPRDRPFVCAPSSKTG |
| | TGFBR1 | SVTTTYCCNQDHCNKIELPTTGPFSVKSSPGLGPVELAAVIAGPVCF |
| | isoform 2 | VCISLMLMVYICHNRTVIHHRVPNEEDPSLDRPFISEGTTLKDLIYD |
| | (P36897-2) | MTTSGSGSGLPLLVQRTIARTIVLQESIGKGRFGEVWRGKWRGEEV |
| | | AVKIFSSREERSWFREAEIYQTVMLRHENILGFIAADNKDNGTWTQ |
| | | LWLVSDYHEHGSLFDYLNRYTVTVEGMIKLALSTASGLAHLHMEI |
| | | VGTQGKPAIAHRDLKSKNILVKKNGTCCIADLGLAVRHDSATDTIDI |
| | | APNHRVGTKRYMAPEVLDDSINMKHFESFKRADIYAMGLVFWEIA |
| | | RRCSIGGIHEDYQLPYYDLVPSDPSVEEMRKVVCEQKLRPNIPNRW |
| | | QSCEALRVMAKIMRECWYANGAARLTALRIKKTLSQLSQQEGIKM |
| SEQ ID | Human | LQCFCHLCTKDNFTCVTDGLCFVSVTETTDKVIHNSMCIAEIDLIPR |
| NO: 121 | TGFBR1 | DRPFVCAPSSKTGSVTTTYCCNQDHCNKIELPTTGPFSVKSSPGLGP |
| | isoform 2 | VELAAVIAGPVCFVCISLMLMVYICHNRTVIHHRVPNEEDPSLDRPFI |
| | (P36897-2) | SEGTTLKDLIYDMTTSGSGSGLPLLVQRTIARTIVLQESIGKGRFGEV |
| | | WRGKWRGEEVAVKIFSSREERSWFREAEIYQTVMLRHENILGFIAA |
| | | DNKDNGTWTQLWLVSDYHEHGSLFDYLNRYTVTVEGMIKLALST |
| | | ASGLAHLHMEIVGTQGKPAIAHRDLKSKNILVKKNGTCCIADLGLA |
| | | VRHDSATDTIDIAPNHRVGTKRYMAPEVLDDSINMKHFESFKRADI |
| | | YAMGLVFWEIARRCSIGGIHEDYQLPYYDLVPSDPSVEEMRKVVCE |
| | | QKLRPNIPNRWQSCEALRVMAKIMRECWYANGAARLTALRIKKTL |
| | | SQLSQQEGIKM |
| SEQ ID | Immature | MEAAVAAPRPRLLLLVLAAAAAAAAALLPGATALQCFCHLCTKDN |
| NO: 97 | human | FTCVTDGLCFVSVTETTDKVIHNSMCIAEIDLIPRDRPFVCAPSSKTG |
| | TGFBR1 | SVTTTYCCNQDHCNKIELPTTGLPLLVQRTIARTIVLQESIGKGRFGE |
| | isoform 3 | VWRGKWRGEEVAVKIFSSREERSWFREAEIYQTVMLRHENILGFIA |
| | (P36897-3) | ADNKDNGTWTQLWLVSDYHEHGSLFDYLNRYTVTVEGMIKLALS |
| | | TASGLAHLHMEIVGTQGKPAIAHRDLKSKNILVKKNGTCCIADLGL |
| | | AVRHDSATDTIDIAPNHRVGTKRYMAPEVLDDSINMKHFESFKRAD |
| | | IYAMGLVFWEIARRCSIGGIHEDYQLPYYDLVPSDPSVEEMRKVVC |
| | | EQKLRPNIPNRWQSCEALRVMAKIMRECWYANGAARLTALRIKKT |
| | | LSQLSQQEGIKM |
| SEQ ID | Human | LQCFCHLCTKDNFTCVTDGLCFVSVTETTDKVIHNSMCIAEIDLIPR |
| NO: 122 | TGFBR1 | DRPFVCAPSSKTGSVTTTYCCNQDHCNKIELPTTGLPLLVQRTIARTI |
| | isoform 3 | VLQESIGKGRFGEVWRGKWRGEEVAVKIFSSREERSWFREAEIYQT |
| | (P36897-3) | VMLRHENILGFIAADNKDNGTWTQLWLVSDYHEHGSLFDYLNRYT |
| | | VTVEGMIKLALSTASGLAHLHMEIVGTQGKPAIAHRDLKSKNILVK |
| | | KNGTCCIADLGLAVRHDSATDTIDIAPNHRVGTKRYMAPEVLDDSI |
| | | NMKHFESFKRADIYAMGLVFWEIARRCSIGGIHEDYQLPYYDLVPS |
| | | DPSVEEMRKVVCEQKLRPNIPNRWQSCEALRVMAKIMRECWYAN |
| | | GAARLTALRIKKTLSQLSQQEGIKM |
| SEQ ID | Human | LQCFCHLCTKDNFTCVTDGLCFVSVTETTDKVIHNSMCIAEIDLIPR |
| NO: 104 | TGFBR1 | DRPFVCAPSSKTGSVTTTYCCNQDHCNKIELPTTVKSSPGLGPVEL |
| | fragment 1 | |
| SEQ ID | Human | ALQCFCHLCTKDNFTCVTDGLCFVSVTETTDKVIHNSMCIAEIDLIP |
| NO: 105 | TGFBR1 | RDRPFVCAPSSKTGSVTTTYCCNQDHCNKIEL |
| | fragment 2 | |
| SEQ ID | Immature | MGRGLLRGLWPLHIVLWTRIASTIPPHVQKSVNNDMIVTDNNGAV |
| NO: 98 | human | KFPQLCKFCDVRFSTCDNQKSCMSNCSITSICEKPQEVCVAVWRKN |
| | TGFBR2 | DENITLETVCHDPKLPYHDFILEDAASPKCIMKEKKKPGETFFMCSC |
| | isoform B | SSDECNDNIIFSEEYNTSNPDLLLVIFQVTGISLLPPLGVAISVIIIFYCY |
| | (short | RVNRQQKLSSTWETGKTRKLMEFSEHCAIILEDDRSDISSTCANNIN |
| | isoform) | HNTELLPIELDTLVGKGRFAEVYKAKLKQNTSEQFETVAVKIFPYEE |
| | (P37173-1) | YASWKTEKDIFSDINLKHENILQFLTAEERKTELGKQYWLITAFHAK |
| | | GNLQEYLTRHVISWEDLRKLGSSLARGIAHLHSDHTPCGRPKMPIV |
| | | HRDLKSSNILVKNDLTCCLCDFGLSLRLDPTLSVDDLANSGQVGTA |
| | | RYMAPEVLESRMNLENVESFKQTDVYSMALVLWEMTSRCNAVGE |
| | | VKDYEPPFGSKVREHPCVESMKDNVLRDRGRPEIPSFWLNHQGIQM |
| | | VCETLTECWDHDPEARLTAQCVAERFSELEHLDRLSGRSCSEEKIPE |
| | | DGSLNTTK |
| SEQ ID | Human | TIPPHVQKSVNNDMIVTDNNGAVKFPQLCKFCDVRFSTCDNQKSC |
| NO: 123 | TGFBR2 | MSNCSITSICEKPQEVCVAVWRKNDENITLETVCHDPKLPYHDFILE |
| | isoform B | DAASPKCIMKEKKKPGETFFMCSCSSDECNDNIIFSEEYNTSNPDLLL |
| | (short | VIFQVTGISLLPPLGVAISVIIIFYCYRVNRQQKLSSTWETGKTRKLM |
| | isoform) | EFSEHCAIILEDDRSDISSTCANNINHNTELLPIELDTLVGKGRFAEVY |
| | (P37173-1) | KAKLKQNTSEQFETVAVKIFPYEEYASWKTEKDIFSDINLKHENILQ |
| | | FLTAEERKTELGKQYWLITAFHAKGNLQEYLTRHVISWEDLRKLGS |
| | | SLARGIAHLHSDHTPCGRPKMPIVHRDLKSSNILVKNDLTCCLCDFG |
| | | LSLRLDPTLSVDDLANSGQVGTARYMAPEVLESRMNLENVESFKQT |
| | | DVYSMALVLWEMTSRCNAVGEVKDYEPPFGSKVREHPCVESMKD |
| | | NVLRDRGRPEIPSFWLNHQGIQMVCETLTECWDHDPEARLTAQCV |
| | | AERFSELEHLDRLSGRSCSEEKIPEDGSLNTTK |
| SEQ ID | Immature | MGRGLLRGLWPLHIVLWTRIASTIPPHVQKSDVEMEAQKDEIICPSC |
| NO: 99 | human | NRTAHPLRHINNDMIVTDNNGAVKFPQLCKFCDVRFSTCDNQKSC |
| | TGFBR2 | MSNCSITSICEKPQEVCVAVWRKNDENITLETVCHDPKLPYHDFILE |
| | isoform A | DAASPKCIMKEKKKPGETFFMCSCSSDECNDNIIFSEEYNTSNPDLLL |
| | (long | VIFQVTGISLLPPLGVAISVIIIFYCYRVNRQQKLSSTWETGKTRKLM |
| | isoform) | EFSEHCAIILEDDRSDISSTCANNINHNTELLPIELDTLVGKGRFAEVY |
| | (P37173-2) | KAKLKQNTSEQFETVAVKIFPYEEYASWKTEKDIFSDINLKHENILQ |
| | | FLTAEERKTELGKQYWLITAFHAKGNLQEYLTRHVISWEDLRKLGS |
| | | SLARGIAHLHSDHTPCGRPKMPIVHRDLKSSNILVKNDLTCCLCDFG |
| | | LSLRLDPTLSVDDLANSGQVGTARYMAPEVLESRMNLENVESFKQT |
| | | DVYSMALVLWEMTSRCNAVGEVKDYEPPFGSKVREHPCVESMKD |
| | | NVLRDRGRPEIPSFWLNHQGIQMVCETLTECWDHDPEARLTAQCV |
| | | AERFSELEHLDRLSGRSCSEEKIPEDGSLNTTK |
| SEQ ID | Human | TIPPHVQKSDVEMEAQKDEIICPSCNRTAHPLRHINNDMIVTDNNGA |
| NO: 124 | TGFBR2 | VKFPQLCKFCDVRFSTCDNQKSCMSNCSITSICEKPQEVCVAVWRK |
| | isoform A | NDENITLETVCHDPKLPYHDFILEDAASPKCIMKEKKKPGETFFMCS |
| | (long | CSSDECNDNIIFSEEYNTSNPDLLLVIFQVTGISLLPPLGVAISVIIIFYC |
| | isoform) | YRVNRQQKLSSTWETGKTRKLMEFSEHCAIILEDDRSDISSTCANNI |
| | (P37173-2) | NHNTELLPIELDTLVGKGRFAEVYKAKLKQNTSEQFETVAVKIFPYE |
| | | EYASWKTEKDIFSDINLKHENILQFLTAEERKTELGKQYWLITAFHA |
| | | KGNLQEYLTRHVISWEDLRKLGSSLARGIAHLHSDHTPCGRPKMPI |
| | | VHRDLKSSNILVKNDLTCCLCDFGLSLRLDPTLSVDDLANSGQVGT |
| | | ARYMAPEVLESRMNLENVESFKQTDVYSMALVLWEMTSRCNAVG |
| | | EVKDYEPPFGSKVREHPCVESMKDNVLRDRGRPEIPSFWLNHQGIQ |
| | | MVCETLTECWDHDPEARLTAQCVAERFSELEHLDRLSGRSCSEEKI |
| | | PEDGSLNTTK |
| SEQ ID | Human | TIPPHVQKSVNNDMIVTDNNGAVKFPQLCKFCDVRFSTCDNQKSC |
| NO: 100 | TGFBR2 | MSNCSITSICEKPQEVCVAVWRKNDENITLETVCHDPKLPYHDFILE |
| | fragment 1 | DAASPKCIMKEKKKPGETFFMCSCSSDECNDNIIFSEEYNTSNPD |
| | (ECD of | |
| | human | |
| | TGFBR2 | |
| | isoform B) | |
| SEQ ID | Human | IPPHVQKSVNNDMIVTDNNGAVKFPQLCKFCDVRFSTCDNQKSCMS |
| NO: 101 | TGFBR2 | NCSITSICEKPQEVCVAVWRKNDENITLETVCHDPKLPYHDFILEDA |
| | fragment 2 | ASPKCIMKEKKKPGETFFMCSCSSDECNDNIIFSEEYNTSNPD |
| SEQ ID | Human | TIPPHVQKSDVEMEAQKDEIICPSCNRTAHPLRHINNDMIVTDNNGA |
| NO: 102 | TGFBR2 | VKFPQLCKFCDVRFSTCDNQKSCMSNCSITSICEKPQEVCVAVWRK |
| | fragment 3 | NDENITLETVCHDPKLPYHDFILEDAASPKCIMKEKKKPGETFFMCS |
| | (ECD of | CSSDECNDNIIFSEEYNTSNPD |
| | human | |
| | TGFBR2 | |
| | isoform A) | |
| SEQ ID | Human | QLCKFCDVRFSTCDNQKSCMSNCSITSICEKPQEVCVAVWRKNDEN |
| NO: 103 | TGFBR2 | ITLETVCHDPKLPYHDFILEDAASPKCIMKEKKKPGETFFMCSCSSD |
| | fragment 4 | ECNDNIIF |
| SEQ ID | Immature | MTSHYVIAIFALMSSCLATAGPEPGALCELSPVSASHPVQALMESFT |
| NO: 106 | human | VLSGCASRGTTGLPQEVHVLNLRTAGQGPGQLQREVTLHLNPISSV |
| | TGFBR3 | HIHHKSVVFLLNSPHPLVWHLKTERLATGVSRLFLVSEGSVVQFSSA |
| | isoform 1 | NFSLTAETEERNFPHGNEHLLNWARKEYGAVTSFTELKIARNIYIKV |
| | (Q03167-1) | GEDQVFPPKCNIGKNFLSLNYLAEYLQPKAAEGCVMSSQPQNEEVH |
| | | IIELITPNSNPYSAFQVDITIDIRPSQEDLEVVKNLILILKCKKSVNWVI |
| | | KSFDVKGSLKIIAPNSIGFGKESERSMTMTKSIRDDIPSTQGNLVKW |
| | | ALDNGYSPITSYTMAPVANRFHLRLENNAEEMGDEEVHTIPPELRIL |
| | | LDPGALPALQNPPIRGGEGQNGGLPFPFPDISRRVWNEEGEDGLPRP |
| | | KDPVIPSIQLFPGLREPEEVQGSVDIALSVKCDNEKMIVAVEKDSFQ |
| | | ASGYSGMDVTLLDPTCKAKMNGTHFVLESPLNGCGTRPRWSALDG |
| | | VVYYNSIVIQVPALGDSSGWPDGYEDLESGDNGFPGDMDEGDASLF |
| | | TRPEIVVFNCSLQQVRNPSSFQEQPHGNITFNMELYNTDLFLVPSQG |
| | | VFSVPENGHVYVEVSVTKAEQELGFAIQTCFISPYSNPDRMSHYTIIE |
| | | NICPKDESVKFYSPKRVHFPIPQADMDKKRFSFVFKPVFNTSLLFLQ |
| | | CELTLCTKMEKHPQKLPKCVPPDEACTSLDASIIWAMMQNKKTFTK |
| | | PLAVIHHEAESKEKGPSMKEPNPISPPIFHGLDTLTVMGIAFAAFVIG |
| | | ALLTGALWYIYSHTGETAGRQQVPTSPPASENSSAAHSIGSTQSTPC |
| | | SSSSTA |
| SEQ ID | Human | GPEPGALCELSPVSASHPVQALMESFTVLSGCASRGTTGLPQEVHVL |
| NO: 125 | TGFBR3 | NLRTAGQGPGQLQREVTLHLNPISSVHIHHKSVVFLLNSPHPLVWH |
| | isoform 1 | LKTERLATGVSRLFLVSEGSVVQFSSANFSLTAETEERNFPHGNEHL |
| | (Q03167-1) | LNWARKEYGAVTSFTELKIARNIYIKVGEDQVFPPKCNIGKNFLSLN |
| | | YLAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGFGK |
| | | ESERSMTMTKSIRDDIPSTQGNLVKWALDNGYSPITSYTMAPVANR |
| | | FHLRLENNAEEMGDEEVHTIPPELRILLDPGALPALQNPPIRGGEGQ |
| | | NGGLPFPFPDISRRVWNEEGEDGLPRPKDPVIPSIQLFPGLREPEEVQ |
| | | GSVDIALSVKCDNEKMIVAVEKDSFQASGYSGMDVTLLDPTCKAK |
| | | MNGTHFVLESPLNGCGTRPRWSALDGVVYYNSIVIQVPALGDSSG |
| | | WPDGYEDLESGDNGFPGDMDEGDASLFTRPEIVVFNCSLQQVRNPS |
| | | SFQEQPHGNITFNMELYNTDLFLVPSQGVFSVPENGHVYVEVSVTK |
| | | AEQELGFAIQTCFISPYSNPDRMSHYTIIENICPKDESVKFYSPKRVHF |
| | | PIPQADMDKKRFSFVFKPVFNTSLLFLQCELTLCTKMEKHPQKLPKC |
| | | VPPDEACTSLDASIIWAMMQNKKTFTKPLAVIHHEAESKEKGPSMK |
| | | EPNPISPPIFHGLDTLTVMGIAFAAFVIGALLTGALWYIYSHTGETAG |
| | | RQQVPTSPPASENSSAAHSIGSTQSTPCSSSSTA |
| SEQ ID | Immature | MTSHYVIAIFALMSSCLATAGPEPGALCELSPVSASHPVQALMESFT |
| NO: 107 | human | VLSGCASRGTTGLPQEVHVLNLRTAGQGPGQLQREVTLHLNPISSV |
| | TGFBR3 | HIHHKSVVFLLNSPHPLVWHLKTERLATGVSRLFLVSEGSVVQFSSA |
| | isoform 2 | NFSLTAETEERNFPHGNEHLLNWARKEYGAVTSFTELKIARNIYIKV |
| | (Q03167-2) | GEDQVFPPKCNIGKNFLSLNYLAEYLQPKAAEGCVMSSQPQNEEVH |
| | | IIELITPNSNPYSAFQVDITIDIRPSQEDLEVVKNLILILKCKKSVNWVI |
| | | KSFDVKGSLKIIAPNSIGFGKESERSMTMTKSIRDDIPSTQGNLVKW |
| | | ALDNGYSPITSYTMAPVANRFHLRLENNEEMGDEEVHTIPPELRILL |
| | | DPGALPALQNPPIRGGEGQNGGLPFPFPDISRRVWNEEGEDGLPRPK |
| | | DPVIPSIQLFPGLREPEEVQGSVDIALSVKCDNEKMIVAVEKDSFQAS |
| | | GYSGMDVTLLDPTCKAKMNGTHFVLESPLNGCGTRPRWSALDGV |
| | | VYYNSIVIQVPALGDSSGWPDGYEDLESGDNGFPGDMDEGDASLFT |
| | | RPEIVVFNCSLQQVRNPSSFQEQPHGNITFNMELYNTDLFLVPSQGV |
| | | FSVPENGHVYVEVSVTKAEQELGFAIQTCFISPYSNPDRMSHYTIIEN |
| | | ICPKDESVKFYSPKRVHFPIPQADMDKKRFSFVFKPVFNTSLLFLQCE |
| | | LTLCTKMEKHPQKLPKCVPPDEACTSLDASIIWAMMQNKKTFTKPL |
| | | AVIHHEAESKEKGPSMKEPNPISPPIFHGLDTLTVMGIAFAAFVIGAL |
| | | LTGALWYIYSHTGETAGRQQVPTSPPASENSSAAHSIGSTQSTPCSSS |
| | | STA |
| SEQ ID | Human | GPEPGALCELSPVSASHPVQALMESFTVLSGCASRGTTGLPQEVHVL |
| NO: 126 | TGFB R3 | NLRTAGQGPGQLQREVTLHLNPISSVHIHHKSVVFLLNSPHPLVWH |
| | isoform 2 | LKTERLATGVSRLFLVSEGSVVQFSSANFSLTAETEERNFPHGNEHL |
| | (Q03167-2) | LNWARKEYGAVTSFTELKIARNIYIKVGEDQVFPPKCNIGKNFLSLN |
| | | YLAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGFGK |
| | | ESERSMTMTKSIRDDIPSTQGNLVKWALDNGYSPITSYTMAPVANR |
| | | FHLRLENNEEMGDEEVHTIPPELRILLDPGALPALQNPPIRGGEGQN |
| | | GGLPFPFPDISRRVWNEEGEDGLPRPKDPVIPSIQLFPGLREPEEVQG |
| | | SVDIALSVKCDNEKMIVAVEKDSFQASGYSGMDVTLLDPTCKAKM |
| | | NGTHFVLESPLNGCGTRPRWSALDGVVYYNSIVIQVPALGDSSGWP |
| | | DGYEDLESGDNGFPGDMDEGDASLFTRPEIVVFNCSLQQVRNPSSF |
| | | QEQPHGNITFNMELYNTDLFLVPSQGVFSVPENGHVYVEVSVTKAE |
| | | QELGFAIQTCFISPYSNPDRMSHYTIIENICPKDESVKFYSPKRVHFPI |
| | | PQADMDKKRFSFVFKPVFNTSLLFLQCELTLCTKMEKHPQKLPKCV |
| | | PPDEACTSLDASIIWAMMQNKKTFTKPLAVIHHEAESKEKGPSMKE |
| | | PNPISPPIFHGLDTLTVMGIAFAAFVIGALLTGALWYIYSHTGETAGR |
| | | QQVPTSPPASENSSAAHSIGSTQSTPCSSSSTA |
| SEQ ID | Human | GPEPGALCELSPVSASHPVQALMESFTVLSGCASRGTTGLPQEVHVL |
| NO: 108 | TGFB R3 | NLRTAGQGPGQLQREVTLHLNPISSVHIHHKSVVFLLNSPHPLVWH |
| | fragment 1 | LKTERLATGVSRLFLVSEGSVVQFSSANFSLTAETEERNFPHGNEHL |
| | | LNWARKEYGAVTSFTELKIARNIYIKVGEDQVFPPKCNIGKNFLSLN |
| | | YLAEYLQPKAAEGCVMSSQPQNEEVHIIELITPNSNPYSAFQVDITID |
| | | IRPSQEDLEVVKNLILILKCKKSVNWVIKSFDVKGSLKIIAPNSIGFGK |
| | | ESERSMTMTKSIRDDIPSTQGNLVKWALDNGYSPITSYTMAPVANR |
| | | FHLRLENNAEEMGDEEVHTIPPELRILLDPGALPALQNPPIRGGEGQ |
| | | NGGLPFPFPDISRRVWNEEGEDGLPRPKDPVIPSIQLFPGLREPEEVQ |
| | | GSVDIALSVKCDNEKMIVAVEKDSFQASGYSGMDVTLLDPTCKAK |
| | | MNGTHFVLESPLNGCGTRPRWSALDGVVYYNSIVIQVPALGDSSG |
| | | WPDGYEDLESGDNGFPGDMDEGDASLFTRPEIVVFNCSLQQVRNPS |
| | | SFQEQPHGNITFNMELYNTDLFLVPSQGVFSVPENGHVYVEVSVTK |
| | | AEQELGFAIQTCFISPYSNPDRMSHYTIIENICPKDESVKFYSPKRVHF |
| | | PIPQADMDKKRFSFVFKPVFNTSLLFLQCELTLCTKMEKHPQKLPKC |
| | | VPPDEACTSLDASIIWAMMQNKKTFTKPLAVIHHEAESKEKGPSMK |
| | | EPNPISPPIFHGLDTLTV |
| SEQ ID | hCH1- | ASTKGPSVFPLAPSSKSTSGGTAALGCLVKDYFPEPVTVSWNSGALT |
| NO: 192 | hFc_Hole- | SGVHTFPAVLQSSGLYSLSSVVTVPSSSLGTQTYICNVNHKPSNTKV |
| | 3x4GS- | DKRVEPKSCDKTHTCPPCPAPELLGGPSVFLFPPKPKDTLMISRTPEV |
| | TGFbR2 | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQVC |
| | | TLPPSREEMTKNQVSLSCAVKGFYPSDIAVEWESNGQPENNYKTTP |
| | | PVLDSDGSFFLVSKLTVDKSRWQQGNVFSCSVMHEALHNHYTQKS |
| | | LSLSPGXGGGGSGGGGSGGGGSIPPHVQKSVNNDMIVTDNNGAVK |
| | | FPQLCKFCDVRFSTCDNQKSCMSNCSITSICEKPQEVCVAVWRKND |
| | | ENITLETVCHDPKLPYHDFILEDAASPKCIMKEKKKPGETFFMCSCS |
| | | SDECNDNIIFSEEYNTSNPD, wherein X is K or absent |
| SEQ ID | hCH1- | ASTKGPSVFPLAPSSKSTSGGTAALGCLVKDYFPEPVTVSWNSGALT |
| NO: 193 | hFc_Knob- | SGVHTFPAVLQSSGLYSLSSVVTVPSSSLGTQTYICNVNHKPSNTKV |
| | 3x4GS- | DKRVEPKSCDKTHTCPPCPAPELLGGPSVFLFPPKPKDTLMISRTPEV |
| | TGFbR2 | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQVY |
| | | TLPPCREEMTKNQVSLWCLVKGFYPSDIAVEWESNGQPENNYKTTP |
| | | PVLDSDGSFFLYSKLTVDKSRWQQGNVFSCSVMHEALHNHYTQKS |
| | | LSLSPGXGGGGSGGGGSGGGGSIPPHVQKSVNNDMIVTDNNGAVK |
| | | FPQLCKFCDVRFSTCDNQKSCMSNCSITSICEKPQEVCVAVWRKND |
| | | ENITLETVCHDPKLPYHDFILEDAASPKCIMKEKKKPGETFFMCSCS |
| | | SDECNDNIIFSEEYNTSNPD, wherein X is K or absent |
| SEQ ID | hFc_Hole- | DKTHTCPPCPAPELLGGPSVFLFPPKPKDTLMISRTPEVTCVVVDVS |
| NO: 194 | 3x4GS- | HEDPEVKFNWYVDGVEVHNAKTKPREEQYNSTYRVVSVLTVLHQ |
| | TGFbR2 | DWLNGKEYKCKVSNKALPAPIEKTISKAKGQPREPQVCTLPPSREE |
| | | MTKNQVSLSCAVKGFYPSDIAVEWESNGQPENNYKTTPPVLDSDGS |
| | | FFLVSKLTVDKSRWQQGNVFSCSVMHEALHNHYTQKSLSLSPGXG |
| | | GGGSGGGGSGGGGSIPPHVQKSVNNDMIVTDNNGAVKFPQLCKFC |
| | | DVRFSTCDNQKSCMSNCSITSICEKPQEVCVAVWRKNDENITLETV |
| | | CHDPKLPYHDFILEDAASPKCIMKEKKKPGETFFMCSCSSDECNDNI |
| | | IFSEEYNTSNPD, wherein X is K or absent |
| SEQ ID | hFc_Knob- | DKTHTCPPCPAPELLGGPSVFLFPPKPKDTLMISRTPEVTCVVVDVS |
| NO: 195 | 3x4GS- | HEDPEVKFNWYVDGVEVHNAKTKPREEQYNSTYRVVSVLTVLHQ |
| | TGFbR2 | DWLNGKEYKCKVSNKALPAPIEKTISKAKGQPREPQVYTLPPCREE |
| | | MTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGX |
| | | GGGGSGGGGSGGGGSIPPHVQKSVNNDMIVTDNNGAVKFPQLCKF |
| | | CDVRFSTCDNQKSCMSNCSITSICEKPQEVCVAVWRKNDENITLET |
| | | VCHDPKLPYHDFILEDAASPKCIMKEKKKPGETFFMCSCSSDECND |
| | | NIIFSEEYNTSNPD, wherein X is K or absent |
| SEQ ID | TGFbR2- | IPPHVQKSVNNDMIVTDNNGAVKFPQLCKFCDVRFSTCDNQKSCMS |
| NO: 196 | 3x4GS- | NCSITSICEKPQEVCVAVWRKNDENITLETVCHDPKLPYHDFILEDA |
| | hCH1- | ASPKCIMKEKKKPGETFFMCSCSSDECNDNIIFSEEYNTSNPDGGGG |
| | hFc_Hole | SGGGGSGGGGSASTKGPSVFPLAPSSKSTSGGTAALGCLVKDYFPEP |
| | | VTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKP |
| | | KDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISK |
| | | AKGQPREPQVCTLPPSREEMTKNQVSLSCAVKGFYPSDIAVEWESN |
| | | GQPENNYKTTPPVLDSDGSFFLVSKLTVDKSRWQQGNVFSCSVMH |
| | | EALHNHYTQKSLSLSPGX, wherein X is K or absent |
| SEQ ID | TGFbR2- | IPPHVQKSVNNDMIVTDNNGAVKFPQLCKFCDVRFSTCDNQKSCMS |
| NO: 197 | 3x4GS- | NCSITSICEKPQEVCVAVWRKNDENITLETVCHDPKLPYHDFILEDA |
| | hCH1- | ASPKCIMKEKKKPGETFFMCSCSSDECNDNIIFSEEYNTSNPDGGGG |
| | hFc_Knob | SGGGGSGGGGSASTKGPSVFPLAPSSKSTSGGTAALGCLVKDYFPEP |
| | | VTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKP |
| | | KDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISK |
| | | AKGQPREPQVYTLPPCREEMTKNQVSLWCLVKGFYPSDIAVEWES |
| | | NGQPENNYKTTPPVLDSDGSFFLYSKLTVDKSRWQQGNVFSCSVM |
| | | HEALHNHYTQKSLSLSPGX, wherein X is K or absent |
| SEQ ID | TGFbR2- | IPPHVQKSVNNDMIVTDNNGAVKFPQLCKFCDVRFSTCDNQKSCMS |
| NO: 198 | 3x4GS- | NCSITSICEKPQEVCVAVWRKNDENITLETVCHDPKLPYHDFILEDA |
| | hCLIg_vl | ASPKCIMKEKKKPGETFFMCSCSSDECNDNIIFSEEYNTSNPDGGGG |
| | | SGGGGSGGGGSGQPKANPTVTLFPPSSEELQANKATLVCLISDFYPG |
| | | AVTVAWKADGSPVKAGVETTKPSKQSNNKYAASSYLSLTPEQWKS |
| | | HRSYSCQVTHEGSTVEKTVAPTECS |
| SEQ ID | TGFβR2- | IPPHVQKSVNNDMIVTDNNGAVKFPQLCKFCDVRFSTCDNQKSCMS |
| NO: 199 | 3x4GS- | NCSITSICEKPQEVCVAVWRKNDENITLETVCHDPKLPYHDFILEDA |
| | hCLIg_vk | ASPKCIMKEKKKPGETFFMCSCSSDECNDNIIFSEEYNTSNPDGGGG |
| | | SGGGGSGGGGSRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |

### Nucleic Acids

The invention also features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In another aspect, the application features host cells and vectors containing the nucleic acids described herein. The nucleic acids may be present in a single vector or separate vectors present in the same host cell or separate host cell, as described in more detail herein below.

### Vectors

Further provided herein are vectors comprising the nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating a cancer in a subject by using a multispecific molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is a hematological cancer or a metastatic lesion. In some embodiments, the hematological cancer is one or more of a Hodgkin's lymphoma, Non-Hodgkin's lymphoma, B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia, acute myeloid leukemia (AML), chronic myeloid leukemia, myelodysplastic syndrome (MDS), multiple myeloma, or acute lymphocytic leukemia.

In embodiments, the multispecific molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multispecific molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation. In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific molecules disclosed herein can be used in combination with a second therapeutic agent or procedure.

In embodiments, the multispecific molecule and the second therapeutic agent or procedure are administered/performed after a subject has been diagnosed with a cancer, e.g., before the cancer has been eliminated from the subject. In embodiments, the multispecific molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific molecule and the therapy, e.g., the cancer therapy, can be sequential (with or without overlap) or simultaneous. Administration of the multispecific molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, Astra7eneca Pharmaceuticals, LP, Wilmington, Del.; an estrogen-receptor antagonist used to treat breast cancer); ARIMIDEX® (anastrozole, Astra7eneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multispecific molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP ASO4 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multispecific molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-ß inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific molecule. The methods can be used in a therapeutic protocol in vivo.

In embodiments, an immune checkpoint inhibitor inhibits a checkpoint molecule.

Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GALS, VISTA, BTLA, TIGIT, LAIR1, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgGlk monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of a heavy chain). In embodiments, the PD-1 inhibitor is AMP-224 (B7-DCIg, e.g., described in WO2011/066342and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.570. The YW243.55.570 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.570, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S. Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### EXAMPLES

The following examples are intended to be illustrative, and are not meant in any way to be limiting.

### Example 1. Generation of Multiple αCCR2/αCSF1R Bispecific Antibody Molecules

### 1. Construction of the Plasmids.

The DNA encoding the protein sequences was optimized for expression in Cricetulus griseus, synthesized, and cloned into the pcDNA3.4-TOPO (Life Technologies A14697) using Gateway cloning. All constructs contained an Ig Kappa leader sequence (ATGGAAACCGACACACTGCTGCTGTGGGTGCTGCTCTTGTGGGTGCCAGGATCTAC AGGA (SEQ ID NO: 115), SEQ ID NO: METDTLLLWVLLLWVPGSTG (SEQ ID NO: 116)). The nucleic acid sequences used are shown in Table 5.

TABLE 5
Exemplary nucleic acid sequences of antibodies

| SEQ ID | | |
|---|---|---|
| NO | Description | Nucleic Acid Sequence |
| SEQ ID | αCCR2 | CAGGTCCAGCTGCAAGAGTCTGGCCCTGGACTGGTTCAGCCCTC |
| NO: 1 | MC12 VH | TCAGACCCTGTCTCTGACCTGTACCGTGTCCGGCTTCTCCCTGAC |
| | | CGACTTCTCTGTGCACTGGGTCCGACAGCCTCCAGGCAAAGGAC |
| | | TGGAATGGATGGGCAGAATCAGATCCGAGGGCAACACCGACTA |
| | | CAACAGCGCCCTGAAGTCCCGGCTGTCTATCAGCAGAGACACC |
| | | TCCAAGAGCCAGGTGTTCCTGAAGATGAACTCCCTGCAGACCG |
| | | AGGACACCGCCATCTATTTCTGCACCAGAGGCGACATCCTCGGC |
| | | TTCGGCTATTGGGGACAGGGCGTGATGGTCACCGTTAGCTCT |
| SEQ ID | αCCR2 | GACATCGTGATGACCCAGTCTCCACTGTCCGTGTCTGTGACCCC |
| NO: 2 | MC12 VL | TGGCGAGTCTGCCTCCATCTCCTGCAGATCCTCCAAGAGCCTGC |
| | | TGCACTTCAAGGGCATCACCTTCGTGTACTGGTATCTGCAGAAG |
| | | CCCGGCCAGTCTCCTCAGCTGCTGATCTTCAGAATGTCCAGCCT |
| | | GGCCTCTGGCGTGCCCGATAGATTTTCTGGCTCCGGCTCCGAGA |
| | | CAGACTTCACCCTGAAGATCTCCAGAGTGGAAGCCGAGGACGT |
| | | GGGCACCTACTATTGTGGCCAGCTGCTGGAAAACCCCTACACCT |
| | | TTGGCGCTGGCACCAAGCTGGAACTGAAG |
| SEQ ID | R2b CH1 | GCTCAGACCACCGCTCCTAGCGTGTACCCTTTGGCTCCTGGCTG |
| NO: 3 | | TGGCGACACCACCTCTTCTACAGTGACCCTGGGCTGTCTGGTCA |
| | | AGGGCTACTTTCCTGAGCCTGTGACCGTGACCTGGAACTCTGGT |
| | | GCCCTGTCCTCCGACGTGCACACCTTTCCAGCTGTGCTGCAGTC |
| | | CGGCCTGTACACCCTGACATCCTCCGTGACCTCTTCCACCTGGC |
| | | CTAGCCAGACCGTGACATGCAATGTGGCTCACCCTGCCTCCAGC |
| | | ACCAAGGTGGACAAGAAGGTGGAACGGCGG |
| SEQ ID | R2b CL | AGAGCTGACGCTGCCCCTACCGTGTCTATCTTCCCTCCATCCAT |
| NO: 4 | | GGAACAGCTGACCTCTGGCGGAGCTACCGTCGTGTGCTTCGTGA |
| | | ACAACTTCTACCCTCGGGACATCTCCGTGAAGTGGAAGATCGAC |
| | | GGCTCTGAGCAGCGAGATGGCGTGCTGGATTCTGTGACCGACC |
| | | AGGACTCCAAGGACAGCACCTACTCCATGTCTAGCACCCTGAG |
| | | CCTGACCAAGGTGGAATACGAGCGGCACAACCTGTATACCTGC |
| | | GAGGTGGTGCACAAGACCTCCAGCTCTCCCGTGGTCAAGTCCTT |
| | | CAACCGGAACGAGTGC |
| SEQ ID | αmCSF1R | CAGGTCCAGTTGCAGCAGTCTGGCGCTGAGCTGGTCAAGCCTG |
| NO: 5 | VH | GATCCTCCGTGAAGATCTCCTGCAAGGCCTCCGGCTACACCTTC |
| | | ACCTCCAACTTCATGCACTGGATCAAGCAGCAGCCCGGCAACG |
| | | GCCTGGAATGGATCGGATGGATCTATCCTGGCGACGGCGACAC |
| | | CGAGTACAACCAGAAGTTCAACGGCAAGGCTACCCTGACCGCC |
| | | GACAAGTCCTCTTCCACCGCTTACATGCAGCTGTCCAGCCTGAC |
| | | CTCTGAGGACTCCGCCGTGTACTTCTGCGCCGTGAATTATGGCG |
| | | GCTACGTGCTGGATGCTTGGGGCCAAGGCGCTTCTGTGACAGTG |
| | | TCCTCT |
| SEQ ID | R2a CH1 | GCCGAGACAACCGCTCCTAGCGTTTACCCTCTGGCTCCTGGCAC |
| NO: 6 | | AGCCCTGAAGTCCAACTCTATGGTCACCCTGGGCTGCCTGGTCA |
| | | AGGGCTACTTTCCTGAGCCTGTGACCGTGACCTGGAACTCTGGT |
| | | GCTCTGTCTAGCGGCGTGCACACCTTTCCAGCTGTGCTGCAGAG |
| | | CGGCCTGTACACCCTGACATCTAGCGTGACCGTGCCTTCCAGCA |
| | | CCTGGTCTAGTCAGGCTGTGACCTGCAACGTGGCCCATCCTGCC |
| | | TCTTCTACCAAGGTGGACAAGAAAATCGTGCCCAGAGAGTGCA |
| | | AC |
| SEQ ID | αmCSF1R | GAGATCGTGCTGACCCAGTCTCCTACCACCATGGCTGCTAGCCC |
| NO: 7 | VL | TGGCGAGAAAGTGACAATTACCTGCCGGGCCTCCTCCTCCACCA |
| | | ACTACATGTCCTGGTATCAGCAGAAGTCCGGCGCCTCTCCTAAG |
| | | CCTTGGATCTACGAGACATCCAAGCTGGCCTCTGGCGTGCCCGA |
| | | TAGATTTTCCGGCTCTGGCTCCGGCACCTCCTACAGCTTCACCA |
| | | TCTCCAGCATGGAAACAGAGGACGCCGCCACCTACTACTGCCA |
| | | CCAGTGGTCATCTACCCCTCTGACCTTTGGCAGCGGCACCAAGC |
| | | TGGAAATCAAG |
| SEQ ID | R2a CL | AGAGCTGACGCCGCTCCTACCGTGTCTATCTTCCCTCCATCCAT |
| NO: 8 | | GGAACAGCTGACCTCCGGCGGAGCTACCGTCGTGTGTTTCGTGA |
| | | ACAACTTCTACCCTCGGGACATCTCCGTGAAGTGGAAGATCGAC |
| | | GGCTCTGAGCAGCGAGATGGCGTGCTGGATTCTGTGACCGACC |
| | | AGGACTCCAAGGACAGCACCTACTCCATGTCTAGCACCCTGAG |
| | | CCTGACCAAGGTGGAATACGAGCGGCACAACCTGTATACCTGC |
| | | GAGGTGGTGCACAAGACCTCCAGCTCTCCCGTGGTCAAGTCCTT |
| | | CAACCGGAACGAGTGC |
| SEQ ID | mFc Knob | ACCATTAAGCCTTGTCCTCCATGCAAGTGCCCCGCTCCTAATCT |
| NO: 9 | | GCTCGGAGGCCCTTCCGTGTTCATCTTTCCACCTAAGATCAAGG |
| | | ACGTGCTGATGATCTCCCTGTCTCCTATCGTGACCTGCGTGGTG |
| | | GTGGACGTGTCCGAGGATGATCCTGACGTGCAGATCAGTTGGTT |
| | | CGTGAACAACGTGGAAGTGCACACCGCTCAGACCCAGACACAC |
| | | AGAGAGGACTACAACTCTACCCTGAGAGTGGTGTCTGCCCTGCC |
| | | TATCCAGCATCAGGACTGGATGTCCGGCAAAGAATTCAAGTGC |
| | | AAAGTGAACAACAAGGACCTGCCTGCTCCAATCGAGCGGACCA |
| | | TCTCTAAGCCTAAGGGCTCTGTCAGGGCCCCTCAGGTGTACGTT |
| | | CTGCCTCCTTGCGAGGAAGAGATGACCAAGAAACAAGTGACAC |
| | | TGTGGTGCATGGTCACAGACTTCATGCCCGAGGACATCTACGTG |
| | | GAATGGACCAACAACGGCAAGACCGAGCTGAACTACAAGAAC |
| | | ACCGAGCCTGTGCTGGACTCCGACGGCTCCTACTTCATGTACTC |
| | | CAAGCTGCGCGTCGAGAAGAAGAACTGGGTCGAGAGAAACTCC |
| | | TACTCCTGCTCCGTGGTGCACGAGGGCCTGCACAATCACCACAC |
| | | CACCAAGTCCTTCTCTCGGACCCCTGGCAAG |
| SEQ ID | mFc Hole | ACCATCAAGCCCTGTCCTCCATGCAAGTGCCCCGCTCCTAATCT |
| NO: 10 | | GCTCGGAGGCCCTTCCGTGTTCATCTTCCCACCTAAGATCAAGG |
| | | ACGTGCTGATGATCTCCCTGTCTCCTATCGTGACCTGCGTGGTG |
| | | GTGGACGTGTCCGAGGATGATCCTGACGTGCAGATCAGTTGGTT |
| | | CGTGAACAACGTGGAAGTGCACACCGCTCAGACCCAGACACAC |
| | | AGAGAGGACTACAACAGCACCCTGAGAGTGGTGTCTGCCCTGC |
| | | CAATCCAGCACCAGGATTGGATGTCCGGCAAAGAATTCAAGTG |
| | | CAAAGTGAACAACAAGGACCTGCCTGCTCCAATCGAGCGGACC |
| | | ATCTCTAAGCCTAAGGGCTCTGTGCGGGCTCCCCAAGTTTGTGT |
| | | TCTGCCTCCACCTGAGGAAGAGATGACCAAGAAACAAGTGACC |
| | | CTGTCTTGTGCCGTGACCGACTTCATGCCCGAGGACATCTACGT |
| | | GGAATGGACCAACAATGGCAAGACCGAGCTGAACTACAAGAAC |
| | | ACCGAGCCTGTGCTGGACTCCGACGGCTCCTACTTCATGGTGTC |
| | | TAAGCTGCGCGTCGAGAAGAAGAACTGGGTCGAGAGAAACTCC |
| | | TACTCCTGCTCCGTGGTGCACGAGGGCCTGCACAATCACCACAC |
| | | CACCAAGTCCTTCTCTCGGACCCCTGGCAAG |
| SEQ ID | αhCCR2 | GAGGTGCAGCTGGTTGAATCTGGCGGAGGACTGGTTAAGCCTG |
| NO: 11 | plozalizumab | GCGGCTCTCTGAGACTGTCTTGTGCCGCTTCTGGCTTCACCTTCT |
| | VH | CCGCCTACGCCATGAACTGGGTCCGACAGGCTCCTGGCAAAGG |
| | | CCTGGAATGGGTCGGAAGAATCCGGACCAAGAACAACAACTAC |
| | | GCCACCTACTACGCCGACTCCGTGAAGGACCGGTTCACCATCTC |
| | | TCGGGACGACTCCAAGAACACCCTGTACCTGCAGATGAACTCC |
| | | CTGAAAACCGAGGACACCGCCGTGTACTACTGCACCACCTTCTA |
| | | CGGCAATGGCGTGTGGGGACAGGGCACACTGGTTACCGTTTCTT |
| | | CCGCCTCCACCAAGGGACCCTCTGTGTTTCCTCTGGCTCCCTCC |
| | | AGCAAGTCTACCTCTGGTGGAACAGCTGCCCTGGGCTGCCTGGT |
| | | CAAGGATTACTTTCCTGAGCCTGTGACCGTGTCCTGG |
| SEQ ID | hCH1 | GCTTCTACCAAGGGACCCAGCGTGTTCCCTCTGGCTCCTTCCAG |
| NO: 12 | | CAAGTCTACCTCTGGCGGAACAGCTGCTCTGGGCTGCCTGGTCA |
| | | AGGACTACTTTCCTGAGCCTGTGACCGTGTCTTGGAACTCTGGC |
| | | GCTCTGACATCCGGCGTGCACACATTTCCAGCTGTGCTGCAGTC |
| | | CTCCGGCCTGTACTCTCTGTCCTCTGTCGTGACCGTGCCTTCCAG |
| | | CTCTCTGGGAACCCAGACCTACATCTGCAATGTGAACCACAAGC |
| | | CTTCCAACACCAAGGTGGACAAGAGAGTGGAACCCAAGTCCTG |
| | | C |
| SEQ ID | hFc Knob | GATAAGACCCACACATGTCCTCCATGCCCTGCCCCTGAGCTGCT |
| NO: 13 | | GGGCGGACCTTCCGTGTTCCTGTTCCCTCCAAAGCCCAAGGACA |
| | | CCCTGATGATCAGCCGGACCCCTGAAGTGACCTGCGTGGTGGTG |
| | | GATGTGTCCCACGAGGATCCCGAAGTGAAGTTCAATTGGTACGT |
| | | GGACGGCGTGGAAGTGCACAACGCCAAGACCAAGCCCAGAGA |
| | | GGAACAGTACAACAGCACCTACCGGGTGGTGTCCGTGCTGACC |
| | | GTGCTGCATCAGGACTGGCTGAACGGCAAAGAGTACAAGTGCA |
| | | AGGTGTCCAACAAGGCCCTGCCTGCCCCTATCGAGAAAACCAT |
| | | CAGCAAGGCCAAGGGCCAGCCCCGCGAACCTCAGGTGTACACA |
| | | CTGCCTCCCTGCCGGGAAGAGATGACCAAGAACCAGGTGTCCC |
| | | TGTGGTGCCTGGTCAAGGGCTTCTACCCCTCCGATATCGCCGTG |
| | | GAATGGGAGAGCAACGGCCAGCCCGAGAACAACTACAAGACC |
| | | ACCCCTCCCGTGCTGGACAGCGACGGCAGCTTCTTCCTGTACTC |
| | | CAAACTGACCGTGGACAAGAGCCGGTGGCAGCAGGGCAATGTG |
| | | TTCAGCTGTAGCGTGATGCACGAGGCCCTGCACAACCACTACAC |
| | | CCAGAAGTCCCTGAGCCTGTCTCCTGGCAAA |
| SEQ ID | αhCCR2 | GACGTGGTCATGACACAGAGCCCTCTGTCTCTGCCCGTGACATT |
| NO: 14 | plozalizumab | GGGACAGCCTGCCTCCATCTCCTGCAAGTCCTCTCAGTCCCTGC |
| | VL | TGGACTCTGACGGCAAGACCTTCCTGAACTGGTTCCAGCAGCGG |
| | | CCTGGCCAGTCTCCTAGAAGGCTGATCTACCTGGTGTCCAAGCT |
| | | GGATTCTGGCGTGCCCGACAGATTCTCCGGCTCTGGCTCTGGCA |
| | | CCGACTTCACCCTGAAGATCTCCAGAGTGGAAGCCGAGGACGT |
| | | GGGCGTGTACTACTGTTGGCAGGGCACCCACTTTCCATACACCT |
| | | TCGGCCAGGGCACCAGACTGGAAATCAAG |
| SEQ ID | hCL (kappa) | AGAACAGTGGCCGCTCCTTCCGTGTTCATCTTCCCACCCTCCGA |
| NO: 15 | | CGAGCAGCTGAAGTCCGGCACCGCTTCTGTCGTGTGCCTGCTCA |
| | | ACAACTTCTACCCTCGGGAAGCCAAGGTGCAGTGGAAGGTGGA |
| | | CAACGCCCTGCAGTCCGGCAACTCCCAGGAATCCGTCACCGAG |
| | | CAGGACTCCAAGGACAGCACCTACTCCCTGTCCTCCACCCTGAC |
| | | CCTGTCCAAGGCCGACTACGAGAAGCACAAGGTGTACGCCTGC |
| | | GAAGTGACCCACCAGGGCCTGAGCAGCCCCGTGACCAAGTCCT |
| | | TCAACCGGGGCGAGTGC |
| SEQ ID | αhCCR2 D1 | GAAGTGCAGCTGGTTCAGTCTGGCGCCGAAGTGAAGAAACCTG |
| NO: 16 | VH | GCGCCTCTGTGAAGGTGTCCTGCAAGGCTTCTGGCTACACCTTT |
| | | ACCGGCTACCACATGCACTGGGTCCGACAGGCTCCAGGACAAG |
| | | GCTTGGAATGGATGGGCTGGATCAACCCCAACTCCGGCGTGAC |
| | | CAAATACGCCCAGAAATTCCAGGGCAGAGTGACCATGACCAGA |
| | | GACACCTCCATCAACACCGCCTACATGGAACTGTCCCGGCTGAG |
| | | ATTCGACGACACCGACGTGTACTACTGTGCCACCGGCGGCTTTG |
| | | GCTATTGGGGAGAGGGAACACTGGTCACCGTGTCCTCC |
| SEQ ID | αhCCR2 D1 | CTGCCCGTGTTGACCCAGCCTCCTAGCGTTTCCAAGGGCCTGAG |
| NO: 17 | VL | ACAGACCGCCACACTGACCTGTACCGGCAACTCTAACAACGTG |
| | | GGCAATCAGGGCGCTGCCTGGTTGCAGCAGCATCAGGGACAGC |
| | | CTCCAAAGCTGCTGTCCTACCGGAACCACAACAGACCTAGCGG |
| | | CGTGTCCGAGCGGTTCAGCCCTTCTAGATCTGGCGACACCTCCA |
| | | GCCTGACCATCACTGGACTGCAGCCTGAGGACGAGGCCGACTA |
| | | CTATTGTCTGGCCTGGGACAGCTCCCTGCGGGCCTTTGTTTTTGG |
| | | CACCGGCACCAAGCTGACCGTGCTG |
| SEQ ID | hCL | GGACAACCTAAGGCCAATCCTACCGTGACACTGTTCCCTCCATC |
| NO: 18 | (lambda) | CTCCGAGGAACTGCAGGCCAACAAGGCTACCCTCGTGTGCCTG |
| | | ATCTCCGACTTTTACCCTGGCGCTGTGACCGTGGCCTGGAAGGC |
| | | TGATGGATCTCCTGTGAAGGCTGGCGTGGAAACCACCAAGCCTT |
| | | CCAAGCAGTCCAACAACAAATACGCCGCCTCCTCCTACCTGTCT |
| | | CTGACCCCTGAACAGTGGAAGTCCCACCGGTCCTACAGCTGCCA |
| | | AGTGACCCATGAGGGCTCCACCGTGGAAAAGACCGTGGCTCCT |
| | | ACCGAGTGCTCC |
| SEQ ID | αhCCR2 | CAGGTGCAGCTGGTTCAGTCTGGCGCCGAAGTGAAGAAACCTG |
| NO: 19 | 42G7 VH | GCGCCTCTGTGAAGGTGTCCTGCAAGGCTTCCGGCTACACCTTC |
| | | TCCAGCTACTACATGCACTGGGTCCGACAGGCCCCTGGACAAG |
| | | GATTGGAGTGGATGGGCATCATCAACCCCTCTGGCGGCAACAC |
| | | CTCTTACGCCCAGAAATTCCAGGGCAGAGTGACCATGACCAGA |
| | | GACACCTCCACCAGCACCGTGTACATGGAACTGTCCAGCCTGA |
| | | GATCCGAGGACACCGCCGTGTACTACTGTGCCAGAGGCGGATA |
| | | CCAGCTGCCTCACGGTAGAGCCAGAGCCTTCGATATGTGGGGC |
| | | CAGGGCACAATGGTCACCGTGTCCTCT |
| SEQ ID | αhCCR2 | GCCATCAGAATGACCCAGTCTCCACTGAGCCTGCCTGTGACATT |
| NO: 20 | 42G7 VL | GGGCCAGCCTGCCTCTATCTCCTGCACCTCCTCTCAGTCTCTGGT |
| | | GTACAGAGATGGCACCACCTACCTGAACTGGTTCCAGCAGAGG |
| | | CCTGGCCAGTCTCCTAGACGGCTGATCTACAAGGTGTCCAACAG |
| | | AGACTCTGGCGTGCCCGACAGATTCACCGGCTCTGGCTCTGGCA |
| | | CCACATTCACCCTGACCATCTCCAGAGTGGAAGCCGAGGACGT |
| | | GGGCATCTACTACTGTATGCAGGGCACCCACTGGCCTCTGACCT |
| | | TTGGCCAGGGAACAAAGGTGGAAATCAAG |
| SEQ ID | αhCCR2 | GAGGTGCAGCTGGTTGAATCTGGCGGAGGATTGGTTCAGCCTG |
| NO: 21 | 43G12 VH | GCGGCTCTCTGAGACTGTCTTGTGTGGCCTCTGGCTTCACCTTCT |
| | | CCGACTACTGGATGTCCTGGGTCCGACAGGCTCCTGGCAAAGG |
| | | ACTGGAATGGGTCGCCAACATCAAGAAAGACGGCTCCGTGAAC |
| | | TACTACGTGGACTCCGTGAAGGGCAGATTCACCATCTCTCGGGA |
| | | CAACGCCAAGAACTCCCTGTACCTGCAGATGAACAGCCTGAGA |
| | | GCCGAGGACACCGCCGTGTACTACTGCACCAGATTCGATTACTG |
| | | GGGCCAGGGCACCCTGGTCACAGTGTCCTCT |
| SEQ ID | αhCCR2 | CAGGCTGGCTTGACCCAGCCTCCTAGCGTTTCCAAGGGCCTGAG |
| NO: 22 | 43G12 VL | ACAGACCGCCACACTGACCTGTACCGGCAACTCTAACAACGTG |
| | | GGCAATCAGGGCGCTGCCTGGTTGCAGCAGCATCAGGGACATC |
| | | CTCCAAAGCTGCTGTTCTACCGGAACAACAACAGAGCCTCCGG |
| | | CATCTCCGAGCGGCTGTCTGCTTCTAGATCCGGCAATACCGCCA |
| | | GCCTGACCATCACTGGACTGCAGCCTGAGGACGAGGCCGACTA |
| | | CTATTGCCTGACCTGGGACTCCTCTCTGTCCGTGGTGGTTTTTGG |
| | | CGGAGGCACCAAGCTGACAGTGCTG |
| SEQ ID | αhCSF1R | CAGGTGCAGCTGGTTCAGTCTGGCGCCGAAGTGAAGAAACCTG |
| NO: 23 | emactuzumab | GCGCCTCTGTGAAGGTGTCCTGCAAGGCTTCCGGCTACACCTTT |
| | VH | ACCAGCTACGACATCTCCTGGGTCCGACAGGCTCCTGGACAAG |
| | | GCTTGGAATGGATGGGCGTGATCTGGACCGATGGCGGCACCAA |
| | | TTACGCCCAGAAACTGCAGGGCAGAGTGACCATGACCACCGAC |
| | | ACCTCTACCTCCACCGCCTACATGGAACTGCGGTCCCTGAGATC |
| | | TGACGACACCGCCGTGTACTACTGCGCCAGAGATCAGCGGCTG |
| | | TACTTCGATGTGTGGGGCCAGGGCACAACCGTGACAGTGTCCTC |
| | | T |
| SEQ ID | αhCSF1R | GACATCCAGATGACCCAGTCTCCATCCTCTCTGTCCGCCTCTGT |
| NO: 24 | emactuzumab | GGGCGACAGAGTGACCATCACCTGTAGAGCCTCCGAGGACGTG |
| | VL | AACACCTACGTGTCCTGGTATCAGCAGAAGCCCGGCAAGGCTC |
| | | CCAAGCTGCTGATCTACGCCGCCTCTAACAGATACACCGGCGTG |
| | | CCCTCTAGATTCTCCGGCTCTGGCTCTGGCACCGACTTTACCCTG |
| | | ACAATCTCCAGCCTGCAGCCTGAGGACTTCGCCACCTACTACTG |
| | | CCAGCAGTCCTTCAGCTACCCCACCTTTGGCCAGGGCACCAAGC |
| | | TGGAAATCAAG |
| SEQ ID | hFc Hole | GATAAGACCCACACCTGTCCTCCCTGCCCTGCCCCTGAACTGCT |
| NO: 25 | | GGGCGGACCTAGCGTGTTCCTGTTCCCTCCAAAGCCCAAGGACA |
| | | CCCTGATGATCAGCCGGACCCCTGAAGTGACCTGCGTGGTGGTG |
| | | GATGTGTCCCACGAGGATCCCGAAGTGAAGTTCAATTGGTACGT |
| | | GGACGGCGTGGAAGTGCACAACGCCAAGACCAAGCCCAGAGA |
| | | GGAACAGTACAACAGCACCTACCGGGTGGTGTCCGTGCTGACC |
| | | GTGCTGCACCAGGACTGGCTGAACGGCAAAGAGTACAAGTGCA |
| | | AGGTGTCCAACAAGGCCCTGCCAGCCCCTATCGAGAAAACCAT |
| | | CAGCAAGGCCAAGGGCCAGCCTAGAGAGCCTCAGGTCTGCACC |
| | | CTGCCTCCCAGCCGGGAAGAGATGACCAAGAACCAGGTGTCCC |
| | | TGAGCTGCGCCGTGAAGGGCTTCTACCCCTCCGATATCGCCGTG |
| | | GAATGGGAGAGCAACGGCCAGCCCGAGAACAACTACAAGACC |
| | | ACCCCTCCCGTGCTGGACAGCGACGGCAGCTTCTTCCTGGTGTC |
| | | CAAACTGACCGTGGACAAGAGCCGGTGGCAGCAGGGCAATGTG |
| | | TTCAGCTGTAGCGTGATGCACGAGGCCCTGCACAACCACTACAC |
| | | CCAGAAGTCTCTGAGCCTGAGCCCTGGCAAA |
| SEQ ID | αhCSF1R | CAGGTGCAGCTGGTTCAGTCTGGCGCCGAAGTGAAGAAACCTG |
| NO: 26 | cabiralizumab | GCTCCTCCGTGAAGGTGTCCTGCAAGGCTTCTGGCTACACCTTT |
| | VH | ACCGACAACTACATGATCTGGGTCCGACAGGCTCCTGGACAGG |
| | | GACTTGAGTGGATGGGCGACATCAACCCTTACAACGGCGGCAC |
| | | CACCTTCAACCAGAAATTCAAGGGCAGAGTGACCATCACCGCC |
| | | GACAAGTCTACCTCCACCGCCTACATGGAACTGTCCAGCCTGAG |
| | | ATCTGAGGACACCGCCGTGTACTACTGCGCCAGAGAGTCCCCTT |
| | | ACTTCTCCAACCTGTACGTGATGGACTACTGGGGCCAGGGCACA |
| | | CTGGTCACAGTGTCCTCT |
| SEQ ID | αhCSF1R | GAGATCGTGCTGACCCAGTCTCCTGCCACACTGTCACTGTCTCC |
| NO: 27 | cabiralizumab | AGGCGAGAGAGCTACCCTGTCCTGCAAGGCTTCTCAGTCCGTGG |
| | VL | ACTACGACGGCGACAACTACATGAACTGGTATCAGCAGAAGCC |
| | | CGGCCAGGCTCCTAGACTGCTGATCTACGCCGCCTCCAACCTGG |
| | | AATCTGGCATCCCCGCTAGATTCTCCGGCTCTGGCTCTGGCACA |
| | | GACTTTACCCTGACCATCTCCAGCCTGGAACCTGAGGACTTCGC |
| | | CGTGTACTACTGCCACCTGTCCAACGAGGACCTGTCCACATTTG |
| | | GCGGAGGCACCAAGGTGGAAATCAAG |

TABLE 6
Sequences used to construct ORFs.

| Sequence ID | Variable | Constant | Fc |
|---|---|---|---|
| SEQ ID NO: 28 | SEQ ID NO: 1 | SEQ ID NO: 3 | SEQ ID NO: 9 |
| SEQ ID NO: 29 | SEQ ID NO: 2 | SEQ ID NO: 4 | |
| SEQ ID NO: 30 | SEQ ID NO: 5 | SEQ ID NO: 6 | SEQ ID NO: 10 |
| SEQ ID NO: 31 | SEQ ID NO: 7 | SEQ ID NO: 8 | |
| SEQ ID NO: 32 | SEQ ID NO: 11 | SEQ ID NO: 12 | SEQ ID NO: 13 |
| SEQ ID NO: 33 | SEQ ID NO: 14 | SEQ ID NO: 15 | |
| SEQ ID NO: 34 | SEQ ID NO: 16 | SEQ ID NO: 12 | SEQ ID NO: 13 |
| SEQ ID NO: 35 | SEQ ID NO: 17 | SEQ ID NO: 18 | |
| SEQ ID NO: 36 | SEQ ID NO: 19 | SEQ ID NO: 12 | SEQ ID NO: 13 |
| SEQ ID NO: 37 | SEQ ID NO: 20 | SEQ ID NO: 15 | |
| SEQ ID NO: 38 | SEQ ID NO: 21 | SEQ ID NO: 12 | SEQ ID NO: 13 |
| SEQ ID NO: 39 | SEQ ID NO: 22 | SEQ ID NO: 18 | |
| SEQ ID NO: 40 | SEQ ID NO: 23 | SEQ ID NO: 12 | SEQ ID NO: 25 |
| SEQ ID NO: 41 | SEQ ID NO: 24 | SEQ ID NO: 15 | |
| SEQ ID NO: 42 | SEQ ID NO: 26 | SEQ ID NO: 12 | SEQ ID NO: 25 |
| SEQ ID NO: 43 | SEQ ID NO: 27 | SEQ ID NO: 15 | |

TABLE 7
Nucleic acid sequences of ORFs.

| SEQ ID | |
|---|---|
| NO | Nucleic Acid Sequence |
| SEQ ID | ATGGAAACCGACACACTGCTGCTGTGGGTGCTGCTCTTGTGGGTGCCAGGATCTACAGG |
| NO: 28 | ACAGGTCCAGCTGCAAGAGTCTGGCCCTGGACTGGTTCAGCCCTCTCAGACCCTGTCTC |
| | TGACCTGTACCGTGTCCGGCTTCTCCCTGACCGACTTCTCTGTGCACTGGGTCCGACAG |
| | CCTCCAGGCAAAGGACTGGAATGGATGGGCAGAATCAGATCCGAGGGCAACACCGACT |
| | ACAACAGCGCCCTGAAGTCCCGGCTGTCTATCAGCAGAGACACCTCCAAGAGCCAGGT |
| | GTTCCTGAAGATGAACTCCCTGCAGACCGAGGACACCGCCATCTATTTCTGCACCAGAG |
| | GCGACATCCTCGGCTTCGGCTATTGGGGACAGGGCGTGATGGTCACCGTTAGCTCTGCT |
| | CAGACCACCGCTCCTAGCGTGTACCCTTTGGCTCCTGGCTGTGGCGACACCACCTCTTC |
| | TACAGTGACCCTGGGCTGTCTGGTCAAGGGCTACTTTCCTGAGCCTGTGACCGTGACCT |
| | GGAACTCTGGTGCCCTGTCCTCCGACGTGCACACCTTTCCAGCTGTGCTGCAGTCCGGC |
| | CTGTACACCCTGACATCCTCCGTGACCTCTTCCACCTGGCCTAGCCAGACCGTGACATG |
| | CAATGTGGCTCACCCTGCCTCCAGCACCAAGGTGGACAAGAAGGTGGAACGGCGGACC |
| | ATTAAGCCTTGTCCTCCATGCAAGTGCCCCGCTCCTAATCTGCTCGGAGGCCCTTCCGT |
| | GTTCATCTTTCCACCTAAGATCAAGGACGTGCTGATGATCTCCCTGTCTCCTATCGTGAC |
| | CTGCGTGGTGGTGGACGTGTCCGAGGATGATCCTGACGTGCAGATCAGTTGGTTCGTGA |
| | ACAACGTGGAAGTGCACACCGCTCAGACCCAGACACACAGAGAGGACTACAACTCTAC |
| | CCTGAGAGTGGTGTCTGCCCTGCCTATCCAGCATCAGGACTGGATGTCCGGCAAAGAAT |
| | TCAAGTGCAAAGTGAACAACAAGGACCTGCCTGCTCCAATCGAGCGGACCATCTCTAA |
| | GCCTAAGGGCTCTGTCAGGGCCCCTCAGGTGTACGTTCTGCCTCCTTGCGAGGAAGAGA |
| | TGACCAAGAAACAAGTGACACTGTGGTGCATGGTCACAGACTTCATGCCCGAGGACAT |
| | CTACGTGGAATGGACCAACAACGGCAAGACCGAGCTGAACTACAAGAACACCGAGCCT |
| | GTGCTGGACTCCGACGGCTCCTACTTCATGTACTCCAAGCTGCGCGTCGAGAAGAAGA |
| | ACTGGGTCGAGAGAAACTCCTACTCCTGCTCCGTGGTGCACGAGGGCCTGCACAATCA |
| | CCACACCACCAAGTCCTTCTCTCGGACCCCTGGCAAGTGATGA |
| SEQ ID | ATGGAAACCGACACACTGCTGCTGTGGGTGCTGCTCTTGTGGGTGCCAGGATCTACCGG |
| NO: 29 | CGACATCGTGATGACCCAGTCTCCACTGTCCGTGTCTGTGACCCCTGGCGAGTCTGCCT |
| | CCATCTCCTGCAGATCCTCCAAGAGCCTGCTGCACTTCAAGGGCATCACCTTCGTGTAC |
| | TGGTATCTGCAGAAGCCCGGCCAGTCTCCTCAGCTGCTGATCTTCAGAATGTCCAGCCT |
| | GGCCTCTGGCGTGCCCGATAGATTTTCTGGCTCCGGCTCCGAGACAGACTTCACCCTGA |
| | AGATCTCCAGAGTGGAAGCCGAGGACGTGGGCACCTACTATTGTGGCCAGCTGCTGGA |
| | AAACCCCTACACCTTTGGCGCTGGCACCAAGCTGGAACTGAAGAGAGCTGACGCTGCC |
| | CCTACCGTGTCTATCTTCCCTCCATCCATGGAACAGCTGACCTCTGGCGGAGCTACCGT |
| | CGTGTGCTTCGTGAACAACTTCTACCCTCGGGACATCTCCGTGAAGTGGAAGATCGACG |
| | GCTCTGAGCAGCGAGATGGCGTGCTGGATTCTGTGACCGACCAGGACTCCAAGGACAG |
| | CACCTACTCCATGTCTAGCACCCTGAGCCTGACCAAGGTGGAATACGAGCGGCACAAC |
| | CTGTATACCTGCGAGGTGGTGCACAAGACCTCCAGCTCTCCCGTGGTCAAGTCCTTCAA |
| | CCGGAACGAGTGCTGATGA |
| SEQ ID | ATGGAAACCGACACACTGCTGCTGTGGGTGCTGCTCTTGTGGGTGCCAGGATCTACAGG |
| NO: 30 | ACAGGTCCAGTTGCAGCAGTCTGGCGCTGAGCTGGTCAAGCCTGGATCCTCCGTGAAG |
| | ATCTCCTGCAAGGCCTCCGGCTACACCTTCACCTCCAACTTCATGCACTGGATCAAGCA |
| | GCAGCCCGGCAACGGCCTGGAATGGATCGGATGGATCTATCCTGGCGACGGCGACACC |
| | GAGTACAACCAGAAGTTCAACGGCAAGGCTACCCTGACCGCCGACAAGTCCTCTTCCA |
| | CCGCTTACATGCAGCTGTCCAGCCTGACCTCTGAGGACTCCGCCGTGTACTTCTGCGCC |
| | GTGAATTATGGCGGCTACGTGCTGGATGCTTGGGGCCAAGGCGCTTCTGTGACAGTGTC |
| | CTCTGCCGAGACAACCGCTCCTAGCGTTTACCCTCTGGCTCCTGGCACAGCCCTGAAGT |
| | CCAACTCTATGGTCACCCTGGGCTGCCTGGTCAAGGGCTACTTTCCTGAGCCTGTGACC |
| | GTGACCTGGAACTCTGGTGCTCTGTCTAGCGGCGTGCACACCTTTCCAGCTGTGCTGCA |
| | GAGCGGCCTGTACACCCTGACATCTAGCGTGACCGTGCCTTCCAGCACCTGGTCTAGTC |
| | AGGCTGTGACCTGCAACGTGGCCCATCCTGCCTCTTCTACCAAGGTGGACAAGAAAATC |
| | GTGCCCAGAGAGTGCAACACCATCAAGCCCTGTCCTCCATGCAAGTGCCCCGCTCCTAA |
| | TCTGCTCGGAGGCCCTTCCGTGTTCATCTTCCCACCTAAGATCAAGGACGTGCTGATGA |
| | TCTCCCTGTCTCCTATCGTGACCTGCGTGGTGGTGGACGTGTCCGAGGATGATCCTGAC |
| | GTGCAGATCAGTTGGTTCGTGAACAACGTGGAAGTGCACACCGCTCAGACCCAGACAC |
| | ACAGAGAGGACTACAACAGCACCCTGAGAGTGGTGTCTGCCCTGCCAATCCAGCACCA |
| | GGATTGGATGTCCGGCAAAGAATTCAAGTGCAAAGTGAACAACAAGGACCTGCCTGCT |
| | CCAATCGAGCGGACCATCTCTAAGCCTAAGGGCTCTGTGCGGGCTCCCCAAGTTTGTGT |
| | TCTGCCTCCACCTGAGGAAGAGATGACCAAGAAACAAGTGACCCTGTCTTGTGCCGTG |
| | ACCGACTTCATGCCCGAGGACATCTACGTGGAATGGACCAACAATGGCAAGACCGAGC |
| | TGAACTACAAGAACACCGAGCCTGTGCTGGACTCCGACGGCTCCTACTTCATGGTGTCT |
| | AAGCTGCGCGTCGAGAAGAAGAACTGGGTCGAGAGAAACTCCTACTCCTGCTCCGTGG |
| | TGCACGAGGGCCTGCACAATCACCACACCACCAAGTCCTTCTCTCGGACCCCTGGCAAG |
| | TGATGA |
| SEQ ID | ATGGAAACCGACACACTGCTGCTGTGGGTGCTGCTCTTGTGGGTGCCAGGATCTACAGG |
| NO: 31 | CGAGATCGTGCTGACCCAGTCTCCTACCACCATGGCTGCTAGCCCTGGCGAGAAAGTG |
| | ACAATTACCTGCCGGGCCTCCTCCTCCACCAACTACATGTCCTGGTATCAGCAGAAGTC |
| | CGGCGCCTCTCCTAAGCCTTGGATCTACGAGACATCCAAGCTGGCCTCTGGCGTGCCCG |
| | ATAGATTTTCCGGCTCTGGCTCCGGCACCTCCTACAGCTTCACCATCTCCAGCATGGAA |
| | ACAGAGGACGCCGCCACCTACTACTGCCACCAGTGGTCATCTACCCCTCTGACCTTTGG |
| | CAGCGGCACCAAGCTGGAAATCAAGAGAGCTGACGCCGCTCCTACCGTGTCTATCTTCC |
| | CTCCATCCATGGAACAGCTGACCTCCGGCGGAGCTACCGTCGTGTGTTTCGTGAACAAC |
| | TTCTACCCTCGGGACATCTCCGTGAAGTGGAAGATCGACGGCTCTGAGCAGCGAGATG |
| | GCGTGCTGGATTCTGTGACCGACCAGGACTCCAAGGACAGCACCTACTCCATGTCTAGC |
| | ACCCTGAGCCTGACCAAGGTGGAATACGAGCGGCACAACCTGTATACCTGCGAGGTGG |
| | TGCACAAGACCTCCAGCTCTCCCGTGGTCAAGTCCTTCAACCGGAACGAGTGCTGATGA |
| SEQ ID | ATGGAAACCGACACACTGCTGCTGTGGGTGCTGCTCTTGTGGGTGCCAGGATCTACAGG |
| NO: 32 | CGAGGTGCAGCTGGTTGAATCTGGCGGAGGACTGGTTAAGCCTGGCGGCTCTCTGAGA |
| | CTGTCTTGTGCCGCTTCTGGCTTCACCTTCTCCGCCTACGCCATGAACTGGGTCCGACAG |
| | GCTCCTGGCAAAGGCCTGGAATGGGTCGGAAGAATCCGGACCAAGAACAACAACTACG |
| | CCACCTACTACGCCGACTCCGTGAAGGACCGGTTCACCATCTCTCGGGACGACTCCAAG |
| | AACACCCTGTACCTGCAGATGAACTCCCTGAAAACCGAGGACACCGCCGTGTACTACT |
| | GCACCACCTTCTACGGCAATGGCGTGTGGGGACAGGGCACACTGGTTACCGTTTCTTCC |
| | GCCTCCACCAAGGGACCCTCTGTGTTTCCTCTGGCTCCCTCCAGCAAGTCTACCTCTGGT |
| | GGAACAGCTGCCCTGGGCTGCCTGGTCAAGGATTACTTTCCTGAGCCTGTGACCGTGTC |
| | CTGGAACTCTGGCGCTCTGACATCTGGCGTGCACACCTTTCCAGCTGTGCTGCAGTCCT |
| | CTGGCCTGTACTCTCTGTCCTCCGTCGTGACCGTGCCTTCTAGCTCTCTGGGCACCCAGA |
| | CCTACATCTGCAATGTGAACCACAAGCCTTCCAACACCAAGGTGGACAAGAGAGTGGA |
| | ACCCAAGTCCTGCGACAAGACCCACACCTGTCCTCCATGTCCTGCTCCAGAACTGCTCG |
| | GCGGACCTTCCGTGTTCCTGTTTCCTCCAAAGCCTAAGGACACCCTGATGATCTCTCGG |
| | ACCCCTGAAGTGACCTGCGTGGTGGTGGATGTGTCTCACGAGGATCCCGAAGTGAAGT |
| | TCAATTGGTACGTGGACGGCGTGGAAGTGCACAACGCCAAGACCAAGCCTAGAGAGGA |
| | ACAGTACAACTCCACCTACAGAGTGGTGTCCGTGCTGACCGTGCTGCACCAGGATTGGC |
| | TGAACGGCAAAGAGTACAAGTGCAAGGTGTCCAACAAGGCCCTGCCTGCTCCTATCGA |
| | AAAGACCATCTCCAAGGCCAAGGGCCAGCCTAGGGAACCCCAGGTTTACACCCTGCCT |
| | CCATGCCGGGAAGAGATGACCAAGAATCAGGTGTCCCTGTGGTGCCTCGTGAAGGGCT |
| | TCTACCCTTCCGATATCGCCGTGGAATGGGAGAGCAATGGCCAGCCTGAGAACAACTA |
| | CAAGACAACCCCTCCTGTGCTGGACTCCGACGGCTCATTCTTCCTGTACTCCAAGCTGA |
| | CAGTGGACAAGTCCAGATGGCAGCAGGGCAACGTGTTCTCCTGCTCCGTGATGCACGA |
| | GGCCCTGCACAATCACTACACCCAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 33 | CGACGTGGTCATGACACAGAGCCCTCTGTCTCTGCCCGTGACATTGGGACAGCCTGCCT |
| | CCATCTCCTGCAAGTCCTCTCAGTCCCTGCTGGACTCTGACGGCAAGACCTTCCTGAAC |
| | TGGTTCCAGCAGCGGCCTGGCCAGTCTCCTAGAAGGCTGATCTACCTGGTGTCCAAGCT |
| | GGATTCTGGCGTGCCCGACAGATTCTCCGGCTCTGGCTCTGGCACCGACTTCACCCTGA |
| | AGATCTCCAGAGTGGAAGCCGAGGACGTGGGCGTGTACTACTGTTGGCAGGGCACCCA |
| | CTTTCCATACACCTTCGGCCAGGGCACCAGACTGGAAATCAAGAGAACCGTGGCCGCT |
| | CCTTCCGTGTTCATCTTCCCACCTTCCGACGAGCAGCTGAAGTCCGGCACAGCTTCTGTC |
| | GTGTGCCTGCTGAACAACTTCTACCCTCGGGAAGCCAAGGTGCAGTGGAAGGTGGACA |
| | ATGCCCTGCAGTCCGGCAACTCCCAAGAGTCTGTGACCGAGCAGGACTCCAAGGACAG |
| | CACCTACAGCCTGTCCAGCACACTGACCCTGTCCAAGGCCGACTACGAGAAGCACAAG |
| | GTGTACGCCTGCGAAGTGACCCATCAGGGCCTGTCTAGCCCTGTGACCAAGTCTTTCAA |
| | CCGGGGCGAGTGCTGATGA |
| SEQ ID | ATGGAAACCGACACACTGCTGCTGTGGGTGCTGCTCTTGTGGGTGCCAGGATCTACAGG |
| NO: 34 | CGAAGTGCAGCTGGTTCAGTCTGGCGCCGAAGTGAAGAAACCTGGCGCCTCTGTGAAG |
| | GTGTCCTGCAAGGCTTCTGGCTACACCTTTACCGGCTACCACATGCACTGGGTCCGACA |
| | GGCTCCAGGACAAGGCTTGGAATGGATGGGCTGGATCAACCCCAACTCCGGCGTGACC |
| | AAATACGCCCAGAAATTCCAGGGCAGAGTGACCATGACCAGAGACACCTCCATCAACA |
| | CCGCCTACATGGAACTGTCCCGGCTGAGATTCGACGACACCGACGTGTACTACTGTGCC |
| | ACCGGCGGCTTTGGCTATTGGGGAGAGGGAACACTGGTCACCGTGTCCTCCGCTTCTAC |
| | CAAGGGACCCTCCGTGTTTCCTCTGGCTCCTTCCAGCAAGTCTACCTCCGGTGGAACAG |
| | CTGCTCTGGGCTGCCTGGTCAAGGACTACTTTCCTGAGCCTGTGACCGTGTCTTGGAAC |
| | TCTGGCGCTCTGACATCCGGCGTGCACACCTTTCCAGCTGTGCTGCAATCCTCCGGCCT |
| | GTACTCTCTGTCCTCCGTCGTGACCGTGCCTTCTAGCTCTCTGGGCACCCAGACCTACAT |
| | CTGCAATGTGAACCACAAGCCTTCCAACACCAAGGTGGACAAGAGAGTGGAACCCAAG |
| | TCCTGCGACAAGACCCACACCTGTCCTCCATGTCCTGCTCCAGAACTGCTCGGCGGACC |
| | TTCTGTGTTCCTGTTTCCTCCAAAGCCTAAGGACACCCTGATGATCTCTCGGACCCCTGA |
| | AGTGACCTGCGTGGTGGTGGATGTGTCTCACGAGGACCCAGAAGTGAAGTTCAATTGG |
| | TACGTGGACGGCGTGGAAGTGCACAACGCCAAGACCAAGCCTAGAGAGGAACAGTAC |
| | AACTCCACCTACAGAGTGGTGTCCGTGCTGACCGTGCTGCACCAGGATTGGCTGAACG |
| | GCAAAGAGTACAAGTGCAAGGTGTCCAACAAGGCCCTGCCTGCTCCTATCGAAAAGAC |
| | CATCTCCAAGGCCAAGGGCCAGCCTAGGGAACCCCAGGTTTACACCCTGCCTCCATGCC |
| | GGGAAGAGATGACCAAGAACCAGGTGTCCCTGTGGTGCCTCGTGAAGGGCTTCTACCC |
| | TTCCGATATCGCCGTGGAATGGGAGAGCAATGGCCAGCCTGAGAACAACTACAAGACA |
| | ACCCCTCCTGTGCTGGACTCCGACGGCTCATTCTTCCTGTACTCCAAGCTGACAGTGGA |
| | CAAGTCCAGATGGCAGCAGGGCAACGTGTTCTCCTGCTCCGTGATGCACGAGGCCCTG |
| | CACAATCACTACACACAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 35 | ACTGCCCGTGTTGACCCAGCCTCCTAGCGTTTCCAAGGGCCTGAGACAGACCGCCACAC |
| | TGACCTGTACCGGCAACTCTAACAACGTGGGCAATCAGGGCGCTGCCTGGTTGCAGCA |
| | GCATCAGGGACAGCCTCCAAAGCTGCTGTCCTACCGGAACCACAACAGACCTAGCGGC |
| | GTGTCCGAGCGGTTCAGCCCTTCTAGATCTGGCGACACCTCCAGCCTGACCATCACTGG |
| | ACTGCAGCCTGAGGACGAGGCCGACTACTATTGTCTGGCCTGGGACAGCTCCCTGCGG |
| | GCCTTTGTTTTTGGCACCGGCACCAAGCTGACCGTGCTGGGACAACCTAAGGCCAATCC |
| | TACCGTGACACTGTTCCCTCCATCCTCCGAGGAACTGCAGGCCAACAAGGCTACCCTCG |
| | TGTGCCTGATCTCCGACTTTTACCCTGGCGCTGTGACCGTGGCCTGGAAGGCTGATGGA |
| | TCTCCTGTGAAGGCTGGCGTGGAAACCACCAAGCCTTCCAAGCAGTCCAACAACAAAT |
| | ACGCCGCCTCCTCCTACCTGTCTCTGACCCCTGAACAGTGGAAGTCCCACCGGTCCTAC |
| | AGCTGCCAAGTGACCCATGAGGGCTCCACCGTGGAAAAGACCGTGGCTCCTACCGAGT |
| | GCTCCTGATGA |
| SEQ ID | ATGGAAACCGACACACTGCTGCTGTGGGTGCTGCTCTTGTGGGTGCCAGGATCTACAGG |
| NO: 36 | ACAGGTGCAGCTGGTTCAGTCTGGCGCCGAAGTGAAGAAACCTGGCGCCTCTGTGAAG |
| | GTGTCCTGCAAGGCTTCCGGCTACACCTTCTCCAGCTACTACATGCACTGGGTCCGACA |
| | GGCCCCTGGACAAGGATTGGAGTGGATGGGCATCATCAACCCCTCTGGCGGCAACACC |
| | TCTTACGCCCAGAAATTCCAGGGCAGAGTGACCATGACCAGAGACACCTCCACCAGCA |
| | CCGTGTACATGGAACTGTCCAGCCTGAGATCCGAGGACACCGCCGTGTACTACTGTGCC |
| | AGAGGCGGATACCAGCTGCCTCACGGTAGAGCCAGAGCCTTCGATATGTGGGGCCAGG |
| | GCACAATGGTCACCGTGTCCTCTGCTTCCACCAAGGGACCCTCTGTGTTCCCTCTGGCTC |
| | CTTCCAGCAAGTCCACATCCGGTGGAACAGCTGCTCTGGGCTGCCTGGTCAAGGACTAC |
| | TTTCCTGAGCCTGTGACCGTGTCTTGGAACTCTGGCGCTCTGACATCCGGCGTGCACAC |
| | ATTTCCAGCTGTGCTGCAGTCCTCCGGCCTGTACTCTCTGTCCTCTGTCGTGACCGTGCC |
| | TTCCAGCTCTCTGGGAACCCAGACCTACATCTGCAATGTGAACCACAAGCCTTCCAACA |
| | CCAAGGTGGACAAGAGAGTGGAACCCAAGTCCTGCGACAAGACCCACACCTGTCCACC |
| | ATGTCCTGCTCCAGAACTGCTCGGCGGACCTTCCGTGTTCCTGTTTCCTCCAAAGCCTAA |
| | GGACACCCTGATGATCTCTCGGACCCCTGAAGTGACCTGCGTGGTGGTGGATGTGTCCC |
| | ACGAGGACCCAGAAGTGAAGTTCAATTGGTACGTGGACGGCGTGGAAGTGCACAACGC |
| | CAAGACCAAGCCTAGAGAGGAACAGTACAACTCCACCTACAGAGTGGTGTCCGTGCTG |
| | ACCGTGCTGCACCAGGATTGGCTGAACGGCAAAGAGTACAAGTGCAAGGTGTCCAACA |
| | AGGCCCTGCCTGCTCCTATCGAAAAGACCATCTCCAAGGCCAAGGGCCAGCCTAGGGA |
| | ACCCCAGGTTTACACCCTGCCTCCATGCCGGGAAGAGATGACCAAGAACCAGGTGTCC |
| | CTGTGGTGCCTCGTGAAGGGCTTCTACCCTTCCGATATCGCCGTGGAATGGGAGAGCAA |
| | TGGCCAGCCAGAGAACAACTACAAGACAACCCCTCCTGTGCTGGACTCCGACGGCTCA |
| | TTCTTCCTGTACTCCAAGCTGACAGTGGACAAGTCCAGATGGCAGCAGGGCAACGTGTT |
| | CTCCTGCTCCGTGATGCACGAGGCCCTGCACAATCACTACACACAGAAGTCCCTGTCTC |
| | TGTCCCCTGGCAAGTGATGA |
| SEQ ID | ATGGAAACCGACACACTGCTGCTGTGGGTGCTGCTCTTGTGGGTGCCAGGATCTACCGG |
| NO: 37 | CGCCATCAGAATGACCCAGTCTCCACTGAGCCTGCCTGTGACATTGGGCCAGCCTGCCT |
| | CTATCTCCTGCACCTCCTCTCAGTCTCTGGTGTACAGAGATGGCACCACCTACCTGAAC |
| | TGGTTCCAGCAGAGGCCTGGCCAGTCTCCTAGACGGCTGATCTACAAGGTGTCCAACA |
| | GAGACTCTGGCGTGCCCGACAGATTCACCGGCTCTGGCTCTGGCACCACATTCACCCTG |
| | ACCATCTCCAGAGTGGAAGCCGAGGACGTGGGCATCTACTACTGTATGCAGGGCACCC |
| | ACTGGCCTCTGACCTTTGGCCAGGGAACAAAGGTGGAAATCAAGCGGACCGTGGCCGC |
| | TCCTTCCGTGTTCATCTTCCCACCTTCCGACGAGCAGCTGAAGTCTGGCACAGCCTCTGT |
| | CGTGTGCCTGCTGAACAACTTCTACCCTCGGGAAGCCAAGGTGCAGTGGAAGGTGGAC |
| | AATGCCCTGCAGTCCGGCAACTCCCAAGAGTCTGTGACCGAGCAGGACTCCAAGGACA |
| | GCACCTACAGCCTGTCCTCCACACTGACCCTGTCCAAGGCCGACTACGAGAAGCACAA |
| | GGTGTACGCCTGCGAAGTGACCCATCAGGGCCTGTCTAGCCCTGTGACCAAGTCTTTCA |
| | ACCGGGGCGAGTGCTGATGA |
| SEQ ID | ATGGAAACCGACACACTGCTGCTGTGGGTGCTGCTCTTGTGGGTGCCAGGATCTACAGG |
| NO: 38 | CGAGGTGCAGCTGGTTGAATCTGGCGGAGGATTGGTTCAGCCTGGCGGCTCTCTGAGA |
| | CTGTCTTGTGTGGCCTCTGGCTTCACCTTCTCCGACTACTGGATGTCCTGGGTCCGACAG |
| | GCTCCTGGCAAAGGACTGGAATGGGTCGCCAACATCAAGAAAGACGGCTCCGTGAACT |
| | ACTACGTGGACTCCGTGAAGGGCAGATTCACCATCTCTCGGGACAACGCCAAGAACTC |
| | CCTGTACCTGCAGATGAACAGCCTGAGAGCCGAGGACACCGCCGTGTACTACTGCACC |
| | AGATTCGATTACTGGGGCCAGGGCACCCTGGTCACAGTGTCCTCTGCTTCTACCAAGGG |
| | ACCCAGCGTGTTCCCTCTGGCTCCTTCCAGCAAGTCTACCTCTGGCGGAACAGCTGCTC |
| | TGGGCTGCCTGGTCAAGGACTACTTTCCTGAGCCTGTGACCGTGTCCTGGAACTCTGGC |
| | GCTCTGACATCTGGCGTGCACACCTTTCCAGCTGTGCTGCAGTCCTCCGGCCTGTACTCT |
| | CTGTCCTCTGTCGTGACCGTGCCTTCCAGCTCTCTGGGAACCCAGACCTACATCTGCAA |
| | TGTGAACCACAAGCCTTCCAACACCAAGGTGGACAAGAGAGTGGAACCCAAGTCCTGC |
| | GACAAGACCCACACCTGTCCTCCATGTCCTGCTCCAGAACTGCTCGGCGGACCTTCCGT |
| | GTTCCTGTTTCCTCCAAAGCCTAAGGACACCCTGATGATCTCTCGGACCCCTGAAGTGA |
| | CCTGCGTGGTGGTGGATGTGTCTCACGAGGATCCCGAAGTGAAGTTCAATTGGTACGTG |
| | GACGGCGTGGAAGTGCACAATGCCAAGACCAAGCCTAGAGAGGAACAGTACAACTCC |
| | ACCTACAGAGTGGTGTCCGTGCTGACCGTGCTGCACCAGGATTGGCTGAACGGCAAAG |
| | AGTACAAGTGCAAGGTGTCCAACAAGGCCCTGCCTGCTCCTATCGAAAAGACCATCTC |
| | CAAGGCCAAGGGCCAGCCTAGGGAACCCCAGGTTTACACCCTGCCTCCATGCCGGGAA |
| | GAGATGACCAAGAACCAGGTGTCCCTGTGGTGCCTGGTTAAGGGCTTCTACCCCTCCGA |
| | TATCGCCGTGGAATGGGAGTCTAATGGCCAGCCAGAGAACAACTACAAGACAACCCCT |
| | CCTGTGCTGGACTCCGACGGCTCATTCTTCCTGTACTCCAAGCTGACAGTGGACAAGTC |
| | CAGATGGCAGCAGGGCAACGTGTTCTCCTGCTCCGTGATGCACGAGGCCCTGCACAAT |
| | CACTACACCCAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACAGG |
| NO: 39 | ACAGGCTGGCTTGACCCAGCCTCCTAGCGTTTCCAAGGGCCTGAGACAGACCGCCACA |
| | CTGACCTGTACCGGCAACTCTAACAACGTGGGCAATCAGGGCGCTGCCTGGTTGCAGC |
| | AGCATCAGGGACATCCTCCAAAGCTGCTGTTCTACCGGAACAACAACAGAGCCTCCGG |
| | CATCTCCGAGCGGCTGTCTGCTTCTAGATCCGGCAATACCGCCAGCCTGACCATCACTG |
| | GACTGCAGCCTGAGGACGAGGCCGACTACTATTGCCTGACCTGGGACTCCTCTCTGTCC |
| | GTGGTGGTTTTTGGCGGAGGCACCAAGCTGACAGTGCTGGGACAGCCTAAGGCCAATC |
| | CTACCGTGACACTGTTCCCTCCATCCTCCGAGGAACTGCAGGCCAACAAGGCTACCCTC |
| | GTGTGCCTGATCTCCGACTTTTACCCTGGCGCTGTGACCGTGGCCTGGAAGGCTGATGG |
| | ATCTCCTGTGAAGGCTGGCGTGGAAACCACCAAGCCTTCCAAGCAGTCCAACAACAAA |
| | TACGCCGCCTCCTCCTACCTGTCTCTGACCCCTGAACAGTGGAAGTCCCACCGGTCCTA |
| | CAGCTGCCAAGTGACCCATGAGGGCTCCACCGTGGAAAAGACCGTGGCTCCTACCGAG |
| | TGCTCCTGATGA |
| SEQ ID | ATGGAAACCGACACACTGCTGCTGTGGGTGCTGCTCTTGTGGGTGCCAGGATCTACAGG |
| NO: 40 | ACAGGTGCAGCTGGTTCAGTCTGGCGCCGAAGTGAAGAAACCTGGCGCCTCTGTGAAG |
| | GTGTCCTGCAAGGCTTCCGGCTACACCTTTACCAGCTACGACATCTCCTGGGTCCGACA |
| | GGCTCCTGGACAAGGCTTGGAATGGATGGGCGTGATCTGGACCGATGGCGGCACCAAT |
| | TACGCCCAGAAACTGCAGGGCAGAGTGACCATGACCACCGACACCTCTACCTCCACCG |
| | CCTACATGGAACTGCGGTCCCTGAGATCTGACGACACCGCCGTGTACTACTGCGCCAGA |
| | GATCAGCGGCTGTACTTCGATGTGTGGGGCCAGGGCACAACCGTGACAGTGTCCTCTGC |
| | TTCCACCAAGGGACCCAGCGTTTTCCCTCTGGCTCCATCCTCCAAGTCTACCTCTGGCG |
| | GAACAGCTGCTCTGGGCTGCCTGGTCAAGGACTACTTTCCTGAGCCTGTGACCGTGTCC |
| | TGGAACTCTGGCGCTCTGACATCTGGCGTGCACACATTCCCTGCTGTGCTGCAGTCCTC |
| | CGGCCTGTACTCTCTGTCCTCTGTGGTTACCGTGCCTTCCTCTAGCCTGGGCACCCAGAC |
| | CTACATCTGCAATGTGAACCACAAGCCTTCCAACACCAAGGTGGACAAGAGAGTGGAA |
| | CCCAAGTCCTGCGACAAGACCCACACCTGTCCACCATGTCCTGCTCCAGAACTGCTCGG |
| | CGGACCTTCCGTGTTCCTGTTTCCTCCAAAGCCTAAGGACACCCTGATGATCTCTCGGA |
| | CCCCTGAAGTGACCTGCGTGGTGGTGGATGTGTCTCACGAGGACCCAGAAGTGAAGTT |
| | CAATTGGTACGTGGACGGCGTGGAAGTGCACAACGCCAAGACCAAGCCTAGAGAGGA |
| | ACAGTACAACTCCACCTACAGAGTGGTGTCCGTGCTGACCGTGCTGCACCAGGATTGGC |
| | TGAACGGCAAAGAGTACAAGTGCAAGGTGTCCAACAAGGCCCTGCCTGCTCCTATCGA |
| | AAAGACCATCTCCAAGGCCAAGGGCCAGCCTCGGGAACCTCAAGTCTGTACCCTGCCT |
| | CCTAGCCGGGAAGAGATGACCAAGAACCAGGTGTCCCTGAGCTGCGCCGTGAAGGGCT |
| | TCTACCCTTCTGATATCGCCGTGGAATGGGAGAGCAACGGCCAGCCTGAGAACAACTA |
| | CAAGACAACCCCTCCTGTGCTGGACTCCGACGGCTCATTCTTCCTGGTGTCCAAGCTGA |
| | CAGTGGACAAGTCCAGATGGCAGCAGGGCAACGTGTTCTCCTGCTCCGTGATGCACGA |
| | GGCCCTGCACAATCACTACACACAGAAGTCCCTGTCTCTGTCCCCTGGCAAGTGATGA |
| SEQ ID | ATGGAAACCGACACACTGCTGCTGTGGGTGCTGCTCTTGTGGGTGCCAGGATCTACCGG |
| NO: 41 | CGACATCCAGATGACCCAGTCTCCATCCTCTCTGTCCGCCTCTGTGGGCGACAGAGTGA |
| | CCATCACCTGTAGAGCCTCCGAGGACGTGAACACCTACGTGTCCTGGTATCAGCAGAA |
| | GCCCGGCAAGGCTCCCAAGCTGCTGATCTACGCCGCCTCTAACAGATACACCGGCGTG |
| | CCCTCTAGATTCTCCGGCTCTGGCTCTGGCACCGACTTTACCCTGACAATCTCCAGCCTG |
| | CAGCCTGAGGACTTCGCCACCTACTACTGCCAGCAGTCCTTCAGCTACCCCACCTTTGG |
| | CCAGGGCACCAAGCTGGAAATCAAGCGGACAGTGGCCGCTCCTTCCGTGTTCATCTTCC |
| | CACCTTCCGACGAGCAGCTGAAGTCCGGCACAGCTTCTGTCGTGTGCCTGCTGAACAAC |
| | TTCTACCCTCGGGAAGCCAAGGTGCAGTGGAAGGTGGACAATGCCCTGCAGTCCGGCA |
| | ACTCCCAAGAGTCTGTGACCGAGCAGGACTCCAAGGACAGCACCTACAGCCTGTCCTC |
| | CACACTGACCCTGTCCAAGGCCGACTACGAGAAGCACAAGGTGTACGCCTGCGAAGTG |
| | ACCCATCAGGGCCTGTCTAGCCCTGTGACCAAGTCTTTCAACCGGGGCGAGTGCTGATG |
| | A |
| SEQ ID | ATGGAAACCGACACACTGCTGCTGTGGGTGCTGCTCTTGTGGGTGCCAGGATCTACAGG |
| NO: 42 | ACAGGTGCAGCTGGTTCAGTCTGGCGCCGAAGTGAAGAAACCTGGCTCCTCCGTGAAG |
| | GTGTCCTGCAAGGCTTCTGGCTACACCTTTACCGACAACTACATGATCTGGGTCCGACA |
| | GGCTCCTGGACAGGGACTTGAGTGGATGGGCGACATCAACCCTTACAACGGCGGCACC |
| | ACCTTCAACCAGAAATTCAAGGGCAGAGTGACCATCACCGCCGACAAGTCTACCTCCA |
| | CCGCCTACATGGAACTGTCCAGCCTGAGATCTGAGGACACCGCCGTGTACTACTGCGCC |
| | AGAGAGTCCCCTTACTTCTCCAACCTGTACGTGATGGACTACTGGGGCCAGGGCACACT |
| | GGTCACAGTGTCCTCTGCTTCCACCAAGGGACCCAGCGTTTTCCCTCTGGCTCCATCCTC |
| | CAAGTCCACCTCTGGTGGAACAGCTGCTCTGGGCTGCCTGGTCAAGGACTACTTTCCTG |
| | AGCCTGTGACCGTGTCCTGGAACTCTGGCGCTCTGACATCTGGCGTGCACACCTTTCCA |
| | GCTGTGCTGCAGTCCTCCGGCCTGTACTCTCTGTCCTCTGTCGTGACCGTGCCTTCCAGC |
| | TCTCTGGGAACCCAGACCTACATCTGCAATGTGAACCACAAGCCTTCCAACACCAAGGT |
| | CGACAAGAGAGTGGAACCCAAGTCCTGCGACAAGACCCACACCTGTCCACCTTGTCCT |
| | GCTCCAGAACTGCTCGGCGGACCTTCCGTGTTCCTGTTTCCTCCAAAGCCTAAGGACAC |
| | CCTGATGATCTCTCGGACCCCTGAAGTGACCTGCGTGGTGGTGGATGTGTCTCACGAGG |
| | ACCCAGAAGTGAAGTTCAATTGGTACGTGGACGGCGTGGAAGTGCACAACGCCAAGAC |
| | CAAGCCTAGAGAGGAACAGTACAACTCCACCTACAGAGTGGTGTCCGTGCTGACCGTG |
| | CTGCACCAGGATTGGCTGAACGGCAAAGAGTACAAGTGCAAGGTGTCCAACAAGGCCC |
| | TGCCTGCTCCTATCGAAAAGACCATCTCCAAGGCCAAGGGCCAGCCTCGGGAACCTCA |
| | AGTCTGTACCCTGCCTCCTAGCCGGGAAGAGATGACCAAGAACCAGGTGTCCCTGAGC |
| | TGCGCCGTGAAGGGCTTCTACCCTTCTGATATCGCCGTGGAATGGGAGAGCAACGGCC |
| | AGCCAGAGAACAACTACAAGACAACCCCTCCTGTGCTGGACTCCGACGGCTCATTCTTC |
| | CTGGTGTCCAAGCTGACAGTGGACAAGTCCAGATGGCAGCAGGGCAACGTGTTCTCCT |
| | GCTCCGTGATGCACGAGGCCCTGCACAATCACTACACACAGAAGTCTCTGTCTCTGAGC |
| | CCCGGCAAGTGATGA |
| SEQ ID | ATGGAAACCGACACACTGCTGCTGTGGGTGCTGCTCTTGTGGGTGCCAGGATCTACAGG |
| NO: 43 | CGAGATCGTGCTGACCCAGTCTCCTGCCACACTGTCACTGTCTCCAGGCGAGAGAGCTA |
| | CCCTGTCCTGCAAGGCTTCTCAGTCCGTGGACTACGACGGCGACAACTACATGAACTGG |
| | TATCAGCAGAAGCCCGGCCAGGCTCCTAGACTGCTGATCTACGCCGCCTCCAACCTGGA |
| | ATCTGGCATCCCCGCTAGATTCTCCGGCTCTGGCTCTGGCACAGACTTTACCCTGACCA |
| | TCTCCAGCCTGGAACCTGAGGACTTCGCCGTGTACTACTGCCACCTGTCCAACGAGGAC |
| | CTGTCCACATTTGGCGGAGGCACCAAGGTGGAAATCAAGCGGACAGTGGCCGCTCCTT |
| | CCGTGTTCATCTTCCCACCTTCCGACGAGCAGCTGAAGTCTGGCACCGCTTCTGTCGTGT |
| | GCCTGCTGAACAACTTCTACCCTCGGGAAGCCAAGGTGCAGTGGAAGGTGGACAATGC |
| | CCTGCAGTCCGGCAACTCCCAAGAGTCTGTGACCGAGCAGGACTCCAAGGACAGCACC |
| | TACAGCCTGTCCTCCACACTGACCCTGTCCAAGGCCGACTACGAGAAGCACAAGGTGT |
| | ACGCCTGCGAAGTGACCCATCAGGGCCTGTCTAGCCCTGTGACCAAGTCTTTCAACCGG |
| | GGCGAGTGCTGATGA |

### 2. Expression and Purification.

The plasmids were co-transfected into either Expi293 cells (Life Technologies A14527) or ExpiCHO cells (Life Technologies A29127). Transfections were performed using 1 mg of total DNA for a multispecific construct with a 1:1 knob to hole heavy chain ratio and 3:2 light chain to heavy chain ratio. When biotinylation was required, 250 μg of BirA was added per liter in addition to the multispecific construct DNA. Transfection in Expi293 cells was done using linear 25,000 Da polyethylenimine (PEI, Polysciences Inc 23966) in a 3:1 ratio with the total DNA. The DNA and PEI were each added to 50 mL of OptiMem (Life Technologies 31985088) medium and sterile filtered. The DNA and PEI were combined for 10 minutes and added to the Expi293 cells with a cell density of 1.8-2.8×10⁶ cells/mL and a viability of at least 95%. The ExpiCHO transfection was performed according to the manufacturer's instructions. Expi293 cells were grown in a humidified incubator at 37° C. with 8% CO₂ for 5-7 days after transfection and ExpiCHO cells were grown for 14 days at 32° C. with 5% CO₂. The cells were pelleted by centrifugation at 4500×g and the supernatant was filtered through a 0.2 μm membrane. Protein A resin (GE 17-1279-03) was added to the filtered supernatant and incubated for 1-3 hours at room temperature. The resin was packed into a column, washed with 3×10 column volumes of Dulbecco's phosphate-buffered saline (DPBS, Life Technologies 14190-144). The bound protein was eluted from the column with 20 mM citrate, 100 mM NaCl, pH 2.9. When necessary, the proteins were further purified using ligand affinity and/or size exclusion chromatography on a Superdex 200 column with a running buffer of DPBS.

TABLE 8
Amino Acid Sequences.

| SEQ | | |
|---|---|---|
| ID NO | Description | Amino Acid Sequence |
| SEQ ID | αCCR2 | QVQLQESGPGLVQPSQTLSLTCTVSGFSLTDFSVHWVRQPPGKGLEW |
| NO: 44 | MC12 VH | MGRIRSEGNTDYNSALKSRLSISRDTSKSQVFLKMNSLQTEDTAIYFC |
| | | TRGDILGFGYWGQGVMVTVSS |
| SEQ ID | αCCR2 | DIVMTQSPLSVSVTPGESASISCRSSKSLLHFKGITFVYWYLQKPGQSP |
| NO: 45 | MC12 VL | QLLIFRMSSLASGVPDRFSGSGSETDFTLKISRVEAEDVGTYYCGQLLE |
| | | NPYTFGAGTKLELK |
| SEQ ID | R2b CH1 | AQTTAPSVYPLAPGCGDTTSSTVTLGCLVKGYFPEPVTVTWNSGALS |
| NO: 46 | | SDVHTFPAVLQSGLYTLTSSVTSSTWPSQTVTCNVAHPASSTKVDKK |
| | | VERR |
| SEQ ID | R2b CL | RADAAPTVSIFPPSMEQLTSGGATVVCFVNNFYPRDISVKWKIDGSEQ |
| NO: 47 | | RDGVLDSVTDQDSKDSTYSMSSTLSLTKVEYERHNLYTCEVVHKTSS |
| | | SPVVKSFNRNEC |
| SEQ ID | αmCSF1R | QVQLQQSGAELVKPGSSVKISCKASGYTFTSNFMHWIKQQPGNGLEW |
| NO: 48 | VH | IGWIYPGDGDTEYNQKFNGKATLTADKSSSTAYMQLSSLTSEDSAVY |
| | | FCAVNYGGYVLDAWGQGASVTVSS |
| SEQ ID | R2a CH1 | AETTAPSVYPLAPGTALKSNSMVTLGCLVKGYFPEPVTVTWNSGALS |
| NO: 49 | | SGVHTFPAVLQSGLYTLTSSVTVPSSTWSSQAVTCNVAHPASSTKVD |
| | | KKIVPRECN |
| SEQ ID | αhCCR2 | EVQLVESGGGLVQPGGSLRLSCVASGFTFSDYWMSWVRQAPGKGLE |
| NO: 64 | 43G12 VH | WVANIKKDGSVNYYVDSVKGRFTISRDNAKNSLYLQMNSLRAEDTA |
| | | VYYCTRFDYWGQGTLVTVSS |
| SEQ ID | αhCCR2 | QAGLTQPPSVSKGLRQTATLTCTGNSNNVGNQGAAWLQQHQGHPPK |
| NO: 65 | 43G12 VL | LLFYRNNNRASGISERLSASRSGNTASLTITGLQPEDEADYYCLTWDS |
| | | SLSVVVFGGGTKLTVL |
| SEQ ID | αhCSF1R | QVQLVQSGAEVKKPGASVKVSCKASGYTFTSYDISWVRQAPGQGLE |
| NO: 66 | emactuzumab | WMGVIWTDGGTNYAQKLQGRVTMTTDTSTSTAYMELRSLRSDDTA |
| | VH | VYYCARDQRLYFDVWGQGTTVTVSS |
| SEQ ID | αhCSF1R | DIQMTQSPSSLSASVGDRVTITCRASEDVNTYVSWYQQKPGKAPKLLI |
| NO: 67 | emactuzumab | YAASNRYTGVPSRFSGSGSGTDFTLTISSLQPEDFATYYCQQSFSYPTF |
| | VL | GQGTKLEIK |
| SEQ ID | hFc Hole | DKTHTCPPCPAPELLGGPSVFLFPPKPKDTLMISRTPEVTCVVVDVSHE |
| NO: 68 | | DPEVKFNWYVDGVEVHNAKTKPREEQYNSTYRVVSVLTVLHQDWL |
| | | NGKEYKCKVSNKALPAPIEKTISKAKGQPREPQVCTLPPSREEMTKNQ |
| | | VSLSCAVKGFYPSDIAVEWESNGQPENNYKTTPPVLDSDGSFFLVSKL |
| | | TVDKSRWQQGNVFSCSVMHEALHNHYTQKSLSLSPGX, wherein X is |
| | | K or absent |
| SEQ ID | αhCSF1R | QVQLVQSGAEVKKPGSSVKVSCKASGYTFTDNYMIWVRQAPGQGLE |
| NO: 69 | cabiralizumab | WMGDINPYNGGTTFNQKFKGRVTITADKSTSTAYMELSSLRSEDTAV |
| | VH | YYCARESPYFSNLYVMDYWGQGTLVTVSS |
| SEQ ID | αhCSF1R | EIVLTQSPATLSLSPGERATLSCKASQSVDYDGDNYMNWYQQKPGQ |
| NO: 70 | cabiralizumab | APRLLIYAASNLESGIPARFSGSGSGTDFTLTISSLEPEDFAVYYCHLSN |
| | VL | EDLSTFGGGTKVEIK |

TABLE 9
Protein sequences for full heavy and light chains.

| Sequence ID | Variable | Constant | Fc |
|---|---|---|---|
| SEQ ID NO: 71 | SEQ ID NO: 44 | SEQ ID NO: 46 | SEQ ID NO: 52 |
| SEQ ID NO: 72 | SEQ ID NO: 45 | SEQ ID NO: 47 | |
| SEQ ID NO: 73 | SEQ ID NO: 48 | SEQ ID NO: 49 | SEQ ID NO: 53 |
| SEQ ID NO: 74 | SEQ ID NO: 50 | SEQ ID NO: 51 | |
| SEQ ID NO: 75 | SEQ ID NO: 54 | SEQ ID NO: 55 | SEQ ID NO: 56 |
| SEQ ID NO: 76 | SEQ ID NO: 57 | SEQ ID NO: 58 | |
| SEQ ID NO: 77 | SEQ ID NO: 59 | SEQ ID NO: 55 | SEQ ID NO: 56 |
| SEQ ID NO: 78 | SEQ ID NO: 60 | SEQ ID NO: 61 | |
| SEQ ID NO: 79 | SEQ ID NO: 62 | SEQ ID NO: 55 | SEQ ID NO: 56 |
| SEQ ID NO: 80 | SEQ ID NO: 63 | SEQ ID NO: 58 | |
| SEQ ID NO: 81 | SEQ ID NO: 64 | SEQ ID NO: 55 | SEQ ID NO: 56 |
| SEQ ID NO: 82 | SEQ ID NO: 65 | SEQ ID NO: 58 | |
| SEQ ID NO: 83 | SEQ ID NO: 66 | SEQ ID NO: 55 | SEQ ID NO: 68 |
| SEQ ID NO: 84 | SEQ ID NO: 67 | SEQ ID NO: 58 | |
| SEQ ID NO: 85 | SEQ ID NO: 69 | SEQ ID NO: 55 | SEQ ID NO: 68 |
| SEQ ID NO: 86 | SEQ ID NO: 70 | SEQ ID NO: 58 | |

TABLE 10
Amino acid sequences of the chains used to construct multispecific molecules.

| Sequence ID | Amino Acid Sequence |
|---|---|
| SEQ ID NO: 71 | QVQLQESGPGLVQPSQTLSLTCTVSGFSLTDFSVHWVRQPPGKGLEWMGRIRSEGNT |
| | DYNSALKSRLSISRDTSKSQVFLKMNSLQTEDTAIYFCTRGDILGFGYWGQGVMVTV |
| | SSAQTTAPSVYPLAPGCGDTTSSTVTLGCLVKGYFPEPVTVTWNSGALSSDVHTFPA |
| | VLQSGLYTLTSSVTSSTWPSQTVTCNVAHPASSTKVDKKVERRTIKPCPPCKCPAPNL |
| | LGGPSVFIFPPKIKDVLMISLSPIVTCVVVDVSEDDPDVQISWFVNNVEVHTAQTQTH |
| | REDYNSTLRVVSALPIQHQDWMSGKEFKCKVNNKDLPAPIERTISKPKGSVRAPQVY |
| | VLPPCEEEMTKKQVTLWCMVTDFMPEDIYVEWTNNGKTELNYKNTEPVLDSDGSY |
| | FMYSKLRVEKKNWVERNSYSCSVVHEGLHNHHTTKSFSRTPGK |
| SEQ ID NO: 72 | DIVMTQSPLSVSVTPGESASISCRSSKSLLHFKGITFVYWYLQKPGQSPQLLIFRMSSL |
| | ASGVPDRFSGSGSETDFTLKISRVEAEDVGTYYCGQLLENPYTFGAGTKLELKRADA |
| | APTVSIFPPSMEQLTSGGATVVCFVNNFYPRDISVKWKIDGSEQRDGVLDSVTDQDS |
| | KDSTYSMSSTLSLTKVEYERHNLYTCEVVHKTSSSPVVKSFNRNEC |
| SEQ ID NO: 73 | QVQLQQSGAELVKPGSSVKISCKASGYTFTSNFMHWIKQQPGNGLEWIGWIYPGDG |
| | DTEYNQKFNGKATLTADKSSSTAYMQLSSLTSEDSAVYFCAVNYGGYVLDAWGQG |
| | ASVTVSSAETTAPSVYPLAPGTALKSNSMVTLGCLVKGYFPEPVTVTWNSGALSSGV |
| | HTFPAVLQSGLYTLTSSVTVPSSTWSSQAVTCNVAHPASSTKVDKKIVPRECNTIKPC |
| | PPCKCPAPNLLGGPSVFIFPPKIKDVLMISLSPIVTCVVVDVSEDDPDVQISWFVNNVE |
| | VHTAQTQTHREDYNSTLRVVSALPIQHQDWMSGKEFKCKVNNKDLPAPIERTISKPK |
| | GSVRAPQVCVLPPPEEEMTKKQVTLSCAVTDFMPEDIYVEWTNNGKTELNYKNTEP |
| | VLDSDGSYFMVSKLRVEKKNWVERNSYSCSVVHEGLHNHHTTKSFSRTPGK |
| SEQ ID NO: 74 | EIVLTQSPTTMAASPGEKVTITCRASSSTNYMSWYQQKSGASPKPWIYETSKLASGV |
| | PDRFSGSGSGTSYSFTISSMETEDAATYYCHQWSSTPLTFGSGTKLEIKRADAAPTVSI |
| | FPPSMEQLTSGGATVVCFVNNFYPRDISVKWKIDGSEQRDGVLDSVTDQDSKDSTYS |
| | MSSTLSLTKVEYERHNLYTCEVVHKTSSSPVVKSFNRNEC |
| SEQ ID NO: 75 | EVQLVESGGGLVKPGGSLRLSCAASGFTFSAYAMNWVRQAPGKGLEWVGRIRTKN |
| | NNYATYYADSVKDRFTISRDDSKNTLYLQMNSLKTEDTAVYYCTTFYGNGVWGQG |
| | TLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKTH |
| | TCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTIS |
| | KAKGQPREPQVYTLPPCREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPGX, |
| | wherein X is K or absent |
| SEQ ID NO: 76 | DVVMTQSPLSLPVTLGQPASISCKSSQSLLDSDGKTFLNWFQQRPGQSPRRLIYLVSK |
| | LDSGVPDRFSGSGSGTDFTLKISRVEAEDVGVYYCWQGTHFPYTFGQGTRLEIKRTV |
| | AAPSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| SEQ ID NO: 77 | EVQLVQSGAEVKKPGASVKVSCKASGYTFTGYHMHWVRQAPGQGLEWMGWINPN |
| | SGVTKYAQKFQGRVTMTRDTSINTAYMELSRLRFDDTDVYYCATGGFGYWGEGTL |
| | VTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKRVEPKSCDKTHTCP |
| | PCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAK |
| | GQPREPQVYTLPPCREEMTKNQVSLWCLVKGFYPSDIAVEWESNGQPENNYKTTPP |
| | VLDSDGSFFLYSKLTVDKSRWQQGNVFSCSVMHEALHNHYTQKSLSLSPGX, |
| | wherein X is K or absent |
| SEQ ID NO: 78 | LPVLTQPPSVSKGLRQTATLTCTGNSNNVGNQGAAWLQQHQGQPPKLLSYRNHNRP |
| | SGVSERFSPSRSGDTSSLTITGLQPEDEADYYCLAWDSSLRAFVFGTGTKLTVLGQPK |
| | ANPTVTLFPPSSEELQANKATLVCLISDFYPGAVTVAWKADGSPVKAGVETTKPSKQ |
| | SNNKYAASSYLSLTPEQWKSHRSYSCQVTHEGSTVEKTVAPTECS |
| SEQ ID NO: 79 | QVQLVQSGAEVKKPGASVKVSCKASGYTFSSYYMHWVRQAPGQGLEWMGIINPSG |
| | GNTSYAQKFQGRVTMTRDTSTSTVYMELSSLRSEDTAVYYCARGGYQLPHGRARA |
| | FDMWGQGTMVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKRVEP |
| | KSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALP |
| | APIEKTISKAKGQPREPQVYTLPPCREEMTKNQVSLWCLVKGFYPSDIAVEWESNGQ |
| | PENNYKTTPPVLDSDGSFFLYSKLTVDKSRWQQGNVFSCSVMHEALHNHYTQKSLS |
| | LSPGX, wherein X is K or absent |
| SEQ ID NO: 80 | AIRMTQSPLSLPVTLGQPASISCTSSQSLVYRDGTTYLNWFQQRPGQSPRRLIYKVSN |
| | RDSGVPDRFTGSGSGTTFTLTISRVEAEDVGIYYCMQGTHWPLTFGQGTKVEIKRTV |
| | AAPSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC |
| SEQ ID NO: 81 | EVQLVESGGGLVQPGGSLRLSCVASGFTFSDYWMSWVRQAPGKGLEWVANIKKDG |
| | SVNYYVDSVKGRFTISRDNAKNSLYLQMNSLRAEDTAVYYCTRFDYWGQGTLVTV |
| | SSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKRVEPKSCDKTHTCPPCP |
| | APELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQ |
| | PREPQVYTLPPCREEMTKNQVSLWCLVKGFYPSDIAVEWESNGQPENNYKTTPPVL |
| | DSDGSFFLYSKLTVDKSRWQQGNVFSCSVMHEALHNHYTQKSLSLSPGX, wherein X |
| | is K or absent |
| SEQ ID NO: 82 | QAGLTQPPSVSKGLRQTATLTCTGNSNNVGNQGAAWLQQHQGHPPKLLFYRNNNR |
| | ASGISERLSASRSGNTASLTITGLQPEDEADYYCLTWDSSLSVVVFGGGTKLTVLGQP |
| | KANPTVTLFPPSSEELQANKATLVCLISDFYPGAVTVAWKADGSPVKAGVETTKPSK |
| | QSNNKYAASSYLSLTPEQWKSHRSYSCQVTHEGSTVEKTVAPTECS |
| SEQ ID NO: 83 | QVQLVQSGAEVKKPGASVKVSCKASGYTFTSYDISWVRQAPGQGLEWMGVIWTDG |
| | GTNYAQKLQGRVTMTTDTSTSTAYMELRSLRSDDTAVYYCARDQRLYFDVWGQG |
| | TTVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKTH |
| | TCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTIS |
| | KAKGQPREPQVCTLPPSREEMTKNQVSLSCAVKGFYPSDIAVEWESNGQPENNYKT |
| | TPPVLDSDGSFFLVSKLTVDKSRWQQGNVFSCSVMHEALHNHYTQKSLSLSPGX, |
| | wherein X is K or absent |
| SEQ ID NO: 84 | DIQMTQSPSSLSASVGDRVTITCRASEDVNTYVSWYQQKPGKAPKLLIYAASNRYTG |
| | VPSRFSGSGSGTDFTLTISSLQPEDFATYYCQQSFSYPTFGQGTKLEIKRTVAAPSVFIF |
| | PPSDEQLKSGTASVVCLLNNFYPREAKVQWKVDNALQSGNSQESVTEQDSKDSTYS |
| | LSSTLTLSKADYEKHKVYACEVTHQGLSSPVTKSFNRGEC |
| SEQ ID NO: 85 | QVQLVQSGAEVKKPGSSVKVSCKASGYTFTDNYMIWVRQAPGQGLEWMGDINPYN |
| | GGTTFNQKFKGRVTITADKSTSTAYMELSSLRSEDTAVYYCARESPYFSNLYVMDY |
| | WGQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKRVEPKSCD |
| | KTHTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVCTLPPSREEMTKNQVSLSCAVKGFYPSDIAVEWESNGQPENN |
| | YKTTPPVLDSDGSFFLVSKLTVDKSRWQQGNVFSCSVMHEALHNHYTQKSLSLSPG |
| | X, wherein X is K or absent |
| SEQ ID NO: 86 | EIVLTQSPATLSLSPGERATLSCKASQSVDYDGDNYMNWYQQKPGQAPRLLIYAAS |
| | NLESGIPARFSGSGSGTDFTLTISSLEPEDFAVYYCHLSNEDLSTFGGGTKVEIKRTVA |
| | APSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |

TABLE 11
Sequences used to generate αCCR2/αCSF1R multispecific molecules.

| Multispecific | | | | |
|---|---|---|---|---|
| Molecule | Heavy Chain 1 | Light Chain 1 | Heavy Chain 2 | Light Chain 2 |
| 1 | SEQ ID NO: 71 | SEQ ID NO: 72 | SEQ ID NO: 73 | SEQ ID NO: 74 |
| 2 | SEQ ID NO: 75 | SEQ ID NO: 76 | SEQ ID NO: 83 | SEQ ID NO: 84 |
| 3 | SEQ ID NO: 75 | SEQ ID NO: 76 | SEQ ID NO: 85 | SEQ ID NO: 86 |
| 4 | SEQ ID NO: 77 | SEQ ID NO: 78 | SEQ ID NO: 83 | SEQ ID NO: 84 |
| 5 | SEQ ID NO: 77 | SEQ ID NO: 78 | SEQ ID NO: 85 | SEQ ID NO: 86 |
| 6 | SEQ ID NO: 79 | SEQ ID NO: 80 | SEQ ID NO: 83 | SEQ ID NO: 84 |
| 7 | SEQ ID NO: 79 | SEQ ID NO: 80 | SEQ ID NO: 85 | SEQ ID NO: 86 |
| 8 | SEQ ID NO: 81 | SEQ ID NO: 82 | SEQ ID NO: 83 | SEQ ID NO: 84 |
| 9 | SEQ ID NO: 81 | SEQ ID NO: 82 | SEQ ID NO: 85 | SEQ ID NO: 86 |

### Example 2. Generation of Multiple αCCR2/αCSF1R Bispecific Antibody Molecules

### 1. Construction of the Plasmids.

The DNA encoding the protein sequences was optimized for expression in Cricetulus griseus, synthesized, and cloned into the pcDNA3.4-TOPO (Life Technologies A14697) using Gateway cloning. All constructs contained an Ig Kappa leader sequence METDTLLLWVLLLWVPGSTG (SEQ ID NO: 116).

### 2. Expression and Purification.

The plasmids were co-transfected into either Expi293 cells (Life Technologies A14527) or ExpiCHO cells (Life Technologies A29127). Transfections were performed using 1 mg of total DNA for a multispecific construct with a 1:1 knob to hole heavy chain ratio and 3:2 light chain to heavy chain ratio. When biotinylation was required, 250 μg of BirA was added per liter in addition to the multispecific construct DNA. Transfection in Expi293 cells was done using linear 25,000 Da polyethylenimine (PEI, Polysciences Inc 23966) in a 3:1 ratio with the total DNA. The DNA and PEI were each added to 50 mL of OptiMem (Life Technologies 31985088) medium and sterile filtered. The DNA and PEI were combined for 10 minutes and added to the Expi293 cells with a cell density of 1.8-2.8×10⁶ cells/mL and a viability of at least 95%. The ExpiCHO transfection was performed according to the manufacturer's instructions. Expi293 cells were grown in a humidified incubator at 37° C. with 8% CO₂ for 5-7 days after transfection and ExpiCHO cells were grown for 14 days at 32° C. with 5% CO₂. The cells were pelleted by centrifugation at 4500×g and the supernatant was filtered through a 0.2 μm membrane. Protein A resin (GE 17-1279-03) was added to the filtered supernatant and incubated for 1-3 hours at room temperature. The resin was packed into a column, washed with 3×10 column volumes of Dulbecco's phosphate-buffered saline (DPBS, Life Technologies 14190-144). The bound protein was eluted from the column with 20 mM citrate, 100 mM NaCl, pH 2.9. When necessary, the proteins were further purified using ligand affinity and/or size exclusion chromatography on a Superdex 200 column with a running buffer of DPBS.

TABLE 12
Amino Acid Sequences.

| SEQ ID | | |
|---|---|---|
| NO | Description | Amino Acid Sequence |
| SEQ ID | Ig Kappa | METDTLLLWVLLLWVPGSTG |
| NO: 116 | Signal | |
| | Peptide | |
| SEQ ID | hCH1 | ASTKGPSVFPLAPSSKSTSGGTAALGCLVKDYFPEPVTVSWNSGALT |
| NO: 55 | | SGVHTFPAVLQSSGLYSLSSVVTVPSSSLGTQTYICNVNHKPSNTKVD |
| | | KRVEPKSC |
| SEQ ID | hFc Knob | DKTHTCPPCPAPELLGGPSVFLFPPKPKDTLMISRTPEVTCVVVDVSH |
| NO: 56 | | EDPEVKFNWYVDGVEVHNAKTKPREEQYNSTYRVVSVLTVLHQDW |
| | | LNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFL |
| | | YSKLTVDKSRWQQGNVFSCSVMHEALHNHYTQKSLSLSPGX, |
| | | wherein X is K or absent |
| SEQ ID | G Linker | G |
| NO: 127 | | |
| SEQ ID | 4GS Linker | GGGGS |
| NO: 128 | | |
| SEQ ID | GGSGG | GGSGG |
| NO: 129 | Linker | |
| SEQ ID | GGSGGS | GGSGGS |
| NO: 130 | Linker | |
| SEQ ID | GGGSGG | GGGSGGG |
| NO: 131 | G Linker | |
| SEQ ID | GGGGSG | GGGGSGGGG |
| NO: 132 | GGG | |
| SEQ ID | 3x4GS | GGGGSGGGGSGGGGS |
| NO: 133 | Linker | |
| SEQ ID | 4x4GS | GGGGSGGGGSGGGGSGGGGS |
| NO: 134 | Linker | |
| SEQ ID | hCLIg_vk | RTVAAPSVFIFPPSDEQLKSGTASVVCLLNNFYPREAKVQWKVDNAL |
| NO: 58 | | QSGNSQESVTEQDSKDSTYSLSSTLTLSKADYEKHKVYACEVTHQG |
| | | LSSPVTKSFNRGEC |
| SEQ ID | hCLIg_vl | GQPKANPTVTLFPPSSEELQANKATLVCLISDFYPGAVTVAWKADGS |
| NO: 61 | | PVKAGVETTKPSKQSNNKYAASSYLSLTPEQWKSHRSYSCQVTHEG |
| | | STVEKTVAPTECS |
| SEQ ID | α-hCSF1R | QVQLVQSGAEVKKPGASVKVSCKASGYTFTSYDISWVRQAPGQGLE |
| NO: 66 | emactuzumab | WMGVIWTDGGTNYAQKLQGRVTMTTDTSTSTAYMELRSLRSDDTA |
| | VH | VYYCARDQRLYFDVWGQGTTVTVSS |
| SEQ ID | α-hCSF1R | DIQMTQSPSSLSASVGDRVTITCRASEDVNTYVSWYQQKPGKAPKLL |
| NO: 67 | emactuzumab | IYAASNRYTGVPSRFSGSGSGTDFTLTISSLQPEDFATYYCQQSFSYPT |
| | VL | FGQGTKLEIK |
| SEQ ID | hFc Hole | DKTHTCPPCPAPELLGGPSVFLFPPKPKDTLMISRTPEVTCVVVDVSH |
| NO: 68 | | EDPEVKFNWYVDGVEVHNAKTKPREEQYNSTYRVVSVLTVLHQDW |
| | | LNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLV |
| | | SKLTVDKSRWQQGNVFSCSVMHEALHNHYTQKSLSLSPGX, wherein |
| | | X is K or absent |
| SEQ ID | α-hCSF1R | QVQLVQSGAEVKKPGSSVKVSCKASGYTFTDNYMIWVRQAPGQGL |
| NO: 69 | cabiralizumab | EWMGDINPYNGGTTFNQKFKGRVTITADKSTSTAYMELSSLRSEDT |
| | VH | AVYYCARESPYFSNLYVMDYWGQGTLVTVSS |
| SEQ ID | α-hCSF1R | EIVLTQSPATLSLSPGERATLSCKASQSVDYDGDNYMNWYQQKPGQ |
| NO: 70 | cabiralizumab | APRLLIYAASNLESGIPARFSGSGSGTDFTLTISSLEPEDFAVYYCHLS |
| | VL | NEDLSTFGGGTKVEIK |
| SEQ ID | α-hPDL1 | EVQLLESGGGLVQPGGSLRLSCAASGFTFSSYIMMWVRQAPGKGLE |
| NO: 113 | avelumab | WVSSIYPSGGITFYADTVKGRFTISRDNSKNTLYLQMNSLRAEDTAV |
| | VH | YYCARIKLGTVTTVDYWGQGTLVTVSS |
| SEQ ID | α-hPDL1 | QSALTQPASVSGSPGQSITISCTGTSSDVGGYNYVSWYQQHPGKAPK |
| NO: 114 | avelumab | LMIYDVSNRPSGVSNRFSGSKSGNTASLTISGLQAEDEADYYCSSYTS |
| | VL | SSTRVFGTGTKVTVL |
| SEQ ID | TGFβR1 | MEAAVAAPRPRLLLLVLAAAAAAAAA |
| NO: 191 | leader | |
| | sequence | |
| SEQ ID | TGFβR1 | LLPGATALQCFCHLCTKDNFTCVTDGLCFVSVTETTDKVIHNSMCIA |
| NO: 135 | | EIDLIPRDRPFVCAPSSKTGSVTTTYCCNQDHCNKIELPTTVKSSPGLG |
| | | PVE |
| SEQ ID | TGFβR2 | MGRGLLRGLWPLHIVLWTRIAS |
| NO: 136 | leader | |
| | sequence | |
| SEQ ID | TGFβR2 | TIPPHVQKSVNNDMIVTDNNGAVKFPQLCKFCDVRFSTCDNQKSCM |
| NO: 100 | | SNCSITSICEKPQEVCVAVWRKNDENITLETVCHDPKLPYHDFILEDA |
| | | ASPKCIMKEKKKPGETFFMCSCSSDECNDNIIFSEEYNTSNPD |
| SEQ ID | α-PDL1 | EVQLVESGGGLVQPGGSLRLSCAASGFTFSRYWMSWVRQAPGKGL |
| NO: 109 | durvalumab | EWVANIKQDGSEKYYVDSVKGRFTISRDNAKNSLYLQMNSLRAEDT |
| | VH | AVYYCAREGGWFGELAFDYWGQGTLVTVSS |
| SEQ ID | α-PDL1 | EIVLTQSPGTLSLSPGERATLSCRASQRVSSSYLAWYQQKPGQAPRLL |
| NO: 110 | durvalumab | IYDASSRATGIPDRFSGSGSGTDFTLTISRLEPEDFAVYYCQQYGSLP |
| | VL | WTFGQGTKVEIK |
| SEQ ID | α-PDL1 | EVQLVESGGGLVQPGGSLRLSCAASGFTFSDSWIHWVRQAPGKGLE |
| NO: | atezolizumab | WVAWISPYGGSTYYADSVKGRFTISADTSKNTAYLQMNSLRAEDTA |
| 111 | VH | VYYCARRHWPGGFDYWGQGTLVTVSS |
| SEQ ID | α-PDL1 | DIQMTQSPSSLSASVGDRVTITCRASQDVSTAVAWYQQKPGKAPKL |
| NO: 112 | atezolizumab | LIYSASFLYSGVPSRFSGSGSGTDFTLTISSLQPEDFATYYCQQYLYHP |
| | VL | ATFGQGTKVEIK |
| SEQ ID | TGFβR3 | GPEPGALCELSPVSASHPVQALMESFTVLSGCASRGTTGLPQEVHVL |
| NO: 108 | | NLRTAGQGPGQLQREVTLHLNPISSVHIHHKSVVFLLNSPHPLVWHL |
| | | KTERLATGVSRLFLVSEGSVVQFSSANFSLTAETEERNFPHGNEHLLN |
| | | WARKEYGAVTSFTELKIARNIYIKVGEDQVFPPKCNIGKNFLSLNYL |
| | | AEYLQPKAAEGCVMSSQPQNEEVHIIELITPNSNPYSAFQVDITIDIRP |
| | | SQEDLEVVKNLILILKCKKSVNWVIKSFDVKGSLKIIAPNSIGFGKESE |
| | | RSMTMTKSIRDDIPSTQGNLVKWALDNGYSPITSYTMAPVANRFHLR |
| | | LENNAEEMGDEEVHTIPPELRILLDPGALPALQNPPIRGGEGQNGGLP |
| | | FPFPDISRRVWNEEGEDGLPRPKDPVIPSIQLFPGLREPEEVQGSVDIA |
| | | LSVKCDNEKMIVAVEKDSFQASGYSGMDVTLLDPTCKAKMNGTHF |
| | | VLESPLNGCGTRPRWSALDGVVYYNSIVIQVPALGDSSGWPDGYED |
| | | LESGDNGFPGDMDEGDASLFTRPEIVVFNCSLQQVRNPSSFQEQPHG |
| | | NITFNMELYNTDLFLVPSQGVFSVPENGHVYVEVSVTKAEQELGFAI |
| | | QTCFISPYSNPDRMSHYTIIENICPKDESVKFYSPKRVHFPIPQADMDK |
| | | KRFSFVFKPVFNTSLLFLQCELTLCTKMEKHPQKLPKCVPPDEACTSL |
| | | DASIIWAMMQNKKTFTKPLAVIHHEAESKEKGPSMKEPNPISPPIFHG |
| | | LDTLTV |

TABLE 13
Amino acid sequences of the chains used to construct multispecific molecules.

| SEQ | | |
|---|---|---|
| ID NO | Description | Amino Acid Sequence |
| SEQ | α-hPDL1 | EVQLLESGGGLVQPGGSLRLSCAASGFTFSSYIMMWVRQAPGKGLEWV |
| ID NO: | avelumab VH- | SSIYPSGGITFYADTVKGRFTISRDNSKNTLYLQMNSLRAEDTAVYYC |
| 137 | hCH1-hFc_Knob- | ARIKLGTVTTVDYWGQGTLVTVSSASTKGPSVFPLAPSSKSTSGGTAA |
| | 3x4GS linker-α- | LGCLVKDYFPEPVTVSWNSGALTSGVHTFPAVLQSSGLYSLSSVVTVP |
| | hCSF1R | SSSLGTQTYICNVNHKPSNTKVDKRVEPKSCDKTHTCPPCPAPELLGG |
| | emactuzumab | PSVFLFPPKPKDTLMISRTPEVTCVVVDVSHEDPEVKFNWYVDGVEVH |
| | VH-4x4GS linker- | NAKTKPREEQYNSTYRVVSVLTVLHQDWLNGKEYKCKVSNKALPAPIE |
| | α-hCSF1R | KTISKAKGQPREPQVYTLPPCREEMTKNQVSLWCLVKGFYPSDIAVEW |
| | emactuzumab VL | ESNGQPENNYKTTPPVLDSDGSFFLYSKLTVDKSRWQQGNVFSCSVMH |
| | | EALHNHYTQKSLSLSPGKGGGGSGGGGSGGGGSQVQLVQSGAEVKKPG |
| | | ASVKVSCKASGYTFTSYDISWVRQAPGQGLEWMGVIWTDGGTNYAQKL |
| | | QGRVTMTTDTSTSTAYMELRSLRSDDTAVYYCARDQRLYFDVWGQGTT |
| | | VTVSSGGGGSGGGGSGGGGSGGGGSDIQMTQSPSSLSASVGDRVTITC |
| | | RASEDVNTYVSWYQQKPGKAPKLLIYAASNRYTGVPSRFSGSGSGTDF |
| | | TLTISSLQPEDFATYYCQQSFSYPTFGQGTKLEIK |
| SEQ | α-hPDL1 | QSALTQPASVSGSPGQSITISCTGTSSDVGGYNYVSWYQQHPGKAPKL |
| ID NO: | avelumab VL- | MIYDVSNRPSGVSNRFSGSKSGNTASLTISGLQAEDEADYYCSSYTSS |
| 138 | hCLIg_vl | STRVFGTGTKVTVLGQPKANPTVTLFPPSSEELQANKATLVCLISDFY |
| | | PGAVTVAWKADGSPVKAGVETTKPSKQSNNKYAASSYLSLTPEQWKSH |
| | | RSYSCQVTHEGSTVEKTVAPTECS |
| SEQ | α-hPDL1 VH- | EVQLLESGGGLVQPGGSLRLSCAASGFTFSSYIMMWVRQAPGKGLEWV |
| ID NO: | hCH1-hFc_Knob- | SSIYPSGGITFYADTVKGRFTISRDNSKNTLYLQMNSLRAEDTAVYYC |
| 139 | 3x4GS linker-α- | ARIKLGTVTTVDYWGQGTLVTVSSASTKGPSVFPLAPSSKSTSGGTAA |
| | hCSF1R | LGCLVKDYFPEPVTVSWNSGALTSGVHTFPAVLQSSGLYSLSSVVTVP |
| | cabiralizumab | SSSLGTQTYICNVNHKPSNTKVDKRVEPKSCDKTHTCPPCPAPELLGG |
| | VH-4x4GS linker- | PSVFLFPPKPKDTLMISRTPEVTCVVVDVSHEDPEVKFNWYVDGVEVH |
| | α-hCSF1R | NAKTKPREEQYNSTYRVVSVLTVLHQDWLNGKEYKCKVSNKALPAPIE |
| | cabiralizumab VL | KTISKAKGQPREPQVYTLPPCREEMTKNQVSLWCLVKGFYPSDIAVEW |
| | | ESNGQPENNYKTTPPVLDSDGSFFLYSKLTVDKSRWQQGNVFSCSVMH |
| | | EALHNHYTQKSLSLSPGKGGGGSGGGGSGGGGSQVQLVQSGAEVKKPG |
| | | SSVKVSCKASGYTFTDNYMIWVRQAPGQGLEWMGDINPYNGGTTFNQK |
| | | FKGRVTITADKSTSTAYMELSSLRSEDTAVYYCARESPYFSNLYVMDY |
| | | WGQGTLVTVSSGGGGSGGGGSGGGGSGGGGSEIVLTQSPATLSLSPGE |
| | | RATLSCKASQSVDYDGDNYMNWYQQKPGQAPRLLIYAASNLESGIPAR |
| | | FSGSGSGTDFTLTISSLEPEDFAVYYCHLSNEDLSTFGGGTKVEIK |
| SEQ | TGFβR1-4GS | LLPGATALQCFCHLCTKDNFTCVTDGLCFVSVTETTDKVIHNSMCIAE |
| ID NO: | linker-hCH1- | IDLIPRDRPFVCAPSSKTGSVTTTYCCNQDHCNKIELPTTVKSSPGLG |
| 140 | hFc_Hole | PVEGGGGSASTKGPSVFPLAPSSKSTSGGTAALGCLVKDYFPEPVTVS |
| | | WNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVC |
| | | TLPPSREEMTKNQVSLSCAVKGFYPSDIAVEWESNGQPENNYKTTPPV |
| | | LDSDGSFFLVSKLTVDKSRWQQGNVFSCSVMHEALHNHYTQKSLSLSP |
| | | GX, wherein X is K or absent |
| SEQ | TGFβR2-4GS- | TIPPHVQKSVNNDMIVTDNNGAVKFPQLCKFCDVRFSTCDNQKSCMSN |
| ID NO: | hCLIg_vk | CSITSICEKPQEVCVAVWRKNDENITLETVCHDPKLPYHDFILEDAAS |
| 141 | | PKCIMKEKKKPGETFFMCSCSSDECNDNIIFSEEYNTSNPDGGGGSRT |
| | | VAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPV |
| | | TKSFNRGEC |
| SEQ | TGFβR1-4GS- | LLPGATALQCFCHLCTKDNFTCVTDGLCFVSVTETTDKVIHNSMCIAE |
| ID NO: | hFc_Knob-3x4GS | IDLIPRDRPFVCAPSSKTGSVTTTYCCNQDHCNKIELPTTVKSSPGLG |
| 142 | linker-α-hPDL1 | PVEGGGGSDKTHTCPPCPAPELLGGPSVFLFPPKPKDTLMISRTPEVT |
| | VH-4x4GS linker- | CVVVDVSHEDPEVKFNWYVDGVEVHNAKTKPREEQYNSTYRVVSVLTV |
| | α-hPDL1 VL | LHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGKGGGGS |
| | | GGGGSGGGGSEVQLLESGGGLVQPGGSLRLSCAASGFTFSSYIMMWVR |
| | | QAPGKGLEWVSSIYPSGGITFYADTVKGRFTISRDNSKNTLYLQMNSL |
| | | RAEDTAVYYCARIKLGTVTTVDYWGQGTLVTVSSGGGGSGGGGSGGGG |
| | | SGGGGSQSALTQPASVSGSPGQSITISCTGTSSDVGGYNYVSWYQQHP |
| | | GKAPKLMIYDVSNRPSGVSNRFSGSKSGNTASLTISGLQAEDEADYYC |
| | | SSYTSSSTRVFGTGTKVTVL |
| SEQ | TGFβR2-4GS- | TIPPHVQKSVNNDMIVTDNNGAVKFPQLCKFCDVRFSTCDNQKSCMSN |
| ID NO: | hFc_Hole-3x4GS- | CSITSICEKPQEVCVAVWRKNDENITLETVCHDPKLPYHDFILEDAAS |
| 143 | α-hCSF1R | PKCIMKEKKKPGETFFMCSCSSDECNDNIIFSEEYNTSNPDGGGGSDK |
| | emactuzumab | THTCPPCPAPELLGGPSVFLFPPKPKDTLMISRTPEVTCVVVDVSHED |
| | VH-4x4GS-α- | PEVKFNWYVDGVEVHNAKTKPREEQYNSTYRVVSVLTVLHQDWLNGKE |
| | hCSF1R | YKCKVSNKALPAPIEKTISKAKGQPREPQVCTLPPSREEMTKNQVSLS |
| | emactuzumab VL | CAVKGFYPSDIAVEWESNGQPENNYKTTPPVLDSDGSFFLVSKLTVDK |
| | | SRWQQGNVFSCSVMHEALHNHYTQKSLSLSPGKGGGGSGGGGSGGGGS |
| | | QVQLVQSGAEVKKPGASVKVSCKASGYTFTSYDISWVRQAPGQGLEWM |
| | | GVIWTDGGTNYAQKLQGRVTMTTDTSTSTAYMELRSLRSDDTAVYYCA |
| | | RDQRLYFDVWGQGTTVTVSSGGGGSGGGGSGGGGSGGGGSDIQMTQSP |
| | | SSLSASVGDRVTITCRASEDVNTYVSWYQQKPGKAPKLLIYAASNRYT |
| | | GVPSRFSGSGSGTDFTLTISSLQPEDFATYYCQQSFSYPTFGQGTKLE |
| | | IK |
| SEQ | TGFβR2-4GS- | TIPPHVQKSVNNDMIVTDNNGAVKFPQLCKFCDVRFSTCDNQKSCMSN |
| ID NO: | hFc_Hole-3x4GS- | CSITSICEKPQEVCVAVWRKNDENITLETVCHDPKLPYHDFILEDAAS |
| 144 | α-hCSF1R | PKCIMKEKKKPGETFFMCSCSSDECNDNIIFSEEYNTSNPDGGGGSDK |
| | cabiralizumab | THTCPPCPAPELLGGPSVFLFPPKPKDTLMISRTPEVTCVVVDVSHED |
| | VH-4x4GS-α- | PEVKFNWYVDGVEVHNAKTKPREEQYNSTYRVVSVLTVLHQDWLNGKE |
| | hCSF1R | YKCKVSNKALPAPIEKTISKAKGQPREPQVCTLPPSREEMTKNQVSLS |
| | cabiralizumab VL | CAVKGFYPSDIAVEWESNGQPENNYKTTPPVLDSDGSFFLVSKLTVDK |
| | | SRWQQGNVFSCSVMHEALHNHYTQKSLSLSPGKGGGGSGGGGSGGGGS |
| | | QVQLVQSGAEVKKPGSSVKVSCKASGYTFTDNYMIWVRQAPGQGLEWM |
| | | GDINPYNGGTTFNQKFKGRVTITADKSTSTAYMELSSLRSEDTAVYYC |
| | | ARESPYFSNLYVMDYWGQGTLVTVSSGGGGSGGGGSGGGGSGGGGSEI |
| | | VLTQSPATLSLSPGERATLSCKASQSVDYDGDNYMNWYQQKPGQAPRL |
| | | LIYAASNLESGIPARFSGSGSGTDFTLTISSLEPEDFAVYYCHLSNED |
| | | LSTFGGGTKVEIK |
| SEQ | α-hCSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 145 | VH-hCH1- | RDQRLYFDVWGQGTTVTVSSASTKGPSVFPLAPSSKSTSGGTAALGCL |
| | hFc_Knob-3x4GS- | VKDYFPEPVTVSWNSGALTSGVHTFPAVLQSSGLYSLSSVVTVPSSSL |
| | α-PDL1 VH- | GTQTYICNVNHKPSNTKVDKRVEPKSCDKTHTCPPCPAPELLGGPSVF |
| | 4x4GS-α-PDL1 VL | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPCREEMTKNQVSLWCLVKGFYPSDIAVEWESNG |
| | | QPENNYKTTPPVLDSDGSFFLYSKLTVDKSRWQQGNVFSCSVMHEALH |
| | | NHYTQKSLSLSPGKGGGGSGGGGSGGGGSEVQLLESGGGLVQPGGSLR |
| | | LSCAASGFTFSSYIMMWVRQAPGKGLEWVSSIYPSGGITFYADTVKGR |
| | | FTISRDNSKNTLYLQMNSLRAEDTAVYYCARIKLGTVTTVDYWGQGTL |
| | | VTVSSGGGGSGGGGSGGGGSGGGGSQSALTQPASVSGSPGQSITISCT |
| | | GTSSDVGGYNYVSWYQQHPGKAPKLMIYDVSNRPSGVSNRFSGSKSGN |
| | | TASLTISGLQAEDEADYYCSSYTSSSTRVFGTGTKVTVL |
| SEQ | α-hCSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 146 | VH-hCH1- | ARESPYFSNLYVMDYWGQGTLVTVSSASTKGPSVFPLAPSSKSTSGGT |
| | hFc_Knob-3x4GS- | AALGCLVKDYFPEPVTVSWNSGALTSGVHTFPAVLQSSGLYSLSSVVT |
| | α-PDL1 VH- | VPSSSLGTQTYICNVNHKPSNTKVDKRVEPKSCDKTHTCPPCPAPELL |
| | 4x4GS-α-PDL1 VL | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVYTLPPCREEMTKNQVSLWCLVKGFYPSDIAV |
| | | EWESNGQPENNYKTTPPVLDSDGSFFLYSKLTVDKSRWQQGNVFSCSV |
| | | MHEALHNHYTQKSLSLSPGKGGGGSGGGGSGGGGSEVQLLESGGGLVQ |
| | | PGGSLRLSCAASGFTFSSYIMMWVRQAPGKGLEWVSSIYPSGGITFYA |
| | | DTVKGRFTISRDNSKNTLYLQMNSLRAEDTAVYYCARIKLGTVTTVDY |
| | | WGQGTLVTVSSGGGGSGGGGSGGGGSGGGGSQSALTQPASVSGSPGQS |
| | | ITISCTGTSSDVGGYNYVSWYQQHPGKAPKLMIYDVSNRPSGVSNRFS |
| | | GSKSGNTASLTISGLQAEDEADYYCSSYTSSSTRVFGTGTKVTVL |
| SEQ | α-hCSF1R | DIQMTQSPSSLSASVGDRVTITCRASEDVNTYVSWYQQKPGKAPKLLI |
| ID NO: | emactuzumab | YAASNRYTGVPSRFSGSGSGTDFTLTISSLQPEDFATYYCQQSFSYPT |
| 147 | CLIg_vk | FGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSFNRGEC |
| SEQ | α-hCSF1R | EIVLTQSPATLSLSPGERATLSCKASQSVDYDGDNYMNWYQQKPGQAP |
| ID NO: | cabiralizumab | RLLIYAASNLESGIPARFSGSGSGTDFTLTISSLEPEDFAVYYCHLSN |
| 148 | CLIg_vk | EDLSTFGGGTKVEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| SEQ | α-hPDL1 | EVQLVESGGGLVQPGGSLRLSCAASGFTFSRYWMSWVRQAPGKGLEWV |
| ID NO: | durvalumab VH- | ANIKQDGSEKYYVDSVKGRFTISRDNAKNSLYLQMNSLRAEDTAVYYC |
| 149 | hCH1-hFc_Knob- | AREGGWFGELAFDYWGQGTLVTVSSASTKGPSVFPLAPSSKSTSGGTA |
| | 3x4GS linker-α- | ALGCLVKDYFPEPVTVSWNSGALTSGVHTFPAVLQSSGLYSLSSVVTV |
| | hCSF1R | PSSSLGTQTYICNVNHKPSNTKVDKRVEPKSCDKTHTCPPCPAPELLG |
| | emactuzumab | GPSVFLFPPKPKDTLMISRTPEVTCVVVDVSHEDPEVKFNWYVDGVEV |
| | VH-4x4GS linker- | HNAKTKPREEQYNSTYRVVSVLTVLHQDWLNGKEYKCKVSNKALPAPI |
| | α-hCSF1R | EKTISKAKGQPREPQVYTLPPCREEMTKNQVSLWCLVKGFYPSDIAVE |
| | emactuzumab VL | WESNGQPENNYKTTPPVLDSDGSFFLYSKLTVDKSRWQQGNVFSCSVM |
| | | HEALHNHYTQKSLSLSPGKGGGGSGGGGSGGGGSQVQLVQSGAEVKKP |
| | | GASVKVSCKASGYTFTSYDISWVRQAPGQGLEWMGVIWTDGGTNYAQK |
| | | LQGRVTMTTDTSTSTAYMELRSLRSDDTAVYYCARDQRLYFDVWGQGT |
| | | TVTVSSDIQMTQSPSSLSASVGDRVTITCRASEDVNTYVSWYQQKPGK |
| | | APKLLIYAASNRYTGVPSRFSGSGSGTDFTLTISSLQPEDFATYYCQQ |
| | | SFSYPTFGQGTKLEIK |
| SEQ | α-hPDL1 | EIVLTQSPGTLSLSPGERATLSCRASQRVSSSYLAWYQQKPGQAPRLL |
| ID NO: | durvalumab VL- | IYDASSRATGIPDRFSGSGSGTDFTLTISRLEPEDFAVYYCQQYGSLP |
| 150 | hCLIg_vk | WTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKHKV |
| | | YACEVTHQGLSSPVTKSFNRGEC |
| SEQ | α-hPDL1 | EVQLVESGGGLVQPGGSLRLSCAASGFTFSDSWIHWVRQAPGKGLEWV |
| ID NO: | atezolizumab VH- | AWISPYGGSTYYADSVKGRFTISADTSKNTAYLQMNSLRAEDTAVYYC |
| 151 | hCH1-hFc_Knob- | ARRHWPGGFDYWGQGTLVTVSSASTKGPSVFPLAPSSKSTSGGTAALG |
| | 3x4GS linker-α- | CLVKDYFPEPVTVSWNSGALTSGVHTFPAVLQSSGLYSLSSVVTVPSS |
| | hCSF1R | SLGTQTYICNVNHKPSNTKVDKRVEPKSCDKTHTCPPCPAPELLGGPS |
| | emactuzumab | VFLFPPKPKDTLMISRTPEVTCVVVDVSHEDPEVKFNWYVDGVEVHNA |
| | VH-4x4GS linker- | KTKPREEQYNSTYRVVSVLTVLHQDWLNGKEYKCKVSNKALPAPIEKT |
| | α-hCSF1R | ISKAKGQPREPQVYTLPPCREEMTKNQVSLWCLVKGFYPSDIAVEWES |
| | emactuzumab VL | NGQPENNYKTTPPVLDSDGSFFLYSKLTVDKSRWQQGNVFSCSVMHEA |
| | | LHNHYTQKSLSLSPGKGGGGSGGGGSGGGGSQVQLVQSGAEVKKPGAS |
| | | VKVSCKASGYTFTSYDISWVRQAPGQGLEWMGVIWTDGGTNYAQKLQG |
| | | RVTMTTDTSTSTAYMELRSLRSDDTAVYYCARDQRLYFDVWGQGTTVT |
| | | VSSDIQMTQSPSSLSASVGDRVTITCRASEDVNTYVSWYQQKPGKAPK |
| | | LLIYAASNRYTGVPSRFSGSGSGTDFTLTISSLQPEDFATYYCQQSFS |
| | | YPTFGQGTKLEIK |
| SEQ | α-hPDL1 | DIQMTQSPSSLSASVGDRVTITCRASQDVSTAVAWYQQKPGKAPKLLI |
| ID NO: | atezolizumab VL- | YSASFLYSGVPSRFSGSGSGTDFTLTISSLQPEDFATYYCQQYLYHPA |
| 152 | hCLIg_vk | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSFNRGEC |
| SEQ | α-CSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 153 | VH-hCH1- | RDQRLYFDVWGQGTTVTVSSASTKGPSVFPLAPSSKSTSGGTAALGCL |
| | hFc_knob- | VKDYFPEPVTVSWNSGALTSGVHTFPAVLQSSGLYSLSSVVTVPSSSL |
| | 3x4GS-α-PDL1 | GTQTYICNVNHKPSNTKVDKRVEPKSCDKTHTCPPCPAPELLGGPSVF |
| | durvalumab VH- | LFPPKPKDTLMISRTPEVTCVVVDVSHEDPEVKFNWYVDGVEVHNAKT |
| | 4x4GS-α-PDL1 | KPREEQYNSTYRVVSVLTVLHQDWLNGKEYKCKVSNKALPAPIEKTIS |
| | durvalumab VL | KAKGQPREPQVYTLPPCREEMTKNQVSLWCLVKGFYPSDIAVEWESNG |
| | | QPENNYKTTPPVLDSDGSFFLYSKLTVDKSRWQQGNVFSCSVMHEALH |
| | | NHYTQKSLSLSPGKGGGGSGGGGSGGGGSEVQLVESGGGLVQPGGSLR |
| | | LSCAASGFTFSRYWMSWVRQAPGKGLEWVANIKQDGSEKYYVDSVKGR |
| | | FTISRDNAKNSLYLQMNSLRAEDTAVYYCAREGGWFGELAFDYWGQGT |
| | | LVTVSSGGGGSGGGGSGGGGSGGGGSEIVLTQSPGTLSLSPGERATLS |
| | | CRASQRVSSSYLAWYQQKPGQAPRLLIYDASSRATGIPDRFSGSGSGT |
| | | DFTLTISRLEPEDFAVYYCQQYGSLPWTFGQGTKVEIK |
| SEQ | α-CSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 154 | VH-hCH1- | RDQRLYFDVWGQGTTVTVSSASTKGPSVFPLAPSSKSTSGGTAALGCL |
| | hFc_knob- | VKDYFPEPVTVSWNSGALTSGVHTFPAVLQSSGLYSLSSVVTVPSSSL |
| | 3x4GS-α-PDL1 | GTQTYICNVNHKPSNTKVDKRVEPKSCDKTHTCPPCPAPELLGGPSVF |
| | atezolizumab VH- | LFPPKPKDTLMISRTPEVTCVVVDVSHEDPEVKFNWYVDGVEVHNAKT |
| | 4x4GS-α-PDL1 | KPREEQYNSTYRVVSVLTVLHQDWLNGKEYKCKVSNKALPAPIEKTIS |
| | atezolizumab VL | KAKGQPREPQVYTLPPCREEMTKNQVSLWCLVKGFYPSDIAVEWESNG |
| | | QPENNYKTTPPVLDSDGSFFLYSKLTVDKSRWQQGNVFScSVMHEALH |
| | | NHYTQKSLSLSPGKGGGGSGGGGSGGGGSEVQLVESGGGLVQPGGSLR |
| | | LSCAASGFTFSDSWIHWVRQAPGKGLEWVAWISPYGGSTYYADSVKGR |
| | | FTISADTSKNTAYLQMNSLRAEDTAVYYCARRHWPGGFDYWGQGTLVT |
| | | VSSGGGGSGGGGSGGGGSGGGGSDIQMTQSPSSLSASVGDRVTITCRA |
| | | SQDVSTAVAWYQQKPGKAPKLLIYSASFLYSGVPSRFSGSGSGTDFTL |
| | | TISSLQPEDFATYYCQQYLYHPATFGQGTKVEIK |
| SEQ | α-CSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 155 | VH-hCH1- | ARESPYFSNLYVMDYWGQGTLVTVSSASTKGPSVFPLAPSSKSTSGGT |
| | hFc_knob- | AALGCLVKDYFPEPVTVSWNSGALTSGVHTFPAVLQSSGLYSLSSVVT |
| | 3x4GS-α-PDL1 | VPSSSLGTQTYICNVNHKPSNTKVDKRVEPKSCDKTHTCPPCPAPELL |
| | durvalumab VH- | GGPSVFLFPPKPKDTLMISRTPEVTCVVVDVSHEDPEVKFNWYVDGVE |
| | 4x4GS-α-PDL1 | VHNAKTKPREEQYNSTYRVVSVLTVLHQDWLNGKEYKCKVSNKALPAP |
| | durvalumab VL | IEKTISKAKGQPREPQVYTLPPCREEMTKNQVSLWCLVKGFYPSDIAV |
| | | EWESNGQPENNYKTTPPVLDSDGSFFLYSKLTVDKSRWQQGNVFSCSV |
| | | MHEALHNHYTQKSLSLSPGKGGGGSGGGGSGGGGSEVQLVESGGGLVQ |
| | | PGGSLRLSCAASGFTFSRYWMSWVRQAPGKGLEWVANIKQDGSEKYYV |
| | | DSVKGRFTISRDNAKNSLYLQMNSLRAEDTAVYYCAREGGWFGELAFD |
| | | YWGQGTLVTVSSGGGGSGGGGSGGGGSGGGGSEIVLTQSPGTLSLSPG |
| | | ERATLSCRASQRVSSSYLAWYQQKPGQAPRLLIYDASSRATGIPDRFS |
| | | GSGSGTDFTLTISRLEPEDFAVYYCQQYGSLPWTFGQGTKVEIK |
| SEQ | α-CSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 156 | VH-hCH1- | ARESPYFSNLYVMDYWGQGTLVTVSSASTKGPSVFPLAPSSKSTSGGT |
| | hFc_knob- | AALGCLVKDYFPEPVTVSWNSGALTSGVHTFPAVLQSSGLYSLSSVVT |
| | 3x4GS-α-PDL1 | VPSSSLGTQTYICNVNHKPSNTKVDKRVEPKSCDKTHTCPPCPAPELL |
| | atezolizumab VH- | GGPSVFLFPPKPKDTLMISRTPEVTCVVVDVSHEDPEVKFNWYVDGVE |
| | 4x4GS-α-PDL1 | VHNAKTKPREEQYNSTYRVVSVLTVLHQDWLNGKEYKCKVSNKALPAP |
| | atezolizumab VL | IEKTISKAKGQPREPQVYTLPPCREEMTKNQVSLWCLVKGFYPSDIAV |
| | | EWESNGQPENNYKTTPPVLDSDGSFFLYSKLTVDKSRWQQGNVFSCSV |
| | | MHEALHNHYTQKSLSLSPGKGGGGSGGGGSGGGGSEVQLVESGGGLVQ |
| | | PGGSLRLSCAASGFTFSDSWIHWVRQAPGKGLEWVAWISPYGGSTYYA |
| | | DSVKGRFTISADTSKNTAYLQMNSLRAEDTAVYYCARRHWPGGFDYWG |
| | | QGTLVTVSSGGGGSGGGGSGGGGSGGGGSDIQMTQSPSSLSASVGDRV |
| | | TITCRASQDVSTAVAWYQQKPGKAPKLLIYSASFLYSGVPSRFSGSGS |
| | | GTDFTLTISSLQPEDFATYYCQQYLYHPATFGQGTKVEIK |
| SEQ | TFGβR1-4GS- | LLPGATALQCFCHLCTKDNFTCVTDGLCFVSVTETTDKVIHNSMCIAE |
| ID NO: | hFc_Knob-3x4GS- | IDLIPRDRPFVCAPSSKTGSVTTTYCCNQDHCNKIELPTTVKSSPGLG |
| 157 | α-PDL1 | PVEGGGGSDKTHTCPPCPAPELLGGPSVFLFPPKPKDTLMISRTPEVT |
| | durvalumab VH- | CVVVDVSHEDPEVKFNWYVDGVEVHNAKTKPREEQYNSTYRVVSVLTV |
| | 4x4GS-αPDL1 | LHQDWLNGKEYKCKVSNKALPAPIEKTISKAKGQPREPQVYTLPPCRE |
| | durvalumab VL | EMTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGKEVQLV |
| | | ESGGGLVQPGGSLRLSCAASGFTFSRYWMSWVRQAPGKGLEWVANIKQ |
| | | DGSEKYYVDSVKGRFTISRDNAKNSLYLQMNSLRAEDTAVYYCAREGG |
| | | WFGELAFDYWGQGTLVTVSSGGGGSGGGGSGGGGSGGGGSEIVLTQSP |
| | | GTLSLSPGERATLSCRASQRVSSSYLAWYQQKPGQAPRLLIYDASSRA |
| | | TGIPDRFSGSGSGTDFTLTISRLEPEDFAVYYCQQYGSLPWTFGQGTK |
| | | VEIK |
| SEQ | TFGβR1-4GS- | LLPGATALQCFCHLCTKDNFTCVTDGLCFVSVTETTDKVIHNSMCIAE |
| ID NO: | hFc_Knob-3x4GS- | IDLIPRDRPFVCAPSSKTGSVTTTYCCNQDHCNKIELPTTVKSSPGLG |
| 158 | α-PDL1 | PVEGGGGSDKTHTCPPCPAPELLGGPSVFLFPPKPKDTLMISRTPEVT |
| | atezolizumab VH- | CVVVDVSHEDPEVKFNWYVDGVEVHNAKTKPREEQYNSTYRVVSVLTV |
| | 4x4GS-αPDL1 | LHQDWLNGKEYKCKVSNKALPAPIEKTISKAKGQPREPQVYTLPPCRE |
| | atezolizumab VL | EMTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGKGGGGS |
| | | GGGGSGGGGSEVQLVESGGGLVQPGGSLRLSCAASGFTFSDSWIHWVR |
| | | QAPGKGLEWVAWISPYGGSTYYADSVKGRFTISADTSKNTAYLQMNSL |
| | | RAEDTAVYYCARRHWPGGFDYWGQGTLVTVSSGGGGSGGGGSGGGGSG |
| | | GGGSDIQMTQSPSSLSASVGDRVTITCRASQDVSTAVAWYQQKPGKAP |
| | | KLLIYSASFLYSGVPSRFSGSGSGTDFTLTISSLQPEDFATYYCQQYL |
| | | YHPATFGQGTKVEIK |
| SEQ | TFGβR1-4GS- | LLPGATALQCFCHLCTKDNFTCVTDGLCFVSVTETTDKVIHNSMCIAE |
| ID NO: | hFc_Knob-3x4GS- | IDLIPRDRPFVCAPSSKTGSVTTTYCCNQDHCNKIELPTTVKSSPGLG |
| 159 | α-CSF1R | PVEGGGGSDKTHTCPPCPAPELLGGPSVFLFPPKPKDTLMISRTPEVT |
| | emactizumab | CVVVDVSHEDPEVKFNWYVDGVEVHNAKTKPREEQYNSTYRVVSVLTV |
| | VH-4x4GS- | LHQDWLNGKEYKCKVSNKALPAPIEKTISKAKGQPREPQVCTLPPSRE |
| | αCSF1R | EMTKNQVSLSCAVKGFYPSDIAVEWESNGQPENNYKTTPPVLDSDGSF |
| | emactizumab VL | FLVSKLTVDKSRWQQGNVFSCSVMHEALHNHYTQKSLSLSPGKGGGGS |
| | | GGGGSGGGGSQVQLVQSGAEVKKPGASVKVSCKASGYTFTSYDISWVR |
| | | QAPGQGLEWMGVIWTDGGTNYAQKLQGRVTMTTDTSTSTAYMELRSLR |
| | | SDDTAVYYCARDQRLYFDVWGQGTTVTVSSGGGGSGGGGSGGGGSGGG |
| | | GSDIQMTQSPSSLSASVGDRVTITCRASEDVNTYVSWYQQKPGKAPKL |
| | | LIYAASNRYTGVPSRFSGSGSGTDFTLTISSLQPEDFATYYCQQSFSY |
| | | PTFGQGTKLEIK |
| SEQ | TFGβR1-4GS- | LLPGATALQCFCHLCTKDNFTCVTDGLCFVSVTETTDKVIHNSMCIAE |
| ID NO: | hFc_Knob-3x4GS- | IDLIPRDRPFVCAPSSKTGSVTTTYCCNQDHCNKIELPTTVKSSPGLG |
| 160 | α-CSF1R | PVEGGGGSDKTHTCPPCPAPELLGGPSVFLFPPKPKDTLMISRTPEVT |
| | cabiralizumab | CVVVDVSHEDPEVKFNWYVDGVEVHNAKTKPREEQYNSTYRVVSVLTV |
| | VH-4x4GS- | LHQDWLNGKEYKCKVSNKALPAPIEKTISKAKGQPREPQVCTLPPSRE |
| | αCSF1R | EMTKNQVSLSCAVKGFYPSDIAVEWESNGQPENNYKTTPPVLDSDGSF |
| | cabiralizumab VL | FLVSKLTVDKSRWQQGNVFSCSVMHEALHNHYTQKSLSLSPGKGGGGS |
| | | GGGGSGGGGSQVQLVQSGAEVKKPGSSVKVSCKASGYTFTDNYMIWVR |
| | | QAPGQGLEWMGDINPYNGGTTFNQKFKGRVTITADKSTSTAYMELSSL |
| | | RSEDTAVYYCARESPYFSNLYVMDYWGQGTLVTVSSGGGGSGGGGSGG |
| | | GGSGGGGSEIVLTQSPATLSLSPGERATLSCKASQSVDYDGDNYMNWY |
| | | QQKPGQAPRLLIYAASNLESGIPARFSGSGSGTDFTLTISSLEPEDFA |
| | | VYYCHLSNEDLSTFGGGTKVEIK |
| SEQ | TGFβR1-4GS- | LLPGATALQCFCHLCTKDNFTCVTDGLCFVSVTETTDKVIHNSMCIAE |
| ID NO: | CLIg_vk | IDLIPRDRPFVCAPSSKTGSVTTTYCCNQDHCNKIELPTTVKSSPGLG |
| 161 | | PVEGGGGSRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| SEQ | TGFβR2-4GS | TIPPHVQKSVNNDMIVTDNNGAVKFPQLCKFCDVRFSTCDNQKSCMSN |
| ID NO: | linker-hCH1- | CSITSICEKPQEVCVAVWRKNDENITLETVCHDPKLPYHDFILEDAAS |
| 162 | hFc_Hole | PKCIMKEKKKPGETFFMCSCSSDECNDNIIFSEEYNTSNPDGGGGSAS |
| | | TKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRV |
| | | EPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFSCSVMHEALHNHYTQKSLSLSPGX, |
| | | wherein X is K or absent |
| SEQ | TGFβR2-4GS- | TIPPHVQKSVNNDMIVTDNNGAVKFPQLCKFCDVRFSTCDNQKSCMSN |
| ID NO: | hFc_Knob-3x4GS- | CSITSICEKPQEVCVAVWRKNDENITLETVCHDPKLPYHDFILEDAAS |
| 163 | αPDL1 avelumab | PKCIMKEKKKPGETFFMCSCSSDECNDNIIFSEEYNTSNPDGGGGSDK |
| | VH-4x4GS- | THTCPPCPAPELLGGPSVFLFPPKPKDTLMISRTPEVTCVVVDVSHED |
| | αPDL1 avelumab | PEVKFNWYVDGVEVHNAKTKPREEQYNSTYRVVSVLTVLHQDWLNGKE |
| | VL | YKCKVSNKALPAPIEKTISKAKGQPREPQVYTLPPCREEMTKNQVSLW |
| | | CLVKGFYPSDIAVEWESNGQPENNYKTTPPVLDSDGSFFLYSKLTVDK |
| | | SRWQQGNVFSCSVMHEALHNHYTQKSLSLSPGKGGGGSGGGGSGGGGS |
| | | EVQLLESGGGLVQPGGSLRLSCAASGFTFSSYIMMWVRQAPGKGLEWV |
| | | SSIYPSGGITFYADTVKGRFTISRDNSKNTLYLQMNSLRAEDTAVYYC |
| | | ARIKLGTVTTVDYWGQGTLVTVSSGGGGSGGGGSGGGGSGGGGSQSAL |
| | | TQPASVSGSPGQSITISCTGTSSDVGGYNYVSWYQQHPGKAPKLMIYD |
| | | VSNRPSGVSNRFSGSKSGNTASLTISGLQAEDEADYYCSSYTSSSTRV |
| | | FGTGTKVTVL |
| SEQ | TGFβR2-4GS- | TIPPHVQKSVNNDMIVTDNNGAVKFPQLCKFCDVRFSTCDNQKSCMSN |
| ID NO: | hFc_Knob-3x4GS- | CSITSICEKPQEVCVAVWRKNDENITLETVCHDPKLPYHDFILEDAAS |
| 164 | αPDL1 | PKCIMKEKKKPGETFFMCSCSSDECNDNIIFSEEYNTSNPDGGGGSDK |
| | durvalumab VH- | THTCPPCPAPELLGGPSVFLFPPKPKDTLMISRTPEVTCVVVDVSHED |
| | 4x4GS-αPDL1 | PEVKFNWYVDGVEVHNAKTKPREEQYNSTYRVVSVLTVLHQDWLNGKE |
| | durvalumab VL | YKCKVSNKALPAPIEKTISKAKGQPREPQVYTLPPCREEMTKNQVSLW |
| | | CLVKGFYPSDIAVEWESNGQPENNYKTTPPVLDSDGSFFLYSKLTVDK |
| | | SRWQQGNVFSCSVMHEALHNHYTQKSLSLSPGKGGGGSGGGGSGGGGS |
| | | EVQLVESGGGLVQPGGSLRLSCAASGFTFSRYWMSWVRQAPGKGLEWV |
| | | ANIKQDGSEKYYVDSVKGRFTISRDNAKNSLYLQMNSLRAEDTAVYYC |
| | | AREGGWFGELAFDYWGQGTLVTVSSGGGGSGGGGSGGGGSGGGGSEIV |
| | | LTQSPGTLSLSPGERATLSCRASQRVSSSYLAWYQQKPGQAPRLLIYD |
| | | ASSRATGIPDRFSGSGSGTDFTLTISRLEPEDFAVYYCQQYGSLPWTF |
| | | GQGTKVEIK |
| SEQ | TGFβR2-4GS- | TIPPHVQKSVNNDMIVTDNNGAVKFPQLCKFCDVRFSTCDNQKSCMSN |
| ID NO: | hFc_Knob-3x4GS- | CSITSICEKPQEVCVAVWRKNDENITLETVCHDPKLPYHDFILEDAAS |
| 165 | αPDL1 | PKCIMKEKKKPGETFFMCSCSSDECNDNIIFSEEYNTSNPDGGGGSDK |
| | atezolizumab VH- | THTCPPCPAPELLGGPSVFLFPPKPKDTLMISRTPEVTCVVVDVSHED |
| | 4x4GS-αPDL1 | PEVKFNWYVDGVEVHNAKTKPREEQYNSTYRVVSVLTVLHQDWLNGKE |
| | atezolizumab VL | YKCKVSNKALPAPIEKTISKAKGQPREPQVYTLPPCREEMTKNQVSLW |
| | | CLVKGFYPSDIAVEWESNGQPENNYKTTPPVLDSDGSFFLYSKLTVDK |
| | | SRWQQGNVFSCSVMHEALHNHYTQKSLSLSPGKGGGGSGGGGSGGGGS |
| | | EVQLVESGGGLVQPGGSLRLSCAASGFTFSDSWIHWVRQAPGKGLEWV |
| | | AWISPYGGSTYYADSVKGRFTISADTSKNTAYLQMNSLRAEDTAVYYC |
| | | ARRHWPGGFDYWGQGTLVTVSSGGGGSGGGGSGGGGSGGGGSDIQMTQ |
| | | SPSSLSASVGDRVTITCRASQDVSTAVAWYQQKPGKAPKLLIYSASFL |
| | | YSGVPSRFSGSGSGTDFTLTISSLQPEDFATYYCQQYLYHPATFGQGT |
| | | KVEIK |
| SEQ | TFGβR1-4GS- | LLPGATALQCFCHLCTKDNFTCVTDGLCFVSVTETTDKVIHNSMCIAE |
| ID NO: | hCH1-hFc_Knob- | IDLIPRDRPFVCAPSSKTGSVTTTYCCNQDHCNKIELPTTVKSSPGLG |
| 166 | 3x4GS-α-PDL1 | PVEGGGGSASTKGPSVFPLAPSSKSTSGGTAALGCLVKDYFPEPVTVS |
| | avelumab VH- | WNSGALTSGVHTFPAVLQSSGLYSLSSVVTVPSSSLGTQTYICNVNHK |
| | 4x4GS-α-PDL1 | PSNTKVDKRVEPKSCDKTHTCPPCPAPELLGGPSVFLFPPKPKDTLMI |
| | avelumab VL | SRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVY |
| | | TLPPCREEMTKNQVSLWCLVKGFYPSDIAVEWESNGQPENNYKTTPPV |
| | | LDSDGSFFLYSKLTVDKSRWQQGNVFSCSVMHEALHNHYTQKSLSLSP |
| | | GKGGGGSGGGGSGGGGSEVQLLESGGGLVQPGGSLRLSCAASGFTFSS |
| | | YIMMWVRQAPGKGLEWVSSIYPSGGITFYADTVKGRFTISRDNSKNTL |
| | | YLQMNSLRAEDTAVYYCARIKLGTVTTVDYWGQGTLVTVSSGGGGSGG |
| | | GGSGGGGSGGGGSQSALTQPASVSGSPGQSITISCTGTSSDVGGYNYV |
| | | SWYQQHPGKAPKLMIYDVSNRPSGVSNRFSGSKSGNTASLTISGLQAE |
| | | DEADYYCSSYTSSSTRVFGTGTKVTVL |
| SEQ | TFGβR1-4GS- | LLPGATALQCFCHLCTKDNFTCVTDGLCFVSVTETTDKVIHNSMCIAE |
| ID NO: | hCH1-hFc_Knob- | IDLIPRDRPFVCAPSSKTGSVTTTYCCNQDHCNKIELPTTVKSSPGLG |
| 167 | 3x4GS-α-PDL1 | PVEGGGGSASTKGPSVFPLAPSSKSTSGGTAALGCLVKDYFPEPVTVS |
| | durvalumab VH- | WNSGALTSGVHTFPAVLQSSGLYSLSSVVTVPSSSLGTQTYICNVNHK |
| | 4x4GS-α-PDL1- | PSNTKVDKRVEPKSCDKTHTCPPCPAPELLGGPSVFLFPPKPKDTLMI |
| | durvalumab VL | SRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVY |
| | | TLPPCREEMTKNQVSLWCLVKGFYPSDIAVEWESNGQPENNYKTTPPV |
| | | LDSDGSFFLYSKLTVDKSRWQQGNVFSCSVMHEALHNHYTQKSLSLSP |
| | | GKGGGGSGGGGSGGGGSEVQLVESGGGLVQPGGSLRLSCAASGFTFSR |
| | | YWMSWVRQAPGKGLEWVANIKQDGSEKYYVDSVKGRFTISRDNAKNSL |
| | | YLQMNSLRAEDTAVYYCAREGGWFGELAFDYWGQGTLVTVSSGGGGSG |
| | | GGGSGGGGSGGGGSEIVLTQSPGTLSLSPGERATLSCRASQRVSSSYL |
| | | AWYQQKPGQAPRLLIYDASSRATGIPDRFSGSGSGTDFTLTISRLEPE |
| | | DFAVYYCQQYGSLPWTFGQGTKVEIK |
| SEQ | TFGβR1-4GS- | LLPGATALQCFCHLCTKDNFTCVTDGLCFVSVTETTDKVIHNSMCIAE |
| ID NO: | hCH1-hFc_Knob- | IDLIPRDRPFVCAPSSKTGSVTTTYCCNQDHCNKIELPTTVKSSPGLG |
| 168 | 3x4GS-α-PDL1 | PVEGGGGSASTKGPSVFPLAPSSKSTSGGTAALGCLVKDYFPEPVTVS |
| | atezolizumab VH- | WNSGALTSGVHTFPAVLQSSGLYSLSSVVTVPSSSLGTQTYICNVNHK |
| | 4x4GS-α-PDL1 | PSNTKVDKRVEPKSCDKTHTCPPCPAPELLGGPSVFLFPPKPKDTLMI |
| | atezolizumab VL | SRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVY |
| | | TLPPCREEMTKNQVSLWCLVKGFYPSDIAVEWESNGQPENNYKTTPPV |
| | | LDSDGSFFLYSKLTVDKSRWQQGNVFSCSVMHEALHNHYTQKSLSLSP |
| | | GKGGGGSGGGGSGGGGSEVQLVESGGGLVQPGGSLRLSCAASGFTFSD |
| | | SWIHWVRQAPGKGLEWVAWISPYGGSTYYADSVKGRFTISADTSKNTA |
| | | YLQMNSLRAEDTAVYYCARRHWPGGFDYWGQGTLVTVSSGGGGSGGGG |
| | | SGGGGSGGGGSDIQMTQSPSSLSASVGDRVTITCRASQDVSTAVAWYQ |
| | | QKPGKAPKLLIYSASFLYSGVPSRFSGSGSGTDFTLTISSLQPEDFAT |
| | | YYCQQYLYHPATFGQGTKVEIK |
| SEQ | TFGβR2-4GS- | TIPPHVQKSVNNDMIVTDNNGAVKFPQLCKFCDVRFSTCDNQKSCMSN |
| ID NO: | hCH1-hFc_Knob- | CSITSICEKPQEVCVAVWRKNDENITLETVCHDPKLPYHDFILEDAAS |
| 169 | 3x4GS-α-PDL1 | PKCIMKEKKKPGETFFMCSCSSDECNDNIIFSEEYNTSNPDGGGGSAS |
| | avelumab VH- | TKGPSVFPLAPSSKSTSGGTAALGCLVKDYFPEPVTVSWNSGALTSGV |
| | 4x4GS-α-PDL1 | HTFPAVLQSSGLYSLSSVVTVPSSSLGTQTYICNVNHKPSNTKVDKRV |
| | avelumab VL | EPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPCREEMT |
| | | KNQVSLWCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGG |
| | | GSGGGGSEVQLLESGGGLVQPGGSLRLSCAASGFTFSSYIMMWVRQAP |
| | | GKGLEWVSSIYPSGGITFYADTVKGRFTISRDNSKNTLYLQMNSLRAE |
| | | DTAVYYCARIKLGTVTTVDYWGQGTLVTVSSGGGGSGGGGSGGGGSGG |
| | | GGSQSALTQPASVSGSPGQSITISCTGTSSDVGGYNYVSWYQQHPGKA |
| | | PKLMIYDVSNRPSGVSNRFSGSKSGNTASLTISGLQAEDEADYYCSSY |
| | | TSSSTRVFGTGTKVTVL |
| SEQ | TFGβR2-4GS- | TIPPHVQKSVNNDMIVTDNNGAVKFPQLCKFCDVRFSTCDNQKSCMSN |
| ID NO: | hCH1-hFc_Knob- | CSITSICEKPQEVCVAVWRKNDENITLETVCHDPKLPYHDFILEDAAS |
| 170 | 3x4GS-α-PDL1 | PKCIMKEKKKPGETFFMCSCSSDECNDNIIFSEEYNTSNPDGGGGSAS |
| | durvalumab VH- | TKGPSVFPLAPSSKSTSGGTAALGCLVKDYFPEPVTVSWNSGALTSGV |
| | 4x4GS-α-PDL1 | HTFPAVLQSSGLYSLSSVVTVPSSSLGTQTYICNVNHKPSNTKVDKRV |
| | durvalumab VL | EPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPCREEMT |
| | | KNQVSLWCLVKGFYPSDIAVEWESNGQPENNYKTTPPVLDSDGSFFLY |
| | | SKLTVDKSRWQQGNVFSCSVMHEALHNHYTQKSLSLSPGKGGGGSGGG |
| | | GSGGGGSEVQLVESGGGLVQPGGSLRLSCAASGFTFSRYWMSWVRQAP |
| | | GKGLEWVANIKQDGSEKYYVDSVKGRFTISRDNAKNSLYLQMNSLRAE |
| | | DTAVYYCAREGGWFGELAFDYWGQGTLVTVSSGGGGSGGGGSGGGGSG |
| | | GGGSEIVLTQSPGTLSLSPGERATLSCRASQRVSSSYLAWYQQKPGQA |
| | | PRLLIYDASSRATGIPDRFSGSGSGTDFTLTISRLEPEDFAVYYCQQY |
| | | GSLPWTFGQGTKVEIK |
| SEQ | TFGβR2-4GS- | TIPPHVQKSVNNDMIVTDNNGAVKFPQLCKFCDVRFSTCDNQKSCMSN |
| ID NO: | hCH1-hFc_Knob- | CSITSICEKPQEVCVAVWRKNDENITLETVCHDPKLPYHDFILEDAAS |
| 171 | 3x4GS-α-PDL1 | PKCIMKEKKKPGETFFMCSCSSDECNDNIIFSEEYNTSNPDGGGGSAS |
| | atezolizumab VH- | TKGPSVFPLAPSSKSTSGGTAALGCLVKDYFPEPVTVSWNSGALTSGV |
| | 4x4GS-α-PDL1 | HTFPAVLQSSGLYSLSSVVTVPSSSLGTQTYICNVNHKPSNTKVDKRV |
| | atezolizumab VL | EPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPCREEMT |
| | | KNQVSLWCLVKGFYPSDIAVEWESNGQPENNYKTTPPVLDSDGSFFLY |
| | | SKLTVDKSRWQQGNVFScSVMHEALHNHYTQKSLSLSPGKGGGGSGGG |
| | | GSGGGGSEVQLVESGGGLVQPGGSLRLSCAASGFTFSDSWIHWVRQAP |
| | | GKGLEWVAWISPYGGSTYYADSVKGRFTISADTSKNTAYLQMNSLRAE |
| | | DTAVYYCARRHWPGGFDYWGQGTLVTVSSGGGGSGGGGSGGGGSGGGG |
| | | SDIQMTQSPSSLSASVGDRVTITCRASQDVSTAVAWYQQKPGKAPKLL |
| | | IYSASFLYSGVPSRFSGSGSGTDFTLTISSLQPEDFATYYCQQYLYHP |
| | | ATFGQGTKVEIK |
| SEQ | TFGβR1-4GS- | LLPGATALQCFCHLCTKDNFTCVTDGLCFVSVTETTDKVIHNSMCIAE |
| ID NO: | hCH1-hFc_Hole- | IDLIPRDRPFVCAPSSKTGSVTTTYCCNQDHCNKIELPTTVKSSPGLG |
| 172 | 3x4GS-α-CSF1R | PVEGGGGSASTKGPSVFPLAPSSKSTSGGTAALGCLVKDYFPEPVTVS |
| | emactuzumab | WNSGALTSGVHTFPAVLQSSGLYSLSSVVTVPSSSLGTQTYICNVNHK |
| | VH-4x4GS- | PSNTKVDKRVEPKSCDKTHTCPPCPAPELLGGPSVFLFPPKPKDTLMI |
| | aα-CSF1R | SRTPEVTCVVVDVSHEDPEVKFNWYVDGVEVHNAKTKPREEQYNSTYR |
| | emactuzumab VL | VVSVLTVLHQDWLNGKEYKCKVSNKALPAPIEKTISKAKGQPREPQVC |
| | | TLPPSREEMTKNQVSLSCAVKGFYPSDIAVEWESNGQPENNYKTTPPV |
| | | LDSDGSFFLVSKLTVDKSRWQQGNVFSCSVMHEALHNHYTQKSLSLSP |
| | | GKGGGGSGGGGSGGGGSQVQLVQSGAEVKKPGASVKVSCKASGYTFTS |
| | | YDISWVRQAPGQGLEWMGVIWTDGGTNYAQKLQGRVTMTTDTSTSTAY |
| | | MELRSLRSDDTAVYYCARDQRLYFDVWGQGTTVTVSSGGGGSGGGGSG |
| | | GGGSGGGGSDIQMTQSPSSLSASVGDRVTITCRASEDVNTYVSWYQQK |
| | | PGKAPKLLIYAASNRYTGVPSRFSGSGSGTDFTLTISSLQPEDFATYY |
| | | CQQSFSYPTFGQGTKLEIK |
| SEQ | TFGβR1-4GS- | LLPGATALQCFCHLCTKDNFTCVTDGLCFVSVTETTDKVIHNSMCIAE |
| ID NO: | hCH1-hFc_Hole- | IDLIPRDRPFVCAPSSKTGSVTTTYCCNQDHCNKIELPTTVKSSPGLG |
| 173 | 3x4GS-α-CSF1R | PVEGGGGSASTKGPSVFPLAPSSKSTSGGTAALGCLVKDYFPEPVTVS |
| | cabiralizumab | WNSGALTSGVHTFPAVLQSSGLYSLSSVVTVPSSSLGTQTYICNVNHK |
| | VH-4x4GS- | PSNTKVDKRVEPKSCDKTHTCPPCPAPELLGGPSVFLFPPKPKDTLMI |
| | α-CSF1R | SRTPEVTCVVVDVSHEDPEVKFNWYVDGVEVHNAKTKPREEQYNSTYR |
| | cabiralizumab VL | VVSVLTVLHQDWLNGKEYKCKVSNKALPAPIEKTISKAKGQPREPQVC |
| | | TLPPSREEMTKNQVSLSCAVKGFYPSDIAVEWESNGQPENNYKTTPPV |
| | | LDSDGSFFLVSKLTVDKSRWQQGNVFSCSVMHEALHNHYTQKSLSLSP |
| | | GKGGGGSGGGGSGGGGSQVQLVQSGAEVKKPGSSVKVSCKASGYTFTD |
| | | NYMIWVRQAPGQGLEWMGDINPYNGGTTFNQKFKGRVTITADKSTSTA |
| | | YMELSSLRSEDTAVYYCARESPYFSNLYVMDYWGQGTLVTVSSGGGGS |
| | | GGGGSGGGGSGGGGSEIVLTQSPATLSLSPGERATLSCKASQSVDYDG |
| | | DNYMNWYQQKPGQAPRLLIYAASNLESGIPARFSGSGSGTDFTLTISS |
| | | LEPEDFAVYYCHLSNEDLSTFGGGTKVEIK |
| SEQ | TFGβR2-4GS- | TIPPHVQKSVNNDMIVTDNNGAVKFPQLCKFCDVRFSTCDNQKSCMSN |
| ID NO: | hCH1-hFc_Hole- | CSITSICEKPQEVCVAVWRKNDENITLETVCHDPKLPYHDFILEDAAS |
| 174 | 3x4GS-αCSF1R | PKCIMKEKKKPGETFFMCSCSSDECNDNIIFSEEYNTSNPDGGGGSAS |
| | emactuzumab | TKGPSVFPLAPSSKSTSGGTAALGCLVKDYFPEPVTVSWNSGALTSGV |
| | VH-4x4GS- | HTFPAVLQSSGLYSLSSVVTVPSSSLGTQTYICNVNHKPSNTKVDKRV |
| | α-CSF1R | EPKSCDKTHTCPPCPAPELLGGPSVFLFPPKPKDTLMISRTPEVTCVV |
| | emactuzumab VL | VDVSHEDPEVKFNWYVDGVEVHNAKTKPREEQYNSTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFSCSVMHEALHNHYTQKSLSLSPGKGGGGSGGG |
| | | GSGGGGSQVQLVQSGAEVKKPGASVKVSCKASGYTFTSYDISWVRQAP |
| | | GQGLEWMGVIWTDGGTNYAQKLQGRVTMTTDTSTSTAYMELRSLRSDD |
| | | TAVYYCARDQRLYFDVWGQGTTVTVSSGGGGSGGGGSGGGGSGGGGSD |
| | | IQMTQSPSSLSASVGDRVTITCRASEDVNTYVSWYQQKPGKAPKLLIY |
| | | AASNRYTGVPSRFSGSGSGTDFTLTISSLQPEDFATYYCQQSFSYPTF |
| | | GQGTKLEIK |
| SEQ | TFGβR2-4GS- | TIPPHVQKSVNNDMIVTDNNGAVKFPQLCKFCDVRFSTCDNQKSCMSN |
| ID NO: | hCH1-hFc_Hole- | CSITSICEKPQEVCVAVWRKNDENITLETVCHDPKLPYHDFILEDAAS |
| 175 | 3x4GS-α-CSF1R | PKCIMKEKKKPGETFFMCSCSSDECNDNIIFSEEYNTSNPDGGGGSAS |
| | cabiralizumab | TKGPSVFPLAPSSKSTSGGTAALGCLVKDYFPEPVTVSWNSGALTSGV |
| | VH-4x4GS- | HTFPAVLQSSGLYSLSSVVTVPSSSLGTQTYICNVNHKPSNTKVDKRV |
| | α-CSF1R | EPKSCDKTHTCPPCPAPELLGGPSVFLFPPKPKDTLMISRTPEVTCVV |
| | cabiralizumab VL | VDVSHEDPEVKFNWYVDGVEVHNAKTKPREEQYNSTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFScSVMHEALHNHYTQKSLSLSPGKGGGGSGGG |
| | | GSGGGGSQVQLVQSGAEVKKPGSSVKVSCKASGYTFTDNYMIWVRQAP |
| | | GQGLEWMGDINPYNGGTTFNQKFKGRVTITADKSTSTAYMELSSLRSE |
| | | DTAVYYCARESPYFSNLYVMDYWGQGTLVTVSSEIVLTQSPATLSLSP |
| | | GERATLSCKASQSVDYDGDNYMNWYQQKPGQAPRLLIYAASNLESGIP |
| | | ARFSGSGSGTDFTLTISSLEPEDFAVYYCHLSNEDLSTFGGGTKVEIK |
| SEQ | α-PDL1 avelumab | EVQLLESGGGLVQPGGSLRLSCAASGFTFSSYIMMWVRQAPGKGLEWV |
| ID NO: | VH-CH1- | SSIYPSGGITFYADTVKGRFTISRDNSKNTLYLQMNSLRAEDTAVYYC |
| 176 | hFc_Knob-3x4GS- | ARIKLGTVTTVDYWGQGTLVTVSSASTKGPSVFPLAPSSKSTSGGTAA |
| | TGFβR1 | LGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPCREEMTKNQVSLWCLVKGFYPSDIAVEW |
| | | ESNGQPENNYKTTPPVLDSDGSFFLYSKLTVDKSRWQQGNVFSCSVMH |
| | | EALHNHYTQKSLSLSPGKGGGGSGGGGSGGGGSLLPGATALQCFCHLC |
| | | TKDNFTCVTDGLCFVSVTETTDKVIHNSMCIAEIDLIPRDRPFVCAPS |
| | | SKTGSVTTTYCCNQDHCNKIELPTTVKSSPGLGPVE |
| SEQ | α-PDL1 | EVQLVESGGGLVQPGGSLRLSCAASGFTFSRYWMSWVRQAPGKGLEWV |
| ID NO: | durvalumab VH- | ANIKQDGSEKYYVDSVKGRFTISRDNAKNSLYLQMNSLRAEDTAVYYC |
| 177 | CH1-hFc_Knob- | AREGGWFGELAFDYWGQGTLVTVSSASTKGPSVFPLAPSSKSTSGGTA |
| | 3x4GS-TGFβR1 | ALGCLVKDYFPEPVTVSWNSGALTSGVHTFPAVLQSSGLYSLSSVVTV |
| | | PSSSLGTQTYICNVNHKPSNTKVDKRVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPCREEMTKNQVSLWCLVKGFYPSDIAVE |
| | | WESNGQPENNYKTTPPVLDSDGSFFLYSKLTVDKSRWQQGNVFSCSVM |
| | | HEALHNHYTQKSLSLSPGKGGGGSGGGGSGGGGSLLPGATALQCFCHL |
| | | CTKDNFTCVTDGLCFVSVTETTDKVIHNSMCIAEIDLIPRDRPFVCAP |
| | | SSKTGSVTTTYCCNQDHCNKIELPTTVKSSPGLGPVE |
| SEQ | A-PDL1 | EVQLVESGGGLVQPGGSLRLSCAASGFTFSDSWIHWVRQAPGKGLEWV |
| ID NO: | atezolizumab VH- | AWISPYGGSTYYADSVKGRFTISADTSKNTAYLQMNSLRAEDTAVYYC |
| 178 | CH1-hFc_Knob- | ARRHWPGGFDYWGQGTLVTVSSASTKGPSVFPLAPSSKSTSGGTAALG |
| | 3x4GS-TGFβR1 | CLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPCREEMTKNQVSLWCLVKGFYPSDIAVEWES |
| | | NGQPENNYKTTPPVLDSDGSFFLYSKLTVDKSRWQQGNVFSCSVMHEA |
| | | LHNHYTQKSLSLSPGKGGGGSGGGGSGGGGSLLPGATALQCFCHLCTK |
| | | DNFTCVTDGLCFVSVTETTDKVIHNSMCIAEIDLIPRDRPFVCAPSSK |
| | | TGSVTTTYCCNQDHCNKIELPTTVKSSPGLGPVE |
| SEQ | α-PDL1 avelumab | EVQLLESGGGLVQPGGSLRLSCAASGFTFSSYIMMWVRQAPGKGLEWV |
| ID NO: | VH-CH1- | SSIYPSGGITFYADTVKGRFTISRDNSKNTLYLQMNSLRAEDTAVYYC |
| 179 | hFc_Knob-3x4GS- | ARIKLGTVTTVDYWGQGTLVTVSSASTKGPSVFPLAPSSKSTSGGTAA |
| | TGFβR2 | LGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPCREEMTKNQVSLWCLVKGFYPSDIAVEW |
| | | ESNGQPENNYKTTPPVLDSDGSFFLYSKLTVDKSRWQQGNVFSCSVMH |
| | | EALHNHYTQKSLSLSPGKGGGGSGGGGSGGGGSTIPPHVQKSVNNDMI |
| | | VTDNNGAVKFPQLCKFCDVRFSTCDNQKSCMSNCSITSICEKPQEVCV |
| | | AVWRKNDENITLETVCHDPKLPYHDFILEDAASPKCIMKEKKKPGETF |
| | | FMCSCSSDECNDNIIFSEEYNTSNPD |
| SEQ | α-PDL1 | EVQLVESGGGLVQPGGSLRLSCAASGFTFSRYWMSWVRQAPGKGLEWV |
| ID NO: | durvalumab VH- | ANIKQDGSEKYYVDSVKGRFTISRDNAKNSLYLQMNSLRAEDTAVYYC |
| 180 | CH1-hFc_Knob- | AREGGWFGELAFDYWGQGTLVTVSSASTKGPSVFPLAPSSKSTSGGTA |
| | 3x4GS-TGFβR2 | ALGCLVKDYFPEPVTVSWNSGALTSGVHTFPAVLQSSGLYSLSSVVTV |
| | | PSSSLGTQTYICNVNHKPSNTKVDKRVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPCREEMTKNQVSLWCLVKGFYPSDIAVE |
| | | WESNGQPENNYKTTPPVLDSDGSFFLYSKLTVDKSRWQQGNVFSCSVM |
| | | HEALHNHYTQKSLSLSPGKGGGGSGGGGSGGGGSTIPPHVQKSVNNDM |
| | | IVTDNNGAVKFPQLCKFCDVRFSTCDNQKSCMSNCSITSICEKPQEVC |
| | | VAVWRKNDENITLETVCHDPKLPYHDFILEDAASPKCIMKEKKKPGET |
| | | FFMCSCSSDECNDNIIFSEEYNTSNPD |
| SEQ | α-PDL1 | EVQLVESGGGLVQPGGSLRLSCAASGFTFSDSWIHWVRQAPGKGLEWV |
| ID NO: | atezolizumab VH- | AWISPYGGSTYYADSVKGRFTISADTSKNTAYLQMNSLRAEDTAVYYC |
| 181 | CH1-hFc_Knob- | ARRHWPGGFDYWGQGTLVTVSSASTKGPSVFPLAPSSKSTSGGTAALG |
| | 3x4GS-TGFβR2 | CLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPCREEMTKNQVSLWCLVKGFYPSDIAVEWES |
| | | NGQPENNYKTTPPVLDSDGSFFLYSKLTVDKSRWQQGNVFSCSVMHEA |
| | | LHNHYTQKSLSLSPGKGGGGSGGGGSGGGGSTIPPHVQKSVNNDMIVT |
| | | DNNGAVKFPQLCKFCDVRFSTCDNQKSCMSNCSITSICEKPQEVCVAV |
| | | WRKNDENITLETVCHDPKLPYHDFILEDAASPKCIMKEKKKPGETFFM |
| | | CSCSSDECNDNIIFSEEYNTSNPD |
| SEQ | α-PDL1 avelumab | EVQLLESGGGLVQPGGSLRLSCAASGFTFSSYIMMWVRQAPGKGLEWV |
| ID NO: | VH-CH1- | SSIYPSGGITFYADTVKGRFTISRDNSKNTLYLQMNSLRAEDTAVYYC |
| 182 | hFc_Knob-3x4GS- | ARIKLGTVTTVDYWGQGTLVTVSSASTKGPSVFPLAPSSKSTSGGTAA |
| | TGFβR3 | LGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGG |
| | | PSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIE |
| | | KTISKAKGQPREPQVYTLPPCREEMTKNQVSLWCLVKGFYPSDIAVEW |
| | | ESNGQPENNYKTTPPVLDSDGSFFLYSKLTVDKSRWQQGNVFSCSVMH |
| | | EALHNHYTQKSLSLSPGKGGGGSGGGGSGGGGSGPEPGALCELSPVSA |
| | | SHPVQALMESFTVLSGCASRGTTGLPQEVHVLNLRTAGQGPGQLQREV |
| | | TLHLNPISSVHIHHKSVVFLLNSPHPLVWHLKTERLATGVSRLFLVSE |
| | | GSVVQFSSANFSLTAETEERNFPHGNEHLLNWARKEYGAVTSFTELKI |
| | | ARNIYIKVGEDQVFPPKCNIGKNFLSLNYLAEYLQPKAAEGCVMSSQP |
| | | QNEEVHIIELITPNSNPYSAFQVDITIDIRPSQEDLEVVKNLILILKC |
| | | KKSVNWVIKSFDVKGSLKIIAPNSIGFGKESERSMTMTKSIRDDIPST |
| | | QGNLVKWALDNGYSPITSYTMAPVANRFHLRLENNAEEMGDEEVHTIP |
| | | PELRILLDPGALPALQNPPIRGGEGQNGGLPFPFPDISRRVWNEEGED |
| | | GLPRPKDPVIPSIQLFPGLREPEEVQGSVDIALSVKCDNEKMIVAVEK |
| | | DSFQASGYSGMDVTLLDPTCKAKMNGTHFVLESPLNGCGTRPRWSALD |
| | | GVVYYNSIVIQVPALGDSSGWPDGYEDLESGDNGFPGDMDEGDASLFT |
| | | RPEIVVFNCSLQQVRNPSSFQEQPHGNITFNMELYNTDLFLVPSQGVF |
| | | SVPENGHVYVEVSVTKAEQELGFAIQTCFISPYSNPDRMSHYTIIENI |
| | | CPKDESVKFYSPKRVHFPIPQADMDKKRFSFVFKPVFNTSLLFLQCEL |
| | | TLCTKMEKHPQKLPKCVPPDEACTSLDASIIWAMMQNKKTFTKPLAVI |
| | | HHEAESKEKGPSMKEPNPISPPIFHGLDTLTV |
| SEQ | α-PDL1 | EVQLVESGGGLVQPGGSLRLSCAASGFTFSRYWMSWVRQAPGKGLEWV |
| ID NO: | durvalumab VH- | ANIKQDGSEKYYVDSVKGRFTISRDNAKNSLYLQMNSLRAEDTAVYYC |
| 183 | CH1-hFc_Knob- | AREGGWFGELAFDYWGQGTLVTVSSASTKGPSVFPLAPSSKSTSGGTA |
| | 3x4GS-TGFβR3 | ALGCLVKDYFPEPVTVSWNSGALTSGVHTFPAVLQSSGLYSLSSVVTV |
| | | PSSSLGTQTYICNVNHKPSNTKVDKRVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPCREEMTKNQVSLWCLVKGFYPSDIAVE |
| | | WESNGQPENNYKTTPPVLDSDGSFFLYSKLTVDKSRWQQGNVFSCSVM |
| | | HEALHNHYTQKSLSLSPGKGGGGSGGGGSGGGGSGPEPGALCELSPVS |
| | | ASHPVQALMESFTVLSGCASRGTTGLPQEVHVLNLRTAGQGPGQLQRE |
| | | VTLHLNPISSVHIHHKSVVFLLNSPHPLVWHLKTERLATGVSRLFLVS |
| | | EGSVVQFSSANFSLTAETEERNFPHGNEHLLNWARKEYGAVTSFTELK |
| | | IARNIYIKVGEDQVFPPKCNIGKNFLSLNYLAEYLQPKAAEGCVMSSQ |
| | | PQNEEVHIIELITPNSNPYSAFQVDITIDIRPSQEDLEVVKNLILILK |
| | | CKKSVNWVIKSFDVKGSLKIIAPNSIGFGKESERSMTMTKSIRDDIPS |
| | | TQGNLVKWALDNGYSPITSYTMAPVANRFHLRLENNAEEMGDEEVHTI |
| | | PPELRILLDPGALPALQNPPIRGGEGQNGGLPFPFPDISRRVWNEEGE |
| | | DGLPRPKDPVIPSIQLFPGLREPEEVQGSVDIALSVKCDNEKMIVAVE |
| | | KDSFQASGYSGMDVTLLDPTCKAKMNGTHFVLESPLNGCGTRPRWSAL |
| | | DGVVYYNSIVIQVPALGDSSGWPDGYEDLESGDNGFPGDMDEGDASLF |
| | | TRPEIVVFNCSLQQVRNPSSFQEQPHGNITFNMELYNTDLFLVPSQGV |
| | | FSVPENGHVYVEVSVTKAEQELGFAIQTCFISPYSNPDRMSHYTIIEN |
| | | ICPKDESVKFYSPKRVHFPIPQADMDKKRFSFVFKPVFNTSLLFLQCE |
| | | LTLCTKMEKHPQKLPKCVPPDEACTSLDASIIWAMMQNKKTFTKPLAV |
| | | IHHEAESKEKGPSMKEPNPISPPIFHGLDTLTV |
| SEQ | α-PDL1 | EVQLVESGGGLVQPGGSLRLSCAASGFTFSDSWIHWVRQAPGKGLEWV |
| ID NO: | atezolizumab VH- | AWISPYGGSTYYADSVKGRFTISADTSKNTAYLQMNSLRAEDTAVYYC |
| 184 | CH1-hFc_Knob- | ARRHWPGGFDYWGQGTLVTVSSASTKGPSVFPLAPSSKSTSGGTAALG |
| | 3x4GS-TGFβR3 | CLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPCREEMTKNQVSLWCLVKGFYPSDIAVEWES |
| | | NGQPENNYKTTPPVLDSDGSFFLYSKLTVDKSRWQQGNVFSCSVMHEA |
| | | LHNHYTQKSLSLSPGKGGGGSGGGGSGGGGSGPEPGALCELSPVSASH |
| | | PVQALMESFTVLSGCASRGTTGLPQEVHVLNLRTAGQGPGQLQREVTL |
| | | HLNPISSVHIHHKSVVFLLNSPHPLVWHLKTERLATGVSRLFLVSEGS |
| | | VVQFSSANFSLTAETEERNFPHGNEHLLNWARKEYGAVTSFTELKIAR |
| | | NIYIKVGEDQVFPPKCNIGKNFLSLNYLAEYLQPKAAEGCVMSSQPQN |
| | | EEVHIIELITPNSNPYSAFQVDITIDIRPSQEDLEVVKNLILILKCKK |
| | | SVNWVIKSFDVKGSLKIIAPNSIGFGKESERSMTMTKSIRDDIPSTQG |
| | | NLVKWALDNGYSPITSYTMAPVANRFHLRLENNAEEMGDEEVHTIPPE |
| | | LRILLDPGALPALQNPPIRGGEGQNGGLPFPFPDISRRVWNEEGEDGL |
| | | PRPKDPVIPSIQLFPGLREPEEVQGSVDIALSVKCDNEKMIVAVEKDS |
| | | FQASGYSGMDVTLLDPTCKAKMNGTHFVLESPLNGCGTRPRWSALDGV |
| | | VYYNSIVIQVPALGDSSGWPDGYEDLESGDNGFPGDMDEGDASLFTRP |
| | | EIVVFNCSLQQVRNPSSFQEQPHGNITFNMELYNTDLFLVPSQGVFSV |
| | | PENGHVYVEVSVTKAEQELGFAIQTCFISPYSNPDRMSHYTIIENICP |
| | | KDESVKFYSPKRVHFPIPQADMDKKRFSFVFKPVFNTSLLFLQCELTL |
| | | CTKMEKHPQKLPKCVPPDEACTSLDASIIWAMMQNKKTFTKPLAVIHH |
| | | EAESKEKGPSMKEPNPISPPIFHGLDTLTV |
| SEQ | α-CSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 185 | VH-CH1- | RDQRLYFDVWGQGTTVTVSSASTKGPSVFPLAPSSKSTSGGTAALGCL |
| | hFc_Hole-3x4GS- | VKDYFPEPVTVSWNSGALTSGVHTFPAVLQSSGLYSLSSVVTVPSSSL |
| | TGFβR1 | GTQTYICNVNHKPSNTKVDKRVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVCTLPPSREEMTKNQVSLSCAVKGFYPSDIAVEWESNG |
| | | QPENNYKTTPPVLDSDGSFFLVSKLTVDKSRWQQGNVFSCSVMHEALH |
| | | NHYTQKSLSLSPGKGGGGSGGGGSGGGGSLLPGATALQCFCHLCTKDN |
| | | FTCVTDGLCFVSVTETTDKVIHNSMCIAEIDLIPRDRPFVCAPSSKTG |
| | | SVTTTYCCNQDHCNKIELPTTVKSSPGLGPVE |
| SEQ | α-CSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 186 | VH-H1- | ARESPYFSNLYVMDYWGQGTLVTVSSASTKGPSVFPLAPSSKSTSGGT |
| | hFc_Hole-3x4GS- | AALGCLVKDYFPEPVTVSWNSGALTSGVHTFPAVLQSSGLYSLSSVVT |
| | TGFβR1 | VPSSSLGTQTYICNVNHKPSNTKVDKRVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSV |
| | | MHEALHNHYTQKSLSLSPGKGGGGSGGGGSGGGGSLLPGATALQCFCH |
| | | LCTKDNFTCVTDGLCFVSVTETTDKVIHNSMCIAEIDLIPRDRPFVCA |
| | | PSSKTGSVTTTYCCNQDHCNKIELPTTVKSSPGLGPVE |
| SEQ | α-CSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 187 | VH-CH1- | RDQRLYFDVWGQGTTVTVSSASTKGPSVFPLAPSSKSTSGGTAALGCL |
| | hFc_Hole TGFβR2 | VKDYFPEPVTVSWNSGALTSGVHTFPAVLQSSGLYSLSSVVTVPSSSL |
| | | GTQTYICNVNHKPSNTKVDKRVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVCTLPPSREEMTKNQVSLSCAVKGFYPSDIAVEWESNG |
| | | QPENNYKTTPPVLDSDGSFFLVSKLTVDKSRWQQGNVFSCSVMHEALH |
| | | NHYTQKSLSLSPGKGGGGSGGGGSGGGGSTIPPHVQKSVNNDMIVTDN |
| | | NGAVKFPQLCKFCDVRFSTCDNQKSCMSNCSITSICEKPQEVCVAVWR |
| | | KNDENITLETVCHDPKLPYHDFILEDAASPKCIMKEKKKPGETFFMCS |
| | | CSSDECNDNIIFSEEYNTSNPD |
| SEQ | α-CSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 188 | VH-CH1- | ARESPYFSNLYVMDYWGQGTLVTVSSASTKGPSVFPLAPSSKSTSGGT |
| | hFc_Hole-3x4GS- | AALGCLVKDYFPEPVTVSWNSGALTSGVHTFPAVLQSSGLYSLSSVVT |
| | TGFβR2 | VPSSSLGTQTYICNVNHKPSNTKVDKRVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSV |
| | | MHEALHNHYTQKSLSLSPGKGGGGSGGGGSGGGGSTIPPHVQKSVNND |
| | | MIVTDNNGAVKFPQLCKFCDVRFSTCDNQKSCMSNCSITSICEKPQEV |
| | | CVAVWRKNDENITLETVCHDPKLPYHDFILEDAASPKCIMKEKKKPGE |
| | | TFFMCSCSSDECNDNIIFSEEYNTSNPD |
| SEQ | α-CSF1R | QVQLVQSGAEVKKPGASVKVSCKASGYTFTSYDISWVRQAPGQGLEWM |
| ID NO: | emactuzumab | GVIWTDGGTNYAQKLQGRVTMTTDTSTSTAYMELRSLRSDDTAVYYCA |
| 189 | VH-CH1- | RDQRLYFDVWGQGTTVTVSSASTKGPSVFPLAPSSKSTSGGTAALGCL |
| | hFc_Hole-3x4GS- | VKDYFPEPVTVSWNSGALTSGVHTFPAVLQSSGLYSLSSVVTVPSSSL |
| | TGFβR3 | GTQTYICNVNHKPSNTKVDKRVEPKSCDKTHTCPPCPAPELLGGPSVF |
| | | LFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVCTLPPSREEMTKNQVSLSCAVKGFYPSDIAVEWESNG |
| | | QPENNYKTTPPVLDSDGSFFLVSKLTVDKSRWQQGNVFScSVMHEALH |
| | | NHYTQKSLSLSPGKGGGGSGGGGSGGGGSGPEPGALCELSPVSASHPV |
| | | QALMESFTVLSGCASRGTTGLPQEVHVLNLRTAGQGPGQLQREVTLHL |
| | | NPISSVHIHHKSVVFLLNSPHPLVWHLKTERLATGVSRLFLVSEGSVV |
| | | QFSSANFSLTAETEERNFPHGNEHLLNWARKEYGAVTSFTELKIARNI |
| | | YIKVGEDQVFPPKCNIGKNFLSLNYLAEYLQPKAAEGCVMSSQPQNEE |
| | | VHIIELITPNSNPYSAFQVDITIDIRPSQEDLEVVKNLILILKCKKSV |
| | | NWVIKSFDVKGSLKIIAPNSIGFGKESERSMTMTKSIRDDIPSTQGNL |
| | | VKWALDNGYSPITSYTMAPVANRFHLRLENNAEEMGDEEVHTIPPELR |
| | | ILLDPGALPALQNPPIRGGEGQNGGLPFPFPDISRRVWNEEGEDGLPR |
| | | PKDPVIPSIQLFPGLREPEEVQGSVDIALSVKCDNEKMIVAVEKDSFQ |
| | | ASGYSGMDVTLLDPTCKAKMNGTHFVLESPLNGCGTRPRWSALDGVVY |
| | | YNSIVIQVPALGDSSGWPDGYEDLESGDNGFPGDMDEGDASLFTRPEI |
| | | VVFNCSLQQVRNPSSFQEQPHGNITFNMELYNTDLFLVPSQGVFSVPE |
| | | NGHVYVEVSVTKAEQELGFAIQTCFISPYSNPDRMSHYTIIENICPKD |
| | | ESVKFYSPKRVHFPIPQADMDKKRFSFVFKPVFNTSLLFLQCELTLCT |
| | | KMEKHPQKLPKCVPPDEACTSLDASIIWAMMQNKKTFTKPLAVIHHEA |
| | | ESKEKGPSMKEPNPISPPIFHGLDTLTV |
| SEQ | α-CSF1R | QVQLVQSGAEVKKPGSSVKVSCKASGYTFTDNYMIWVRQAPGQGLEWM |
| ID NO: | cabiralizumab | GDINPYNGGTTFNQKFKGRVTITADKSTSTAYMELSSLRSEDTAVYYC |
| 190 | VH-CH1- | ARESPYFSNLYVMDYWGQGTLVTVSSASTKGPSVFPLAPSSKSTSGGT |
| | hFc_Hole-3x4GS- | AALGCLVKDYFPEPVTVSWNSGALTSGVHTFPAVLQSSGLYSLSSVVT |
| | TGFβR3 | VPSSSLGTQTYICNVNHKPSNTKVDKRVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAP |
| | | IEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSV |
| | | MHEALHNHYTQKSLSLSPGKGGGGSGGGGSGGGGSGPEPGALCELSPV |
| | | SASHPVQALMESFTVLSGCASRGTTGLPQEVHVLNLRTAGQGPGQLQR |
| | | EVTLHLNPISSVHIHHKSVVFLLNSPHPLVWHLKTERLATGVSRLFLV |
| | | SEGSVVQFSSANFSLTAETEERNFPHGNEHLLNWARKEYGAVTSFTEL |
| | | KIARNIYIKVGEDQVFPPKCNIGKNFLSLNYLAEYLQPKAAEGCVMSS |
| | | QPQNEEVHIIELITPNSNPYSAFQVDITIDIRPSQEDLEVVKNLILIL |
| | | KCKKSVNWVIKSFDVKGSLKIIAPNSIGFGKESERSMTMTKSIRDDIP |
| | | STQGNLVKWALDNGYSPITSYTMAPVANRFHLRLENNAEEMGDEEVHT |
| | | IPPELRILLDPGALPALQNPPIRGGEGQNGGLPFPFPDISRRVWNEEG |
| | | EDGLPRPKDPVIPSIQLFPGLREPEEVQGSVDIALSVKCDNEKMIVAV |
| | | EKDSFQASGYSGMDVTLLDPTCKAKMNGTHFVLESPLNGCGTRPRWSA |
| | | LDGVVYYNSIVIQVPALGDSSGWPDGYEDLESGDNGFPGDMDEGDASL |
| | | FTRPEIVVFNCSLQQVRNPSSFQEQPHGNITFNMELYNTDLFLVPSQG |
| | | VFSVPENGHVYVEVSVTKAEQELGFAIQTCFISPYSNPDRMSHYTIIE |
| | | NICPKDESVKFYSPKRVHFPIPQADMDKKRFSFVFKPVFNTSLLFLQC |
| | | ELTLCTKMEKHPQKLPKCVPPDEACTSLDASIIWAMMQNKKTFTKPLA |
| | | VIHHEAESKEKGPSMKEPNPISPPIFHGLDTLTV |

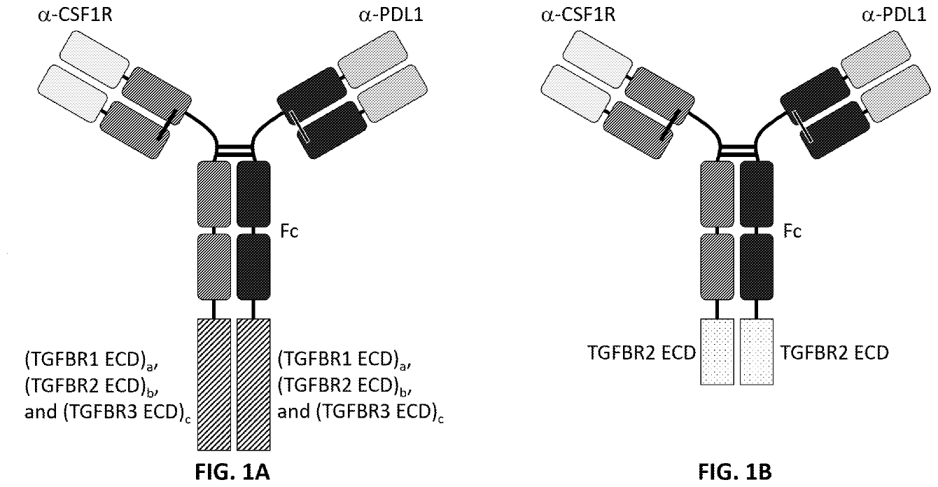

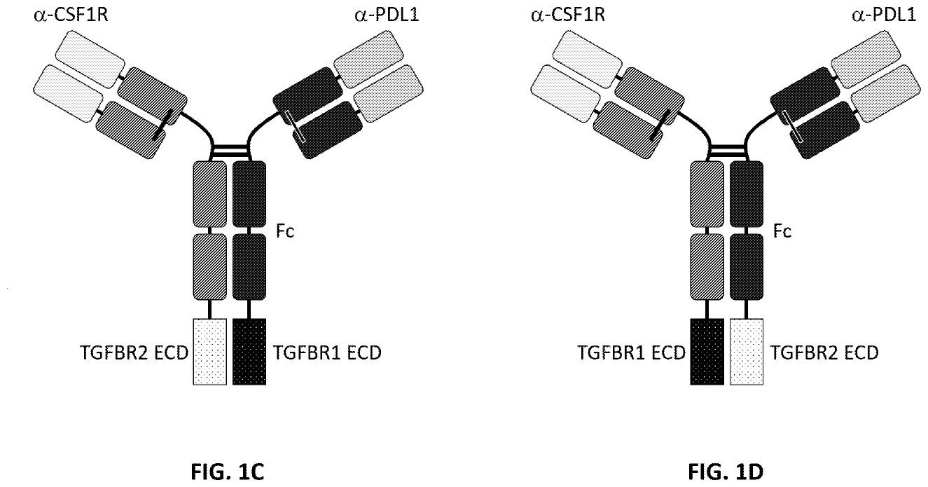

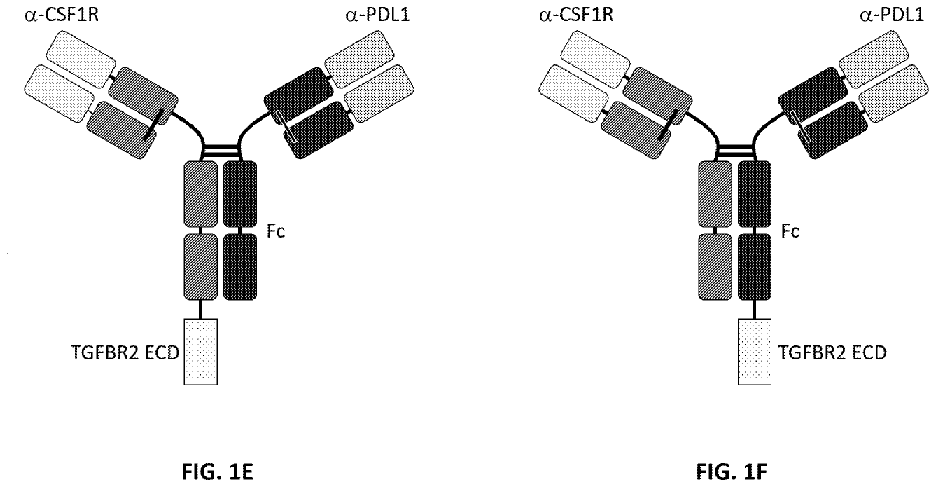

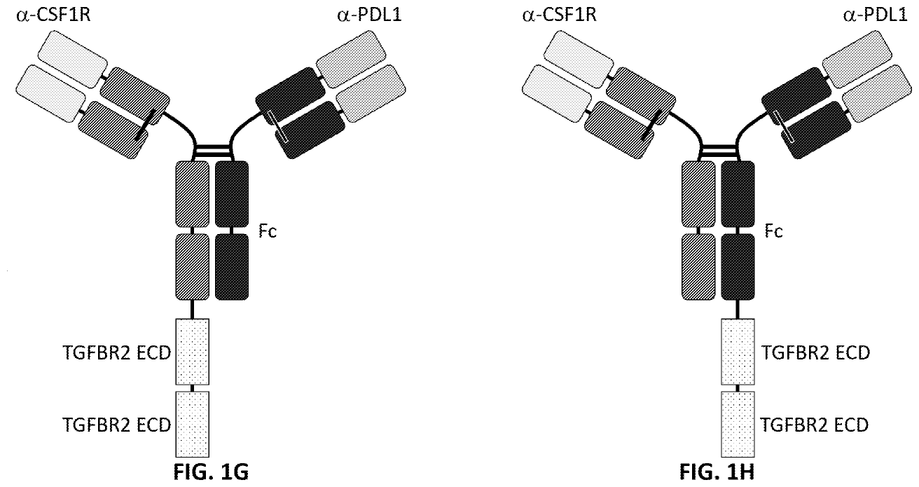

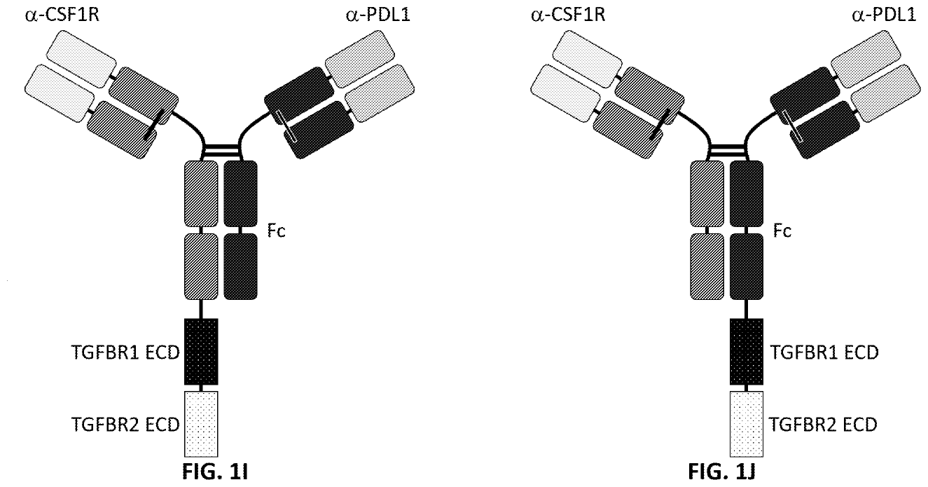

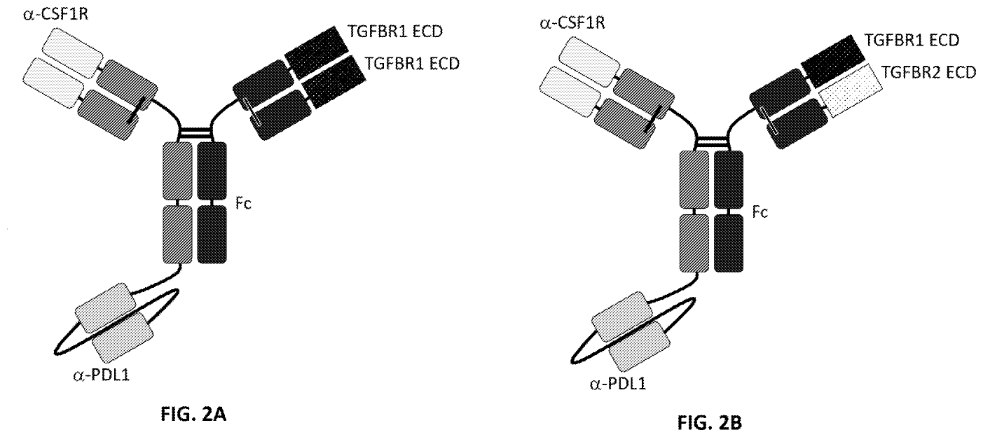

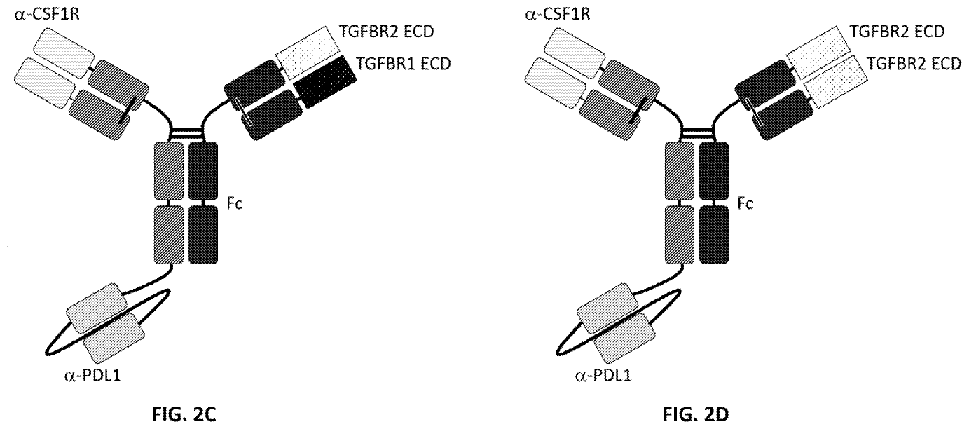

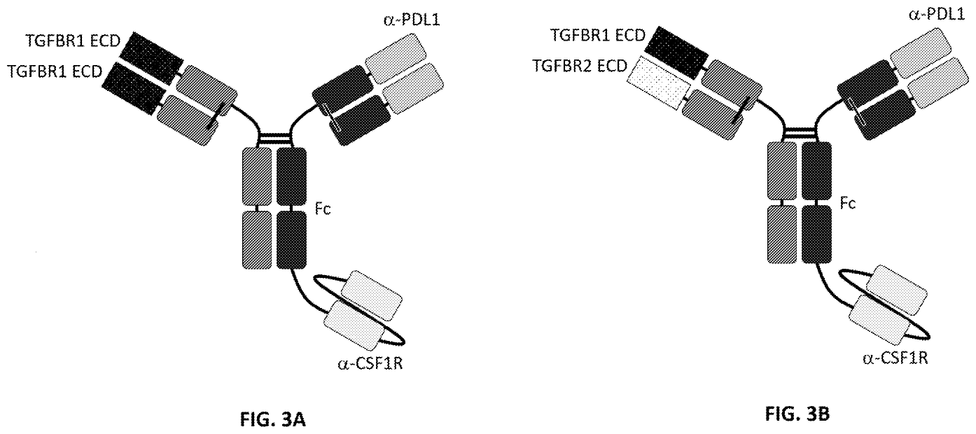

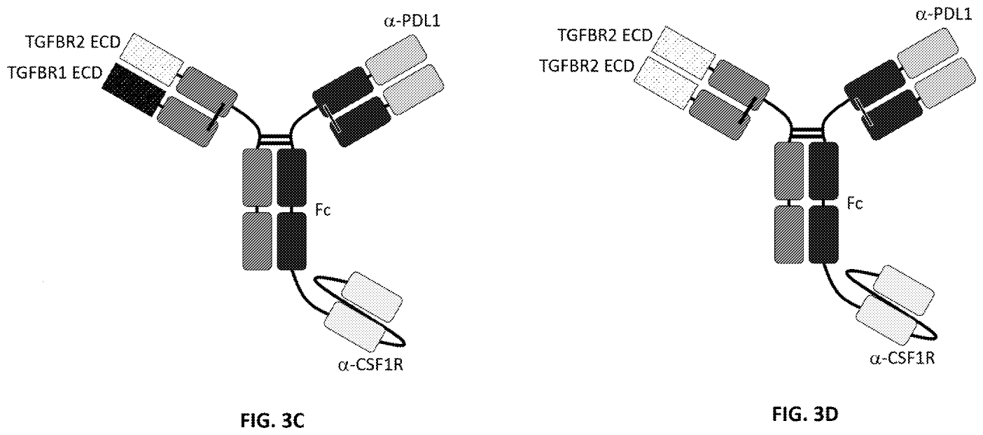

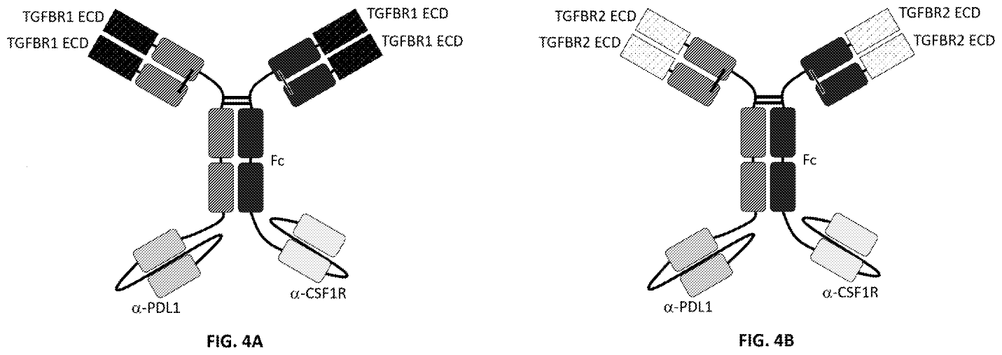

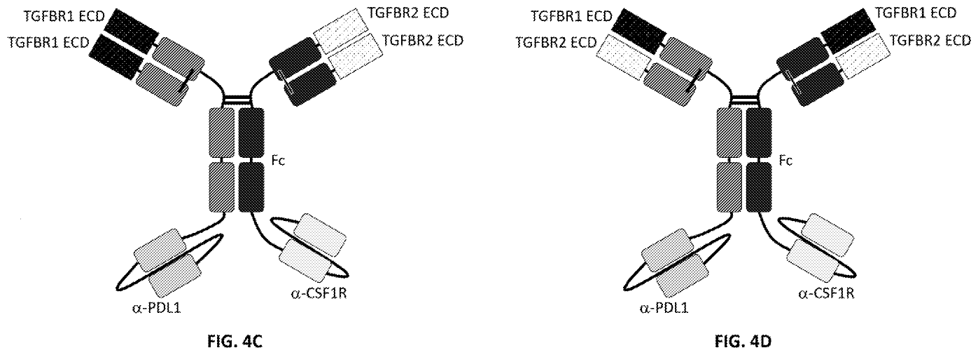

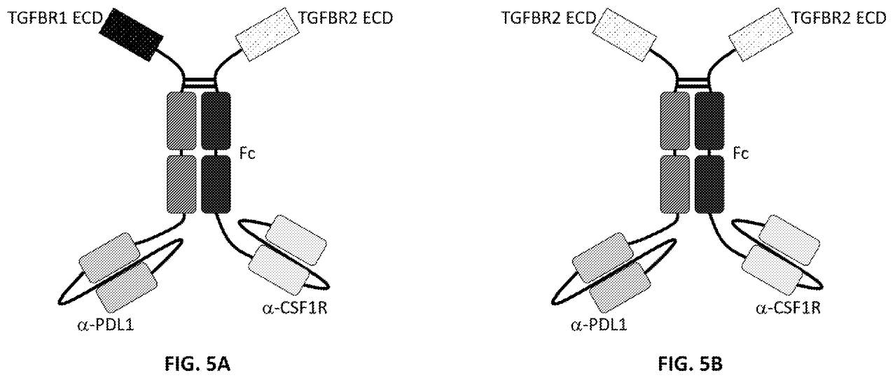

TABLE 14
Sequences used to generate multispecific molecules.

| Multispecific Molecule | | Represented by |
|---|---|---|
| Identification | Comprising SEQ IDs | FIG. Number |
| Multispecific molecule 1 | SEQ ID NOs: 176, 138, 185, 147 | FIG. 1A |
| Multispecific molecule 2 | SEQ ID NOs: 176, 138, 186, 148 | FIG. 1A |
| Multispecific molecule 3 | SEQ ID NOs: 176, 138, 187, 147 | FIG. 1A |
| Multispecific molecule 4 | SEQ ID NOs: 176, 138, 188, 148 | FIG. 1A |
| Multispecific molecule 5 | SEQ ID NOs: 176, 138, 189, 147 | FIG. 1A |
| Multispecific molecule 6 | SEQ ID NOs: 176, 138, 190, 148 | FIG. 1A |
| Multispecific molecule 7 | SEQ ID NOs: 177, 150, 185, 147 | FIG. 1A |
| Multispecific molecule 8 | SEQ ID NOs: 177, 150, 186, 148 | FIG. 1A |
| Multispecific molecule 9 | SEQ ID NOs: 177, 150, 187, 147 | FIG. 1A |
| Multispecific molecule 10 | SEQ ID NOs: 177, 150, 188, 148 | FIG. 1A |
| Multispecific molecule 11 | SEQ ID NOs: 177, 150, 189, 147 | FIG. 1A |
| Multispecific molecule 12 | SEQ ID NOs: 177, 150, 190, 148 | FIG. 1A |
| Multispecific molecule 13 | SEQ ID NOs: 178, 152, 185, 147 | FIG. 1A |
| Multispecific molecule 14 | SEQ ID NOs: 178, 152, 186, 148 | FIG. 1A |
| Multispecific molecule 15 | SEQ ID NOs: 178, 152, 187, 147 | FIG. 1A |
| Multispecific molecule 16 | SEQ ID NOs: 178, 152, 188, 148 | FIG. 1A |
| Multispecific molecule 17 | SEQ ID NOs: 178, 152, 189, 147 | FIG. 1A |
| Multispecific molecule 18 | SEQ ID NOs: 178, 152, 190, 147 | FIG. 1A |
| Multispecific molecule 19 | SEQ ID NOs: 179, 138, 185, 147 | FIG. 1A |
| Multispecific molecule 20 | SEQ ID NOs: 179, 138, 186, 148 | FIG. 1A |
| Multispecific molecule 21 | SEQ ID NOs: 179, 138, 187, 148 | FIG. 1A |
| Multispecific molecule 22 | SEQ ID NOs: 179, 138, 188, 148 | FIG. 1A |
| Multispecific molecule 23 | SEQ ID NOs: 179, 138, 189, 147 | FIG. 1A |
| Multispecific molecule 24 | SEQ ID NOs: 179, 138, 190, 148 | FIG. 1A |
| Multispecific molecule 25 | SEQ ID NOs: 180, 150, 185, 147 | FIG. 1A |
| Multispecific molecule 26 | SEQ ID NOs: 180, 150, 186, 148 | FIG. 1A |
| Multispecific molecule 27 | SEQ ID NOs: 180, 150, 187, 147 | FIG. 1A |
| Multispecific molecule 28 | SEQ ID NOs: 180, 150, 188, 148 | FIG. 1A |
| Multispecific molecule 29 | SEQ ID NOs: 180, 150, 189, 147 | FIG. 1A |
| Multispecific molecule 30 | SEQ ID NOs: 180, 150, 190, 148 | FIG. 1A |
| Multispecific molecule 31 | SEQ ID NOs: 181, 152, 185, 147 | FIG. 1A |
| Multispecific molecule 32 | SEQ ID NOs: 181, 152, 186, 148 | FIG. 1A |
| Multispecific molecule 33 | SEQ ID NOs: 181, 152, 187, 147 | FIG. 1A |
| Multispecific molecule 34 | SEQ ID NOs: 181, 152, 188, 148 | FIG. 1A |
| Multispecific molecule 35 | SEQ ID NOs: 181, 152, 189, 147 | FIG. 1A |
| Multispecific molecule 36 | SEQ ID NOs: 181, 152, 190, 148 | FIG. 1A |
| Multispecific molecule 37 | SEQ ID NOs: 145, 147, 140, 161 | FIG. 2A |
| Multispecific molecule 38 | SEQ ID NOs: 153, 147, 140, 161 | FIG. 2A |
| Multispecific molecule 39 | SEQ ID NOs: 154, 147, 140, 161 | FIG. 2A |
| Multispecific molecule 40 | SEQ ID NOs: 146, 148, 140, 161 | FIG. 2A |
| Multispecific molecule 41 | SEQ ID NOs: 155, 148, 140, 161 | FIG. 2A |
| Multispecific molecule 42 | SEQ ID NOs: 156, 148, 140, 161 | FIG. 2A |
| Multispecific molecule 43 | SEQ ID NOs: 145, 147, 140, 141 | FIG. 2B |
| Multispecific molecule 44 | SEQ ID NOs: 153, 147, 140, 141 | FIG. 2B |
| Multispecific molecule 45 | SEQ ID NOs: 154, 147, 140, 141 | FIG. 2B |
| Multispecific molecule 46 | SEQ ID NOs: 146, 148, 140, 141 | FIG. 2B |
| Multispecific molecule 47 | SEQ ID NOs: 155, 148, 140, 141 | FIG. 2B |
| Multispecific molecule 48 | SEQ ID NOs: 156, 148, 140, 141 | FIG. 2B |
| Multispecific molecule 49 | SEQ ID NOs: 145, 147, 162, 161 | FIG. 2C |
| Multispecific molecule 50 | SEQ ID NOs: 153, 147, 162, 161 | FIG. 2C |
| Multispecific molecule 51 | SEQ ID NOs: 154, 147, 162, 161 | FIG. 2C |
| Multispecific molecule 52 | SEQ ID NOs: 146, 148, 162, 161 | FIG. 2C |
| Multispecific molecule 53 | SEQ ID NOs: 155, 148, 162, 161 | FIG. 2C |
| Multispecific molecule 54 | SEQ ID NOs: 156, 148, 162, 161 | FIG. 2C |
| Multispecific molecule 55 | SEQ ID NOs: 145, 147, 162, 141 | FIG. 2D |
| Multispecific molecule 56 | SEQ ID NOs: 153, 147, 162, 141 | FIG. 2D |
| Multispecific molecule 57 | SEQ ID NOs: 154, 147, 162, 141 | FIG. 2D |
| Multispecific molecule 58 | SEQ ID NOs: 146, 148, 162, 141 | FIG. 2D |
| Multispecific molecule 59 | SEQ ID NOs: 155, 148, 162, 141 | FIG. 2D |
| Multispecific molecule 60 | SEQ ID NOs: 156, 148, 162, 141 | FIG. 2D |
| Multispecific molecule 61 | SEQ ID NOs: 137, 138, 140, 161 | FIG. 3A |
| Multispecific molecule 62 | SEQ ID NOs: 139, 138, 140, 161 | FIG. 3A |
| Multispecific molecule 63 | SEQ ID NOs: 149, 150, 140, 161 | FIG. 3A |
| Multispecific molecule 64 | SEQ ID NOs: 151, 152, 140, 161 | FIG. 3A |
| Multispecific molecule 65 | SEQ ID NOs: 137, 138, 140, 141 | FIG. 3B |
| Multispecific molecule 66 | SEQ ID NOs: 139, 138, 140, 141 | FIG. 3B |
| Multispecific molecule 67 | SEQ ID NOs: 149, 150, 140, 141 | FIG. 3B |
| Multispecific molecule 68 | SEQ ID NOs: 151, 152, 140, 141 | FIG. 3B |
| Multispecific molecule 69 | SEQ ID NOs: 137, 138, 162, 161 | FIG. 3C |
| Multispecific molecule 70 | SEQ ID NOs: 139, 138, 162, 161 | FIG. 3C |
| Multispecific molecule 71 | SEQ ID NOs: 149, 150, 162, 161 | FIG. 3C |
| Multispecific molecule 72 | SEQ ID NOs: 151, 152, 162, 161 | FIG. 3C |
| Multispecific molecule 73 | SEQ ID NOs: 137, 138, 162, 141 | FIG. 3D |
| Multispecific molecule 74 | SEQ ID NOs: 139, 138, 162, 141 | FIG. 3D |
| Multispecific molecule 75 | SEQ ID NOs: 149, 150, 162, 141 | FIG. 3D |
| Multispecific molecule 76 | SEQ ID NOs: 151, 152, 162, 141 | FIG. 3D |
| Multispecific molecule 77 | SEQ ID NOs: 166, 161, 172, 161 | FIG. 4A |
| Multispecific molecule 78 | SEQ ID NOs: 167, 161, 172, 161 | FIG. 4A |
| Multispecific molecule 79 | SEQ ID NOs: 168, 161, 172, 161 | FIG. 4A |
| Multispecific molecule 80 | SEQ ID NOs: 166, 161, 173, 161 | FIG. 4A |
| Multispecific molecule 81 | SEQ ID NOs: 167, 161, 173, 161 | FIG. 4A |
| Multispecific molecule 82 | SEQ ID NOs: 168, 161, 173, 161 | FIG. 4A |
| Multispecific molecule 83 | SEQ ID NOs: 169, 141, 174, 141 | FIG. 4B |
| Multispecific molecule 84 | SEQ ID NOs: 170, 141, 174, 141 | FIG. 4B |
| Multispecific molecule 85 | SEQ ID NOs: 171, 141, 174, 141 | FIG. 4B |
| Multispecific molecule 86 | SEQ ID NOs: 169, 141, 175, 141 | FIG. 4B |
| Multispecific molecule 87 | SEQ ID NOs: 170, 141, 175, 141 | FIG. 4B |
| Multispecific molecule 88 | SEQ ID NOs: 171, 141, 175, 141 | FIG. 4B |
| Multispecific molecule 89 | SEQ ID NOs: 166, 161, 174, 141 | FIG. 4C |
| Multispecific molecule 90 | SEQ ID NOs: 167, 161, 174, 141 | FIG. 4C |
| Multispecific molecule 91 | SEQ ID NOs: 168, 161, 174, 141 | FIG. 4C |
| Multispecific molecule 92 | SEQ ID NOs: 166, 161, 175, 141 | FIG. 4C |
| Multispecific molecule 93 | SEQ ID NOs: 167, 161, 175, 141 | FIG. 4C |
| Multispecific molecule 94 | SEQ ID NOs: 168, 161, 175, 141 | FIG. 4C |
| Multispecific molecule 95 | SEQ ID NOs: 166, 141, 174, 161 | FIG. 4D |
| Multispecific molecule 96 | SEQ ID NOs: 167, 141, 174, 161 | FIG. 4D |
| Multispecific molecule 97 | SEQ ID NOs: 168, 141, 174, 161 | FIG. 4D |
| Multispecific molecule 98 | SEQ ID NOs: 166, 141, 175, 161 | FIG. 4D |
| Multispecific molecule 99 | SEQ ID NOs: 167, 141, 175, 161 | FIG. 4D |
| Multispecific molecule 100 | SEQ ID NOs: 168, 141, 175, 161 | FIG. 4D |
| Multispecific molecule 101 | SEQ ID NOs: 142, 143 | FIG. 5A |
| Multispecific molecule 102 | SEQ ID NOs: 142, 144 | FIG. 5A |
| Multispecific molecule 103 | SEQ ID NOs: 157, 143 | FIG. 5A |
| Multispecific molecule 104 | SEQ ID NOs: 157, 144 | FIG. 5A |
| Multispecific molecule 105 | SEQ ID NOs: 158, 143 | FIG. 5A |
| Multispecific molecule 106 | SEQ ID NOs: 158, 144 | FIG. 5A |
| Multispecific molecule 107 | SEQ ID NOs: 163, 143 | FIG. 5B |
| Multispecific molecule 108 | SEQ ID NOs: 163, 144 | FIG. 5B |
| Multispecific molecule 109 | SEQ ID NOs: 164, 143 | FIG. 5B |
| Multispecific molecule 110 | SEQ ID NOs: 164, 144 | FIG. 5B |
| Multispecific molecule 112 | SEQ ID NOs: 165, 143 | FIG. 5B |
| Multispecific molecule 113 | SEQ ID NOs: 165, 144 | FIG. 5B |

### Example 3. Inhibition of TGFβ Signaling Using TGFβ Trap

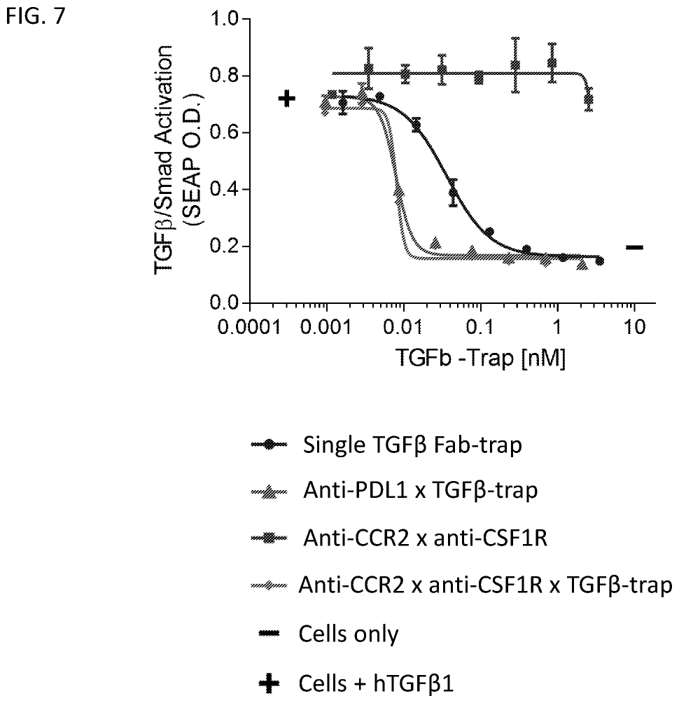

This study examines three TGFβ-trap constructs for their ability to inhibit TGFβ signaling. The first construct, “Single TGFβ Fab-trap” shown in FIG. 7, comprises two chains: the first chain comprises from N-terminus to C-terminus a first TGFBR2 ECD, a first linker, and a heavy chain constant region 1 (CH1); and the second chain comprises from N-terminus to C-terminus a second TGFBR2 ECD, a second linker, and a light chain constant region (CL). This construct does not comprise any targeting domains. The second construct, “Anti-PDL1×TGGβ-trap” shown in FIG. 7, comprises an anti-PDL1 antibody fused, at the C-terminus of its two Fc regions, to a TGFBR2 ECD homodimer. The third construct, “Anti-CCR2×anti-CSF1R×TGFβ-trap” shown in FIG. 7, comprises an anti-CCR2×anti-CSF1R bispecific antibody fused, at the C-terminus of its two Fc regions, to a TGFBR2 ECD homodimer. In addition, a fourth construct, “Anti-CCR2×anti-CSF1R” in FIG. 7, which is an anti-CCR2×anti-CSF1R bispecific antibody without a TGFβ-trap, was used as a negative control.

Briefly, HEK-Blue TGF-b cells were treated with the four constructs described above in a dose dependent manner in the presence of 0.5 ng/ml of TGF-β1 for 20-22 hours. TGF-β1 binds to receptors on HEK-Blue cells and induces activation of the TGF-β/Smad pathway leading to the formation of a Smad3/Smad4 complex. This heterocomplex enters the nucleus and binds SBE (Smad3/4-binding elements) sites inducing production of SEAP (secreted embryonic alkaline phosphatase). SEAP secreted in the supernatant was quantified by colormetric enzymatic assays (QUANTI-Blue). As shown in FIG. 7, TGF-β1-mediated SEAP production was reduced by all three TGFβ-trap constructs tested here. The anti-CCR2×anti-CSF1R bispecific antibody without a TGFβ-trap did not reduce TGF-β1 signaling (FIG. 7).

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. An isolated multispecific, e.g., a bispecific or trispecific, molecule, comprising:
(i) a TGF-beta inhibitor;
(ii) an anti-CSF1R binding moiety (e.g., an anti-CSF1R antibody molecule) or an anti-CCR2 binding moiety (e.g., an anti-CCR2 antibody molecule); and
(iii) a tumor targeting moiety (e.g., a tumor targeting antibody molecule).

2. The multispecific molecule of claim 1, wherein the TGF-beta inhibitor reduces the activity of one, two, or all of:
(i) TGF-beta 1,
(ii) TGF-beta 2, or
(iii) TGF-beta 3, optionally wherein the TGF-beta inhibitor reduces the activity of:
(a) TGF-beta 1 and TGF-beta 3, or
(b) TGF-beta 1, TGF-beta 2, and TGF-beta 3, e.g., as measured using the methods described in Example 3 with respect to FIG. 7.

3. The multispecific molecule of claim 1 or 2, wherein the TGF-beta inhibitor comprises a TGF-beta receptor polypeptide (e.g., an extracellular domain of a TGF-beta receptor, or a functional variant thereof).

4. The multispecific molecule of any one of claims 1-3, wherein the TGF-beta inhibitor comprises one, two, or all of:
(i) a TGFBR1 polypeptide (e.g., 1, 2, 3, or more of a TGFBR1 polypeptide),
(ii) a TGFBR2 polypeptide (e.g., 1, 2, 3, or more of a TGFBR2 polypeptide), or
(iii) a TGFBR3 polypeptide (e.g., 1, 2, 3, or more of a TGFBR3 polypeptide).

5. The multispecific molecule of any one of claims 1-4, wherein the TGF-beta inhibitor comprises a TGFBR1 polypeptide, optionally wherein the TGF-beta inhibitor comprises:
(i) an extracellular domain of TGFBR1 or a sequence substantially identical thereto (e.g., a sequence that is at least 80%, 85%, 90%, or 95% identical thereto),
(ii) an extracellular domain of SEQ ID NO: 95, 96, 97, 120, 121, or 122, or a sequence substantially identical thereto (e.g., a sequence that is at least 80%, 85%, 90%, or 95% identical thereto), or
(iii) the amino acid sequence of SEQ ID NO: 104 or 105, or a sequence substantially identical thereto (e.g., a sequence that is at least 80%, 85%, 90%, or 95% identical thereto).

6. The multispecific molecule of any one of claims 1-5, wherein the TGF-beta inhibitor comprises a TGFBR2 polypeptide, optionally wherein the TGF-beta inhibitor comprises:
(i) an extracellular domain of TGFBR2 or a sequence substantially identical thereto (e.g., a sequence that is at least 80%, 85%, 90%, or 95% identical thereto),
(ii) an extracellular domain of SEQ ID NO: 98, 99, 123, or 124, or a sequence substantially identical thereto (e.g., a sequence that is at least 80%, 85%, 90%, or 95% identical thereto), or
(iii) an amino acid sequence selected from the group consisting of SEQ ID NOs: 100, 101, 102, and 103, or a sequence substantially identical thereto (e.g., a sequence that is at least 80%, 85%, 90%, or 95% identical thereto).

7. The multispecific molecule of any one of claims 1-6, wherein the TGF-beta inhibitor comprises a TGFBR3 polypeptide, optionally wherein the TGF-beta inhibitor comprises:
(i) an extracellular domain of TGFBR3 or a sequence substantially identical thereto (e.g., a sequence that is at least 80%, 85%, 90%, or 95% identical thereto),
(ii) an extracellular domain of SEQ ID NO: 106, 107, 125, or 126, or a sequence substantially identical thereto (e.g., a sequence that is at least 80%, 85%, 90%, or 95% identical thereto), or
(iii) the amino acid sequence of SEQ ID NO: 108, or a sequence substantially identical thereto (e.g., a sequence that is at least 80%, 85%, 90%, or 95% identical thereto).

8. The multispecific molecule of any one of claims 1-7, wherein the TGF-beta inhibitor comprises two TGF-beta receptor polypeptides that form a homodimer, optionally wherein the TGF-beta inhibitor comprises:
(i) two TGFBR1 polypeptides that form a homodimer,
(ii) two TGFBR2 polypeptides that form a homodimer, or
(iii) two TGFBR3 polypeptides that form a homodimer.

9. The multispecific molecule of any one of claims 1-8, wherein the TGF-beta inhibitor comprises two TGF-beta receptor polypeptides that form a heterodimer, optionally wherein the TGF-beta inhibitor comprises:
(i) a TGFBR1 polypeptide and a TGFBR2 polypeptide that form a heterodimer,
(ii) a TGFBR1 polypeptide and a TGFBR3 polypeptide that form a heterodimer, or
(iii) a TGFBR2 polypeptide and a TGFBR3 polypeptide that form a heterodimer.

10. The multispecific molecule of any one of claims 1-9, wherein the TGF-beta inhibitor comprises a first TGF-beta receptor polypeptide and a second TGF-beta receptor polypeptide.

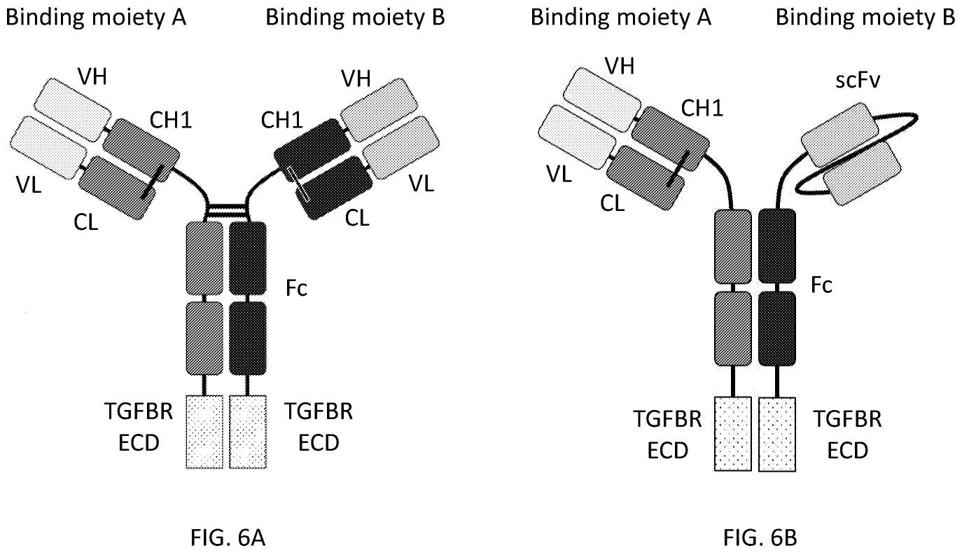

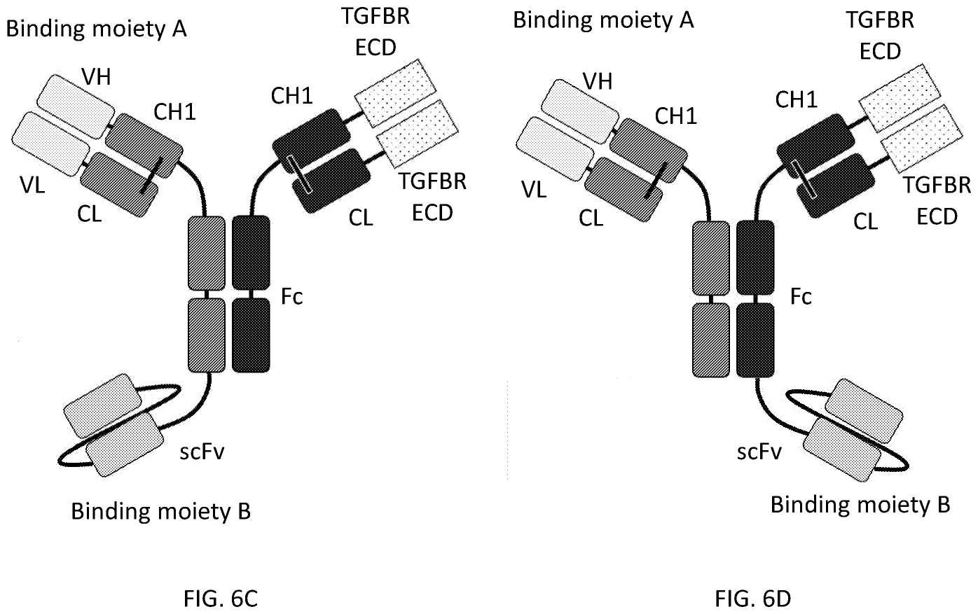

11. The multispecific molecule of claim 10, wherein the multispecific molecule comprises a first Fc region (e.g., a first CH1-Fc region) and a second Fc region (e.g., a second CH1-Fc region), optionally wherein:
(i) the first TGF-beta receptor polypeptide is linked, e.g., via a linker, to the first Fc region (e.g., a first CH1-Fc region), e.g., the C-terminus of the first Fc region (e.g., a first CH1-Fc region), and
(ii) the second TGF-beta receptor polypeptide is linked, e.g., via a linker, to the second Fc region (e.g., a second CH1-Fc region), e.g., the C-terminus of the second Fc region (e.g., a second CH1-Fc region), optionally wherein:
the first TGF-beta receptor polypeptide and the second TGF-beta receptor polypeptide form a homodimer or heterodimer, e.g., a homodimer, optionally wherein:
the first or second TGF-beta receptor polypeptide comprises an extracellular domain of TGFBR1, TGFBR2, or TGFBR3, e.g., an extracellular domain of TGFBR2, optionally wherein:
the multispecific molecule has the configuration of FIG. 6A or 6B.

12. The multispecific molecule of claim 11, wherein the multispecific molecule comprises:
(i) the amino acid sequence of SEQ ID NO: 192 and the amino acid sequence of SEQ ID NO: 193,
(ii) the amino acid sequence of SEQ ID NO: 192 and the amino acid sequence of SEQ ID NO: 195,
(iii) the amino acid sequence of SEQ ID NO: 194 and the amino acid sequence of SEQ ID NO: 193, or
(iv) the amino acid sequence of SEQ ID NO: 194 and the amino acid sequence of SEQ ID NO: 195.

13. The multispecific molecule of claim 10, wherein the multispecific molecule comprises a heavy chain constant region 1 (CH1) and a light chain constant region (CL), optionally wherein:
(i) the first TGF-beta receptor polypeptide is linked, e.g., via a linker, to the CH1, e.g., the N-terminus of the CH1, and
(ii) the second TGF-beta receptor polypeptide is linked, e.g., via a linker, to the CL, e.g., the N-terminus of the CL, optionally wherein:
the first TGF-beta receptor polypeptide and the second TGF-beta receptor polypeptide form a homodimer or heterodimer, e.g., a homodimer, optionally wherein:
the first or second TGF-beta receptor polypeptide comprises an extracellular domain of TGFBR1, TGFBR2, or TGFBR3, e.g., an extracellular domain of TGFBR2, optionally wherein:
the multispecific molecule has the configuration of FIG. 6C or 6D.

14. The multispecific molecule of claim 13, wherein the multispecific molecule comprises:
(i) the amino acid sequence of SEQ ID NO: 196 and the amino acid sequence of SEQ ID NO: 198,
(ii) the amino acid sequence of SEQ ID NO: 196 and the amino acid sequence of SEQ ID NO: 199,
(iii) the amino acid sequence of SEQ ID NO: 197 and the amino acid sequence of SEQ ID NO: 198, or
(iv) the amino acid sequence of SEQ ID NO: 197 and the amino acid sequence of SEQ ID NO: 199.

15. The multispecific molecule of any one of claims 1-14, comprising an anti-CSF1R binding moiety (e.g., an anti-CSF1R antibody molecule).

16. The multispecific molecule of any one of claims 1-15, comprising an anti-CCR2 binding moiety (e.g., an anti-CCR2 antibody molecule).

17. The multispecific molecule of any one of claims 1-16, wherein the tumor targeting moiety (e.g., a tumor targeting antibody molecule) binds to PD-L1, mesothelin, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pme117, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDCl₂7, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Integrins (Integrin alphaVbeta3, Integrin alpha5Beta1), Carbohydrates (Le), IGF1R, EPHA3, TRAILR1, TRAILR2, or RANKL.

18. The multispecific molecule of any one of claims 1-17, wherein the anti-CSF1R antibody molecule, anti-CCR2 antibody molecule, or tumor targeting antibody molecule is, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)).

19. The multispecific molecule of any one of claims 1-18, wherein the anti-CSF1R antibody molecule, anti-CCR2 antibody molecule, or tumor targeting antibody molecule comprises a heavy chain constant region chosen from IgG1, IgG2, IgG3, or IgG4, or a fragment thereof.

20. The multispecific molecule of any one of claims 1-19, wherein the anti-CSF1R antibody molecule, anti-CCR2 antibody molecule, or tumor targeting antibody molecule comprises a light chain constant region chosen from the light chain constant regions of kappa or lambda, or a fragment thereof.

21. The multispecific molecule of any one of claims 1-20, wherein the anti-CSF1R antibody molecule or anti-CCR2 antibody molecule comprises a kappa light chain constant region, or a fragment thereof, and the tumor targeting antibody molecule comprises a lambda light chain constant region, or a fragment thereof.

22. The multispecific molecule of any one of claims 1-20, wherein the anti-CSF1R antibody molecule or anti-CCR2 antibody molecule comprises a lambda light chain constant region, or a fragment thereof, and the tumor targeting antibody molecule comprises a kappa light chain constant region, or a fragment thereof.

23. The multispecific molecule of any one of claims 1-20, wherein the anti-CSF1R antibody molecule or anti-CCR2 antibody molecule and the tumor targeting antibody molecule have a common light chain variable region.

24. The multispecific molecule of any one of claims 1-23, further comprising a heavy chain constant region (e.g., an Fc region) chosen from the heavy chain constant regions of IgG1, IgG2, and IgG4, more particularly, the heavy chain constant region of human IgG1, IgG2 or IgG4.

25. The multispecific molecule of claim 24, wherein the heavy chain constant region (e.g., an Fc region) is linked to, e.g., covalently linked to, anti-CSF1R antibody molecule, anti-CCR2 antibody molecule, or tumor targeting antibody molecule.

26. The multispecific molecule of claim 24 or 25, wherein the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region.

27. The multispecific molecule of any one of claims 1-26, wherein the anti-CSF1R antibody molecule or anti-CCR2 antibody molecule comprises a first heavy chain constant region (e.g., a first Fc region) and the tumor targeting antibody molecule comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region.

28. The multispecific molecule of claim 27, wherein the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions.

29. The multispecific molecule of claim 27 or 28, wherein the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system.

30. The multispecific molecule of any one of claims 27-29, wherein the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

31. The multispecific molecule of any one of claims 1-30, further comprising a linker, optionally wherein the linker is chosen from: a cleavable linker, a non-cleavable linker, a peptide linker, a flexible linker, a rigid linker, a helical linker, or a non-helical linker, optionally wherein the linker is a peptide linker, optionally wherein the peptide linker comprises Gly and Ser.

32. An isolated multispecific molecule comprising:
(i) a CSF1R binding moiety (e.g., an anti-CSF1R antibody molecule),
(ii) a PD-L1 binding moiety (e.g., an anti-PD-L1 antibody molecule), and
(iii) a TGF-beta inhibitor.

33. The multispecific molecule of claim 32, wherein the CSF1R binding moiety (e.g., an anti-CSF1R antibody molecule) and the PD-L1 binding moiety (e.g., an anti-PD-L1 antibody molecule) are, independently, a full antibody (e.g., an antibody that includes at least one, and preferably two, complete heavy chains, and at least one, and preferably two, complete light chains), or an antigen-binding fragment (e.g., a Fab, F(ab′)2, Fv, a scFv, a single domain antibody, or a diabody (dAb)).

34. The multispecific molecule of claim 32 or 33, wherein the CSF1R binding moiety (e.g., an anti-CSF1R antibody molecule) and/or the PD-L1 binding moiety (e.g., an anti-PD-L1 antibody molecule) comprises a light chain constant region chosen from the light chain constant regions of kappa or lambda, or a fragment thereof.

35. The multispecific molecule of any one of claims 32-34, wherein:
(i) the CSF1R binding moiety (e.g., an anti-CSF1R antibody molecule) comprises a kappa light chain constant region, or a fragment thereof, and the PD-L1 binding moiety (e.g., an anti-PD-L1 antibody molecule) comprises a lambda light chain constant region, or a fragment thereof, or
(ii) the CSF1R binding moiety (e.g., an anti-CSF1R antibody molecule) comprises a lambda light chain constant region, or a fragment thereof, and the PD-L1 binding moiety (e.g., an anti-PD-L1 antibody molecule) comprises a kappa light chain constant region, or a fragment thereof.

36. The multispecific molecule of any one of claims 32-35, wherein the CSF1R binding moiety (e.g., an anti-CSF1R antibody molecule) and the PD-L1 binding moiety (e.g., an anti-PD-L1 antibody molecule) have a common light chain variable region.

37. The multispecific molecule of any one of claims 32-36, wherein the CSF1R binding moiety (e.g., an anti-CSF1R antibody molecule) and/or the PD-L1 binding moiety (e.g., an anti-PD-L1 antibody molecule) comprises a heavy chain constant region (e.g., a CH1 region and an Fc region) chosen from IgG1, IgG2, IgG3, or IgG4, or a fragment thereof.

38. The multispecific molecule of claim 37, wherein the heavy chain constant region (e.g., an Fc region) comprises one or more mutations that increase or decrease one or more of: Fc receptor binding, antibody glycosylation, the number of cysteine residues, effector cell function, or complement function, relative to a naturally-existing heavy chain constant region.

39. The multispecific molecule of any one of claims 32-38, wherein the CSF1R binding moiety (e.g., an anti-CSF1R antibody molecule) comprises a first heavy chain constant region (e.g., a first Fc region) and the PD-L1 binding moiety (e.g., an anti-PD-L1 antibody molecule) comprises a second heavy chain constant region (e.g., a second Fc region), wherein the first heavy chain constant region comprises one or more mutations that increase heterodimerization of the first heavy chain constant region and the second heavy chain constant region, relative to a naturally-existing heavy chain constant region, and/or wherein the second heavy chain constant region comprises one or more mutations that increase heterodimerization of the second heavy chain constant region and the first heavy chain constant region, relative to a naturally-existing heavy chain constant region.

40. The multispecific molecule of claim 39, wherein the first and the second heavy chain constant regions (e.g., first and second Fc regions) comprise one or more of: a paired cavity-protuberance (“knob-in-a hole”), an electrostatic interaction, or a strand-exchange, such that a greater ratio of heteromultimer:homomultimer forms, e.g., relative to naturally-existing heavy chain constant regions.

41. The multispecific molecule of claim 39 or 40, wherein the first and/or second heavy chain constant region (e.g., a first and/or second Fc region, e.g., a first and/or second IgG1 Fc region) comprises an amino acid substitution at a position chosen from one or more of 347, 349, 350, 351, 366, 368, 370, 392, 394, 395, 397, 398, 399, 405, 407, or 409, numbered based on the Eu numbering system.

42. The multispecific molecule of any one of claims 39-41, wherein the first and/or second heavy chain constant region (e.g., a first and/or second Fc region, e.g., a first and/or second IgG1 Fc region) comprises an amino acid substitution chosen from: T366S, L368A, Y407V, or Y349C (e.g., corresponding to a cavity or hole), or T366W or S354C (e.g., corresponding to a protuberance or knob), or a combination thereof, numbered based on the Eu numbering system.

43. The multispecific molecule of any one of claims 32-42, wherein the TGF-beta inhibitor reduces the activity of one, two, or all of:
(i) TGF-beta 1,
(ii) TGF-beta 2, or
(iii) TGF-beta 3, optionally wherein the TGF-beta inhibitor reduces the activity of:
(a) TGF-beta 1 and TGF-beta 3, or
(b) TGF-beta 1, TGF-beta 2, and TGF-beta 3.

44. The multispecific molecule of any one of claims 32-43, wherein:
(i) the TGF-beta inhibitor is linked, e.g., via a linker, to the CSF1R binding moiety (e.g., an anti-CSF1R antibody molecule) or the PD-L1 binding moiety (e.g., an anti-PD-L1 antibody molecule); or
(ii) the multispecific molecule comprises a first TGF-beta inhibitor and a second TGF-beta inhibitor, wherein the first TGF-beta inhibitor is linked, e.g., via a linker, to the CSF1R binding moiety (e.g., an anti-CSF1R antibody molecule) and wherein the second TGF-beta inhibitor is linked, e.g., via a linker, to the PD-L1 binding moiety (e.g., an anti-PD-L1 antibody molecule).

45. The multispecific molecule of any one of claims 32-44, wherein:
(i) the CSF1R binding moiety (e.g., an anti-CSF1R antibody molecule) comprises a first heavy chain polypeptide (e.g., a first heavy chain polypeptide comprising a first heavy chain variable region and a first heavy chain constant region (e.g., a first Fc region)) and a first light chain polypeptide (e.g., a first light chain polypeptide comprising a first light chain variable region and a first light chain constant region), and
(ii) the PD-L1 binding moiety (e.g., an anti-PD-L1 antibody molecule) comprises a second heavy chain polypeptide (e.g., a second heavy chain polypeptide comprising a second heavy chain variable region and a second heavy chain constant region (e.g., a second Fc region)) and a second light chain polypeptide (e.g., a second light chain polypeptide comprising a second light chain variable region and a second light chain constant region), wherein:
(a) the TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) or the second heavy chain polypeptide (e.g., the Fc region of the second heavy chain polypeptide, e.g., the C-terminus of the Fc region of the second heavy chain polypeptide),
(b) the multispecific molecule comprises a first TGF-beta inhibitor and a second TGF-beta inhibitor, wherein the first TGF-beta inhibitor is linked, e.g., via a linker, to the first heavy chain polypeptide (e.g., the Fc region of the first heavy chain polypeptide, e.g., the C-terminus of the Fc region of the first heavy chain polypeptide) and wherein the second TGF-beta inhibitor is linked, e.g., via a linker, to the second heavy chain polypeptide (e.g., the Fc region of the second heavy chain polypeptide, e.g., the C-terminus of the Fc region of the second heavy chain polypeptide),
(c) the TGF-beta inhibitor is linked, e.g., via a linker, to the first light chain polypeptide (e.g., the constant region of the first light chain polypeptide, e.g., the C-terminus of the constant region of the first light chain polypeptide) or the second light chain polypeptide (e.g., the constant region of the second light chain polypeptide, e.g., the C-terminus of the constant region of the second light chain polypeptide), or
(d) the multispecific molecule comprises a first TGF-beta inhibitor and a second TGF-beta inhibitor, wherein the first TGF-beta inhibitor is linked, e.g., via a linker, to the first light chain polypeptide (e.g., the constant region of the first light chain polypeptide, e.g., the C-terminus of the constant region of the first light chain polypeptide) and wherein the second TGF-beta inhibitor is linked, e.g., via a linker, to the second light chain polypeptide (e.g., the constant region of the second light chain polypeptide, e.g., the C-terminus of the constant region of the second light chain polypeptide).

46. The multispecific molecule of any one of claims 32-45, comprising:
(i) a first polypeptide comprising a first portion of the CSF1R binding moiety comprising a first VL and a first CL;
(ii) a second polypeptide comprising (1) a second portion of the CSF1R binding moiety comprising a first VH, a first CH1, a first CH2, and a first CH3, and optionally (2) a first TGF-beta inhibitor;
(iii) a third polypeptide comprising (1) a first portion of the PD-L1 binding moiety comprising a second VH, a second CH1, a second CH2, and a second CH3, and optionally (2) a second TGF-beta inhibitor; and
(iv) a fourth polypeptide comprising a second portion of the PD-L1 binding moiety comprising a second VL and a second CL, wherein:
the multispecific molecule comprises at least one of: the first TGF-beta inhibitor or the second TGF-beta inhibitor, optionally wherein the first and the second TGF-beta inhibitors form a homo-dimer or hetero-dimer.

47. The multispecific molecule of any one of claims 32-45, comprising:
(i) a first polypeptide comprising a first portion of the CSF1R binding moiety comprising a first VL and a first CL;
(ii) a second polypeptide comprising (1) a second portion of the CSF1R binding moiety comprising a first VH, a first CH1, a first CH2, and a first CH3, and (2) the PD-L1 binding moiety comprising a second VH and a second VL (e.g., an scFv);
(iii) a third polypeptide comprising a first TGF-beta inhibitor, a second CH1, a second CH2, and a second CH3; and
(iv) a fourth polypeptide comprising a second TGF-beta inhibitor, and a second CL,
optionally wherein the first and the second TGF-beta inhibitors form a homo-dimer or hetero-dimer.

48. The multispecific molecule of any one of claims 32-45, comprising:
(i) a first polypeptide comprising a first portion of the PD-L1 binding moiety comprising a first VL and a first CL;
(ii) a second polypeptide comprising (1) a second portion of the PD-L1 binding moiety comprising a first VH, a first CH1, a first CH2, and a first CH3, and (2) the CSF1R binding moiety comprising a second VH and a second VL (e.g., an scFv);
(iii) a third polypeptide comprising a first TGF-beta inhibitor, a second CH1, a second CH2, and a second CH3; and
(iv) a fourth polypeptide comprising a second TGF-beta inhibitor, and a second CL,
optionally wherein the first and the second TGF-beta inhibitors form a homo-dimer or hetero-dimer.

49. The multispecific molecule of any one of claims 32-45, comprising:
(i) a first polypeptide comprising a first TGF-beta inhibitor and a first CL;
(ii) a second polypeptide comprising (1) a second TGF-beta inhibitor, a first CH1, a first CH2, and a first CH3, and (2) the PD-L1 binding moiety comprising a first VH and a first VL (e.g., a first scFv);
(iii) a third polypeptide comprising (1) a third TGF-beta inhibitor, a second CH1, a second CH2, and a second CH3, and (2) the CSF1R binding moiety comprising a second VH and a second VL (e.g., a second scFv);
(iv) a fourth polypeptide comprising a fourth TGF-beta inhibitor and a second CL,
optionally wherein the first and the second TGF-beta inhibitors form a homo-dimer or hetero-dimer, and/or the third and the fourth TGF-beta inhibitors form a homo-dimer or hetero-dimer.

50. The multispecific molecule of any one of claims 32-45, comprising:
(i) a first polypeptide comprising (1) a first TGF-beta inhibitor, a first CH2, and a first CH3, and (2) the PD-L1 binding moiety comprising a first VH and a first VL (e.g., a first scFv); and
(ii) a second polypeptide comprising (1) a second TGF-beta inhibitor, a second CH2, and a second CH3, and (2) the CSF1R binding moiety comprising a second VH and a second VL (e.g., a second scFv).

51. The multispecific molecule of any one of claims 32-50, wherein the TGF-beta inhibitor, or the first, second, third, or fourth TGF-beta inhibitor comprises a TGF-beta receptor polypeptide (e.g., an extracellular domain of a TGF-beta receptor, or a functional variant thereof).

52. The multispecific molecule of any one of claims 32-51, wherein the TGF-beta inhibitor, or the first, second, third, or fourth TGF-beta inhibitor comprises one, two, or all of:
(i) a TGFBR1 polypeptide (e.g., 1, 2, 3, or more of a TGFBR1 polypeptide),
(ii) a TGFBR2 polypeptide (e.g., 1, 2, 3, or more of a TGFBR2 polypeptide), or
(iii) a TGFBR3 polypeptide (e.g., 1, 2, 3, or more of a TGFBR3 polypeptide).

53. The multispecific molecule of any one of claims 32-52, wherein the TGF-beta inhibitor, or the first, second, third, or fourth TGF-beta inhibitor comprises a TGFBR1 polypeptide, e.g., the TGF-beta inhibitor comprises:
(i) an extracellular domain of TGFBR1 or a sequence substantially identical thereto (e.g., a sequence that is at least 80%, 85%, 90%, or 95% identical thereto),
(ii) an extracellular domain of SEQ ID NO: 95, 96, 97, 120, 121, or 122, or a sequence substantially identical thereto (e.g., a sequence that is at least 80%, 85%, 90%, or 95% identical thereto), or
(iii) the amino acid sequence of SEQ ID NO: 104 or 105, or a sequence substantially identical thereto (e.g., a sequence that is at least 80%, 85%, 90%, or 95% identical thereto).

54. The multispecific molecule of any one of claims 32-53, wherein the TGF-beta inhibitor, or the first, second, third, or fourth TGF-beta inhibitor comprises a TGFBR2 polypeptide, e.g., the TGF-beta inhibitor comprises:
(i) an extracellular domain of TGFBR2 or a sequence substantially identical thereto (e.g., a sequence that is at least 80%, 85%, 90%, or 95% identical thereto),
(ii) an extracellular domain of SEQ ID NO: 98, 99, 123, or 124, or a sequence substantially identical thereto (e.g., a sequence that is at least 80%, 85%, 90%, or 95% identical thereto), or
(iii) an amino acid sequence selected from the group consisting of SEQ ID NOs: 100, 101, 102, and 103, or a sequence substantially identical thereto (e.g., a sequence that is at least 80%, 85%, 90%, or 95% identical thereto).

55. The multispecific molecule of any one of claims 32-54, wherein the TGF-beta inhibitor, or the first, second, third, or fourth TGF-beta inhibitor comprises a TGFBR3 polypeptide, e.g., the TGF-beta inhibitor comprises:
(i) an extracellular domain of TGFBR3 or a sequence substantially identical thereto (e.g., a sequence that is at least 80%, 85%, 90%, or 95% identical thereto),
(ii) an extracellular domain of SEQ ID NO: 106, 107, 125, or 126, or a sequence substantially identical thereto (e.g., a sequence that is at least 80%, 85%, 90%, or 95% identical thereto), or
(iii) the amino acid sequence of SEQ ID NO: 108, or a sequence substantially identical thereto (e.g., a sequence that is at least 80%, 85%, 90%, or 95% identical thereto).

56. The multispecific molecule of any one of claims 32-55, comprising a first, second, third, and fourth non-contiguous polypeptides, wherein the first, second, third, and fourth non-contiguous polypeptides comprise the amino acid sequences of:
SEQ ID NOs: 176, 138, 185, and 147, respectively, or a sequence substantially identical thereto (e.g., 85% to 99% identical thereto);
SEQ ID NOs: 176, 138, 186, and 148, respectively, or a sequence substantially identical thereto (e.g., 85% to 99% identical thereto);
SEQ ID NOs: 176, 138, 187, and 147, respectively, or a sequence substantially identical thereto (e.g., 85% to 99% identical thereto);
SEQ ID NOs: 176, 138, 188, and 148, respectively, or a sequence substantially identical thereto (e.g., 85% to 99% identical thereto);
SEQ ID NOs: 176, 138, 189, and 147, respectively, or a sequence substantially identical thereto (e.g., 85% to 99% identical thereto);
SEQ ID NOs: 176, 138, 190, and 148, respectively, or a sequence substantially identical thereto (e.g., 85% to 99% identical thereto);
SEQ ID NOs: 177, 150, 185, and 147, respectively, or a sequence substantially identical thereto (e.g., 85% to 99% identical thereto);
SEQ ID NOs: 177, 150, 186, and 148, respectively, or a sequence substantially identical thereto (e.g., 85% to 99% identical thereto);
SEQ ID NOs: 177, 150, 187, and 147, respectively, or a sequence substantially identical thereto (e.g., 85% to 99% identical thereto);
SEQ ID NOs: 177, 150, 188, and 148, respectively, or a sequence substantially identical thereto (e.g., 85% to 99% identical thereto);
SEQ ID NOs: 177, 150, 189, and 147, respectively, or a sequence substantially identical thereto (e.g., 85% to 99% identical thereto);
SEQ ID NOs: 177, 150, 190, and 148, respectively, or a sequence substantially identical thereto (e.g., 85% to 99% identical thereto);
SEQ ID NOs: 178, 152, 185, and 147, respectively, or a sequence substantially identical thereto (e.g., 85% to 99% identical thereto);
SEQ ID NOs: 178, 152, 186, and 148, respectively, or a sequence substantially identical thereto (e.g., 85% to 99% identical thereto);
SEQ ID NOs: 178, 152, 187, and 147, respectively, or a sequence substantially identical thereto (e.g., 85% to 99% identical thereto);
SEQ ID NOs: 178, 152, 188, and 148, respectively, or a sequence substantially identical thereto (e.g., 85% to 99% identical thereto);
SEQ ID NOs: 178, 152, 189, and 147, respectively, or a sequence substantially identical thereto (e.g., 85% to 99% identical thereto);
SEQ ID NOs: 178, 152, 190, and 147, respectively, or a sequence substantially identical thereto (e.g., 85% to 99% identical thereto);
SEQ ID NOs: 179, 138, 185, and 147, respectively, or a sequence substantially identical thereto (e.g., 85% to 99% identical thereto);
SEQ ID NOs: 179, 138, 186, and 148, respectively, or a sequence substantially identical thereto (e.g., 85% to 99% identical thereto);
SEQ ID NOs: 179, 138, 187, and 148, respectively, or a sequence substantially identical thereto (e.g., 85% to 99% identical thereto);
SEQ ID NOs: 179, 138, 188, and 148, respectively, or a sequence substantially identical thereto (e.g., 85% to 99% identical thereto);
SEQ ID NOs: 179, 138, 189, and 147, respectively, or a sequence substantially identical thereto (e.g., 85% to 99% identical thereto);
SEQ ID NOs: 179, 138, 190, and 148, respectively, or a sequence substantially identical thereto (e.g., 85% to 99% identical thereto);
SEQ ID NOs: 180, 150, 185, and 147, respectively, or a sequence substantially identical thereto (e.g., 85% to 99% identical thereto);
SEQ ID NOs: 180, 150, 186, and 148, respectively, or a sequence substantially identical thereto (e.g., 85% to 99% identical thereto);
SEQ ID NOs: 180, 150, 187, and 147, respectively, or a sequence substantially identical thereto (e.g., 85% to 99% identical thereto);
SEQ ID NOs: 180, 150, 188, and 148, respectively, or a sequence substantially identical thereto (e.g., 85% to 99% identical thereto);
SEQ ID NOs: 180, 150, 189, and 147, respectively, or a sequence substantially identical thereto (e.g., 85% to 99% identical thereto);
SEQ ID NOs: 180, 150, 190, and 148, respectively, or a sequence substantially identical thereto (e.g., 85% to 99% identical thereto);
SEQ ID NOs: 181, 152, 185, and 147, respectively, or a sequence substantially identical thereto (e.g., 85% to 99% identical thereto);
SEQ ID NOs: 181, 152, 186, and 148, respectively, or a sequence substantially identical thereto (e.g., 85% to 99% identical thereto);
SEQ ID NOs: 181, 152, 187, and 147, respectively, or a sequence substantially identical thereto (e.g., 85% to 99% identical thereto);
SEQ ID NOs: 181, 152, 188, and 148, respectively, or a sequence substantially identical thereto (e.g., 85% to 99% identical thereto);
SEQ ID NOs: 181, 152, 189, and 147, respectively, or a sequence substantially identical thereto (e.g., 85% to 99% identical thereto);
SEQ ID NOs: 181, 152, 190, and 148, respectively, or a sequence substantially identical thereto (e.g., 85% to 99% identical thereto);
SEQ ID NOs: 145, 147, 140, and 161, respectively, or a sequence substantially identical thereto (e.g., 85% to 99% identical thereto);
SEQ ID NOs: 153, 147, 140, and 161, respectively, or a sequence substantially identical thereto (e.g., 85% to 99% identical thereto);
SEQ ID NOs: 154, 147, 140, and 161, respectively, or a sequence substantially identical thereto (e.g., 85% to 99% identical thereto);
SEQ ID NOs: 146, 148, 140, and 161, respectively, or a sequence substantially identical thereto (e.g., 85% to 99% identical thereto);
SEQ ID NOs: 155, 148, 140, and 161, respectively, or a sequence substantially identical thereto (e.g., 85% to 99% identical thereto);
SEQ ID NOs: 156, 148, 140, and 161, respectively, or a sequence substantially identical thereto (e.g., 85% to 99% identical thereto);
SEQ ID NOs: 145, 147, 140, and 141, respectively, or a sequence substantially identical thereto (e.g., 85% to 99% identical thereto);
SEQ ID NOs: 153, 147, 140, and 141, respectively, or a sequence substantially identical thereto (e.g., 85% to 99% identical thereto);
SEQ ID NOs: 154, 147, 140, and 141, respectively, or a sequence substantially identical thereto (e.g., 85% to 99% identical thereto);
SEQ ID NOs: 146, 148, 140, and 141, respectively, or a sequence substantially identical thereto (e.g., 85% to 99% identical thereto);
SEQ ID NOs: 155, 148, 140, and 141, respectively, or a sequence substantially identical thereto (e.g., 85% to 99% identical thereto);
SEQ ID NOs: 156, 148, 140, and 141, respectively, or a sequence substantially identical thereto (e.g., 85% to 99% identical thereto);
SEQ ID NOs: 145, 147, 162, and 161, respectively, or a sequence substantially identical thereto (e.g., 85% to 99% identical thereto);
SEQ ID NOs: 153, 147, 162, and 161, respectively, or a sequence substantially identical thereto (e.g., 85% to 99% identical thereto);
SEQ ID NOs: 154, 147, 162, and 161, respectively, or a sequence substantially identical thereto (e.g., 85% to 99% identical thereto);
SEQ ID NOs: 146, 148, 162, and 161, respectively, or a sequence substantially identical thereto (e.g., 85% to 99% identical thereto);
SEQ ID NOs: 155, 148, 162, and 161, respectively, or a sequence substantially identical thereto (e.g., 85% to 99% identical thereto);
SEQ ID NOs: 156, 148, 162, and 161, respectively, or a sequence substantially identical thereto (e.g., 85% to 99% identical thereto);
SEQ ID NOs: 145, 147, 162, and 141, respectively, or a sequence substantially identical thereto (e.g., 85% to 99% identical thereto);
SEQ ID NOs: 153, 147, 162, and 141, respectively, or a sequence substantially identical thereto (e.g., 85% to 99% identical thereto);
SEQ ID NOs: 154, 147, 162, and 141, respectively, or a sequence substantially identical thereto (e.g., 85% to 99% identical thereto);
SEQ ID NOs: 146, 148, 162, and 141, respectively, or a sequence substantially identical thereto (e.g., 85% to 99% identical thereto);
SEQ ID NOs: 155, 148, 162, and 141, respectively, or a sequence substantially identical thereto (e.g., 85% to 99% identical thereto);
SEQ ID NOs: 156, 148, 162, and 141, respectively, or a sequence substantially identical thereto (e.g., 85% to 99% identical thereto);
SEQ ID NOs: 137, 138, 140, and 161, respectively, or a sequence substantially identical thereto (e.g., 85% to 99% identical thereto);
SEQ ID NOs: 139, 138, 140, and 161, respectively, or a sequence substantially identical thereto (e.g., 85% to 99% identical thereto);
SEQ ID NOs: 149, 150, 140, and 161, respectively, or a sequence substantially identical thereto (e.g., 85% to 99% identical thereto);
SEQ ID NOs: 151, 152, 140, and 161, respectively, or a sequence substantially identical thereto (e.g., 85% to 99% identical thereto);
SEQ ID NOs: 137, 138, 140, and 141, respectively, or a sequence substantially identical thereto (e.g., 85% to 99% identical thereto);
SEQ ID NOs: 139, 138, 140, and 141, respectively, or a sequence substantially identical thereto (e.g., 85% to 99% identical thereto);
SEQ ID NOs: 149, 150, 140, and 141, respectively, or a sequence substantially identical thereto (e.g., 85% to 99% identical thereto);
SEQ ID NOs: 151, 152, 140, and 141, respectively, or a sequence substantially identical thereto (e.g., 85% to 99% identical thereto);
SEQ ID NOs: 137, 138, 162, and 161, respectively, or a sequence substantially identical thereto (e.g., 85% to 99% identical thereto);
SEQ ID NOs: 139, 138, 162, and 161, respectively, or a sequence substantially identical thereto (e.g., 85% to 99% identical thereto);
SEQ ID NOs: 149, 150, 162, and 161, respectively, or a sequence substantially identical thereto (e.g., 85% to 99% identical thereto);
SEQ ID NOs: 151, 152, 162, and 161, respectively, or a sequence substantially identical thereto (e.g., 85% to 99% identical thereto);
SEQ ID NOs: 137, 138, 162, and 141, respectively, or a sequence substantially identical thereto (e.g., 85% to 99% identical thereto);
SEQ ID NOs: 139, 138, 162, and 141, respectively, or a sequence substantially identical thereto (e.g., 85% to 99% identical thereto);
SEQ ID NOs: 149, 150, 162, and 141, respectively, or a sequence substantially identical thereto (e.g., 85% to 99% identical thereto);
SEQ ID NOs: 151, 152, 162, and 141, respectively, or a sequence substantially identical thereto (e.g., 85% to 99% identical thereto);
SEQ ID NOs: 166, 161, 172, and 161, respectively, or a sequence substantially identical thereto (e.g., 85% to 99% identical thereto);
SEQ ID NOs: 167, 161, 172, and 161, respectively, or a sequence substantially identical thereto (e.g., 85% to 99% identical thereto);
SEQ ID NOs: 168, 161, 172, and 161, respectively, or a sequence substantially identical thereto (e.g., 85% to 99% identical thereto);
SEQ ID NOs: 166, 161, 173, and 161, respectively, or a sequence substantially identical thereto (e.g., 85% to 99% identical thereto);
SEQ ID NOs: 167, 161, 173, and 161, respectively, or a sequence substantially identical thereto (e.g., 85% to 99% identical thereto);
SEQ ID NOs: 168, 161, 173, and 161, respectively, or a sequence substantially identical thereto (e.g., 85% to 99% identical thereto);
SEQ ID NOs: 169, 141, 174, and 141, respectively, or a sequence substantially identical thereto (e.g., 85% to 99% identical thereto);
SEQ ID NOs: 170, 141, 174, and 141, respectively, or a sequence substantially identical thereto (e.g., 85% to 99% identical thereto);
SEQ ID NOs: 171, 141, 174, and 141, respectively, or a sequence substantially identical thereto (e.g., 85% to 99% identical thereto);
SEQ ID NOs: 169, 141, 175, and 141, respectively, or a sequence substantially identical thereto (e.g., 85% to 99% identical thereto);
SEQ ID NOs: 170, 141, 175, and 141, respectively, or a sequence substantially identical thereto (e.g., 85% to 99% identical thereto);
SEQ ID NOs: 171, 141, 175, and 141, respectively, or a sequence substantially identical thereto (e.g., 85% to 99% identical thereto);
SEQ ID NOs: 166, 161, 174, and 141, respectively, or a sequence substantially identical thereto (e.g., 85% to 99% identical thereto);
SEQ ID NOs: 167, 161, 174, and 141, respectively, or a sequence substantially identical thereto (e.g., 85% to 99% identical thereto);
SEQ ID NOs: 168, 161, 174, and 141, respectively, or a sequence substantially identical thereto (e.g., 85% to 99% identical thereto);
SEQ ID NOs: 166, 161, 175, and 141, respectively, or a sequence substantially identical thereto (e.g., 85% to 99% identical thereto);
SEQ ID NOs: 167, 161, 175, and 141, respectively, or a sequence substantially identical thereto (e.g., 85% to 99% identical thereto);
SEQ ID NOs: 168, 161, 175, and 141, respectively, or a sequence substantially identical thereto (e.g., 85% to 99% identical thereto);
SEQ ID NOs: 166, 141, 174, and 161, respectively, or a sequence substantially identical thereto (e.g., 85% to 99% identical thereto);
SEQ ID NOs: 167, 141, 174, and 161, respectively, or a sequence substantially identical thereto (e.g., 85% to 99% identical thereto);
SEQ ID NOs: 168, 141, 174, and 161, respectively, or a sequence substantially identical thereto (e.g., 85% to 99% identical thereto);
SEQ ID NOs: 166, 141, 175, and 161, respectively, or a sequence substantially identical thereto (e.g., 85% to 99% identical thereto);
SEQ ID NOs: 167, 141, 175, and 161, respectively, or a sequence substantially identical thereto (e.g., 85% to 99% identical thereto); or
SEQ ID NOs: 168, 141, 175, and 161, respectively, or a sequence substantially identical thereto (e.g., 85% to 99% identical thereto).

57. The multispecific molecule of any one of claims 32-55, comprising a first and a second non-contiguous polypeptides, wherein the first and the second non-contiguous polypeptides comprise the amino acid sequences of:
SEQ ID NOs: 142 and 143, respectively, or a sequence substantially identical thereto (e.g., 85% to 99% identical thereto);
SEQ ID NOs: 142 and 144, respectively, or a sequence substantially identical thereto (e.g., 85% to 99% identical thereto);
SEQ ID NOs: 157 and 143, respectively, or a sequence substantially identical thereto (e.g., 85% to 99% identical thereto);
SEQ ID NOs: 157 and 144, respectively, or a sequence substantially identical thereto (e.g., 85% to 99% identical thereto);
SEQ ID NOs: 158 and 143, respectively, or a sequence substantially identical thereto (e.g., 85% to 99% identical thereto);
SEQ ID NOs: 158 and 144, respectively, or a sequence substantially identical thereto (e.g., 85% to 99% identical thereto);
SEQ ID NOs: 163 and 143, respectively, or a sequence substantially identical thereto (e.g., 85% to 99% identical thereto);
SEQ ID NOs: 163 and 144, respectively, or a sequence substantially identical thereto (e.g., 85% to 99% identical thereto);
SEQ ID NOs: 164 and 143, respectively, or a sequence substantially identical thereto (e.g., 85% to 99% identical thereto);
SEQ ID NOs: 164 and 144, respectively, or a sequence substantially identical thereto (e.g., 85% to 99% identical thereto);
SEQ ID NOs: 165 and 143, respectively, or a sequence substantially identical thereto (e.g., 85% to 99% identical thereto); or
SEQ ID NOs: 165 and 144, respectively, or a sequence substantially identical thereto (e.g., 85% to 99% identical thereto).

58. An isolated nucleic acid molecule encoding the multispecific molecule (e.g., antibody) of any one of claims 1-57.

59. A vector, e.g., an expression vector, comprising the nucleic acid molecule of claim 58.

60. A cell, e.g., a host cell, comprising the nucleic acid molecule of claim 58 or the vector of claim 59.

61. A method of making, e.g., producing, the multispecific molecule of any one of claims 1-57, comprising culturing the cell, e.g., host cell, of claim 60, under suitable conditions, e.g., conditions suitable for gene expression and/or heterodimerization.

62. A pharmaceutical composition comprising the multispecific molecule of any one of claims 1-57 and a pharmaceutically acceptable carrier, excipient, or stabilizer.

63. A method of treating a cancer in a subject, comprising administering to the subject in need thereof the multispecific molecule of any one of claims 1-57, wherein the multispecific molecule is administered in an amount effective to treat the cancer.

64. The method of claim 63, wherein the cancer is a solid tumor cancer or a metastatic lesion, optionally wherein the solid tumor cancer is one or more of pancreatic cancer (e.g., pancreatic adenocarcinoma), breast cancer, colorectal cancer, lung cancer (e.g., small or non-small cell lung cancer), skin cancer (e.g., melanoma), ovarian cancer, liver cancer, or brain cancer (e.g., glioma).

65. The method of claim 63, wherein the cancer is a hematological cancer or a metastatic lesion, optionally wherein the hematological cancer is one or more of a Hodgkin's lymphoma, Non-Hodgkin's lymphoma, B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia, acute myeloid leukemia (AML), chronic myeloid leukemia, myelodysplastic syndrome (MDS), multiple myeloma, or acute lymphocytic leukemia.

66. The method of any one of claims 63-65, further comprising administering a second therapeutic treatment.

67. The method of claim 66, wherein the second therapeutic treatment comprises a therapeutic agent (e.g., a chemotherapeutic agent, a biologic agent, hormonal therapy), radiation, or surgery.

68. The method of claim 67, wherein the therapeutic agent is a checkpoint inhibitor.

69. The method of claim 68, wherein the check point inhibitor is selected from the group consisting of an anti-CTLA4 antibody, an anti-PD1 antibody (e.g., Nivolumab, Pembrolizumab or Pidilizumab), an anti-PD-L1 antibody, an anti-PD-L2 antibody, an anti-TIM3 antibody, an anti-LAG3 antibody, an anti-CD160 antibody, an anti-2B4 antibody, an anti-CD80 antibody, an anti-CD86 antibody, an anti-B7-H3 (CD276) antibody, an anti-B7-H4 (VTCN1) antibody, an anti-HVEM (TNFRSF14 or CD270) antibody, an anti-BTLA antibody, an anti-KM antibody, an anti-MHC class I antibody, an anti-MHC class II antibody, an anti-GALS antibody, an anti-VISTA antibody, an anti-BTLA antibody, an anti-TIGIT antibody, an anti-LAIR1 antibody, and an anti-A2aR antibody.
